(19) United States
(12) Patent Application Publication
(10) Pub. No.: US 2016/0367623 A1
(43) Pub. Date: Dec. 22, 2016

(54) AGONISTS OF GUANYLATE CYCLASE USEFUL FOR THE TREATMENT OF GASTROINTESTINAL DISORDERS, INFLAMMATION, CANCER AND OTHER DISORDERS

(71) Applicant: Synergy Pharmaceuticals, Inc., New York, NY (US)

(72) Inventor: Kunwar SHAILUBHAI, Audubon, PA (US)

(21) Appl. No.: 15/184,574

(22) Filed: Jun. 16, 2016

Related U.S. Application Data
(60) Provisional application No. 62/180,505, filed on Jun. 16, 2015.
(60) Provisional application No. 62/198,657, filed on Jul. 29, 2015.

(51) Int. Cl. A61K 38/10

(52) U.S. Cl. CPC A61K 38/10

(57) ABSTRACT

The invention provides novel guanylate cyclase-C agonist peptides and their use in the treatment of human diseases including gastrointestinal disorders, inflammation or cancer (e.g., a gastrointestinal cancer), a lipid metabolism disorder, a billary disorder, cardiovascular disease, obesity or an endocrine disorder) by administering at least one agonist of guanalyte cyclase receptor either alone or in combination with a compound typically used to treat the disorder and or with an inhibitor of cGMP-dependent phosphodiesterases.

### RELATED APPLICATIONS

This application claims the benefit of priority to U.S. Provisional Application No. 62/180,505 filed on Jun. 16, 2015, and U.S. Provisional Application No. 62/198,657 filed on Jul. 29, 2015, the contents of each of which are incorporated by reference in their entireties.

### FIELD OF THE INVENTION

The present invention relates to the therapeutic use of guanylate cyclase C (GC-C) agonists, particularly the use of plecanatide.

### BACKGROUND OF THE INVENTION

Uroguanylin, guanylin and bacterial ST peptides are structurally related peptides that bind to a guanylate cyclase receptor and stimulate intracellular production of cyclic guanosine monophosphate (cGMP) (1,6). This results in the activation of the cystic fibrosis transmembrane conductance regulator (CFTR), an apical membrane channel for efflux of chloride from enterocytes lining the intestinal tract (1-6). Activation of CFTR and the subsequent enhancement of transepithelial secretion of chloride lead to stimulation of sodium and water secretion into the intestinal lumen (3). Therefore, by serving as paracrine regulators of CFTR activity, cGMP receptor agonists regulate fluid and electrolyte transport in the GI tract (1-6; U.S. Pat. No. 5,489,670). Thus, the cGMP-mediated activation of CFTR and the downstream signaling plays an important role in normal functioning of gut physiology. Therefore, any abnormality in this process could potentially lead to gastrointestinal disorders such as irritable bowel syndrome, inflammatory bowel disease, excessive acidity and cancer (25, 26).

The process of epithelial renewal involves the proliferation, migration, differentiation, senescence, and eventual loss of GI cells in the lumen (7, 8). The GI mucosa can be divided into three distinct zones based on the proliferation index of epithelial cells. One of these zones, the proliferative zone, consists of undifferentiated stem cells responsible for providing a constant source of new cells. The stem cells migrate upward toward the lumen to which they are extruded. As they migrate, the cells lose their capacity to divide and become differentiated for carrying out specialized functions of the GI mucosa (9). Renewal of GI mucosa is very rapid with complete turnover occurring within a 24-48 hour period (9). During this process mutated and unwanted cells are replenished with new cells. Hence, homeostasis of the GI mucosa is regulated by continual maintenance of the balance between proliferation and apoptotic rates (8).

The rates of cell proliferation and apoptosis in the gut epithelium can be increased or decreased in a wide variety of different circumstances, e.g., in response to physiological stimuli such as aging, inflammatory signals, hormones, peptides, growth factors, chemicals and dietary habits. In addition, an enhanced proliferation rate is frequently associated with a reduction in turnover time and an expansion of the proliferative zone (10). The proliferation index has been observed to be much higher in pathological cases of ulcerative colitis and other GI disorders (11). Thus, intestinal hyperplasia is the major promoter of gastrointestinal inflammation and carcinogenesis.

In addition to a role for uroguanylin and guanylin as modulators of intestinal fluid and ion secretion, these peptides may also be involved in the continual renewal of GI mucosa by maintaining the balance between proliferation and apoptosis in cells lining GI mucosa. Therefore, any disruption in this renewal process, due to reduced production of uroguanylin and/or guanylin can lead to GI inflammation and cancer (25, 26). This is consistent with previously published data in WO 01/25266, which suggest a peptide with the active domain of uroguanylin may function as an inhibitor of polyp development in the colon and may constitute a treatment of colon cancer. However, recent data also suggest that uroguanylin also binds to a currently unknown receptor, which is distinct from GC-C receptor (3,4). Knockout mice lacking this guanylate cyclase receptor show resistance to ST peptides in the intestine, but effects of uroguanylin and ST peptides are not disturbed in the kidney in vivo (3). These results were further supported by the fact that membrane depolarization induced by guanylin was blocked by genistein, a tyrosine kinase inhibitor, whereas hyperpolarization induced by uroguanylin was not effected (12, 13). Thus, it is not clear if the anti-colon cancer and anti-inflammatory activities of uroguanylin and its analogs are mediated through binding to one or both of these receptors.

Irritable bowel syndrome (IBS) and chronic idiopathic constipation are pathological conditions that can cause a great deal of intestinal discomfort and distress but unlike the IBD diseases such as ulcerative colitis and Crohn's disease, IBS does not cause the serious inflammation or changes in bowel tissue and it is not thought to increase the risk of colorectal cancer. In the past, inflammatory bowel disease (IBD), celiac disease and irritable bowel syndrome (IBS) were regarded as completely separate disorders. Now, with the description of inflammation, albeit low-grade, in IBS, and of symptom overlap between IBS and celiac disease, this contention has come under question. Acute bacterial gastroenteritis is the strongest risk factor identified to date for the subsequent development of postinfective irritable bowel syndrome (PI-IBS). Clinical risk factors include prolonged acute illness and the absence of vomiting. A genetically determined susceptibility to inflammatory stimuli may also be a risk factor for irritable bowel syndrome. The underlying pathophysiology indicates increased intestinal permeability and low-grade inflammation, as well as altered motility and visceral sensitivity (27). Thus, IBS is now considered as a low grade IBD.

Serotonin (5-hydroxytryptamine [5-HT]) is a key modulator of gut function and is known to play a major role in pathophysiology of IBS. It has been shown that the activity of 5-HT is regulated by cGMP (28). Recent studies have shown measurable improvements in patients with IBS treated with selective serotonin reuptake inhibitors and serotoninergic agents (alosetron, tegaserod) (29, 30). Majority of the serotonin content in the body is found in the gut and not in the central nervous system. This fact raises the question as to whether the modulation of serotonin action in the gut could influence IBS or other functional bowel symptoms. Recently, it has been suggested that mucosal inflammation plays a putative role in the pathophysiology of IBS (31). Therefore, we believe that GC-C agonist might also be useful in treatment of IBS.

Diabetes mellitus (DM) is a group of metabolic diseases characterized by hyperglycemia, resulting from defects in insulin secretion, insulin action, or both. The chronic hyperglycemia of diabetes is associated with long-term damage, dysfunction, and failure of various organs, especially the eyes, kidneys, nerves, heart, and blood vessels. Well-known risk factors of type 2 DM are family history, obesity, age, race, prediabetes [impaired fasting glucose (IFG) and/or impaired glucose tolerance (IGT)], gestational DM, and polycystic ovarian syndrome. A few previous studies have evaluated possible relationships between insulin resistance and serotonin (32). Investigations on diabetic rats have revealed dysfunctions in serotonin receptors in both the large and the small intestines (33). Moreover, an association between insulin resistance and inflammation has been reported (34). In light of these data, we hypothesize that IBS may be associated with levels of glucose tolerance, resulting in type 2 DM. Therefore, GC-C agonists may also be useful in prevention and control of type 2 DM.

Furthermore, there are numerous investigations that have supported the role of chronic inflammation in the pathogenesis of type 2 DM (35-37). In these studies, it was noted that chronic inflammation might accompany increased levels of C-reactive protein and inflammatory cytokines. Data also indicated a correlation that prediabetes was common in patients with IBS, which suggested that the chronic inflammation process might be responsible for the progression to DM. Prediabetes condition has recently been reported to occur more commonly in the IBS group than in the control group (35). HDL and LDL levels were also found to be higher in the IBS group compared with the control group (35). Because prediabetes is a precursor of type 2 DM, patients with IBS may be considered as a high-risk group for type 2 DM. Hence, treatments for IBS might also prevent and control progression of prediabetic condition to type 2 DM.

Hypercholesterolemia has been recognized as a major risk factor for coronary heart disease (CHD). In clinical trials, reducing serum LDL cholesterol has been demonstrated to decrease the incidence of CHD and to reverse atherosclerotic lesions. Two main classes of clinically useful hypocholesterolemic agents are the 3-hydroxy-3-methylglutaryl coenzyme A (HMG-CoA) reductase inhibitors (e.g., statins) and the bile acid sequestrants. Both induce hepatic LDL receptor activity by increasing hepatic cholesterol demand. Because the major determinant of serum cholesterol level is hepatic LDL receptor activity (38), these agents may share a common mechanism leading to reduction in serum cholesterol.

In the case of bile acid sequestrants such as cholestyramine and colestipol, the mechanism of action seems to be due to inhibition of enterohepatic circulation, the transport of bile acids between liver and intestine. Bile acids are synthesized from cholesterol in the liver and secreted into the bile flow to facilitate the digestion and absorption of lipids, followed by nearly quantitative (˜95%) reabsorption from the intestine. The remaining ˜5% of the bile acids enter colon and excreted out. The ileal Na1/bile acid cotransporter (MAT) maintains the reabsorption of bile acids from the intestine and thus, its inhibitor is expected to exhibit pharmacological effects similar to those of bile acid sequestrants. Bile acids are detergent molecules that facilitate biliary excretion of cholesterol, byproduct of metabolism and xenobiotics, and intestinal absorption of fat and fat-soluble nutrients. When food is ingested, the gallbladder is stimulated to contract resulting in secretion of bile into the lumen of small intestine (duodenum), where it acts as a detergent to form micelles of fat soluble nutrients, dietary cholesterol and lipids. Micelles serve an important function in the digestion and in absorption of fat consisting of mainly dietary triglycerides and cholesterol.

The digestive system is largely responsible for the maintenance of cholesterol balance in the body. Bile salts are produced by enzymatic modification of cholesterol and secreted into intestine. The reabsorption of bile salts from the intestine is very efficient and 95-98% of bile salts are recycled back to liver. Thus, only 2-5% of bile salts escape recycling and are excreated out in feces. This amount of loss of bile salts is replenished quickly in liver through enzymatic conversion from cholesterol. Therefore, inhibition of bile salt from the intestine has been used as an approach to reduce serum cholesterol. Moreover, cholesterol absorption inhibitors also reduce the absorption of dietary cholesterol. Known cholesterol absorption inhibitors are plant sterols and stanols. In addition, inhibitors of ileal Na+/bile acid cotransporter (IBAT) are also used for reducing plasma cholesterol. Plasma cholesterol levels can be reduced through inhibition of cholesterol synthesis as well as through inhibition of ileal absorption of dietary cholesterol and reabsorption of bile salts. Enterohepatic cycling thus has a profound impact on plasma cholesterol and body fat.

Prolonged small intestinal transit, like in patients with chronic constipation, IBS-c, and impaired gallbladder emptying, should hinder enterohepatic cycling, which might be associated with increased levels of plasma cholesterol, triglycerides and lipids. In addition, slowed transit through the distal intestine (ileum, caecum and colon) may also lead to increased conversion of bile acids to deoxycholate, which in itself can slow down small intestinal transit. Absorption of deoxycholic acid from colon occurs only by passive diffusion. When radioactive cholic acid is injected into the colon at a laparotomy most of it was absorbed and resecreted in the bile largely as deoxycholate during the first 24 hours but its absorption from colon continued for several days (39). Moreover, prolonged presence of deoxycholate at higher level in colon can also cause inflammatory diseases and cancer.

Given the prevalence of diseases associated with hypercholesterolemia, obesity and inflammatory conditions, inhibition of ileal absorption of cholesterol and reduction in reabsorption of biles salts from intestine could be highly useful as improve the treatment options for obesity, cardiovascular diseases, diabetes type 2, gallstone and liver diseases.

### SUMMARY OF THE INVENTION

The present invention is based upon the development of an agonist of guanylate cyclase receptor, Plecanatide (SEQ ID NO:1). The agonist is an analog of uroguanylin, guanylin, lymphoguanylin and ST peptides and has superior properties such as for example high resistance to degradation at the N-terminus and C-terminus from carboxypeptidases and/or by other proteolytic enzymes present in the stimulated human intestinal juices and human gastric juices.

The peptide of the invention may be used to treat any condition that responds to enhanced intracellular levels of cGMP. Intracellular levels of cGMP can be increased by enhancing intracellular production of cGMP and/or by inhibition of its degradation by cGMP-specific phosphodiesterases. Among the specific conditions that can be treated or prevented are lipid metabolism disorders, biliary disorders, gastrointestinal disorders, inflammatory disorders, lung disorders, cancer, cardiac disorders including cardiovascular disorders, eye disorders, oral disorders, blood disorders, liver disorders, skin disorders, prostate disorders, endocrine disorders, increasing gastrointestinal motility and obesity. Lipid metabolism disorder including, but not limited to, dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, erectile dysfunction, fatty liver disease, and hepatitis. Billary disorders include gallbladder disorders such as for example, gallstones, gall bladder cancer cholangitis, or primary sclerosing cholangitis; or bile duct disorders such as for example, cholecystitis, bile duct cancer or fascioliasis. Gastrointestinal disorders include for example, irritable bowel syndrome (IBS), non-ulcer dyspepsia, chronic intestinal pseudo-obstruction, functional dyspepsia, colonic pseudo-obstruction, duodenogastric reflux, gastroesophageal reflux disease (GERD), ileus inflammation (e.g., post-operative ileus), gastroparesis, heartburn (high acidity in the GI tract), constipation (e.g., constipation associated with use of medications such as opioids, osteoarthritis drugs, osteoporosis drugs; post surgical constipation, constipation associated with neuropathic disorders, chronic idiopathic constipation). Inflammatory disorders include tissue and organ inflammation such as kidney inflammation (e.g., nephritis), gastrointestinal system inflammation (e.g., Crohn's disease and ulcerative colitis); necrotizing enterocolitis (NEC); pancreatic inflammation (e.g., pancreatis), lung inflammation (e.g., bronchitis or asthma) or skin inflammation (e.g., psoriasis, eczema). Lung Disorders include for example chronic obstructive pulmonary disease (COPD), and fibrosis. Cancer includes tissue and organ carcinogenesis including metatases such as for example gastrointestinal cancer, (e.g., gastric cancer, esophageal cancer, pancreatic cancer colorectal cancer, intestinal cancer, anal cancer, liver cancer, gallbladder cancer, or colon cancer; lung cancer; thyroid cancer; skin cancer (e.g., melanoma); oral cancer; urinary tract cancer (e.g. bladder cancer or kidney cancer); blood cancer (e.g. myeloma or leukemia) or prostate cancer. Cardiac disorders include for example, congestive heart failure, trachea cardia hypertension, high cholesterol, or high triglycerides. Cardiovascular disorders include for example aneurysm, angina, atherosclerosis, cerebrovascular accident (stroke), cerebrovasculardisease, congestive heart failure, coronary artery disease, myocardial infarction (heart attack), or peripheral vascular disease. Liver disorders include for example cirrhosis and fibrosis. In addition, GC-C agonist may also be useful to facilitate liver regeneration in liver transplant patients. Eye disorders include for example increased intra-ocular pressure, glaucoma, dry eyes retinal degeneration, disorders of tear glands or eye inflammation. Skin disorders include for example xerosis. Oral disorders include for example dry mouth (xerostomia), Sjögren's syndrome, gum diseases (e.g., periodontal disease), or salivary gland duct blockage or malfunction. Prostate disorders include for example benign prostatic hyperplasia (BPH). Endocrine disorders include for example diabetes mellitus, hyperthyroidism, hypothyroidism, and cystic fibrosis.

The peptide may be in a pharmaceutical composition in unit dose form, together with one or more pharmaceutically acceptable carrier, excipients or diluents. The term “unit dose form” refers to a single drug delivery entity, e.g., a tablet, capsule, solution or inhalation formulation. The amount of peptide present should be sufficient to have a positive therapeutic effect when administered to a patient (typically, between 100 μg and 3 g). Preferably, the amount of peptide administered is 0.3 mg, 1.0 mg, or 3.0 mg, 6.0 mg, 9.0 mg or 10 mg per day. Most preferably the peptide is administered at 3.0 mg or 6.0 mg, taken once a day. What constitutes a “positive therapeutic effect” will depend upon the particular condition being treated and will include any significant improvement in a condition readily recognized by one of skill in the art. For example, it may constitute a reduction in inflammation, shrinkage of polyps or tumors, a reduction in metastatic lesions, etc.

In yet another aspect, an invention provides administering to said patient an effective dose of an inhibitor of cGMP-specific phosphodiesterase (cGMP-PDE), a fibrate, a lipid altering agent, a HMG-CoA reductase inhibitor, an anti-diabetic agent, an anti-obesity agent either concurrently or sequentially with said guanylate cyclase receptor agonist. The cGMP-PDE inhibitor includes for example suldinac sulfone, zaprinast, and motapizone, vardenifil, and sildenafil. In addition, GC-C agonist peptide may be used in combination with inhibitors of cyclic nucleotide transporters.

Other features and advantages of the invention will be apparent from and are encompassed by the following detailed description and claims.

### DETAILED DESCRIPTION

The present invention is based upon the development of an agonist of guanylate cyclase-C (GC-C), Plecanatide (SEQ ID NO: 1). This agonist is an analog of uroguanylin, guanylin, lymphoguanylin and ST peptided and has superior properties such as for example high resistance to degradation at the N-terminus and C-terminus from carboxypeptidases and/or by other proteolytic enzymes such as those present in the stimulated human intestinal fluid (SIF) and simulated human gastric fluid (SGF).

The GC-C is expressed on various cells including on gastrointestinal epithelial cells, and on extra-intestinal tissues including kidney, lung, pancreas, pituitary, adrenal, developing liver, heart and male and female reproductive tissues (reviewed in Vaandrager 2002 Mol Cell Biochem 230:73-83). The GC-C is a key regulator of fluid and electrolyte balance in the intestine and kidney. In the intestine, when stimulated, the GC-C causes an increase in intestinal epithelial cGMP. This increase in cGMP causes a decrease in water and sodium absorption and an increase in chloride and potassium ion secretion, leading to changes in intestinal fluid and electrolyte transport and increased intestinal motility.

The invention also relates in part to the use of this GC-C agonist to treat chronic idiopathic constipation (CIC).

| | Position of | | SEQ |
|---|---|---|---|
| | Disulfide | | ID |
| Name | bonds | Structure | NO |
| SP-304 | C4:C12, | Asn¹-Asp²-Glu³-Cys⁴- | 1 |
| (Plecanatide) | C7:C15 | Glu⁵-Leu⁶-Cys⁷-Val⁸- | |
| | | Asn⁹-Val¹⁰-Ala¹¹- | |
| | | Cys¹²-Thr¹³-Gly¹⁴- | |
| | | Cys¹⁵-Leu¹⁶ | |

The GCRA peptide described herein binds the guanylate cyclase C (GC-C) and stimulates intracellular production of cyclic guanosine monophosphate (cGMP). Optionally, the GCRA peptide induces apoptosis. In some aspects, the GCRA peptide stimulates intracellular cGMP production at higher levels than naturally occurring GC-C agonists (e.g., uroguanylin, guanylin, lymphoguanylin and ST peptides).

For example, the GCRA peptide of the invention stimulates 5, 10%, 20%, 30%, 40%, 50%, 75%, 90% or more intracellular cGMP compared to naturally occurring GC-C angonists. The terms induced and stimulated are used interchangeably throughout the specification. The GCRA peptide described herein is more stable than naturally occurring GC-C agonists. By more stable it is meant that the peptide degrade less and/or more slowly in simulated gastrointestinal fluid and/or simulated intestinal fluid compared to naturally occurring GC-C angonists and/or SP-304. For example, the GCRA peptide of the invention degrades 2%, 3%, 5%, 10%, 15%, 20%, 30%, 40%, 50%, 75%, 90% or less compared to naturally occurring GC-C angonists.

The GCRA peptide described herein has therapeutic value in the treatment of a wide variety of disorders and conditions including for example lipid metabolism disorders, biliary disorders, gastrointestinal disorders, inflammatory disorders, lung disorders, cancer, cardiac disorders including cardiovascular disorders, eye disorders, oral disorders, blood disorders, liver disorders, skin disorders, prostate disorders, endocrine disorders, increasing gastrointestinal motility and obesity. Lipid metabolism disorder including, but not limited to, dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, erectile dysfunction, fatty liver disease, and hepatitis. Billary disorders include gallbladder disorders such as for example, gallstones, gall bladder cancer cholangitis, or primary sclerosing cholangitis; or bile duct disorders such as for example, cholecystitis, bile duct cancer or fascioliasis. Gastrointestinal disorders include for example, irritable bowel syndrome (IBS), non-ulcer dyspepsia, chronic intestinal pseudo-obstruction, functional dyspepsia, colonic pseudo-obstruction, duodenogastric reflux, gastroesophageal reflux disease (GERD), ileus inflammation (e.g., post-operative ileus), gastroparesis, heartburn (high acidity in the GI tract), constipation (e.g., constipation associated with use of medications such as opioids, osteoarthritis drugs, osteoporosis drugs; post surgical constipation, constipation associated with neuropathic disorders, chronic idiopathic constipation). Inflammatory disorders include tissue and organ inflammation such as kidney inflammation (e.g., nephritis), gastrointestinal system inflammation (e.g., Crohn's disease and ulcerative colitis); necrotizing enterocolitis (NEC); pancreatic inflammation (e.g., pancreatis), lung inflammation (e.g., bronchitis or asthma) or skin inflammation (e.g., psoriasis, eczema). Lung Disorders include for example chronic obstructive pulmonary disease (COPD), and fibrosis. Cancer includes tissue and organ carcinogenesis including metatases such as for example gastrointestinal cancer, (e.g., gastric cancer, esophageal cancer, pancreatic cancer colorectal cancer, intestinal cancer, anal cancer, liver cancer, gallbladder cancer, or colon cancer; lung cancer; thyroid cancer; skin cancer (e.g., melanoma); oral cancer; urinary tract cancer (e.g. bladder cancer or kidney cancer); blood cancer (e.g. myeloma or leukemia) or prostate cancer. Cardiac disorders include for example, congestive heart failure, trachea cardia hypertension, high cholesterol, or high triglycerides. Cardiovascular disorders include for example aneurysm, angina, atherosclerosis, cerebrovascular accident (stroke), cerebrovasculardisease, congestive heart failure, coronary artery disease, myocardial infarction (heart attack), or peripheral vascular disease. Liver disorders include for example cirrhosis and fibrosis. In addition, GC-C agonist may also be useful to facilitate liver regeneration in liver transplant patients. Eye disorders include for example increased intra-ocular pressure, glaucoma, dry eyes retinal degeneration, disorders of tear glands or eye inflammation. Skin disorders include for example xerosis. Oral disorders include for example dry mouth (xerostomia), Sjögren's syndrome, gum diseases (e.g., periodontal disease), or salivary gland duct blockage or malfunction. Prostate disorders include for example benign prostatic hyperplasia (BPH). Endocrine disorders include for example diabetes mellitus, hyperthyroidism, hypothyroidism, and cystic fibrosis.

As used herein, the term “guanylate cyclase receptor (GCR)” refers to the class of guanylate cyclase C receptor on any cell type to which the inventive agonist peptides or natural agonists described herein bind. As used herein, “intestinal guanylate cyclase receptor” is found exclusively on epithelial cells lining the GI mucosa. Uroguanylin, guanylin, and ST peptides are expected to bind to these receptors and may induce apoptosis. The possibility that there may be different receptors for each agonist peptide is not excluded. Hence, the term refers to the class of guanylate cyclase receptors on epithelial cells.

As used herein, the term “GCR agonist” is meant to refer to peptides and/or other compounds that bind to an intestinal guanylate cyclase receptor and stimulate fluid and electrolyte transport. This term also covers fragments and pro-peptides that bind to GCR and stimulate fluid and water secretion.

As used herein, the term “substantially equivalent” is meant to refer to a peptide that has an amino acid sequence equivalent to that of the binding domain where certain residues may be deleted or replaced with other amino acids without impairing the peptide's ability to bind to an intestinal guanylate cyclase receptor and stimulate fluid and electrolyte transport.

Addition of carriers (e.g., phosphate-buffered saline or PBS) and other components to the composition of the present invention is well within the level of skill in this art. In addition to the compound, such compositions may contain pharmaceutically acceptable carriers and other ingredients known to facilitate administration and/or enhance uptake. Other formulations, such as microspheres, nanoparticles, liposomes, and immunologically-based systems may also be used in accordance with the present invention. Other examples include formulations with polymers (e.g., 20% w/v polyethylene glycol) or cellulose, or enteric formulations.

The present invention is based upon several concepts. The first is that there is a cGMP-dependent mechanism which regulates the balance between cellular proliferation and apoptosis and that a reduction in cGMP levels, due to a deficiency of uroguanylin/guanylin and/or due to the activation of cGMP-specific phosphodiesterases, is an early and critical step in neoplastic transformation. A second concept is that the release of arachidonic acid from membrane phospholipids, which leads to the activation of cytoplasmic phospholipase A2 (cPLA2), cyclooxygenase-2 (COX-2) and possibly 5-lipoxygenase (5-LO) during the process of inflammation, is down-regulated by a cGMP-dependent mechanism, leading to reduced levels of prostaglandins and leukotrienes, and that increasing intracellular levels of cGMP may therefore produce an anti-inflammatory response. In addition, a cGMP-dependent mechanism is thought to be involved in the control of proinflammatory processes. Therefore, elevating intracellular levels of cGMP may be used as a means of treating and controlling lipid metabolism disorders, biliary disorders, gastrointestinal disorders, inflammatory disorders, lung disorders, cancer, cardiac disorders including cardiovascular disorders, eye disorders, oral disorders, blood disorders, liver disorders, skin disorders, prostate disorders, endocrine disorders, increasing gastrointestinal motility and obesity. Lipid metabolism disorder including, but not limited to, dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, erectile dysfunction, fatty liver disease, and hepatitis. Billary disorders include gallbladder disorders such as for example, gallstones, gall bladder cancer cholangitis, or primary sclerosing cholangitis; or bile duct disorders such as for example, cholecystitis, bile duct cancer or fascioliasis. Gastrointestinal disorders include for example, irritable bowel syndrome (IBS), non-ulcer dyspepsia, chronic intestinal pseudo-obstruction, functional dyspepsia, colonic pseudo-obstruction, duodenogastric reflux, gastroesophageal reflux disease (GERD), ileus inflammation (e.g., post-operative ileus), gastroparesis, heartburn (high acidity in the GI tract), constipation (e.g., constipation associated with use of medications such as opioids, osteoarthritis drugs, osteoporosis drugs; post surgical constipation, constipation associated with neuropathic disorders. Inflammatory disorders include tissue and organ inflammation such as kidney inflammation (e.g., nephritis), gastrointestinal system inflammation (e.g., Crohn's disease and ulcerative colitis); necrotizing enterocolitis (NEC); pancreatic inflammation (e.g., pancreatis), lung inflammation (e.g., bronchitis or asthma) or skin inflammation (e.g., psoriasis, eczema). Lung Disorders include for example chronic obstructive pulmonary disease (COPD), and fibrosis. Cancer includes tissue and organ carcinogenesis including metatases such as for example gastrointestinal cancer, (e.g., gastric cancer, esophageal cancer, pancreatic cancer colorectal cancer, intestinal cancer, anal cancer, liver cancer, gallbladder cancer, or colon cancer; lung cancer; thyroid cancer; skin cancer (e.g., melanoma); oral cancer; urinary tract cancer (e.g. bladder cancer or kidney cancer); blood cancer (e.g. myeloma or leukemia) or prostate cancer. Cardiac disorders include for example, congestive heart failure, trachea cardia hypertension, high cholesterol, or high triglycerides. Cardiovascular disorders include for example aneurysm, angina, atherosclerosis, cerebrovascular accident (stroke), cerebrovasculardisease, congestive heart failure, coronary artery disease, myocardial infarction (heart attack), or peripheral vascular disease. Liver disorders include for example cirrhosis and fibrosis. In addition, GC-C agonist may also be useful to facilitate liver regeneration in liver transplant patients. Eye disorders include for example increased intra-ocular pressure, glaucoma, dry eyes retinal degeneration, disorders of tear glands or eye inflammation. Skin disorders include for example xerosis. Oral disorders include for example dry mouth (xerostomia), Sjögren's syndrome, gum diseases (e.g., periodontal disease), or salivary gland duct blockage or malfunction. Prostate disorders include for example benign prostatic hyperplasia (BPH). Endocrine disorders include for example diabetes mellitus, hyperthyroidism, hypothyroidism, and cystic fibrosis.

Without intending to be bound by any theory, it is envisioned that ion transport across the plasma membrane may prove to be an important regulator of the balance between cell proliferation and apoptosis that will be affected by agents altering cGMP concentrations. Uroguanylin has been shown to stimulate K+efflux, Ca++influx and water transport in the gastrointestinal tract (3). Moreover, atrial natriuretic peptide (ANP), a peptide that also binds to a specific guanylate cyclase receptor, has also been shown to induce apoptosis in rat mesangial cells, and to induce apoptosis in cardiac myocytes by a cGMP mechanism (21-24).

Binding of the present agonist to a guanylate cyclase receptor stimulates production of cGMP. This ligand-receptor interaction, via activation of a cascade of cGMP-dependent protein kinases and CFTR, induces apoptosis in target cells. Therefore, administration of the peptide of the present invention is useful in eliminating or, at least retarding, the onset of lipid metabolism disorders, biliary disorders, gastrointestinal disorders, inflammatory disorders, lung disorders, cancer, cardiac disorders including cardiovascular disorders, eye disorders, oral disorders, blood disorders, liver disorders, skin disorders, prostate disorders, endocrine disorders, increasing gastrointestinal motility and obesity. Lipid metabolism disorder including, but not limited to, dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, erectile dysfunction, fatty liver disease, and hepatitis. Billary disorders include gallbladder disorders such as for example, gallstones, gall bladder cancer cholangitis, or primary sclerosing cholangitis; or bile duct disorders such as for example, cholecystitis, bile duct cancer or fascioliasis. Gastrointestinal disorders include for example, irritable bowel syndrome (IBS), non-ulcer dyspepsia, chronic intestinal pseudo-obstruction, functional dyspepsia, colonic pseudo-obstruction, duodenogastric reflux, gastroesophageal reflux disease (GERD), ileus inflammation (e.g., post-operative ileus), gastroparesis, heartburn (high acidity in the GI tract), constipation (e.g., constipation associated with use of medications such as opioids, osteoarthritis drugs, osteoporosis drugs; post surgical constipation, constipation associated with neuropathic disorders. Inflammatory disorders include tissue and organ inflammation such as kidney inflammation (e.g., nephritis), gastrointestinal system inflammation (e.g., Crohn's disease and ulcerative colitis); necrotizing enterocolitis (NEC); pancreatic inflammation (e.g., pancreatis), lung inflammation (e.g., bronchitis or asthma) or skin inflammation (e.g., psoriasis, eczema). Lung Disorders include for example chronic obstructive pulmonary disease (COPD), and fibrosis. Cancer includes tissue and organ carcinogenesis including metatases such as for example gastrointestinal cancer, (e.g., gastric cancer, esophageal cancer, pancreatic cancer colorectal cancer, intestinal cancer, anal cancer, liver cancer, gallbladder cancer, or colon cancer; lung cancer; thyroid cancer; skin cancer (e.g., melanoma); oral cancer; urinary tract cancer (e.g. bladder cancer or kidney cancer); blood cancer (e.g. myeloma or leukemia) or prostate cancer. Cardiac disorders include for example, congestive heart failure, trachea cardia hypertension, high cholesterol, or high triglycerides. Cardiovascular disorders include for example aneurysm, angina, atherosclerosis, cerebrovascular accident (stroke), cerebrovasculardisease, congestive heart failure, coronary artery disease, myocardial infarction (heart attack), or peripheral vascular disease. Liver disorders include for example cirrhosis and fibrosis. In addition, GC-C agonist may also be useful to facilitate liver regeneration in liver transplant patients. Eye disorders include for example increased intra-ocular pressure, glaucoma, dry eyes retinal degeneration, disorders of tear glands or eye inflammation. Skin disorders include for example xerosis. Oral disorders include for example dry mouth (xerostomia), Sjögren's syndrome, gum diseases (e.g., periodontal disease), or salivary gland duct blockage or malfunction. Prostate disorders include for example benign prostatic hyperplasia (BPH). Endocrine disorders include for example diabetes mellitus, hyperthyroidism, hypothyroidism, and cystic fibrosis.

Uroguanylin is a circulating peptide hormone with natriuretic activity and has been found to stimulate fluid and electrolyte transport in a manner similar to another family of heat stable enterotoxins (ST peptides) secreted by pathogenic strains of E. coli and other enteric bacteria that activate guanylate cyclase receptor and cause secretory diarrhea. Unlike bacterial ST peptides, the binding of uroguanylin to guanylate cyclase receptor is dependent on the physiological pH of the gut. Therefore, uroguanylin is expected to regulate fluid and electrolyte transport in a pH dependent manner and without causing severe diarrhea.

GCRA Peptide

In one aspect, the invention provides a GCRA peptide. The GCRA peptide is SEQ ID NO: 1 that is a (C4:C12, C7:C15) bicycle peptide.

By inducing cGMP production is meant that the GCRA peptide induces the production of intracellular cGMP. Intracellular cGMP is measured by methods known in the art. For example, the GCRA peptide of the invention stimulate 5%, 10%, 20%, 30%, 40%, 50%, 75%, 90% or more intracellular cGMP compared to naturally occurring GC-C agonists. In further embodiments, the GCRA peptide stimulates apoptosis, e.g., programmed cell death or activates the cystic fibrosis transmembrane conductance regulator (CFTR). In some embodiments the GCRA peptide described herein is more stable than naturally occurring GC-C agonists. By more stable it is meant that the peptide degrade less and/or more slowly in simulated gastric fluid and/or simulated intestinal fluid compared to naturally occurring GC-C agonists. For example, the GCRA peptide of the invention degrades 2%, 3%, 5%, 10%, 15%, 20%, 30%, 40%, 50%, 75%, 90% or less compared to naturally occurring GC-C angonists.

Preparation of GCRA Peptides

GCRA peptides are easily prepared using modern cloning techniques, or may be synthesized by solid state methods or by site-directed mutagenesis. A GCRA peptide may include dominant negative forms of a polypeptide.

Chemical synthesis may generally be performed using standard solution phase or solid phase peptide synthesis techniques, in which a peptide linkage occurs through the direct condensation of the amino group of one amino acid with the carboxy group of the other amino acid with the elimination of a water molecule. Peptide bond synthesis by direct condensation, as formulated above, requires suppression of the reactive character of the amino group of the first and of the carboxyl group of the second amino acid. The masking substituents must permit their ready removal, without inducing breakdown of the labile peptide molecule.

In solution phase synthesis, a wide variety of coupling methods and protecting groups may be used (See, Gross and Meienhofer, eds., “The Peptides: Analysis, Synthesis, Biology,” Vol. 1-4 (Academic Press, 1979); Bodansky and Bodansky, “The Practice of Peptide Synthesis,” 2d ed. (Springer Verlag, 1994)). In addition, intermediate purification and linear scale up are possible. Those of ordinary skill in the art will appreciate that solution synthesis requires consideration of main chain and side chain protecting groups and activation method. In addition, careful segment selection is necessary to minimize racemization during segment condensation. Solubility considerations are also a factor. Solid phase peptide synthesis uses an insoluble polymer for support during organic synthesis. The polymer-supported peptide chain permits the use of simple washing and filtration steps instead of laborious purifications at intermediate steps. Solid-phase peptide synthesis may generally be performed according to the method of Merrifield et al., J. Am. Chem. Soc., 1963, 85:2149, which involves assembling a linear peptide chain on a resin support using protected amino acids. Solid phase peptide synthesis typically utilizes either the Boc or Fmoc strategy, which is well known in the art.

Those of ordinary skill in the art will recognize that, in solid phase synthesis, deprotection and coupling reactions must go to completion and the side-chain blocking groups must be stable throughout the synthesis. In addition, solid phase synthesis is generally most suitable when peptides are to be made on a small scale.

Acetylation of the N-terminal can be accomplished by reacting the final peptide with acetic anhydride before cleavage from the resin. C-amidation is accomplished using an appropriate resin such as methylbenzhydrylamine resin using the Boc technology.

Alternatively the GCRA peptides are produced by modern cloning techniques. For example, the GCRA peptides are produced either in bacteria including, without limitation, E. coli, or in other existing systems for polypeptide or protein production (e.g., Bacillus subtilis, baculovirus expression systems using Drosophila Sf9 cells, yeast or filamentous fungal expression systems, mammalian cell expression systems), or they can be chemically synthesized. If the GCRA peptide or variant peptide is to be produced in bacteria, e.g., E. coli, the nucleic acid molecule encoding the polypeptide may also encode a leader sequence that permits the secretion of the mature polypeptide from the cell. Thus, the sequence encoding the polypeptide can include the pre sequence and the pro sequence of, for example, a naturally-occurring bacterial ST polypeptide. The secreted, mature polypeptide can be purified from the culture medium.

The sequence encoding a GCRA peptide described herein can be inserted into a vector capable of delivering and maintaining the nucleic acid molecule in a bacterial cell. The DNA molecule may be inserted into an autonomously replicating vector (suitable vectors include, for example, pGEM3Z and pcDNA3, and derivatives thereof). The vector nucleic acid may be a bacterial or bacteriophage DNA such as bacteriophage lambda or M13 and derivatives thereof. Construction of a vector containing a nucleic acid described herein can be followed by transformation of a host cell such as a bacterium. Suitable bacterial hosts include but are not limited to, E. coli, B subtilis, Pseudomonas, Salmonella. The genetic construct also includes, in addition to the encoding nucleic acid molecule, elements that allow expression, such as a promoter and regulatory sequences. The expression vectors may contain transcriptional control sequences that control transcriptional initiation, such as promoter, enhancer, operator, and repressor sequences.

A variety of transcriptional control sequences are well known to those in the art. The expression vector can also include a translation regulatory sequence (e.g., an untranslated 5′ sequence, an untranslated 3′ sequence, or an internal ribosome entry site). The vector can be capable of autonomous replication or it can integrate into host DNA to ensure stability during polypeptide production.

The protein coding sequence that includes a GCRA peptide described herein can also be fused to a nucleic acid encoding a polypeptide affinity tag, e.g., glutathione S-transferase (GST), maltose E binding protein, protein A, FLAG tag, hexa-histidine, myc tag or the influenza HA tag, in order to facilitate purification. The affinity tag or reporter fusion joins the reading frame of the polypeptide of interest to the reading frame of the gene encoding the affinity tag such that a translational fusion is generated. Expression of the fusion gene results in translation of a single polypeptide that includes both the polypeptide of interest and the affinity tag. In some instances where affinity tags are utilized, DNA sequence encoding a protease recognition site will be fused between the reading frames for the affinity tag and the polypeptide of interest.

Genetic constructs and methods suitable for production of immature and mature forms of the GCRA peptides and variants described herein in protein expression systems other than bacteria, and well known to those skilled in the art, can also be used to produce polypeptides in a biological system.

The peptides disclosed herein may be modified by attachment of a second molecule that confers a desired property upon the peptide, such as increased half-life in the body, for example, pegylation. Such modifications also fall within the scope of the term “variant” as used herein.

Therapeutic Methods

The present invention provides for both prophylactic and therapeutic methods of treating a subject at risk of (or susceptible to) a disorder or having a disorder associated that is mediated by guanylate cyclase receptor agonists. Disorders mediated by the guanylate cyclase receptor agonists include specifically hypercholesterolemia, atherosclesis, obesity, diabetes type 2 and liver diseases. Disorders mediated by the guanylate cyclase receptor agonists include lipid metabolism disorders, biliary disorders, gastrointestinal disorders, inflammatory disorders, lung disorders, cancer, cardiac disorders including cardiovascular disorders, eye disorders, oral disorders, blood disorders, liver disorders, skin disorders, prostate disorders, endocrine disorders, increasing gastrointestinal motility and obesity. Lipid metabolism disorder including, but not limited to, dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, erectile dysfunction, fatty liver disease, and hepatitis. Billary disorders include gallbladder disorders such as for example, gallstones, gall bladder cancer cholangitis, or primary sclerosing cholangitis; or bile duct disorders such as for example, cholecystitis, bile duct cancer or fascioliasis. Gastrointestinal disorders include for example, irritable bowel syndrome (IBS), non-ulcer dyspepsia, chronic intestinal pseudo-obstruction, functional dyspepsia, colonic pseudo-obstruction, duodenogastric reflux, gastroesophageal reflux disease (GERD), ileus inflammation (e.g., post-operative ileus), gastroparesis, heartburn (high acidity in the GI tract), constipation (e.g., constipation associated with use of medications such as opioids, osteoarthritis drugs, osteoporosis drugs; post surgical constipation, constipation associated with neuropathic disorders, chronic idiopathic constipation). Inflammatory disorders include tissue and organ inflammation such as kidney inflammation (e.g., nephritis), gastrointestinal system inflammation (e.g., Crohn's disease and ulcerative colitis); necrotizing enterocolitis (NEC); pancreatic inflammation (e.g., pancreatis), lung inflammation (e.g., bronchitis or asthma) or skin inflammation (e.g., psoriasis, eczema). Lung Disorders include for example chronic obstructive pulmonary disease (COPD), and fibrosis. Cancer includes tissue and organ carcinogenesis including metatases such as for example gastrointestinal cancer, (e.g., gastric cancer, esophageal cancer, pancreatic cancer colorectal cancer, intestinal cancer, anal cancer, liver cancer, gallbladder cancer, or colon cancer; lung cancer; thyroid cancer; skin cancer (e.g., melanoma); oral cancer; urinary tract cancer (e.g. bladder cancer or kidney cancer); blood cancer (e.g. myeloma or leukemia) or prostate cancer. Cardiac disorders include for example, congestive heart failure, trachea cardia hypertension, high cholesterol, or high triglycerides. Cardiovascular disorders include for example aneurysm, angina, atherosclerosis, cerebrovascular accident (stroke), cerebrovasculardisease, congestive heart failure, coronary artery disease, myocardial infarction (heart attack), or peripheral vascular disease. Liver disorders include for example cirrhosis and fibrosis. In addition, GC-C agonist may also be useful to facilitate liver regeneration in liver transplant patients. Eye disorders include for example increased intra-ocular pressure, glaucoma, dry eyes retinal degeneration, disorders of tear glands or eye inflammation. Skin disorders include for example xerosis. Oral disorders include for example dry mouth (xerostomia), Sjögren's syndrome, gum diseases (e.g., periodontal disease), or salivary gland duct blockage or malfunction. Prostate disorders include for example benign prostatic hyperplasia (BPH). Endocrine disorders include for example diabetes mellitus, hyperthyroidism, hypothyroidism, and cystic fibrosis.

Normal healthy adults synthesize cholesterol at a rate of approximately 1 g/day and consume about 0.3 g/day. A relatively constant level of cholesterol in the body (150-200 mg/dL) is maintained by its de novo synthesis in the liver, absorption in the gut and by removal of cholesterol from the blood. The liver plays a central role in the maintenance of sterol balance across the whole body because not only is it the organ that receives most of the cholesterol absorbed by the small intestine, but it is also the site for the degradation and excretion of cholesterol through the bile.

It is well documented that dietary or pharmacological manipulation of the enterohepatic circulation of either cholesterol or bile acids can potentially cause marked changes in the rate at which the liver synthesizes cholesterol, converts cholesterol to bile acids, incorporates cholesterol into very low-density lipoproteins, esterifies and stores cholesterol, or secretes unesterified cholesterol directly into bile. Such changes in the enterohepatic handling of cholesterol may lead to clinically significant shifts in the circulating low-density lipoprotein-cholesterol (LDL-C) concentration and in the degree of biliary cholesterol saturation. The intestine plays a major role in regulating cholesterol homeostasis and about 36% reduction in plasma cholesterol could be achieved by total inhibition of cholesterol from the gut. In healthy humans and rodents, more than 90% of bile acids are reabsorbed by the small intestine and return to the liver to be secreted again into the bile. This efficient bile acid recycling mainly takes place in the ileum through an active process involving a 48 kDa integral brush border membrane glycoprotein termed apical sodium dependent bile acid transporter (ASBT), or ileal-bile acid transporter (IBAT).

Absorption of cholesterol is a multi-step process, in which cholesterol is first micellized by bile acids in the intestinal lumen and then it is absorbed by enterocytes. Thus, bile acids play important role in absorption of dietary cholesterol. Ezetimibe (Zetia®) is a well-known inhibitor of bile acid transporter and has been widely used as an effective cholesterol-lowering drug for treating patients with hypercholesterolemia.

The term “treatment” refers to reducing or alleviating symptoms in a subject, preventing symptoms from worsening or progressing, and/or preventing disease in a subject who is free therefrom. For a given subject, improvement in a symptom, its worsening, regression, or progression may be determined by any objective or subjective measure. Efficacy of the treatment may be measured as an improvement in morbidity or mortality (e.g., lengthening of survival curve for a selected population). Thus, effective treatment would include therapy of existing disease, control of disease by slowing or stopping its progression, prevention of disease occurrence, reduction in the number or severity of symptoms, or a combination thereof. The effect may be shown in a controlled study using one or more statistically significant criteria.

Intracellular cGMP produced by exposing, e.g., contacting a tissue (e.g., gastrointestinals tissue) or cell with GCRA agonists. By inducing is meant an increase in cGMP production compared to a tissue or cell that has not been in contact with GCRA peptide or variant. Tissues or cells are directly contacted with a GCRA peptide or variant. Alternatively, the GCRA peptide or variant is administered systemically. GCRA peptide or variant are administered in an amount sufficient to increase intracellular cGMP concentration. cGMP production is measured by a cell-based assay known in the art (25).

Disorders are treated, prevented or alleviated by administering to a subject, e.g., a mammal such as a human in need thereof, a therapeutically effective dose of a GCRA peptide. The GCRA peptides may be in a pharmaceutical composition in unit dose form, together with one or more pharmaceutically acceptable excipients. The term “unit dose form” refers to a single drug delivery entity, e.g., a tablet, capsule, solution or inhalation formulation. The amount of peptide present should be sufficient to have a positive therapeutic effect when administered to a patient (typically, between 10 μg and 3 g). What constitutes a “positive therapeutic effect” will depend upon the particular condition being treated and will include any significant improvement in a condition readily recognized by one of skill in the art.

The GCRA peptides can be administered alone or in combination with other agents. For example the GCRA peptides can be administered in combination with inhibitors of cGMP dependent phosphodiesterase, such as, for example, suldinac sulfone, zaprinast, motapizone, vardenafil or sildenifil; one or more other chemotherapeutic agents; or anti-inflammatory drugs such as, for example, steroids or non-steroidal anti-inflammatory drugs (NSAIDS), such as aspirin.

Combination therapy can be achieved by administering two or more agents, e.g., a GCRA peptide described herein and another compound, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

The GCRA peptides described herein may be combined with phosphodiesterase inhibitors, e.g., sulindae sulfone, Zaprinast, sildenafil, vardenafil or tadalafil to further enhance levels of cGMP in the target tissues or organs.

Combination therapy can also include two or more administrations of one or more of the agents used in the combination. For example, if agent X and agent Y are used in a combination, one could administer them sequentially in any combination one or more times, e.g., in the order X—Y—X, X—X—Y, Y—X—Y, Y—Y—X, X—X—Y—Y, etc.

Combination therapy can also include the administration of one of the GC-C agonist with azothioprine and/or other immunomodulating agents. The immunomodulating agents may include small molecule drugs and biologics such as Remicade, Humaira, Cimzia etc.

Combination therapy can also include the administration of two or more agents via different routes or locations. For example, (a) one agent is administered orally and another agents is administered intravenously or (b) one agent is administered orally and another is administered locally. In each case, the agents can either simultaneously or sequentially. Approximated dosages for some of the combination therapy agents described herein are found in the “BNF Recommended Dose” column of tables on pages 11-17 of WO01/76632 (the data in the tables being attributed to the March 2000 British National Formulary) and can also be found in other standard formularies and other drug prescribing directories. For some drugs, the customary prescribed dose for an indication will vary somewhat from country to country.

The GCRA peptides, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose (e.g. celphere, Celphere Beads®), diluents, lubricants, binders, disintegrating agents, and the like), etc. If desired, tablet dosages of the disclosed compositions may be coated by standard aqueous or nonaqueous techniques.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a GCRA agonist) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. Such as mannitol, fructooligosaccharides, polyethylene glycol and other excepients. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated fully herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

The pharmaceutical compositions can be included in a container, pack, or dispenser together with instructions for administration.

Compositions of the present invention may also optionally include other therapeutic ingredients, anti-caking agents, preservatives, sweetening agents, colorants, flavors, desiccants, plasticizers, dyes, glidants, anti-adherents, anti-static agents, surfactants (wetting agents), antioxidants, film-coating agents, and the like. Any such optional ingredient must be compatible with the compound described herein to insure the stability of the formulation.

The composition may contain other additives as needed, including for example lactose, glucose, fructose, galactose, trehalose, sucrose, maltose, raffnose, maltitol, melezitose, stachyose, lactitol, palatinite, starch, xylitol, mannitol, myoinositol, and the like, and hydrates thereof, and amino acids, for example alanine, glycine and betaine, and polypeptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as: BINDERS: corn starch, potato starch, other starches, gelatin, natural and synthetic gums such as acacia, xanthan, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (e.g., povidone, crospovidone, copovidone, etc), methyl cellulose, Methocel, pre-gelatinized starch (e.g., STARCH 1500® and STARCH 1500 LM®, sold by Colorcon, Ltd.), hydroxypropyl methyl cellulose, microcrystalline cellulose (FMC Corporation, Marcus Hook, Pa., USA), or mixtures thereof, FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, dextrose, fructose, honey, lactose anhydrate, lactose monohydrate, lactose and aspartame, lactose and cellulose, lactose and microcrystalline cellulose, maltodextrin, maltose, mannitol, microcrystalline cellulose &amp; guar gum, molasses, sucrose, or mixtures thereof, DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums (like gellan), low-substituted hydroxypropyl cellulose, or mixtures thereof, LUBRICANTS: calcium stearate, magnesium stearate, mineral oil, light mineral oil, glycerin, sorbitol, mannitol, polyethylene glycol, other glycols, stearic acid, sodium lauryl sulfate, sodium stearyl fumarate, vegetable based fatty acids lubricant, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Piano, Tex. USA), a pyrogenic silicon dioxide (CAB-O-SIL, Cabot Co., Boston, Mass. USA), or mixtures thereof, ANTI-CAKING AGENTS: calcium silicate, magnesium silicate, silicon dioxide, colloidal silicon dioxide, talc, or mixtures thereof, ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and COATING AGENTS: sodium carboxymethyl cellulose, cellulose acetate phthalate, ethylcellulose, gelatin, pharmaceutical glaze, hydroxypropyl cellulose, hydroxypropyl methylcellulose (hypromellose), hydroxypropyl methyl cellulose phthalate, methylcellulose, polyethylene glycol, polyvinyl acetate phthalate, shellac, sucrose, titanium dioxide, carnauba wax, microcrystalline wax, gellan gum, maltodextrin, methacrylates, microcrystalline cellulose and carrageenan or mixtures thereof.

The formulation can also include other excipients and categories thereof including but not limited to L-histidine, Pluronic®, Poloxamers (such as Lutrol® and Poloxamer 188), ascorbic acid, glutathione, permeability enhancers (e.g. lipids, sodium cholate, acylcarnitine, salicylates, mixed bile salts, fatty acid micelles, chelators, fatty acid, surfactants, medium chain glycerides), protease inhibitors (e.g. soybean trypsin inhibitor, organic acids), pH lowering agents and absorption enhancers effective to promote bioavailability (including but not limited to those described in U.S. Pat. No. 6,086,918 and U.S. Pat. No. 5,912,014), creams and lotions (like maltodextrin and carrageenans); materials for chewable tablets (like dextrose, fructose, lactose monohydrate, lactose and aspartame, lactose and cellulose, maltodextrin, maltose, mannitol, microcrystalline cellulose and guar gum, sorbitol crystalline); parenterals (like mannitol and povidone); plasticizers (like dibutyl sebacate, plasticizers for coatings, polyvinylacetate phthalate); powder lubricants (like glyceryl behenate); soft gelatin capsules (like sorbitol special solution); spheres for coating (like sugar spheres); spheronization agents (like glyceryl behenate and microcrystalline cellulose); suspending/gelling agents (like carrageenan, gellan gum, mannitol, microcrystalline cellulose, povidone, sodium starch glycolate, xanthan gum); sweeteners (like aspartame, aspartame and lactose, dextrose, fructose, honey, maltodextrin, maltose, mannitol, molasses, sorbitol crystalline, sorbitol special solution, sucrose); wet granulation agents (like calcium carbonate, lactose anhydrous, lactose monohydrate, maltodextrin, mannitol, microcrystalline cellulose, povidone, starch), caramel, carboxymethylcellulose sodium, cherry cream flavor and cherry flavor, citric acid anhydrous, citric acid, confectioner's sugar, D&C Red No. 33, D&C Yellow #10 Aluminum Lake, disodium edetate, ethyl alcohol 15%, FD&C Yellow No. 6 aluminum lake, FD&C Blue #1 Aluminum Lake, FD&C Blue No. 1, FD&C blue no. 2 aluminum lake, FD&C Green No. 3, FD&C Red No. 40, FD&C Yellow No. 6 Aluminum Lake, FD&C Yellow No. 6, FD&C Yellow No. 10, glycerol palmitostearate, glyceryl monostearate, indigo carmine, lecithin, manitol, methyl and propyl parabens, mono ammonium glycyrrhizinate, natural and artificial orange flavor, pharmaceutical glaze, poloxamer 188, Polydextrose, polysorbate 20, polysorbate 80, polyvidone, pregelatinized corn starch, pregelatinized starch, red iron oxide, saccharin sodium, sodium carboxymethyl ether, sodium chloride, sodium citrate, sodium phosphate, strawberry flavor, synthetic black iron oxide, synthetic red iron oxide, titanium dioxide, and white wax.

Solid oral dosage forms may optionally be treated with coating systems (e.g. Opadry® fx film coating system, for example Opadry® blue (OY-LS-20921), Opadry® white (YS-2-7063), Opadry® white (YS-1-7040), and black ink (S-1-8 106).

The agents either in their free form or as a salt can be combined with a polymer such as polylactic-glycolic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(ε-caprolactone) and poly(alkylene oxide) (U.S. 20030068384) to create a sustained release formulation. Such formulations can be used to implants that release a polypeptide or another agent over a period of a few days, a few weeks or several months depending on the polymer, the particle size of the polymer, and the size of the implant (See, e.g., U.S. Pat. No. 6,620,422). Other sustained release formulations and polymers for use in are described in EP 0 467 389 A2, WO 93/24150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. 20030171296, U.S. 20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. 5,980,945, WO 02/058672, WO 97/26015, WO 97/04744, and US20020019446. In such sustained release formulations microparticles (Delie and Blanco-Prieto 2005 Molecule 10:65-80) of polypeptide are combined with microparticles of polymer. One or more sustained release implants can be placed in the large intestine, the small intestine or both. U.S. 6,011,0 1 and WO 94/06452 describe a sustained release formulation providing either polyethylene glycols (i.e. PEG 300 and PEG 400) or triacetin. WO 03/053401 describes a formulation which may both enhance bioavailability and provide controlled release of the agent within the GI tract. Additional controlled release formulations are described in WO 02/38129, EP 326151, U.S. Pat. No. 5,236,704, WO 02/30398, WO 98/13029; U.S. 20030064105, U.S. 20030138488A1, U.S. 20030216307A1, U.S. Pat. No. 6,667,060, WO 01/49249, WO 01/49311, WO 01/49249, WO 01/49311, and U.S. 5,877,224 materials which may include those described in WO04041195 (including the seal and enteric coating described therein) and pH-sensitive coatings that achieve delivery in the colon including those described in U.S. Pat. No. 4,910,021 and WO9001329. U.S. Pat. No. 4,910,021 describes using a pH-sensitive material to coat a capsule. WO9001329 describes using pH-sensitive coatings on beads containing acid, where the acid in the bead core prolongs dissolution of the pH-sensitive coating. U.S. Pat. No. 5,175,003 discloses a dual mechanism polymer mixture composed of pH-sensitive enteric materials and film-forming plasticizers capable of conferring permeability to the enteric material, for use in drug-delivery systems; a matrix pellet composed of a dual mechanism polymer mixture permeated with a drug and sometimes covering a pharmaceutically neutral nucleus; a membrane-coated pellet comprising a matrix pellet coated with a dual mechanism polymer mixture envelope of the same or different composition; and a pharmaceutical dosage form containing matrix pellets. The matrix pellet releases acid-soluble drugs by diffusion in acid pH and by disintegration at pH levels of nominally about 5.0 or higher.

The GCRA peptides described herein may be formulated in the pH triggered targeted control release systems described in WO04052339. The agents described herein may be formulated according to the methodology described in any of WO03105812 (extruded hydratable polymers); WO0243767 (enzyme cleavable membrane translocators); WO03007913 and WO03086297 (mucoadhesive systems); WO02072075 (bilayer laminated formulation comprising pH lowering agent and absorption enhancer); WO04064769 (amidated polypeptides); WO05063156 (solid lipid suspension with pseudotropic and/or thixotropic properties upon melting); WO03035029 and WO03035041 (erodible, gastric retentive dosage forms); U.S. Pat. No. 5,007,790 and U.S. Pat. No. 5,972,389 (sustained release dosage forms); WO041 1271 1 (oral extended release compositions); WO05027878, WO02072033, and WO02072034 (delayed release compositions with natural or synthetic gum); WO05030182 (controlled release formulations with an ascending rate of release); WO05048998 (microencapsulation system); U.S. Pat. No. 5,952,314 (biopolymer); U.S. Pat. No. 5,108,758 (glassy amylose matrix delivery); U.S. Pat. No. 5,840,860 (modified starch based delivery). JP10324642 (delivery system comprising chitosan and gastric resistant material such as wheat gliadin or zein); U.S. Pat. No. 5,866,619 and U.S. Pat. No. 6,368,629 (saccharide containing polymer); U.S. Pat. No. 6,531,152 (describes a drug delivery system containing a water soluble core (Ca pectinate or other water-insoluble polymers) and outer coat which bursts (e.g. hydrophobic polymer-Eudragrit)); U.S. Pat. No. 6,234,464; U.S. Pat. No. 6,403,130 (coating with polymer containing casein and high methoxy pectin; WO0174 175 (Maillard reaction product); WO05063206 (solubility increasing formulation); WO040 19872 (transferring fusion proteins).

The GCRA peptides described herein may be formulated using gastrointestinal retention system technology (GIRES; Merrion Pharmaceuticals). GIRES comprises a controlled-release dosage form inside an inflatable pouch, which is placed in a drug capsule for oral administration. Upon dissolution of the capsule, a gas-generating system inflates the pouch in the stomach where it is retained for 16-24 hours, all the time releasing agents described herein.

The GCRA peptides described herein can be formulated in an osmotic device including the ones disclosed in U.S. Pat. No. 4,503,030, U.S. Pat. No. 5,609,590 and U.S. Pat. No. 5,358,502. U.S. Pat. No. 4,503,030 discloses an osmotic device for dispensing a drug to certain pH regions of the gastrointestinal tract. More particularly, the invention relates to an osmotic device comprising a wall formed of a semi-permeable pH sensitive composition that surrounds a compartment containing a drug, with a passageway through the wall connecting the exterior of the device with the compartment. The device delivers the drug at a controlled rate in the region of the gastrointestinal tract having a pH of less than 3.5, and the device self-destructs and releases all its drug in the region of the gastrointestinal tract having a pH greater than 3.5, thereby providing total availability for drug absorption. U.S. Pat. Nos. 5,609,590 and 5,358,502 disclose an osmotic bursting device for dispensing a beneficial agent to an aqueous environment. The device comprises a beneficial agent and osmagent surrounded at least in part by a semi-permeable membrane. The beneficial agent may also function as the osmagent. The semi-permeable membrane is permeable to water and substantially impermeable to the beneficial agent and osmagent. A trigger means is attached to the semi-permeable membrane (e.g., joins two capsule halves). The trigger means is activated by a pH of from 3 to 9 and triggers the eventual, but sudden, delivery of the beneficial agent. These devices enable the pH-triggered release of the beneficial agent core as a bolus by osmotic bursting.

Exemplary Agents for Combination Therapy

### Analgesic Agents

The GCRA peptide described herein can be used in combination therapy with an analgesic agent, e.g., an analgesic compound or an analgesic polypeptide. These polypeptides and compounds can be administered with the GCRA peptide described herein (simultaneously or sequentially). They can also be optionally covalently linked or attached to an agent described herein to create therapeutic conjugates. Among the useful analgesic agents are: Calcium channel blockers, 5HT receptor antagonists (for example 5HT3, 5HT4 and 5HT1 receptor antagonists), opioid receptor agonists (loperamide, fedotozine, and fentanyl), NK1 receptor antagonists, CCK receptor agonists (e.g., loxiglumide), NK1 receptor antagonists, NK3 receptor antagonists, norepinephrine-serotonin reuptake inhibitors (NSRI), vanilloid and cannabanoid receptor agonists, and sialorphin. Analgesics agents in the various classes are described in the literature.

Among the useful analgesic polypeptides are sialorphin-related polypeptides, including those comprising the amino acid sequence QHNPR (SEQ ID NO: 2), including: VQHNPR (SEQ ID NO: 3); VRQHNPR (SEQ ID NO: 4); VRGQHNPR (SEQ ID NO: 5); VRGPQHNPR (SEQ ID NO: 6); VRGPRQHNPR (SEQ ID NO: 7); VRGPRRQHNPR (SEQ ID NO: 8); and RQHNPR (SEQ ID NO: 9). Sialorphin-related polypeptides bind to neprilysin and inhibit neprilysin-mediated breakdown of substance P and Met-enkephalin. Thus, compounds or polypeptides that are inhibitors of neprilysin are useful analgesic agents which can be administered with the polypeptides described herein in a co-therapy or linked to the polypeptides described herein, e.g., by a covalent bond. Sialophin and related polypeptides are described in U.S. Pat. No. 6,589,750; U.S. 20030078200 A1; and WO 02/051435 A2.

Opioid receptor antagonists and agonists can be administered with the GCRA peptides described herein in co-therapy or linked to the agent described herein, e.g., by a covalent bond. For example, opioid receptor antagonists such as naloxone, naltrexone, methyl nalozone, nalmefene, cypridime, beta funaltrexamine, naloxonazine, naltrindole, and nor-binaltorphimine are thought to be useful in the treatment of IBS. It can be useful to formulate opioid antagonists of this type is a delayed and sustained release formulation such that initial release of the antagonist is in the mid to distal small intestine and/or ascending colon. Such antagonists are described in WO 01/32180 A2. Enkephalin pentapeptide (HOE825; Tyr-D-Lys-Gly-Phe-L-homoserine) (SEQ ID NO: 10) is an agonist of the mu and delta opioid receptors and is thought to be useful for increasing intestinal motility {Eur. J. Pharm. 219:445, 1992), and this polypeptide can be used in conjunction with the polypeptides described herein. Also useful is trimebutine which is thought to bind to mu/delta/kappa opioid receptors and activate release of motilin and modulate the release of gastrin, vasoactive intestinal polypeptide, gastrin and glucagons. Kappa opioid receptor agonists such as fedotozine, asimadoline, and ketocyclazocine, and compounds described in WO03/097051 and WO05/007626 can be used with or linked to the polypeptides described herein. In addition, mu opioid receptor agonists such as morphine, diphenyloxylate, frakefamide (H-Tyr-D-Ala-Phe(F)-Phe-NH2 (SEQ ID NO: 11); WO 01/019849 A1) and loperamide can be used.

Tyr-Arg (kyotorphin) is a dipeptide that acts by stimulating the release of met-enkephalins to elicit an analgesic effect (J. Biol. Chem 262:8165, 1987). Kyotorphin can be used with or linked to the GCRA peptides described herein.

Chromogranin-derived polypeptide (CgA 47-66; See, e.g., Ghia et al. 2004 Regulatory polypeptides 119:199) can be used with or linked to the GCRA peptides described herein.

CCK receptor agonists such as caerulein from amphibians and other species are useful analgesic agents that can be used with or linked to the GCRA peptides described herein.

Conotoxin polypeptides represent a large class of analgesic polypeptides that act at voltage gated calcium channels, NMDA receptors or nicotinic receptors. These polypeptides can be used with or linked to the polypeptides described herein.

Peptide analogs of thymulin (FR Application 2830451) can have analgesic activity and can be used with or linked to the polypeptides described herein.

CCK (CCKa or CCKb) receptor antagonists, including loxiglumide and dexloxiglumide (the R-isomer of loxiglumide) (WO 88/05774) can have analgesic activity and can be used with or linked to the polypeptides described herein.

Other useful analgesic agents include 5-HT4 agonists such as tegaserod (Zelnorm®), mosapride, metoclopramide, zacopride, cisapride, renzapride, benzimidazolone derivatives such as BIMU 1 and BIMU 8, and lirexapride. Such agonists are described in: EP1321 142 A1, WO 03/053432A1, EP 505322 A1, EP 505322 B1, U.S. Pat. No. 5,510,353, EP 507672 A1, EP 507672 B1, and U.S. Pat. No. 5,273,983.

Calcium channel blockers such as ziconotide and related compounds described in, for example, EP625162B1, U.S. Pat. No. 5,364,842, U.S. Pat. No. 5,587,454, U.S. Pat. No. 5,824,645, U.S. Pat. No. 5,859,186, U.S. Pat. No. 5,994,305, U.S. Pat. No. 6,087,091, U.S. Pat. No. 6,136,786, WO 93/13128 A1, EP 1336409 A1, EP 835126 A1, EP 835126 B1, U.S. Pat. No. 5,795,864, U.S. Pat. No. 5,891,849, U.S. Pat. No. 6,054,429, WO 97/01351 A1, can be used with or linked to the polypeptides described herein.

Various antagonists of the NK-I, NK-2, and NK-3 receptors (for a review see Giardina et al. 2003. Drugs 6:758) can be can be used with or linked to the polypeptides described herein.

NK1 receptor antagonists such as: aprepitant (Merck & Co Inc), vofopitant, ezlopitant (Pfizer, Inc.), R-673 (Hoffmann-La Roche Ltd), SR-48968 (Sanofi Synthelabo), CP-122,721 (Pfizer, Inc.), GW679769 (Glaxo Smith Kline), TAK-637 (Takeda/Abbot), SR-14033, and related compounds described in, for example, EP 873753 A1, US 20010006972 A1, US 20030109417 A1, WO 01/52844 A1, can be used with or linked to the polypeptides described herein.

NK-2 receptor antagonists such as nepadutant (Menarini Ricerche SpA), saredutant (Sanofi-Synthelabo), GW597599 (Glaxo Smith Kline), SR-144190 (Sanofi-Synthelabo) and UK-290795 (Pfizer Inc) can be used with or linked to the polypeptides described herein.

NK3 receptor antagonists such as osanetant (SR-142801; Sanoft-Synthelabo), SSR-241586, talnetant and related compounds described in, for example, WO 02/094187 A2, EP 876347 A1, WO 97/21680 A1, U.S. Pat. No. 6,277,862, WO 98/1 1090, WO 95/28418, WO 97/19927, and Boden et al. (J Med Chem. 39:1664-75, 1996) can be used with or linked to the polypeptides described herein.

Norepinephrine-serotonin reuptake inhibitors (NSRI) such as milnacipran and related compounds described in WO 03/077897 A1 can be used with or linked to the polypeptides described herein.

Vanilloid receptor antagonists such as arvanil and related compounds described in WO 01/64212 A1 can be used with or linked to the polypeptides described herein.

The analgesic polypeptides and compounds can be administered with the polypeptides and agonists described herein (simultaneously or sequentially). The analgesic agents can also be covalently linked to the polypeptides and agonists described herein to create therapeutic conjugates. Where the analgesic is a polypeptide and is covalently linked to an agent described herein the resulting polypeptide may also include at least one trypsin cleavage site. When present within the polypeptide, the analgesic polypeptide may be preceded by (if it is at the carboxy terminus) or followed by (if it is at the amino terminus) a trypsin cleavage site that allows release of the analgesic polypeptide.

In addition to sialorphin-related polypeptides, analgesic polypeptides include: AspPhe, endomorphin-1, endomorphin-2, nocistatin, dalargin, lupron, ziconotide, and substance P.

### Agents to Treat Gastrointestinal Disorders

Examples of additional therapeutic agents to treat gastrointestinal and other disorders include agents to treat constipation (e.g., a chloride channel activator such as the bicyclic fatty acid, Lubiprostone (formerly known as SPI-0211; Sucampo Pharmaceuticals, Inc.; Bethesda, Md.), a laxative (e.g. a bulk-forming laxative (e.g. non starch polysaccharides, Colonel Tablet (polycarbophil calcium), Plantago Ovata®, Equalactin® (Calcium Polycarbophil)), fiber (e.g. FIRERCON® (Calcium Polycarbophil), an osmotic laxative, a stimulant laxative (such as diphenyimethanes (e.g. bisacodyl), anthraquinones (e.g. cascara, senna), and surfactant laxatives (e.g. castor oil, docusates), an emollient/lubricating agent (such as mineral oil, glycerine, and docusates), MiraLax (Braintree Laboratories, Braintree Mass.), dexloxiglumide (Forest Laboratories, also known as CR 2017 Rottapharm (Rotta Research Laboratorium SpA)), saline laxatives, enemas, suppositories, and CR 3700 (Rottapharm (Rotta Research Laboratorium SpA); acid reducing agents such as proton pump inhibitors (e.g., omeprazole (Prilosec®), esomeprazole (Nexium®), lansoprazole (Prevacid®), pantoprazole (Protonix®) and rabeprazole (Aciphex®)) and Histamine H2-receptor antagonist (also known as 1-12 receptor blockers including cimetidine, ranitidine, famotidine and nizatidine); prokinetic agents including itopride, octreotide, bethanechol, metoclopramide (Reglan®), domperidone (Motilium®), erythromycin (and derivatives thereof) or cisapride (Propulsid®); Prokineticin poly peptides homologs, variants and chimeras thereof including those described in U.S. Pat. No. 7,052,674 which can be used with or linked to the polypeptides described herein; pro-motility agents such as the vasostatin-derived polypeptide, chromogranin A (4-16) (See, e.g., Ghia et al. 2004 Regulatory polypeptides 121:31) or motilin agonists (e.g., GM-611 or mitemcinal fumarate) or nociceptin/Orphanin FQ receptor modulators (US20050169917); other peptides which can bind to and/or activate GC-C including those described in US20050287067; complete or partial 5HT (e.g. 5HT1, 5HT2, 5HT3, 5HT4) receptor agonists or antagonists (including 5HT1A antagonists (e.g. AGI-OOI (AGI therapeutics), 5HT2B antagonists (e.g. PGN 1091 and PGN1 164 (Pharmagene Laboratories Limited), and 5HT4 receptor agonists (such as tegaserod (ZELNORM®), prucalopride, mosapride, metoclopramide, zacopride, cisapride, renzapride, benzimidazolone derivatives such as BIMU 1 and BIMU 8, and lirexapride). Such agonists/modulators are described in: EP1321142 A1, WO 03/053432A1, EP 505322 A1, EP 505322 B1, U.S. Pat. No. 5,510,353, EP 507672 A1, EP 507672 B1, U.S. Pat. No. 5,273,983, and U.S. Pat. No. 6,951,867); 5HT3 receptor agonists such as MKC-733; and 5HT3 receptor antagonists such as DDP-225 (MCI-225; Dynogen Pharmaceuticals, Inc.), cilansetron (Calmactin®), alosetron (Lotronex®), Ondansetron HCl (Zofran®), Dolasetron (ANZEMET®), palonosetron (Aloxi®), Granisetron (Kytril®), YM060 (ramosetron; Astellas Pharma Inc.; ramosetron may be given as a daily dose of 0.002 to 0.02 mg as described in EP01588707) and ATI-7000 (Aryx Therapeutics, Santa Clara Calif.); muscarinic receptor agonists; anti-inflammatory agents; antispasmodics including but not limited to anticholinergic drugs (like dicyclomine (e.g. Colimex®, Formulex®, Lomine®, Protylol®, Visceral®, Spasmoban®, Bentyl®, Bentylol®), hyoscyamine (e.g. IB-Stat®, Nulev®, Levsin®, Levbid®, Levsinex Timecaps®, Levsin/SL®, Anaspaz®, A-Spas S/L®, Cystospaz®, Cystospaz-M®, Donnamar®, Colidrops Liquid Pediatric®, Gastrosed®, Hyco Elixir®, Hyosol®, Hyospaz®, Hyosyne®, Losamine®, Medispaz®, Neosol®, Spacol®, Spasdel®, Symax®, Symax SL®), Donnatal (e.g. Donnatal Extentabs®), clidinium (e.g. Quarzan, in combination with Librium=Librax), methantheline (e.g. Banthine), Mepenzolate (e.g. Cantil), homatropine (e.g. hycodan, Homapin), Propantheline bromide (e.g. Pro-Banthine), Glycopyrrolate (e.g. Robinul®, Robinul Forte®), scopolamine (e.g. Transderm-Scop®, Transderm-V®), hyosine-N-butylbromide (e.g. Buscopan®), Pirenzepine (e.g. Gastrozepin®) Propantheline Bromide (e.g. Propanthel®), dicycloverine (e.g. Merbentyl®), glycopyrronium bromide (e.g. Glycopyrrolate®), hyoscine hydrobromide, hyoscine methobromide, methanthelinium, and octatropine); peppermint oil; and direct smooth muscle relaxants like cimetropium bromide, mebeverine (DUSPATAL®, DUSPATALIN®, COLOFAC MR®, COLOTAL®), otilonium bromide (octilonium), pinaverium (e.g. Dicetel® (pinaverium bromide, Solvay S. A.)), Spasfon® (hydrated phloroglucinol and trimethylphloroglucinol) and trimebutine (including trimebutine maleate (Modulon®); antidepressants, including but not limited to those listed herein, as well as tricyclic antidepressants like amitriptyline (Elavil®), desipramine (Norpramin®), imipramine (Tofranil®), amoxapine (Asendin®), nortriptyline; the selective serotonin reuptake inhibitors (SSRTs) like paroxetine (Paxil®), fluoxetine (Prozac®), sertraline (Zoloft®), and citralopram (Celexa®); and others like doxepin (Sinequan®) and trazodone (Desyrel®), centrally-acting analgesic agents such as opioid receptor agonists, opioid receptor antagonists (e.g., naltrexone); agents for the treatment of Inflammatory bowel disease, agents for the treatment of Crohn's disease and/or ulcerative colitis (e.g., alequel (Enzo Biochem, Inc.; Farmingsale, N.Y.), the anti-inflammatory polypeptide RDP58 (Genzyme, Inc.; Cambridge, Mass.), and TRAFICET-EN™ (ChemoCentryx, Inc.; San Carlos, Calif.); agents that treat gastrointestinal or visceral pain; agents that increase cGMP levels (as described in US20040121994) like adrenergic receptor antagonists, dopamine receptor agonists and PDE (phosphodiesterase) inhibitors including but not limited to those disclosed herein; purgatives that draw fluids to the intestine (e.g., VISICOL®, a combination of sodium phosphate monobasic monohydrate and sodium phosphate dibasic anhydrate); Corticotropin Releasing Factor (CRF) receptor antagonists (including NBI-34041 (Neurocrine Biosciences, San Diego, Calif.), CRH9-41, astressin, R121919 (Janssen Pharmaceutica), CP154,526, NBI-27914, Antalarmin, DMP696 (Bristol-Myers Squibb) CP-316,311 (Pfizer, Inc), SB723620 (GSK), GW876008 (Neurocrine/Glaxo Smith Kline), ONO-2333Ms (Ono Pharmaceuticals), TS-041 (Janssen), AAG561 (Novartis) and those disclosed in U.S. Pat. No. 5,063,245, U.S. Pat. No. 5,861,398, US20040224964, US20040198726, US20040176400, US20040171607, US200401 10815, US20040006066, and US20050209253); glucagon-like polypeptides (glp-1) and analogues thereof (including exendin-4 and GTP-010 (Gastrotech Pharma A)) and inhibitors of DPP-IV (DPP-IV mediates the inactivation of glp-1); tofisopam, enantiomerically-pure R-totisopam, and pharmaceutically-acceptable salts thereof (US 20040229867); tricyclic anti-depressants of the dibenzothiazepine type including but not limited to Dextofisopam® (Vela Pharmaceuticals), tianeptine (Stablon®) and other agents described in U.S. Pat. No. 6,683,072, (E)-4 (1,3bis(cyclohexylmethyl)-1,2,34,-tetrahydro-2,6-diono-9H-purin-8-yl)cinnamic acid nonaethylene glycol methyl ether ester and related compounds described in WO 02/067942; the probiotic PROBACTRIX® (The BioBalance Corporation; New York, N.Y.) which contains microorganisms useful in the treatment of gastrointestinal disorders; antidiarrheal drugs including but not limited to loperamide (Imodium, Pepto Diarrhea), diphenoxylate with atropine (Lomotil, Lomocot), cholestyramine (Questran, Cholybar), atropine (Co-Phenotrope, Diarsed, Diphenoxylate, Lofene, Logen, Lonox, Vi-Atro, atropine sulfate injection) and Xifaxan® (rifaximin; Salix Pharmaceuticals Ltd), TZP-201(Tranzyme Pharma Inc.), the neuronal acetylcholine receptor (nAChR) blocker AGI-004 (AGI therapeutics), and bismuth subsalicylate (Pepto-bismol); anxiolytic drugs including but not limited toAtivan (lorazepam), alprazolam (Xanax®), chlordiazepoxide/clidinium (Librium®, Librax®), clonazepam (Klonopin®), clorazepate (Tranxene®), diazepam (Valium®), estazolam (ProSom®), flurazepam (Dalmane®), oxazepam (Serax®), prazepam (Centrax®), temazepam (Restoril®), triazolam (Halcion®; Bedelix® (Montmorillonite beidellitic; Ipsen Ltd), Solvay SLV332 (ArQule Inc), YKP (SK Pharma), Asimadoline (Tioga Pharmaceuticals/Merck), AGI-003 (AGI Therapeutics); neurokinin antagonists including those described in US20060040950; potassium channel modulators including those described in U.S. Pat. No. 7,002,015; the serotonin modulator AZD7371 (AstraZeneca PIc); M3 muscarinic receptor antagonists such as darifenacin (Enablex; Novartis AG and zamifenacin (Pfizer); herbal and natural therapies including but not limited to acidophilus, chamomile tea, evening primrose oil, fennel seeds, wormwood, comfrey, and compounds of Bao-Ji-Wan (magnolol, honokiol, imperatorin, and isoimperatorin) as in U.S. Pat. No. 6,923,992; and compositions comprising lysine and an anti-stress agent for the treatment of irritable bowel syndrome as described in EPO 1550443.

### Insulin and Insulin Modulating Agents

The GCRA peptide described herein can be used in combination therapy with insulin and related compounds including primate, rodent, or rabbit insulin including biologically active variants thereof including allelic variants, more preferably human insulin available in recombinant form. Sources of human insulin include pharmaceutically acceptable and sterile formulations such as those available from Eli Lilly (Indianapolis, Ind. 46285) as Humulin™ (human insulin rDNA origin). See, the THE PHYSICIAN'S DESK REFERENCE, 55.sup.th Ed (2001) Medical Economics, Thomson Healthcare (disclosing other suitable human insulins).

The GCRA peptide described herein can also be used in combination therapy with agents that can boost insulin effects or levels of a subject upon administration. e.g. glipizide and/or rosiglitazone. The polypeptides and agonists described herein can be used in combitherapy with SYMLIN® (pramlintide acetate) and Exenatide® (synthetic exendin-4; a 39 aa polypeptide).

### Agents for the Treatment of Postoperative Ileus

The GCRA peptide described herein can also be used in combination therapy with agents (e.g., Entereg™ (alvimopan; formerly called ado lor/ADL 8-2698), conivaptan and related agents describe in U.S. Pat. No. 6,645,959) used for the treatment of postoperative ileus and other disorders.

### Anti-Hypertensive Agents

The GCRA peptide described herein can be used in combination therapy with an anti-hypertensive agent including but not limited to: (1) diuretics, such as thiazides, including chlorthalidone, chlorothiazide, dichlorophenamide, hydroflumethiazide, indapamide, polythiazide, and hydrochlorothiazide; loop diuretics, such as bumetanide, ethacrynic acid, furosemide, and torsemide; potassium sparing agents, such as amiloride, and triamterene; carbonic anhydrase inhibitors, osmotics (such as glycerin) and aldosterone antagonists, such as spironolactone, epirenone, and the like; (2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (4) angiotensin converting enzyme (ACE) inhibitors such as benazepril; captopril; ceranapril; cilazapril; delapril; enalapril; enalopril; fosinopril; imidapril; lisinopril; losinopril; moexipril; quinapril; quinaprilat; ramipril; perindopril; perindropril; quanipril; spirapril; tenocapril; trandolapril, and zofenopril, and the like; (5) neutral endopeptidase inhibitors such as omapatrilat, cadoxatril and ecadotril, fosidotril, sampatrilat, AVE7688, ER4030, and the like; (6) endothelin antagonists such as tezosentan, A308165, and YM62899, and the like; (7) vasodilators such as hydralazine, clonidine, minoxidil, and nicotinyl alcohol, and the like; (8) angiotensin II receptor antagonists such as aprosartan, candesartan, eprosartan, irbesartan, losartan, olmesartan, pratosartan, tasosartan, telmisartan, valsartan, and EXP-3137, FI6828K, and RNH6270, and the like; (9) α/β adrenergic blockers such as nipradilol, arotinolol and amosulalol, and the like; (10) alpha 1 blockers, such as terazosin, urapidil, prazosin, tamsulosin, bunazosin, trimazosin, doxazosin, naftopidil, indoramin, WHP 164, and XENOlO, and the like; (11) alpha 2 agonists such as lofexidine, tiamenidine, moxonidine, rilmenidine and guanobenz, and the like; (12) aldosterone inhibitors, and the like; and (13) angiopoietin-2-binding agents such as those disclosed in WO03/030833. Specific anti-hypertensive agents that can be used in combination with polypeptides and agonists described herein include, but are not limited to: diuretics, such as thiazides (e.g., chlorthalidone, cyclothiazide (CAS RN 2259-96-3), chlorothiazide (CAS RN 72956-09-3, which may be prepared as disclosed in U.S. Pat. No. 2,809,194), dichlorophenamide, hydroflumethiazide, indapamide, polythiazide, bendroflumethazide, methyclothazide, polythiazide, trichlormethazide, chlorthalidone, indapamide, metolazone, quinethazone, althiazide (CAS RN 5588-16-9, which may be prepared as disclosed in British Patent No. 902,658), benzthiazide (CAS RN 91-33-8, which may be prepared as disclosed in U.S. Pat. No. 3,108,097), buthiazide (which may be prepared as disclosed in British Patent Nos. 861,367), and hydrochlorothiazide), loop diuretics (e.g. bumetanide, ethacrynic acid, furosemide, and torasemide), potassium sparing agents (e.g. amiloride, and triamterene (CAS Number 396-01-O)), and aldosterone antagonists (e.g. spironolactone (CAS Number 52-01-7), epirenone, and the like), β-adrenergic blockers such as Amiodarone (Cordarone, Pacerone), bunolol hydrochloride (CAS RN 31969-05-8, Parke-Davis), acebutolol (±N-[3-Acetyl-4-[2-hydroxy-3-[(1 methylethyl)amino]propoxy]phenyl]-butanamide, or (±)-3′-Acetyl-4′-[2-hydroxy-3-(isopropylamino) propoxy]butyranilide), acebutolol hydrochloride (e.g. Sectral®, Wyeth-Ayerst), alprenolol hydrochloride (CAS RN 13707-88-5 see Netherlands Patent Application No. 6,605,692), atenolol (e.g. Tenormin®, AstraZeneca), carteolol hydrochloride (e.g. Cartrol® Filmtab®, Abbott), Celiprolol hydrochloride (CAS RN 57470-78-7, also see in U.S. Pat. No. 4,034,009), cetamolol hydrochloride (CAS RN 77590-95-5, see also U.S. Pat. No. 4,059,622), labetalol hydrochloride (e.g. Normodyne®, Schering), esmolol hydrochloride (e.g. Brevibloc®, Baxter), levobetaxolol hydrochloride (e.g. Betaxon™ Ophthalmic Suspension, Alcon), levobunolol hydrochloride (e.g. Betagan® Liquifilm® with C CAP® Compliance Cap, Allergan), nadolol (e.g. Nadolol, Mylan), practolol (CAS RN 6673-35-4, see also U.S. Pat. No. 3,408,387), propranolol hydrochloride (CAS RN 318-98-9), sotalol hydrochloride (e.g. Betapace AF™, Berlex), timolol (2-Propanol,1-[(1,1-dimethylethyl)amino]-3-[[4-4(4-morpholinyl)-1,2,5-thiadiazol-3-yl]oxy]-, hemihydrate, (S)-, CAS RN 91524-16-2), timolol maleate (S)-I-[(1,1-dimethylethyl)amino]-3-[[4-(4-morpholinyl)-1,2,5-thiadiazol-3-yl]oxy]-2-propanol (Z)-2-butenedioate (1:1) salt, CAS RN 26921-17-5), bisoprolol (2-Propanol, 1-[4-[[2-(1-methylethoxy)ethoxy]-methyl]phenoxyl]-3-[(1-meth-ylethyl)amino]-, (±), CAS RN 66722-44-9), bisoprolol fumarate (such as (±)-1-[4-[[2-(1-Methylethoxy) ethoxy]methyl]phenoxy]-3-[(1-methylethyl)amino]-2-propanol (E)-2-butenedioate (2:1) (salt), e.g., Zebeta™, Lederle Consumer), nebivalol (2H-1-Benzopyran-2-methanol, αα′-[iminobis(methylene)]bis[6-fluoro-3,4-dihydro-, CAS RN 99200-09-6 see also U.S. Pat. No. 4,654,362), cicloprolol hydrochloride, such 2-Propanol, 1-[4-[2-(cyclopropylmethoxy)ethoxy]phenoxy]-3-[1-methylethyl)amino]-, hydrochloride, A.A.S. RN 63686-79-3), dexpropranolol hydrochloride (2-Propanol,1-[1-methylethy)-amino]-3-(1-naphthalenyloxy)-hydrochloride (CAS RN 13071-11-9), diacetolol hydrochloride (Acetamide, N-[3-acetyl-4-[2-hydroxy-3-[(1-methyl-ethyl)amino]propoxy][phenyl]-, monohydrochloride CAS RN 69796-04-9), dilevalol hydrochloride (Benzamide, 2-hydroxy-5-[1-hydroxy-2-[1-methyl-3-phenylpropyl)amino]ethyl]-, monohydrochloride, CAS RN 75659-08-4), exaprolol hydrochloride (2-Propanol, 1-(2-cyclohexylphenoxy)-3-[(1-methylethyl)amino]-, hydrochloride CAS RN 59333-90-3), flestolol sulfate (Benzoic acid, 2-fluoro-,3-[[2-[aminocarbonyl)amino]-dimethylethyl]amino]-2-hydroxypropyl ester, (+)-sulfate (1:1) (salt), CAS RN 88844-73-9; metalol hydrochloride (Methanesulfonamide, N-[4-[1-hydroxy-2-(methylamino)propyl]phenyl]-, monohydrochloride CAS RN 7701-65-7), metoprolol 2-Propanol, 1-[4-(2-methoxyethyl)phenoxy]-3-[1-methylethyl)amino]-; CAS RN 37350-58-6), metoprolol tartrate (such as 2-Propanol, 1-[4-(2-methoxyethyl)phenoxy]-3-[(1-methylethyl)amino]-, e.g., Lopressor®, Novartis), pamatolol sulfate (Carbamic acid, [2-[4-[2-hydroxy-3-[(1-methylethyl)amino]propoxyl]phenyl]-ethyl]-, methyl ester, (±) sulfate (salt) (2:1), CAS RN 59954-01-7), penbutolol sulfate (2-Propanol, 1-(2-cyclopentylphenoxy)-3-[1,1-dimethyle-thyl)amino]1, (S)-, sulfate (2:1) (salt), CAS RN 38363-32-5), practolol (Acetamide, N-[4-[2-hydroxy-3-[(1-methylethyl)amino]-propoxy]phenyl]-, CAS RN 6673-35-4;) tiprenolol hydrochloride (Propanol, 1-[(1-methylethyl)amino]-3-[2-(methylthio)-phenoxy]-, hydrochloride, (±), CAS RN 39832-43-4), tolamolol (Benzamide, 4-[2-[[2-hydroxy-3-(2-methylphenoxy)-propyl]amino]ethoxyl]-, CAS RN 38103-61-6), bopindolol, indenolol, pindolol, propanolol, tertatolol, and tilisolol, and the like; calcium channel blockers such as besylate salt of amlodipine (such as 3-ethyl-5-methyl-2-(2-aminoethoxymethyl)-4-(2-chlorophenyl)-1,4-dihydro-6-methyl-3,5-pyridinedicarboxylate benzenesulphonate, e.g., Norvasc®, Pfizer), clentiazem maleate (1,5-Benzothiazepin-4(5H)-one, 3-(acetyloxy)-8-chloro-5-[2-(dimethylamino)ethyl]-2,3-dihydro-2-(4-methoxyphenyl)-(2S-cis)-, (Z)-2-butenedioate (1:1), see also U.S. Pat. No. 4,567,195), isradipine (3,5-Pyridinedicarboxylic acid, 4-(4-benzofurazanyl)-1,4-dihydro-2,6-dimethyl-, methyl 1-methylethyl ester, (±)-4(4-benzofurazanyl)-1,4-dihydro-2,6-dimethyl-3,5-pyridinedicarboxylate, see also U.S. Pat. No. 4,466,972); nimodipine (such as is isopropyl (2-methoxyethyl) 1, 4-dihydro-2,6-dimethyl-4-(3-nitrophenyl)-3,5-pyridine-dicarboxylate, e.g. Nimotop®, Bayer), felodipine (such as ethyl methyl 4-(2,3-dichlorophenyl)-1,4-dihydro-2,6-dimethyl-3,5-pyridinedicarboxylate-, e.g. Plendil® Extended-Release, AstraZeneca LP), nilvadipine (3,5-Pyridinedicarboxylic acid, 2-cyano-1,4-dihydro-6-methyl-4-(3-nitrophenyl)-,3-methyl 5-(1-methylethyl) ester, also see U.S. Pat. No. 3,799,934), nifedipine (such as 3, 5-pyridinedicarboxylic acid, 1,4-dihydro-2,6-dimethyl-4-(2-nitrophenyl)-, dimethyl ester, e.g., Procardia XL® Extended Release Tablets, Pfizer), diltiazem hydrochloride (such as 1,5-Benzothiazepin-4(5H)-one,3-(acetyloxy)-5[2-(dimethylamino)ethyl]-2,-3-dihydro-2(4-methoxyphenyl)-, monohydrochloride, (+)-cis, e.g., Tiazac®, Forest), verapamil hydrochloride (such as benzeneacetonitrile, (alpha)-[[3-[[2-(3,4-dimethoxyphenyl)ethyl]methylamino]propyl]-3,4-dimethoxy-(alpha)-(1-methylethyl) hydrochloride, e.g., Isoptin® SR, Knoll Labs), teludipine hydrochloride (3,5-Pyridinedicarboxylic acid, 2-[(dimethylamino)methyl]4-[2-[(1E)-3-(1,1-dimethylethoxy)-3-oxo-1-propenyl]phenyl]-1,4-dihydro-6-methyl-, diethyl ester, monohydrochloride) CAS RN 108700-03-4), belfosdil (Phosphonic acid, [2-(2-phenoxy ethyl)-1,3-propane-diyl]bis-, tetrabutyl ester CAS RN 103486-79-9), fostedil (Phosphonic acid, [[4-(2-benzothiazolyl)phenyl]methyl]-, diethyl ester CAS RN 75889-62-2), aranidipine, azelnidipine, barnidipine, benidipine, bepridil, cinaldipine, clevidipine, efonidipine, gallopamil, lacidipine, lemildipine, lercanidipine, monatepil maleate (1-Piperazinebutanamide, N-(6, 11-dihydrodibenzo(b,e)thiepin-11-yl)₄-(4-fluorophenyl)-, (+)-, (Z)-2-butenedioate (1:1) (±)-N-(6,11-Dihydrodibenzo(b,e)thiep-in-11-yl)-4-(p-fluorophenyl)-1-piperazinebutyramide maleate (1:1) CAS RN 132046-06-1), nicardipine, nisoldipine, nitrendipine, manidipine, pranidipine, and the like; T-channel calcium antagonists such as mibefradil; angiotensin convening enzyme (ACE) inhibitors such as benazepril, benazepril hydrochloride (such as 3-[[1-(ethoxycarbonyl)-3-phenyl-(1 S)-propyl]amino]-2,3,4,5-tetrahydro-2-oxo-1H-1-(3 S)-benzazepine-1-acetic acid monohydrochloride, e.g., Lotrel®, Novartis), captopril (such as 1-[(2S)-3-mercapto-2-methylpropionyl]-L-proline, e.g., Captopril, Mylan, CAS RN 62571-86-2 and others disclosed in U.S. Pat. No. 4,046,889), ceranapril (and others disclosed in U.S. Pat. No. 4,452,790), cetapril (alacepril, Dainippon disclosed in Eur. Therap. Res. 39:671 (1986); 40:543 (1986)), cilazapril (Hoffman-LaRoche) disclosed in J. Cardiovasc. Pharmacol. 9:39 (1987), indalapril (delapril hydrochloride (2H-1,2,4-Benzothiadiazine-7-sulfonamide, 3-bicyclo[2.2.1]hept-5-en-2-yl-6-chloro-3,4-dihydro-, 1,1-dioxide CAS RN 2259-96-3); disclosed in U.S. Pat. No. 4,385,051), enalapril (and others disclosed in U.S. Pat. No. 4,374,829), enalopril, enaloprilat, fosinopril, ((such as L-proline, 4-cyclohexyl-1-[[[2-methyl-1-(1-oxopropoxy) propoxy](4-phenylbutyl)phosphinyl]acetyl]-, sodium salt, e.g., Monopril, Bristol-Myers Squibb and others disclosed in U.S. Pat. No. 4,168,267), fosinopril sodium (L-Proline, 4-cyclohexyl-1-[[(R)-[(1S)-2-methyl-1-(1-ox-opropoxy)propox), imidapril, indolapril (Schering, disclosed in J. Cardiovasc. Pharmacol. 5:643, 655 (1983)), lisinopril (Merck), losinopril, moexipril, moexipril hydrochloride (3-Isoquinolinecarboxylic acid, 2-[(2S)-2-[[(1S)-1-(ethoxycarbonyl)-3-phenylpropyl]amino]-1-oxopropyl]-1,-2,3,4-tetrahydro-6,7-dimethoxy-, monohydrochloride, (3S)-CAS RN 82586-52-5), quinapril, quinaprilat, ramipril (Hoechsst) disclosed in EP 79022 and Curr. Ther. Res. 40:74 (1986), perindopril erbumine (such as 2S,3aS,7aS-1-[(S)—N—[(S)-1-Carboxybutyljalanyljhexahydrô-indolinecarboxylic acid, 1-ethyl ester, compound with tert-butylamine (1:1), e.g., Aceon®, Solvay), perindopril (Servier, disclosed in Eur. J. clin. Pharmacol. 31:519 (1987)), quanipril (disclosed in U.S. Pat. No. 4,344,949), spirapril (Schering, disclosed in Acta. Pharmacol. Toxicol. 59 (Supp. 5): 173 (1986)), tenocapril, trandolapril, zofenopril (and others disclosed in U.S. Pat. No. 4,316,906), rentiapril (fentiapril, disclosed in Clin. Exp. Pharmacol. Physiol. 10:131 (1983)), pivopril, YS980, teprotide (Bradykinin potentiator BPP9a CAS RN 35115-60-7), BRL 36,378 (Smith Kline Beecham, see EP80822 and EP60668), MC-838 (Chugai, see CA. 102:72588v and Jap. J. Pharmacol. 40:373 (1986), CGS 14824 (Ciba-Geigy, 3-([1-ethoxycarbonyl-3-phenyl-(1S)-propyl]amino)-2,3,4,5-tetrahydro-2-ox-o-1-(3 S)-benzazepine-1 acetic acid HCl, see U.K. Patent No. 2103614), CGS 16,617 (Ciba-Geigy, 3(S)-[[(1S)-5-amino-1-carboxypentyl]amino]-2,3,4,-5-tetrahydro-2-oxo-1H-1-benzazepine-1-ethanoic acid, see U.S. Pat. No. 4,473,575), Ru 44570 (Hoechst, see Arzneimittelforschung 34:1254 (1985)), R 31-2201 (Hoffman-LaRoche see FEBS Lett. 165:201 (1984)), CI1925 (Pharmacologist 26:243, 266 (1984)), WY-44221 (Wyeth, see J. Med. Chem. 26:394 (1983)), and those disclosed in US2003006922 (paragraph 28), U.S. Pat. No. 4,337,201, U.S. Pat. No. 4,432,971 (phosphonamidates); neutral endopeptidase inhibitors such as omapatrilat (Vanlev®), CGS 30440, cadoxatril and ecadotril, fasidotril (also known as aladotril or alatriopril), sampatrilat, mixanpril, and gemopatrilat, AVE7688, ER4030, and those disclosed in U.S. Pat. No. 5,362,727, U.S. Pat. No. 5,366,973, U.S. Pat. No. 5,225,401, U.S. Pat. No. 4,722,810, U.S. Pat. No. 5,223,516, U.S. Pat. No. 4,749,688, U.S. Pat. No. 5,552,397, U.S. Pat. No. 5,504,080, U.S. Pat. No. 5,612,359, U.S. Pat. No. 5,525,723, EP0599444, EP0481522, EP0599444, EP0595610, EP0534363, EP534396, EP534492, EP0629627, endothelin antagonists such as tezosentan, A308165, and YM62899, and the like; vasodilators such as hydralazine (apresoline), clonidine (clonidine hydrochloride (1H-Imidazol-2-amine, N-(2,6-dichlorophenyl)4,5-dihydro-, monohydrochloride CAS RN 4205-91-8), catapres, minoxidil (loniten), nicotinyl alcohol (roniacol), diltiazem hydrochloride (such as 1,5-Benzothiazepin-, 4(5H)-one,3-(acetyloxy)-5[2-(dimethylamino)ethyl]-2,-3-dihydro-2(4-methoxyphenyl)-, monohydrochloride, (+)-cis, e.g., Tiazac®, Forest), isosorbide dinitrate (such as 1,4:3,6-dianhydro-D-glucitol 2,5-dinitrate e.g., Isordil® Titradose®, Wyeth-Ayerst), sosorbide mononitrate (such as 1,4:3,6-dianhydro-D-glucito-1,5-nitrate, an organic nitrate, e.g., Ismo®, Wyeth-Ayerst), nitroglycerin (such as 2,3 propanetriol trinitrate, e.g., Nitrostat® Parke-Davis), verapamil hydrochloride (such as benzeneacetonitrile, (±)-(alpha)[3-[[2-(3,4 dimethoxyphenyl)ethyl]methylamino]propyl]-3,4-dimethoxy-(alpha)-(1-methylethyl) hydrochloride, e.g., Covera HS® Extended-Release, Searle), chromonar (which may be prepared as disclosed in U.S. Pat. No. 3,282,938), clonitate (Annalen 1870 155), droprenilamine (which may be prepared as disclosed in DE2521113), lidotlazine (which may be prepared as disclosed in U.S. Pat. No. 3,267,104); prenylamine (which may be prepared as disclosed in U.S. Pat. No. 3,152,173), propatyl nitrate (which may be prepared as disclosed in French Patent No. 1,103,113), mioflazine hydrochloride (1-Piperazineacetamide, 3-(aminocarbonyl)₄-[4,4-bis(4-fluorophenyl)butyl]-N-(2,6-dichlorophenyl)-, dihydrochloride CAS RN 83898-67-3), mixidine (Benzeneethanamine, 3,4-dimethoxy-N-(1-methyl-2-pyrrolidinylidene)-Pyrrolidine, 2-[(3,4-dimethoxyphenethyl)imino]-1-methyl-1-Methyl-2-[(3, 4-dimethoxyphenethyl)imino]pyrrolidine CAS RN 27737-38-8), molsidomine (1,2,3-Oxadiazolium, 5-[(ethoxycarbonyl)amino]-3-(4-morpholinyl)-, inner salt CAS RN 25717-80-0), isosorbide mononitrate (D-Glucitol, 1,4:3,6-dianhydro-, 5-nitrate CAS RN 16051-77-7), erythrityl tetranitrate (1,2,3,4-Butanetetrol, tetranitrate, (2R,3S)-rel-CAS RN 7297-25-8), clonitrate(1,2-Propanediol, 3-chloro-, dinitrate (7CI, 8CI, 9CI) CAS RN 2612-33-1), dipyridamole Ethanol, 2,2′,2″,2′″-[(4,8-di-1-piperidinylpyrimido[5,4-d]pyrimidine-2,6-diyl)dinitrilo]tetrakis-CAS RN 58-32-2), nicorandil (CAS RN 65141-46-0 3-), pyridinecarboxamide (N-[2-(nitrooxy)ethyl]-Nisoldipine3,5-Pyridinedicarboxylic acid, 1,4-dihydro-2,6-dimethyl-4-(2-nitrophenyl)-, methyl 2-methylpropyl ester CAS RN 63675-72-9), nifedipine3,5-Pyridinedicarboxylic acid, 1,4-dihydro-2,6-dimethyl-4-(2-nitrophenyl)-, dimethyl ester CAS RN 21829-25-4), perhexiline maleate (Piperidine, 2-(2,2-dicyclohexylethyl)-, (2Z)-2-butenedioate (1:1) CAS RN 6724-53-4), oxprenolol hydrochloride (2-Propanol, 1-[(1-methylethyl)amino]-3-[2-(2-propenyloxy)phenoxy]-, hydrochloride CAS RN 6452-73-9), pentrinitrol (1,3-Propanediol, 2,2-bis[(nitrooxy)methyl]-, mononitrate (ester) CAS RN 1607-17-6), verapamil (Benzeneacetonitrile, α-[3-[[2-(3,4-dimethoxyphenyl)ethyl]-methylamino]propyl]-3, 4-dimethoxy-α-(1-methylethyl)-CAS RN 52-53-9) and the like; angiotensin II receptor antagonists such as, aprosartan, zolasartan, olmesartan, pratosartan, FI6828K, RNH6270, candesartan (1H-Benzimidazole-7-carboxylic acid, 2-ethoxy-1-[[2′-(1H-tetrazol-5-yl)[1,1′-biphenyl]4-yl]methyl]-CAS RN 139481-59-7), candesartan cilexetil ((+/−)-1-(cyclohexylcarbonyloxy)ethyl-2-ethoxy-1-[[2′-(1H-tetrazol-5-yl)biphenyl-4-yl]-1H-benzimidazole carboxylate, CAS RN 145040-37-5, U.S. Pat. No. 5,703,110 and U.S. Pat. No. 5,196,444), eprosartan (3-[1-4-carboxyphenylmethyl)-2-n-butyl-imidazol-5-yl]-(2-thienylmethyl) propenoic acid, U.S. Pat. No. 5,185,351 and U.S. Pat. No. 5,650,650), irbesartan (2-n-butyl-3-[[2′-(1H-tetrazol-5-yl)biphenyl-4-yl]methyl]1,3-diazaspiro[4,4]non-1-en-4-one, U.S. Pat. No. 5,270,317 and U.S. Pat. No. 5,352,788), losartan (2-N-butyl-4-chloro-5-hydroxymethyl-1-[(2′-(1H-tetrazol-5-yl)biphenyl-4-yl)-methyl]imidazole, potassium salt, U.S. Pat. No. 5,138,069, U.S. Pat. No. 5,153,197 and U.S. Pat. No. 5,128,355), tasosartan (5,8-dihydro-2,4-dimethyl-8-[(2′-(1H-tetrazol-5-yl)[1,r-biphenyl]4-yl)methyl]-pyrido[2,3-d]pyrimidin-7(6H)-one, U.S. Pat. No. 5,149,699), telmisartan (4′-[(1,4-dimethyl-2′-propyl-(2,6′-bi-1H-benzimidazol)-r-yl)]-[1,1′-biphenyl]-2-carboxylic acid, CAS RN 144701-48-4, U.S. Pat. No. 5,591,762), milfasartan, abitesartan, valsartan (Diovan® (Novartis), (S)—N-valeryl-N-[[2′-(1H-tetrazol-5-yl)biphenyl-4-yl)methyl]valine, U.S. Pat. No. 5,399,578), EXP-3137 (2-N-butyl-4-chloro-1-[(2′-(1H-tetrazol-5-yl)biphenyl-4-yl)-methyl]imidazole-5-carboxylic acid, U.S. Pat. No. 5,138,069, U.S. Pat. No. 5,153,197 and U.S. Pat. No. 5,128,355), 3-(2′-(tetrazol-5-yl)-1,r-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,r-biphenyl]-2-carboxylic acid, 2-butyl-6-(1-methoxy-1-methylethyl-2-[2′-)1H-tetrazol-5-yl)biphenyl-4-vi methyl]guinazolin-4(3H-)-one, 3-[2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl)[1, 1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophenecarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5 [[2′-(1H-tetrazol-5-yl)[1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo[1,5-c]pyrimidin-2(3H)-one, 2, 7-di ethyl-5-[[2′-(5-tetrazoyl)biphenyl-4-yl]methyl]-5H-pyrazolo[1,5-b][1,2,4]triazole potassium salt, 2-[2-butyl-4, 5-dihydro-4-oxo-3-[2′-(H-tetrazol-5-yl)-4-biphenylmethyl]-3H-imidazol[4,5-c]pyridine-5-ylmethyl]benzoic acid, ethyl ester, potassium salt, 3-methoxy-2,6-dimethyl-4-[[2′(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′ 1 H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6, 7,8-tetrahydro-1-[[2′(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidazole-5-carboxylic acid 1-ethoxycarbonyloxyethyl ester, those disclosed in patent publications EP475206, EP497150, EP539086, EP539713, EP535463, EP535465, EP542059, EP497121, EP535420, EP407342, EP415886, EP424317, EP435827, EP433983, EP475898, EP490820, EP528762, EP324377, EP323841, EP420237, EP500297, EP426021, EP480204, EP429257, EP430709, EP434249, EP446062, EP505954, EP524217, EP514197, EP514198, EP514193, EP514192, EP450566, EP468372, EP485929, EP503162, EP533058, EP467207 EP399731, EP399732, EP412848, EP453210, EP456442, EP470794, EP470795, EP495626, EP495627, EP499414, EP499416, EP499415, EP511791, EP516392, EP520723, EP520724, EP539066, EP438869, EP505893, EP530702, EP400835, EP400974, EP401030, EP407102, EP411766, EP409332, EP412594, EP419048, EP480659, EP481614, EP490587, EP467715, EP479479, EP502725, EP503838, EP505098, EP505111 EP513,979 EP507594, EP510812, EP511767, EP512675, EP512676, EP512870, EP517357, EP537937, EP534706, EP527534, EP540356, EP461040, EP540039, EP465368, EP498723, EP498722, EP498721, EP515265, EP503785, EP501892, EP519831, EP532410, EP498361, EP432737, EP504888, EP508393, EP508445, EP403159, EP403158, EP425211, EP427463, EP437103, EP481448, EP488532, EP501269, EP500409, EP540400, EP005528, EP028834, EP028833, EP411507, EP425921, EP430300, EP434038, EP442473, EP443568, EP445811, EP459136, EP483683, EP518033, EP520423, EP531876, EP531874, EP392317, EP468470, EP470543, EP502314, EP529253, EP543263, EP540209, EP449699, EP465323, EP521768, EP415594, WO92/14468, WO93/08171, WO93/08169, WO91/00277, WO91/00281, WO91/14367, WO92/00067, WO92/00977, WO92/20342, WO93/04045, WO93/04046, WO91/115206, WO92/14714, WO92/09600, WO92/16552, WO93/05025, WO93/03018, WO91/07404, WO92/02508, WO92/13853, WO91/19697, WO91/11909, WO91/12001, WO91/11999, WO91/15209, WO91/15479, WO92/20687, WO92/20662, WO92/20661, WO93/01177, WO91/14679, WO91/13063, WO92/13564, WO91/17148, WO91/18888, WO91/19715, WO92/02257, WO92/04335, WO92/05161, WO92/07852, WO92/15577, WO93/03033, WO91/16313, WO92/00068, WO92/02510, WO92/09278, WO92/10179, WO92/10180, WO92/10186, WO92/10181, WO92/10097, WO92/10183, WO92/10182, WO92/10187, WO92/10184, WO92/10188, WO92/10180, WO92/10185, WO92/20651, WO93/03722, WO93/06828, WO93/03040, WO92/19211, WO92/22533, WO92/06081, WO92/05784, WO93/00341, WO92/04343, WO92/04059, U.S. Pat. No. 5,104,877, U.S. Pat. No. 5,187,168, U.S. Pat. No. 5,149,699, U.S. Pat. No. 5,185,340, U.S. Pat. No. 4,880,804, U.S. Pat. No. 5,138,069, U.S. Pat. No. 4,916,129, U.S. Pat. No. 5,153,197, U.S. Pat. No. 5,173,494, U.S. Pat. No. 5,137,906, U.S. Pat. No. 5,155,126, U.S. Pat. No. 5,140,037, U.S. Pat. No. 5,137,902, U.S. Pat. No. 5,157,026, U.S. Pat. No. 5,053,329, U.S. Pat. No. 5,132,216, U.S. Pat. No. 5,057,522, U.S. Pat. No. 5,066,586, U.S. Pat. No. 5,089,626, U.S. Pat. No. 5,049,565, U.S. Pat. No. 5,087,702, U.S. Pat. No. 5,124,335, U.S. Pat. No. 5,102,880, U.S. Pat. No. 5,128,327, U.S. Pat. No. 5,151,435, U.S. Pat. No. 5,202,322, U.S. Pat. No. 5,187,159, U.S. Pat. No. 5,198,438, U.S. Pat. No. 5,182,288, U.S. Pat. No. 5,036,048, U.S. Pat. No. 5,140,036, U.S. Pat. No. 5,087,634, U.S. Pat. No. 5,196,537, U.S. Pat. No. 5,153,347, U.S. Pat. No. 5,191,086, U.S. Pat. No. 5,190,942, U.S. Pat. No. 5,177,097, U.S. Pat. No. 5,212,177, U.S. Pat. No. 5,208,234, U.S. Pat. No. 5,208,235, U.S. Pat. No. 5,212,195, U.S. Pat. No. 5,130,439, U.S. Pat. No. 5,045,540, U.S. Pat. No. 5,041,152, and U.S. Pat. No. 5,210,204, and pharmaceutically acceptable salts and esters thereof; α/β adrenergic blockers such as nipradilol, arotinolol, amosulalol, bretylium tosylate (CAS RN: 61-75-6), dihydroergotamine mesylate (such as ergotaman-3′,6′,18-trione,9,-10-dihydro-12′-hydroxy-2′-methyl-5′-(phenylmethyl)-,(5′(α))-, monomethanesulfonate, e.g., DHE 45® Injection, Novartis), carvedilol (such as (±)-1-(Carbazol-4-yloxy)-3-[[2-(o-methoxyphenoxy)ethyl]amino]-2-propanol, e.g., Coreg®, SmithKline Beecham), labetalol (such as 5-[1-hydroxy-2-[(1-methyl-3-phenylpropyl)amino]ethyljsalicylamide monohydrochloride, e.g., Normodyne®, Schering), bretylium tosylate (Benzenemethanaminium, 2-bromo-N-ethyl-N,N-dimethyl-, salt with 4-methylbenzenesulfonic acid (1:1) CAS RN 61-75-6), phentolamine mesylate (Phenol, 3-[[(4,5-dihydro-1H-imidazol-2-yl)methyl](4-methylphenyl)amino]-, monomethanesulfonate (salt) CAS RN 65-28-1), solypertine tartrate (5H-1,3-Dioxolo[4,5-f]indole, 7-[2-[4-(2-methoxyphenyl)-1-piperazinyl]ethyl]-, (2R,3R)-2,3-dihydroxybutanedioate (1:1) CAS RN 5591-43-5), zolertine hydrochloride (Piperazine, 1-phenyl4-[2-(1H-tetrazol-5-yl)ethyl]-, monohydrochloride (8Cl, 9Cl) CAS RN 7241-94-3) and the like; α adrenergic receptor blockers, such as alfuzosin (CAS RN. 81403-68-1), terazosin, urapidil, prazosin (Minipress®), tamsulosin, bunazosin, trimazosin, doxazosin, naftopidil, indoramin, WHP 164, XENOlO, fenspiride hydrochloride (which may be prepared as disclosed in U.S. Pat. No. 3,399,192), proroxan (CAS RN 33743-96-3), and labetalol hydrochloride and combinations thereof, α 2 agonists such as methyldopa, methyldopa HCL, lofexidine, tiamenidine, moxonidine, rilmenidine, guanobenz, and the like; aldosterone inhibitors, and the like; renin inhibitors including Aliskiren (SPPlOO; Novartis/Speedel); angiopoietin-2-binding agents such as those disclosed in WO03/030833, anti-angina agents such as ranolazine (hydrochloride 1-Piperazineacetamide, N-(2,6-dimethylphenyl)-4-[2-hydroxy-3-(2-methoxyphenoxy)propyl]-, dihydrochloride CAS RN 95635-56-6), betaxolol hydrochloride (2-Propanol, 1-[4-[2 (cyclopropylmethoxy)ethyl]phenoxy]-3-[(1-methylethyl)amino]-, hydrochloride CAS RN 63659-19-8), butoprozine hydrochloride (Methanone, [4-[3(dibutylamino)propoxy]phenyl](2-ethyl-3-indolizinyl)-, monohydrochloride CAS RN 62134-34-3), cinepazet maleatel-Piperazineacetic acid, 4-[1-oxo-3-(3,4,5-trimethoxy phenyl)-2-propenyl]-, ethyl ester, (2Z)-2-butenedioate (1:1) CAS RN 50679-07-7), tosifen (Benzenesulfonamide, 4-methyl-N-[[[(1S)-1-methyl-2-phenylethyl]amino]carbonyl]-CAS RN 32295-184), verapamilhydrochloride (Benzeneacetonitrile, α-[3-[[2-(3,4-dimethoxyphenyl)ethyl]methylamino]propyl]-3,4-dimethoxy-α-(1-methylethyl)-, monohydrochloride CAS RN 152-114), molsidomine (1,2,3-Oxadiazolium, 5-[(ethoxycarbonyl)amino]-3-(4-morpholinyl)-, inner salt CAS RN 25717-80-0), and ranolazine hydrochloride (1-Piperazineacetamide, N-(2,6-dimethylphenyl)₄-[2-hydroxy-3-(2-meth-oxyphenoxy)propyl]-, dihydrochloride CAS RN 95635-56-6); tosifen (Benzenesulfonamide, 4-methyl-N-[[[(1S)-1-methyl-2-phenylethyl]amino]carbonyl]-CAS RN 32295-184), adrenergic stimulants such as guanfacine hydrochloride (such as N-amidino-2-(2,6-dichlorophenyl) acetamide hydrochloride, e.g., Tenex® Tablets available from Robins); methyldopa-hydrochlorothiazide (such as levo-3-(3,4-dihydroxyphenyl)-2-methylalanine) combined with Hydrochlorothiazide (such as 6-chloro-3,4-dihydro-2H-1,2,4-benzothiadiazine-7-sulfonamide 1,1-dioxide, e.g., the combination as, e.g., Aldoril® Tablets available from Merck), methyldopa-chlorothiazide (such as 6-chloro-2H-1,2,4-benzothiadiazine-7-sulfonamide 1,1-dioxide and methyldopa as described above, e.g., Aldoclor®, Merck), clonidine hydrochloride (such as 2-(2,6-dichlorophenylamino)-2-imidazoline hydrochloride and chlorthalidone (such as 2-chloro-5-(1-hydroxy-3-oxo-1-isoindolinyl)benzenesulfonamide), e.g., Combipres®, Boehringer Ingelheim), clonidine hydrochloride (such as 2-(2,6-dichlorophenylamino)-2-imidazoline hydrochloride, e.g., Catapres®, Boehringer Ingelheim), clonidine (1H-Imidazol-2-amine, N-(2,6-dichlorophenyl)4,5-dihydro-CAS RN 4205-90-7), Hyzaar (Merck; a combination of losartan and hydrochlorothiazide), Co-Diovan (Novartis; a combination of valsartan and hydrochlorothiazide, Lotrel (Novartis; a combination of benazepril and amlodipine) and Caduet (Pfizer; a combination of amlodipine and atorvastatin), and those agents disclosed in US20030069221.

### Agents for the Treatment of Respiratory Disorders

The GCRA peptide described herein can be used in combination therapy with one or more of the following agents useful in the treatment of respiratory and other disorders including but not limited to: (1) β-agonists including but not limited to: albuterol (PRO VENTIL®, S ALBUT AMOl®, VENTOLIN®), bambuterol, bitoterol, clenbuterol, fenoterol, formoterol, isoetharine (BRONKOSOL®, BRONKOMETER®), metaproterenol (ALUPENT®, METAPREL®), pirbuterol (MAXAIR®), reproterol, rimiterol, salmeterol, terbutaline (BRETHAIRE®, BRETHINE®, BRICANYL®), adrenalin, isoproterenol (ISUPREL®), epinephrine bitartrate (PRIMATENE®), ephedrine, orciprenline, fenoterol and isoetharine; (2) steroids, including but not limited to beclomethasone, beclomethasone dipropionate, betamethasone, budesonide, bunedoside, butixocort, dexamethasone, flunisolide, fluocortin, fluticasone, hydrocortisone, methyl prednisone, mometasone, prednisolone, predonisone, tipredane, tixocortal, triamcinolone, and triamcinolone acetonide; (3) β2-agonist-corticosteroid combinations [e.g., salmeterol-fluticasone (ADVAIR®), formoterol-budesonide (S YMBICORT®)]; (4) leukotriene D4 receptor antagonists/leukotriene antagonists/LTD4 antagonists (i.e., any compound that is capable of blocking, inhibiting, reducing or otherwise interrupting the interaction between leukotrienes and the Cys LTI receptor) including but not limited to: zaflhiukast, montelukast, montelukast sodium (SINGULAIR®), pranlukast, iralukast, pobilukast, SKB-106,203 and compounds described as having LTD4 antagonizing activity described in U.S. Pat. No. 5,565,473; (5) 5-lipoxygenase inhibitors and/or leukotriene biosynthesis inhibitors [e.g., zileuton and BAY1005 (CA registry 128253-31-6)]; (6) histamine H1 receptor antagonists/antihistamines (i.e., any compound that is capable of blocking, inhibiting, reducing or otherwise interrupting the interaction between histamine and its receptor) including but not limited to: astemizole, acrivastine, antazoline, azatadine, azelastine, astamizole, bromopheniramine, bromopheniramine maleate, carbinoxamine, carebastine, cetirizine, chlorpheniramine, chloropheniramine maleate, cimetidine clemastine, cyclizine, cyproheptadine, descarboethoxyloratadine, dexchlorpheniramine, dimethindene, diphenhydramine, diphenylpyraline, doxylamine succinate, doxylarnine, ebastine, efletirizine, epinastine, famotidine, fexofenadine, hydroxyzine, hydroxyzine, ketotifen, levocabastine, levocetirizine, levocetirizine, loratadine, meclizine, mepyramine, mequitazine, methdilazine, mianserin, mizolastine, noberastine, norasternizole, noraztemizole, phenindamine, pheniramine, picumast, promethazine, pynlamine, pyrilamine, ranitidine, temelastine, terfenadine, trimeprazine, tripelenamine, and triprolidine; (7) an anticholinergic including but not limited to: atropine, benztropine, biperiden, flutropium, hyoscyamine (e.g. Levsin®; Levbid®, Levsin/SL®, Anaspaz®, Levsinex Timecaps®, NuLev®), ilutropium, ipratropium, ipratropium bromide, methscopolamine, oxybutinin, rispenzepine, scopolamine, and tiotropium; (8) an anti-tussive including but not limited to: dextromethorphan, codeine, and hydromorphone; (9) a decongestant including but not limited to: pseudoephedrine and phenylpropanolamine; (10) an expectorant including but not limited to: guafenesin, guaicolsulfate, terpin, ammonium chloride, glycerol guaicolate, and iodinated glycerol; (11) a bronchodilator including but not limited to: theophylline and aminophylline; (12) an anti-inflammatory including but not limited to: fluribiprofen, diclophenac, indomethacin, ketoprofen, S-ketroprophen, tenoxicam; (13) a PDE (phosphodiesterase) inhibitor including but not limited to those disclosed herein; (14) a recombinant humanized monoclonal antibody [e.g. xolair (also called omalizumab), rhuMab, and talizumab]; (15) a humanized lung surfactant including recombinant forms of surfactant proteins SP-B, SP-C or SP-D [e.g. SURFAXIN®, formerly known as dsc-104 (Discovery Laboratories)], (16) agents that inhibit epithelial sodium channels (ENaC) such as amiloride and related compounds; (17) antimicrobial agents used to treat pulmonary infections such as acyclovir, amikacin, amoxicillin, doxycycline, trimethoprin sulfamethoxazole, amphotericin B, azithromycin, clarithromycin, roxithromycin, clarithromycin, cephalosporins (ceffoxitin, cefmetazole etc), ciprofloxacin, ethambutol, gentimycin, ganciclovir, imipenem, isoniazid, itraconazole, penicillin, ribavirin, rifampin, rifabutin, amantadine, rimantadine, streptomycin, tobramycin, and vancomycin; (18) agents that activate chloride secretion through Ca++ dependent chloride channels (such as purinergic receptor (P2Y(2) agonists); (19) agents that decrease sputum viscosity, such as human recombinant DNase 1, (Pulmozyme®); (20) nonsteroidal anti-inflammatory agents (acemetacin, acetaminophen, acetyl salicylic acid, alclofenac, alminoprofen, apazone, aspirin, benoxaprofen, bezpiperylon, bucloxic acid, carprofen, clidanac, diclofenac, diclofenac, diflunisal, diflusinal, etodolac, fenbufen, fenbufen, fenclofenac, fenclozic acid, fenoprofen, fentiazac, feprazone, flufenamic acid, flufenisal, flufenisal, fluprofen, flurbiprofen, flurbiprofen, furofenac, ibufenac, ibuprofen, indomethacin, indomethacin, indoprofen, isoxepac, isoxicam, ketoprofen, ketoprofen, ketorolac, meclofenamic acid, meclofenamic acid, mefenamic acid, mefenamic acid, miroprofen, mofebutazone, nabumetone oxaprozin, naproxen, naproxen, niflumic acid, oxaprozin, oxpinac, oxyphenbutazone, phenacetin, phenylbutazone, phenylbutazone, piroxicam, piroxicam, pirprofen, pranoprofen, sudoxicamn, tenoxican, sulfasalazine, sulindac, sulindac, suprofen, tiaprofenic acid, tiopinac, tioxaprofen, tolfenamic acid, tolmetin, tolmetin, zidometacin, zomepirac, and zomepirac); and (21) aerosolized antioxidant therapeutics such as S-Nitrosoglutathione.

### Anti-Obesity Agents

The GCRA peptide described herein can be used in combination therapy with an anti-obesity agent. Suitable such agents include, but are not limited to: 1 1β HSD-I (11-beta hydroxy steroid dehydrogenase type 1) inhibitors, such as BVT 3498, BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][1 1]annulene, and those compounds disclosed in WO01/90091, WOO 1/90090, WOO 1/90092 and WO2/072084; 5HT antagonists such as those in WO03/037871, WO03/037887, and the like; 5HTIa modulators such as carbidopa, benserazide and those disclosed in U.S. Pat. No. 6,207,699, WO03/031439, and the like; 5HT2c (serotonin receptor 2c) agonists, such as BVT933, DPCA37215, IK264, PNU 22394, WAY161503, R-1065, SB 243213 (Glaxo Smith Kline) and YM 348 and those disclosed in U.S. Pat. No. 3,914,250, WO00/77010, WO02/36596, WO02/48124, WO2/10169, WO1/66548, WO02/44152, WO02/51844, WO02/40456, and WO02/40457; 5HT6 receptor modulators, such as those in WO03/030901, WO03/035061, WO03/039547, and the like; acyl-estrogens, such as oleoyl-estrone, disclosed in del Mar-Grasa, M. et al, Obesity Research, 9:202-9 (2001) and Japanese Patent Application No. JP 2000256190; anorectic bicyclic compounds such as 1426 (Aventis) and 1954 (Aventis), and the compounds disclosed in WO00/18749, WO01/32638, WO01/62746, WO01/62747, and WO3/015769; CB 1 (cannabanoid-1 receptor) antagonist/inverse agonists such as rimonabant (Acomplia, Sanofi), SR-147778 (Sanofi), SR-141716 (Sanofi), BAY 65-2520 (Bayer), and SLV 319 (Solvay), and those disclosed in patent publications U.S. Pat. No. 4,973,587, U.S. Pat. No. 5,013,837, U.S. Pat. No. 5,081,122, U.S. Pat. No. 5,112,820, U.S. Pat. No. 5,292,736, U.S. Pat. No. 5,532,237, U.S. Pat. No. 5,624,941, U.S. Pat. No. 6,028,084, U.S. Pat. No. 6,509,367, U.S. Pat. No. 6,509,367, WO96/33159, WO97/29079, WO98/31227, WO98/33765, WO98/37061, WO98/41519, WO98/43635, WO98/43636, WO99/02499, WO00/10967, WO00/10968, WO01/09120, WO01/58869, WO01/64632, WO01/64633, WO01/64634, WO01/70700, WO01/96330, WO02/076949, WO03/006007, WO03/007887, WO03/020217, WO03/026647, WO03/026648, WO03/027069, WO03/027076, WO03/027114, WO03/037332, WO03/040107, WO03/086940, WO03/084943 and EP658546; CCK-A (cholecystokinin-A) agonists, such as AR-R 15849, GI 181771 (GSK), JMV-180, A-71378, A-71623 and SR146131 (Sanofi), and those described in U.S. Pat. No. 5,739,106; CNTF (Ciliary neurotrophic factors), such as GI-181771 (Glaxo-SmithKline), SRI 46131 (Sanofi Synthelabo), butabindide, PD 170.292, and PD 149164 (Pfizer), CNTF derivatives, such as Axokine® (Regeneron), and those disclosed in WO94/09134, WO98/22128, and WO99/43813; dipeptidyl peptidase IV (DP-IV) inhibitors, such as isoleucine thiazolidide, valine pyrrolidide, NVP-DPP728, LAF237, P93/01, P 3298, TSL 225 (tryptophyl-1,2,3,4-tetrahydroisoquinoline-3-carboxylic acid; disclosed by Yamada et al, Bioorg. & Med. Chem. Lett. 8 (1998) 1537-1540), TMC-2A/2B/2C, CD26 inhibitors, FE 999011, P9310/K364, VIP 0177, SDZ 274-444, 2-cyanopyrrolidides and 4-cyanopyrrolidides as disclosed by Ashworth et al, Bioorg. & Med. Chem. Lett, Vol 6, No. 22, pp 1163-1166 and 2745-2748 (1996) and the compounds disclosed patent publications. WO99/38501, WO99/46272, WO99/67279 (Probiodrug), WO099/67278 (Probiodrug), WO99/61431 (Probiodrug), WO02083128, WO02/062764, WO003/000180, WO03/000181, WO03/000250, WO03/002530, WO03/002531, WO03/002553, WO03/002593, WO03/004498, WO03/004496, WO03/017936, WO03/024942, WO03/024965, WO03/033524. WO03/037327 and EP1258476, growth hormone secretagogue receptor agonists/antagonists, such as NN703, hexarelin, MK-0677 (Merck), SM-130686, CP-424391 (Pfizer), LY 444,711 (Eli Lilly), L-692,429 and L-163.255, and such as those disclosed in U.S. Ser. No. 09/662,448, U.S. provisional application 60/203,335, U.S. Pat. No. 6,358,951, US2002049196, US2002/022637, WO01/56592 and WO02/32888, H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, O-[3-(1H-imidazol-4-yl)propanol]carbamates (Kiec-Kononowicz, K et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A et al., Arch Pharm. (Weinheim) 334:45-52 (2001)), substituted N-phenylcarbamates (Reidemeister. S. et al., Pharmazie, 55:83-6 (2000)), and proxifan derivatives (Sasse, A. et al., J. Med. Chem. 43:3335-43 (2000)) and histamine H3 receptor modulators such as those disclosed in WO02/15905, WO03/024928 and WO03/024929; leptin derivatives, such as those disclosed in U.S. Pat. No. 5,552,524. U.S. Pat. No. 5,552,523, U.S. Pat. No. 5,552,522, U.S. Pat. No. 5,521,283. WO96/23513, WO96/23514, WO96/23515, WO96/23516, WO96/23517, WO96/23518, WO96/23519, and WO96/23520; leptin, including recombinant human leptin (PEG-OB, Hoffman La Roche) and recombinant methionyl human leptin (Amgen), lipase inhibitors, such as tetrahydrolipstatin (orlistat/Xenical®), Triton WRI 339, RHC80267, lipstatin, teasaponin, diethylumbelliferyl phosphate, FL-386, WAY-121898, Bay-N-3176, valilactone, esteracin, ebelactone A, ebelactone B, and RHC 80267, and those disclosed in patent publications WO01/77094, U.S. Pat. No. 4,598,089, U.S. Pat. No. 4,452,813, U.S. Pat. No. 5,512,565, U.S. Pat. No. 5,391,571, U.S. Pat. No. 5,602,151, U.S. Pat. No. 4,405,644, U.S. Pat. No. 4,189,438, and U.S. Pat. No. 4,242,453; lipid metabolism modulators such as maslinic acid, erythrodiol, ursolic acid uvaol, betulinic acid, betulin, and the like and compounds disclosed in WO03/011267; Mc4r (melanocortin 4 receptor) agonists, such as CHIR86036 (Chiron), ME-10142, ME-10145, and HS-131 (Melacure), and those disclosed in PCT publication Nos. WO99/64002, WO00/74679, WOO 1/991752, WOO 1/25192, WOO 1/52880, WOO 1/74844, WOO 1/70708, WO01/70337, WO01/91752, WO02/059095, WO02/059107, WO02/059108, WO02/059117, WO02/06276, WO02/12166, WO02/11715, WO02/12178, WO02/15909, WO02/38544, WO02/068387, WO02/068388, WO02/067869, WO002/081430, WO03/06604, WO03/007949, WO03/009847, WO03/009850, WO03/013509, and WO03/031410; Me5r (melanocortin 5 receptor) modulators, such as those disclosed in WO97/19952, WO00/15826, WO00/15790, US20030092041, melanin-concentrating hormone 1 receptor (MCHR) antagonists, such as T-226296 (Takeda), SB 568849, SNP-7941 (Synaptic), and those disclosed in patent publications WOO 1/21169, WO01/82925, WO01/87834, WO02/051809, WO02/06245, WO02/076929, WO02/076947, WO02/04433, WO02/51809, WO02/083134, WO02/094799, WO03/004027, WO03/13574, WO03/15769, WO03/028641, WO03/035624, WO03/033476, WO03/033480, JP13226269, and JP1437059; mGluR5 modulators such as those disclosed in WO03/029210, WO03/047581, WO03/048137, WO03/051315, WO03/051833, WO03/053922, WO03/059904, and the like; serotoninergic agents, such as fenfluramine (such as Pondimin® (Benzeneethanamine, N-ethyl-alpha-methyl-3-(trifluoromethyl)-, hydrochloride), Robbins), dexfenfluramine (such as Redux® (Benzeneethanamine, N-ethyl-alpha-methyl-3-(trifluoromethyl)-, hydrochloride), Interneuron) and sibutramine ((Meridia®, Knoll/Reductil™) including racemic mixtures, as optically pure isomers (+) and (−), and pharmaceutically acceptable salts, solvents, hydrates, clathrates and prodrugs thereof including sibutramine hydrochloride monohydrate salts thereof, and those compounds disclosed in U.S. Pat. No. 4,746,680, U.S. Pat. No. 4,806,570, and U.S. Pat. No. 5,436,272, US20020006964, WOO 1/27068, and WOO 1/62341; NE (norepinephrine) transport inhibitors, such as GW 320659, desipramine, talsupram, and nomifensine; NPY 1 antagonists, such as BIBP3226, J-115814, BIBO 3304, LY-357897, CP-671906, GI-264879A, and those disclosed in U.S. Pat. No. 6,001,836, WO96/14307, WO01/23387, WO99/51600, WO01/85690, WO01/85098, WO01/85173, and WO01/89528; NPY5 (neuropeptide Y Y5) antagonists, such as 152,804, GW-569180A, GW-594884A, GW-587081X, GW-548118X, FR235208, FR226928, FR240662, FR252384, 1229U91, GI-264879A, CGP71683A, LY-377897, LY-366377, PD-160170, SR-120562A, SR-120819A, JCF-104, and H409/22 and those compounds disclosed in patent publications U.S. Pat. No. 6,140,354, U.S. Pat. No. 6,191,160, U.S. Pat. No. 6,218,408, U.S. Pat. No. 6,258,837, U.S. Pat. No. 6,313,298, U.S. Pat. No. 6,326,375, U.S. Pat. No. 6,329,395, U.S. Pat. No. 6,335,345, U.S. Pat. No. 6,337,332, U.S. Pat. No. 6,329,395, U.S. Pat. No. 6,340,683, EP01010691, EP-01044970, WO97/19682, WO97/20820, WO97/20821, WO97/20822, WO97/20823, WO98/27063, WO00/107409, WO00/185714. WO00/185730, WO00/64880, WO00/68197, WO00/69849, WO/0113917, WO01/09120, WO01/14376, WO01/85714, WO01/85730, WO01/07409, WO01/02379, WO01/23388, WO01/23389, WOO 1/44201, WO01/62737, WO01/62738, WO01/09120, WO02/20488, WO02/22592, WO02/48152, WO02/49648, WO02/051806, WO02/094789, WO03/009845, WO03/014083, WO03/022849, WO03/028726 and Norman et al, J. Med. Chem. 43:4288-4312 (2000), opioid antagonists, such as nalmefene (REVEX®), 3-methoxynaltrexone, methylnaltrexone, naloxone, and naltrexone (e.g. PT901; Pain Therapeutics, Inc.) and those disclosed in US20050004155 and WO00/21509; orexin antagonists, such as SB-334867-A and those disclosed in patent publications WO01/96302, WO01/68609, WO02/44172, WO02/51232, WO02/51838, WO02/089800, WO02/090355, WO03/023561, WO03/032991, and WO03/037847; PDE inhibitors (e.g. compounds which slow the degradation of cyclic AMP (cAMP) and/or cyclic GMP (cGMP) by inhibition of the phosphodiesterases, which can lead to a relative increase in the intracellular concentration of cAMP and cGMP; possible PDE inhibitors are primarily those substances which are to be numbered among the class consisting of the PDE3 inhibitors, the class consisting of the PDE4 inhibitors and/or the class consisting of the PDE5 inhibitors, in particular those substances which can be designated as mixed types of PDE3/4 inhibitors or as mixed types of PDE3/4/5 inhibitors) such as those disclosed in patent publications DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EPO1 12987, EPO1 16948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. No. 4,963,561, U.S. Pat. No. 5,141,931, WO9117991, WO9200968, WO9212961, WO9307146, WO9315044, WO9315045, WO9318024, WO9319068, WO9319720, WO9319747, WO9319749, WO09319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO09535281, WO9535282, WO9600218, WO9601825, WO9602541, WO9611917, DE3142982, DEl 116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577), WO9307124, EP0163965, EP0393500, EP0510562, EP0553174, WO9501338 and WO9603399, as well as PDE5 inhibitors (such as RX-RA-69, SCH-51866, KT-734, vesnarinone, zaprinast, SKF-96231, ER-21355, BF/GP-385, NM-702 and sildenafil (Viagra™)), PDE4 inhibitors (such as etazolate, IC163197, RP73401, imdazolidinone (RO-20-1724), MEM 1414 (R1533/R1500, Pharmacia Roche), denbufylline, rolipram, oxagrelate, nitraquazone, Y-590, DH-6471, SKF-94120, motapizone, lixazinone, indolidan, olprinone, atizoram, KS-506-G, dipamfylline, BMY-43351, atizoram, arofylline, filaminast, PDB-093, UCB-29646, CDP-840, SKF-107806, piclamilast, RS-17597, RS-25344-000, SB-207499, TIBENELAST, SB-210667, SB-211572, SB-211600, SB-212066, SB-212179, GW-3600, CDP-840, mopidamol, anagrelide, ibudilast, amrinone, pimobendan, cilostazol, quazinone and N-(3,5-dichloropyrid-4-yl)-3-cyclopropylmethoxy4-difluoromethoxybenzamide, PDE3 inhibitors (such as IC1153, 100, bemorandane (RWJ 22867), MCI-154, UD-CG 212, sulmazole, ampizone, cilostamide, carbazeran, piroximone, imazodan, CI-930, siguazodan, adibendan, saterinone, SKF-95654, SDZ-MKS-492, 349-U-85, emoradan, EMD-53998, EMD-57033, NSP-306, NSP-307, revizinone, NM-702, WIN-62582 and WIN-63291, enoximone and milrinone, PDE3/4 inhibitors (such as benafentrine, trequinsin, ORG-30029, zardaverine, L-686398, SDZ-ISQ-844, ORG-20241, EMD-54622, and tolafentrine) and other PDE inhibitors (such as vinpocetin, papaverine, enprofylline, cilomilast, fenoximone, pentoxifylline, roflumilast, tadalafil (Cialis®), theophylline, and vardenafil (Levitra®); Neuropeptide Y2 (NPY2) agonists include but are not limited to: polypeptide YY and fragments and variants thereof (e.g. YY3-36 (PYY3-36) (N. Engl. J. Med. 349,941, 2003; IKPEAPGE DASPEELNRY YASLRHYLNL VTRQRY (SEQ ID NO: 12)) and PYY agonists such as those disclosed in WO02/47712, WO03/026591, WO03/057235, and WO03/027637; serotonin reuptake inhibitors, such as, paroxetine, fluoxetine (Prozac™), fluvoxamine, sertraline, citalopram, and imipramine, and those disclosed in U.S. Pat. No. 6,162,805, U.S. Pat. No. 6,365,633, WO03/00663, WOO 1/27060, and WOO 1/162341, thyroid hormone β agonists, such as KB-2611 (KaroBioBMS), and those disclosed in WO02/15845, WO97/21993, WO99/00353, GB98/284425, U.S. Provisional Application No. 60/183,223, and Japanese Patent Application No. JiP 2000256190; UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid, and those disclosed in WO99/00123; β3 (beta adrenergic receptor 3) agonists, such as AJ9677/TAK677 (Dainippon/Takeda), L750355 (Merck), CP331648 (Pfizer), CL-316,243, SB 418790, BRL-37344, L-796568, BMS-196085, BRL-35135A, CGP12177A, BTA-243, GW 427353, Trecadrine, Zeneca D7114, N-5984 (Nisshin Kyorin), LY-377604 (Lilly), SR 59119A, and those disclosed in U.S. Pat. No. 5,541,204, U.S. Pat. No. 5,770,615, U.S. Pat. No. 5,491,134, U.S. Pat. No. 5,776,983, U.S. Pat. No. 4,880,64, U.S. Pat. No. 5,705,515, U.S. Pat. No. 5,451,677, WO94/18161, WO95/29159, WO97/46556, WO98/04526 and WO98/32753, WO01/74782, WO02/32897, WO03/014113, WO03/016276, WO03/016307, WO03/024948, WO03/024953 and WO03/037881; noradrenergic agents including, but not limited to, diethylpropion (such as Tenuate® (1-propanone, 2-(diethylamino)-1-phenyl-, hydrochloride), Merrell), dextroamphetamine (also known as dextroamphetamine sulfate, dexamphetamine, dexedrine, Dexampex, Ferndex, Oxydess II, Robese, Spancap #1), mazindol ((or 5-(p-chlorophenyl)-2,5-dihydro-3H-imidazo[2,1-a]isoindol-5-ol) such as Sanorex®, Novartis or Mazanor®, Wyeth Ayerst), phenylpropanolamine (or Benzenemethanol, alpha-(1-aminoethyl)-, hydrochloride), phentermine ((or Phenol, 3-[[4,5-duhydro-1H-imidazol-2-yl)ethyl](4-methylphenyl-1)amino], monohydrochloride) such as Adipex-P®, Lemmon, FASTIN®, SmithKline Beecham and Ionamin®, Medeva), phendimetrazine ((or (2S,3S)-3,4-Dimethyl-2phenylmorpholine L-(+)-tartrate (1:1)) such as Metra® (Forest), Plegine® (Wyeth-Ay erst), Prelu-2® (Boehringer Ingelheim), and Statobex® (Lemmon), phendamine tartrate (such as Thephorin® (2,3,4,9-Tetrahydro-2-methyl-9-phenyl-1H-indenol[2,1-c]pyridine L-(+)-tartrate (1:1)), Hoffmann-LaRoche), methamphetamine (such as Desoxyn®, Abbot ((S)-N, (alpha)-dimethylbenzeneethanamine hydrochloride)), and phendimetrazine tartrate (such as Bontril® Slow-Release Capsules, Amarin (-3,4-Dimethyl-2-phenylmorpholine Tartrate); fatty acid oxidation upregulator/inducers such as Famoxin® (Genset); monamine oxidase inhibitors including but not limited to befloxatone, moclobemide, brofaromine, phenoxathine, esuprone, befol, toloxatone, pirlindol, amiflamine, sercloremine, bazinaprine, lazabemide, milacemide, caroxazone and other certain compounds as disclosed by WO01/12176; and other anti-obesity agents such as 5HT-2 agonists, ACC (acetyl-CoA carboxylase) inhibitors such as those described in WO03/072197, alpha-lipoic acid (alpha-LA), AOD9604, appetite suppressants such as those in WO03/40107, ATL-962 (Alizyme PLC), benzocaine, benzphetamine hydrochloride (Didrex), bladderwrack (focus vesiculosus), BRS3 (bombesin receptor subtype 3) agonists, bupropion, caffeine, CCK agonists, chitosan, chromium, conjugated linoleic acid, corticotropin-releasing hormone agonists, dehydroepiandrosterone, DGAT1 (diacylglycerol acyltransferase 1) inhibitors, DGAT2 (diacylglycerol acyltransferase 2) inhibitors, dicarboxylate transporter inhibitors, ephedra, exendin-4 (an inhibitor of glp-1) FAS (fatty acid synthase) inhibitors (such as Cerulenin and C75), fat resorption inhibitors (such as those in WO03/053451, and the like), fatty acid transporter inhibitors, natural water soluble fibers (such as psyllium, plantago, guar, oat, pectin), galanin antagonists, galega (Goat's Rue, French Lilac), garcinia cambogia, germander (teucrium chamaedrys), ghrelin antibodies and ghrelin antagonists (such as those disclosed in WO01/87335, and WO02/08250), polypeptide hormones and variants thereof which affect the islet cell secretion, such as the hormones of the secretin/gastric inhibitory polypeptide (GIP)/vasoactive intestinal polypeptide (VIP)/pituitary adenylate cyclase activating polypeptide (PACAP)/glucagon-like polypeptide II (GLP-II)/glicentin/glucagon gene family and/or those of the adrenomedullin/amylin/calcitonin gene related polypeptide (CGRP) gene family including GLP-1 (glucagon-like polypeptide 1) agonists (e.g. (1) exendin-4, (2) those GLP-I molecules described in US20050130891 including GLP-1(7-34), GLP-1(7-35), GLP-1(7-36) or GLP-1(7-37) in its C-terminally carboxylated or amidated form or as modified GLP-I polypeptides and modifications thereof including those described in paragraphs 17-44 of US20050130891, and derivatives derived from GLP-1-(7-34)COOH and the corresponding acid amide are employed which have the following general formula: R—NH-HAEGTFTSDVSYLEGQAAKEFIAWLVK-CONH₂ wherein R═H or an organic compound having from 1 to 10 carbon atoms (SEQ ID NO: 13). Preferably, R is the residue of a carboxylic acid. Particularly preferred are the following carboxylic acid residues: formyl, acetyl, propionyl, isopropionyl, methyl, ethyl, propyl, isopropyl, n-butyl, sec-butyl, tert-butyl) and glp-1 (glucagon-like polypeptide-1), glucocorticoid antagonists, glucose transporter inhibitors, growth hormone secretagogues (such as those disclosed and specifically described in U.S. Pat. No. 5,536,716), interleukin-6 (IL-6) and modulators thereof (as in WO03/057237, and the like), L-carnitine. Mc3r (melanoconin 3 receptor) agonists, MCH2R (melanin concentrating hormone 2R) agonist/antagonists, melanin concentrating hormone antagonists, melanocortin agonists (such as Melanotan II or those described in WO 99/64002 and WO 00/74679), nomame herba, phosphate transporter inhibitors, phytopharm compound 57 (CP 644,673), pyruvate, SCD-I (stearoyl-CoA desaturase-1) inhibitors, T71 (Tularik, Inc., Boulder Colo.), Topiramate (Topimax®, indicated as an anti-convulsant which has been shown to increase weight loss), transcription factor modulators (such as those disclosed in WO03/026576), β-hydroxy steroid dehydrogenase-1 inhibitors (β-HSD-1), β-hydroxy-β-methylbutyrate, p57 (Pfizer), Zonisamide (Zonegran™, indicated as an anti-epileptic which has been shown to lead to weight loss), and the agents disclosed in US20030119428 paragraphs 20-26.

### Anti-Diabetic Agents

The GCRA peptide described herein can be used in therapeutic combination with one or more anti-diabetic agents, including but not limited to: PPARγ agonists such as glitazones (e.g., WAY-120,744, AD 5075, balaglitazone, ciglitazone, darglitazone (CP-86325. Pfizer), englitazone (CP-68722, Pfizer), isaglitazone (MIT/J&J), MCC-555 (Mitsubishi disclosed in U.S. Pat. No. 5,594,016), pioglitazone (such as such as Actos™ pioglitazone; Takeda), rosiglitazone (Avandia™; Smith Kline Beecham), rosiglitazone maleate, troglitazone (Rezulin®, disclosed in U.S. Pat. No. 4,572,912), rivoglitazone (CS-Ol 1, Sankyo), GL-262570 (Glaxo Welcome), BRL49653 (disclosed in WO98/05331), CLX-0921, 5-BTZD, GW-0207, LG-100641, JJT-501 (JPNT/P&U), L-895645 (Merck), R-119702 (Sankyo/Pfizer), NN-2344 (Dr. Reddy/NN), YM-440 (Yamanouchi), LY-300512, LY-519818, R483 (Roche), T131 (Tularik), and the like and compounds disclosed in U.S. Pat. No. 4,687,777, U.S. Pat. No. 5,002,953, U.S. Pat. No. 5,741,803, U.S. Pat. No. 5,965,584, U.S. Pat. No. 6,150,383, U.S. Pat. No. 6,150,384, U.S. Pat. No. 6,166,042, U.S. Pat. No. 6,166,043, U.S. Pat. No. 6,172,090, U.S. Pat. No. 6,211,205, U.S. Pat. No. 6,271,243, U.S. Pat. No. 6,288,095, U.S. Pat. No. 6,303,640, U.S. Pat. No. 6,329,404, U.S. Pat. No. 5,994,554, WO97/10813, WO97/27857, WO97/28115, WO97/28137, WO97/27847, WO00/76488, WO03/000685, WO03/027112, WO03/035602, WO03/048130, WO03/055867, and pharmaceutically acceptable salts thereof; biguanides such as metformin hydrochloride (N,N-dimethylimidodicarbonimidic diamide hydrochloride, such as Glucophage™, Bristol-Myers Squibb); metformin hydrochloride with glyburide, such as Glucovance™. Bristol-Myers Squibb); buformin (Imidodicarbonimidic diamide, N-butyl-); etoformine (1-Butyl-2-ethylbiguanide, Schering A. G.); other metformin salt forms (including where the salt is chosen from the group of, acetate, benzoate, citrate, ftimarate, embonate, chlorophenoxyacetate, glycolate, palmoate, aspartate, methanesulphonate, maleate, parachlorophenoxyisobutyrate, formate, lactate, succinate, sulphate, tartrate, cyclohexanecarboxylate, hexanoate, octanoate, decanoate, hexadecanoate, octodecanoate, benzenesulphonate, trimethoxybenzoate, paratoluenesulphonate, adamantanecarboxylate, glycoxylate, glutarnate, pyrrolidonecarboxylate, naphthalenesulphonate, 1-glucosephosphate, nitrate, sulphite, dithionate and phosphate), and phenformin; protein tyrosine phosphatase-IB (PTP-IB) inhibitors, such as A-401,674, KR 61639, OC-060062, OC-83839, OC-297962, MC52445, MC52453, ISIS 113715, and those disclosed in WO99/585521, WO99/58518, WO99/58522, WO99/61435, WO03/032916, WO03/032982, WO03/041729, WO03/055883, WO02/26707, WO02/26743, JP2002114768, and pharmaceutically acceptable salts and esters thereof; sulfonylureas such as acetohexamide (e.g. Dymelor, Eli Lilly), carbutamide, chlorpropamide (e.g. Diabinese®, Pfizer), gliamilide (Pfizer), gliclazide (e.g. Diamcron, Servier Canada Inc), glimepiride (e.g. disclosed in U.S. Pat. No. 4,379,785, such as Amaryl, Aventis), glipentide, glipizide (e.g. Glucotrol or Glucotrol XL Extended Release, Pfizer), gliquidone, glisolamide, glyburide/glibenclamide (e.g. Micronase or Glynase Prestab, Pharmacia & Upjohn and Diabeta, Aventis), tolazamide (e.g. Tolinase), and tolbutamide (e.g. Orinase), and pharmaceutically acceptable salts and esters thereof; meglitinides such as repaglinide (e.g. Pranidin®, Novo Nordisk), KAD1229 (PF/Kissei), and nateglinide (e.g. Starlix®, Novartis), and pharmaceutically acceptable salts and esters thereof; a glucoside hydrolase inhibitors (or glucoside inhibitors) such as acarbose (e.g. Precose™, Bayer disclosed in U.S. Pat. No. 4,904,769), miglitol (such as GLYSET™, Pharmacia & Upjohn disclosed in U.S. Pat. No. 4,639,436), camiglibose (Methyl 6-deoxy-6-[(2R,3R,4R,5S)-3,4,5-trihydroxy-2-(hydroxymethyl)piperidino]-alpha-D-glucopyranoside, Marion Merrell Dow), voglibose (Takeda), adiposine, emiglitate, pradimicin-Q, salbostatin, CKD-711, MDL-25,637, MDI-73,945, and MOR 14, and the compounds disclosed in U.S. Pat. No. 4,062,950, U.S. Pat. No. 4,174,439, U.S. Pat. No. 4,254,256, U.S. Pat. No. 4,701,559, U.S. Pat. No. 4,639,436, U.S. Pat. No. 5,192,772, U.S. Pat. No. 4,634,765, U.S. Pat. No. 5,157,116, U.S. Pat. No. 5,504,078, U.S. Pat. No. 5,091,418, U.S. Pat. No. 5,217,877, U.S. Pat. No. 5,1091 and WOO 1/47528 (polyamines); α-amylase inhibitors such as tendamistat, trestatin, and A1-3688, and the compounds disclosed in U.S. Pat. No. 4,451,455, U.S. Pat. No. 4,623,714, and U.S. Pat. No. 4,273,765; SGLT2 inhibitors including those disclosed in U.S. Pat. No. 6,414,126 and U.S. Pat. No. 6,515,117, an aP2 inhibitor such as disclosed in U.S. Pat. No. 6,548,529; insulin secretagogues such as linogliride, A-4166, forskilin, dibutyrl cAMP, isobutylmethylxanthine (IBMX), and pharmaceutically acceptable salts and esters thereof; fatty acid oxidation inhibitors, such as clomoxir, and etomoxir, and pharmaceutically acceptable salts and esters thereof, A2 antagonists, such as midaglizole, isaglidole, deriglidole, idazoxan, earoxan, and fluparoxan, and pharmaceutically acceptable salts and esters thereof; insulin and related compounds (e.g. insulin mimetics) such as biota, LP-100, novarapid, insulin detemir, insulin lispro, insulin glargine, insulin zinc suspension (lente and ultralente), Lys-Pro insulin, GLP-I (1-36) amide, GLP-I (73-7) (insulinotropin, disclosed in U.S. Pat. No. 5,614,492), LY-315902 (Lilly), GLP-I (7-36)-NH2), AL-401 (Autoimmune), certain compositions as disclosed in U.S. Pat. No. 4,579,730, U.S. Pat. No. 4,849,405, U.S. Pat. No. 4,963,526, U.S. Pat. No. 5,642,868, U.S. Pat. No. 5,763,396, U.S. Pat. No. 5,824,638, U.S. Pat. No. 5,843,866, U.S. Pat. No. 6,153,632, U.S. Pat. No. 6,191,105, and WO 85/05029, and primate, rodent, or rabbit insulin including biologically active variants thereof including allelic variants, more preferably human insulin available in recombinant form (sources of human insulin include pharmaceutically acceptable and sterile formulations such as those available from Eli Lilly (Indianapolis, Ind. 46285) as Humulin™ (human insulin rDNA origin), also see the THE PHYSICIAN'S DESK REFERENCE, 55.sup.th Ed. (2001) Medical Economics, Thomson Healthcare (disclosing other suitable human insulins); non-thiazolidinediones such as JT-501 and farglitazar (GW-2570/GI-262579), and pharmaceutically acceptable salts and esters thereof; PPARα/γ dual agonists such as AR-HO39242 (Aztrazeneca), GW-409544 (Glaxo-Welcome), BVT-142, CLX-0940, GW-1536, GW-1929, GW-2433, KRP-297 (Kyorin Merck; 5-[(2,4-Dioxo thiazolidinyl)methyl]methoxy-N-[[4-(trifluoromethyl)phenyl]methylbenzamide), L-796449, LR-90, MK-0767 (Merck/Kyorin/Banyu), SB 219994, muraglitazar (BMS), tesaglitzar (Astrazeneca), reglitazar (JTT-501) and those disclosed in WO99/16758, WO99/19313, WO99/20614, WO99/38850, WO00/23415, WO000/23417, WO00/23445, WO00/50414, WO01/00579, WO01/79150, WO02/062799, WO03/004458, WO03/016265, WO03/018010, WO3/033481, WO03/033450, WO03/033453, WO03/043985, WO 031053976, U.S. application Ser. No. 09/664,598, filed Sep. 18, 2000, Murakami et al. Diabetes 47, 1841-1847 (1998), and pharmaceutically acceptable salts and esters thereof; other insulin sensitizing drugs; VPAC2 receptor agonists; GLK modulators, such as those disclosed in WO03/015774, retinoid modulators such as those disclosed in WO03/000249, GSK 3β/GSK 3 inhibitors such as 4-[2-(2-bromophenyl)-4-(4-fluorophenyl-1H-imidazol-5-yl]pyridine and those compounds disclosed in WO03/024447, WO03/037869, WO03/037877, WO03/037891, WO03/068773, EP1295884, EP1295885, and the like, glycogen phosphorylase (HGLPa) inhibitors such as CP-368,296, CP-316,819, BAYR3401, and compounds disclosed in WOO 1/94300, WO02/20530, WO03/037864, and pharmaceutically acceptable salts or esters thereof; ATP consumption promoters such as those disclosed in WO03/007990; TRB3 inhibitors; vanilloid receptor ligands such as those disclosed in WO03/049702; hypoglycemic agents such as those disclosed in WO03/015781 and WO003/040114; glycogen synthase kinase 3 inhibitors such as those disclosed in WO03/035663 agents such as those disclosed in WO99/51225, US20030134890, WO01/24786, and WO03/059870; insulin-responsive DNA binding protein-1 (IRDBP-I) as disclosed in WO03/057827, and the like, adenosine A2 antagonists such as those disclosed in WO03/035639, WO03/035640, and the like; PPARB agonists such as GW 501516, GW 590735, and compounds disclosed in JP10237049 and WO02/14291; dipeptidyl peptidase IV (DP-IV) inhibitors, such as isoleucine thiazolidide, NVP-DPP728A (1-[[[2-[(5-cyanopyridin-2-yl)amino]ethyl]amino]acetyl]-2-cyano-(S)-pyrrolidine, disclosed by Hughes et al, Biochemistry, 38(36), 11597-11603, 1999), P32/98, NVP-LAF-237, P3298, TSL225 (tryptophyl-1,2,3,4-tetrahydro-isoquinoline-3-carboxylic acid, disclosed by Yamada et al, Bioorg. & Med. Chem. Lett. 8 (1998) 1537-1540), valine pyrrolidine, TMC-2A/2B/2C, CD-26 inhibitors, FE999011, P9310/K364, VIP 0177, DPP4, SDZ 274-444, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996), and the compounds disclosed in U.S. Pat. No. 6,395,767, U.S. Pat. No. 6,573,287, U.S. Pat. No. 6,395,767 (compounds disclosed include BMS-477118, BMS-471211 and BMS 538,305), WO99/38501, WO99/46272, WO99/67279, WO99/67278, WO99/61431 WO03/004498, WO03/004496. EP1258476, WO02/083128, WO02/062764, WO03/000250, WO03/002530, WO03/002531, WO03/002553, WO03/002593, WO03/000180, and WO03/000181, GLP-I agonists such as exendin-3 and exendin-4 (including the 39 as polypeptide synthetic exendin-4 called Exenatide®), and compounds disclosed in US2003087821 and NZ 504256, and pharmaceutically acceptable salts and esters thereof; peptides including amlintide and Symlin® (pramlintide acetate); and glycokinase activators such as those disclosed in US2002103199 (fused heteroaromatic compounds) and WO02/48106 (isoindolin-1-one-substituted propionamide compounds).

Phosphodiesterase Inhibitors

The GCRA peptide described herein can be used in combination therapy with a phosphodiesterase inhibitor. PDE inhibitors are those compounds which slow the degradation of cyclic AMP (cAMP) and/or cyclic GMP (cGMP) by inhibition of the phosphodiesterases, which can lead to a relative increase in the intracellular concentration of c AMP and/or cGMP. Possible PDE inhibitors are primarily those substances which are to be numbered among the class consisting of the PDE3 inhibitors, the class consisting of the PDE4 inhibitors and/or the class consisting of the PDE5 inhibitors, in particular those substances which can be designated as mixed types of PDE3/4 inhibitors or as mixed types of PDE3/4/5 inhibitors. By way of example, those PDE inhibitors may be mentioned such as are described and/or claimed in the following patent applications and patents: DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436. EP0096517, EPO1 12987, EPO1 16948, EP0150937, EP0158380, EP0161632, EP0161918. EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. Nos. 4,963,561, 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DEl 116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577) and WO9307124, EP0163965, EP0393500, EP0510562, EP0553174, WO09501338 and WO9603399. PDE5 inhibitors which may be mentioned by way of example are RX-RA-69, SCH-51866, KT-734, vesnarinone, zaprinast, SKF-96231, ER-21355, BF/GP-385, NM-702 and sildenafil (Viagra®). PDE4 inhibitors which may be mentioned by way of example are RO-20-1724, MEM 1414 (R1533/R1500; Pharmacia Roche), DENBUFYLLINE, ROLIPRAM, OXAGRELATE. NITRAQUAZONE, Y-590, DH-6471, SKF-94120, MOTAPIZONE, LIXAZINONE, INDOLIDAN, OLPRINONE, ATIZORAM, KS-506-G, DIPAMFYLLINE, BMY-43351, ATIZORAM, AROFYLLINE, FILAMINAST, PDB-093, UCB-29646, CDP-840, SKF-107806, PICLAMILAST, RS-17597, RS-25344-000, SB-207499, TIBENELAST, SB-210667, SB-211572, SB-211600, SB-212066, SB-212179, GW-3600, CDP-840, MOPIDAMOL, ANAGRELIDE, IBUDILAST, AMRINONE, PIMOBENDAN, CILOSTAZOL, QUAZINONE and N-(3,5-dichloropyrid-4-yl)-3-cyclopropylmethoxy4-ditfluoromethoxybenzamide, PDE3 inhibitors which may be mentioned by way of example are SULMAZOLE. AMPIZONE, CILOSTAMIDE, CARBAZERAN, PIROXIMONE, IMAZODAN, CI-930, SIGUAZODAN, ADIBENDAN, SATERINONE, SKF-95654, SDZ-MKS-492, 349-U-85, EMORADAN, EMD-53998, EMD-57033, NSP-306, NSP-307, REVIZINONE, NM-702, WIN-62582 and WIN-63291, ENOXIMONE and MILRINONE. PDE3/4 inhibitors which may be mentioned by way of example are BENAFENTRINE, TREQUINSIN, ORG-30029, ZARDAVERINE, L-686398, SDZ-ISQ-844, ORG-20241, EMD-54622, and TOLAFENTRINE. Other PDE inhibitors include, cilomilast, pentoxifylline, roflumilast, tadalaffl (Cialis®), theophylline, and vardenafil (Levitra®), zaprinast (PDE5 specific).

### Anti-Uterine Contractions Agents

The GCRA peptide described herein can be used in combination therapy (for example, in order to decrease or inhibit uterine contractions) with a tocolytic agent including but not limited to beta-adrenergic agents, magnesium sulfate, prostaglandin inhibitors, and calcium channel blockers.

### Anti-Neoplastic Agents

The GCRA peptide described herein can be used in combination therapy with an antineoplastic agents including but not limited to alkylating agents, epipodophyllotoxins, nitrosoureas, antimetabolites, vinca alkaloids, anthracycline antibiotics, nitrogen mustard agents, and the like. Particular anti-neoplastic agents may include tamoxifen, taxol, etoposide and 5-fluorouracil.

The GCRA peptide described herein can be used in combination therapy (for example as in a chemotherapeutic composition) with an antiviral and monoclonal antibody therapies.

### Agents to Treat Congestive Heart Failure

The GCRA peptide described herein can be used in combination therapy (for example, in prevention/treatment of congestive heart failure or another method described herein) with the partial agonist of the nociceptin receptor ORL1 described by Dooley et al. (The Journal of Pharmacology and Experimental Therapeutics, 283 (2): 735-741, 1997). The agonist is a hexapeptide having the amino acid sequence Ac-RYY (RK) (WI) (RK)-NH2 (“the Dooley polypeptide”), where the brackets show allowable variation of amino acid residue. Thus Dooley polypeptide can include but are not limited to KYYRWR (SEQ ID NO: 14), RYYRWR (SEQ ID NO. 15), KWRYYR (SEQ ID NO: 16), RYYRWK (SEQ ID NO. 17), RYYRWK (all-D amin acids) (SEQ ID NO: 18), RYYRIK (SEQ ID NO: 19), RYYRIR (SEQ ID NO: 20), RYYKIK (SEQ ID NO: 21), RYYKIR (SEQ ID NO: 22), RYYKWR (SEQ ID NO: 23), RYYKWK (SEQ ID NO: 24), RYYRWR (SEQ ID NO: 15), RYYRWK (SEQ ID NO: 17), RYYRIK (SEQ ID NO: 19), RYYKWR (SEQ ID NO: 23), RYYKWK (SEQ ID NO: 24), RYYRWK (SEQ ID NO. 17) and KYYRWK (SEQ ID NO: 25), wherein the amino acid residues are in the L-form unless otherwise specified. The GCRA peptides described herein can also be used in combination therapy with polypeptide conjugate modifications of the Dooley polypeptide described in WO0198324.

### Fibrate

The GCRA peptide described herein can be used in combination therapy with a fibrate. The term “fibrate” is also interchangeably used herein and in the art with the term “fibric acid derivative,” and means any of the fibric acid derivatives useful in the methods described herein, e.g., fenofibrate. Fenofibrate is a fibrate compound, other examples of which include, for example, bezafibrate, beclofibrate, benzafibrate, binifibrate, ciprofibrate, clinofibrate, clofibrate, etofibrate, gemcabene, gemfibrozil, lifibrol, nicofibrate, pirifibrate, ronifibrate, simfibrate, theofibrate, etc.

### Lipid Altering Agents

The GCRA peptide described herein can be used in combination therapy with a lipid altering agent. As used herein the term “lipid altering agent” or “dyslipidemia agent” refers to compounds including, but not limited to, bile acid sequestrants such as cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colesevelam hydrochloride (such as WELCHOL® Tablets (polyallylamine hydrochloride) cross-linked with epichlorohydrin and alkylated with 1-bromodecane and (6-bromohexyl)-trimethylammonium bromide) which are available from Sankyo), colestipol (a copolymer of diethylenetriamine and 1-chloro-2,3-epoxypropane, such as COLESTID® tablets which are available from Pharmacia), dialkylaminoalkyl derivatives of a cross-linked dextran, LOCHOLEST®, DEAE-Sephadex (SECHOLEX®, POLICEXIDE®), water soluble derivatives such as 3,3-ioene, N-(cycloalkyl)alkylamines and poliglusam, insoluble quaternized polystyrenes, saponins and mixtures thereof and those bile acid sequestrants disclosed in WO97/11345, WO98/57652, U.S. Pat. No. 3,692,895, and U.S. Pat. No. 5,703,188. Suitable inorganic cholesterol sequestrants include bismuth salicylate plus montmorillonite clay, aluminum hydroxide and calcium carbonate antacids.

### HMG-CoA Reductase Inhibitors

The GCRA peptide described herein can be used in combination therapy with a HMG-CoA reductase inhibitor. HMG-CoA reductase inhibitors are dyslipidemic agents that can be used in therapeutic combinations with compounds described herein. Suitable HMG-CoA reductase inhibitors for use in therapeutic combination with a compounds described herein include: atorvastatin (LIPITOR®; disclosed in U.S. Pat. No. 4,681,893, U.S. Pat. No. 5,385,929 and U.S. Pat. No. 5,686,104), atorvastatin calcium (disclosed in U.S. Pat. No. 5,273,995), dihydrocompactin, (disclosed in U.S. Pat. No. 4,450,171), bervastatin (disclosed in U.S. Pat. No. 5,082,859), carvastatin, cerivastatin (BAYCOL®; disclosed in U.S. Pat. No. 5,006,530, U.S. Pat. No. 5,502,199, and U.S. Pat. No. 5,177,080), crilvastatin, dalvastatin (disclosed in EP738510A2), fluvastatin (LESCOL®; disclosed in U.S. Pat. No. 4,739,073 and US534772), glenvastatin, fluindostatin (disclosed in EP363934A1), velostatin (visinolin; disclosed in U.S. Pat. No. 4,448,784 and U.S. Pat. No. 4,450,171), lovastatin (mevinolin; MEVACOR® (Merck and Co.) and related compounds disclosed in U.S. Pat. No. 4,231,938), mevastatin (and related compound disclosed in U.S. Pat. No. 3,983,140), compactin (and related compounds disclosed in U.S. Pat. No. 4,804,770), pravastatin (also known as NK-104, itavastatin, nisvastatin, nisbastatin disclosed in U.S. Pat. No. 5,102,888), pravastatin (PRAVACHOL® (Bristol Myers Squibb) and related compounds disclosed in U.S. Pat. No. 4,346,227), rivastatin (sodium 7-(4-fluorophenyl)-2,6-diisopropyl-5-methoxymethylpyridin-3-yl)-3,5-dihydroxy-6-heptanoate), rosuvastatin (CRESTOR®; also known as ZD-4522 disclosed in U.S. Pat. No. 5,260,440), atavastatin, visastatin, simvastatin (ZOCOR® (Merck and Co.) and related compounds as disclosed in U.S. Pat. No. 4,448,784 and U.S. Pat. No. 4,450,171), simvastatin, CI-981, compounds disclosed in WO03/033481, U.S. Pat. No. 4,231,938, U.S. Pat. No. 4,444,784, U.S. Pat. No. 4,647,576, U.S. Pat. No. 4,686,237, U.S. Pat. No. 4,499,289, U.S. Pat. No. 4,346,227, U.S. Pat. No. 5,753,675, U.S. Pat. No. 4,613,610, EP0221025, and EP491226, and optical or geometric isomers thereof; and nontoxic pharmaceutically acceptable salts, N-oxides, esters, quaternary ammonium salts, and prodrugs thereof. In HMG-CoA reductase inhibitors where an open-acid form can exist, salt and ester forms may preferably be formed from the open-acid, and all such forms are included within the meaning of the term “HMG-CoA reductase inhibitor” as used herein. Pharmaceutically acceptable salts with respect to the HMG-CoA reductase inhibitor includes non-toxic salts of the compounds which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzim-idazole, diethylamine, piperazine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, pantothenate, phosphate/diphosphate, polygalacturonate, salicylate, stearate, subacetate, succinate, tannate, tartrate, teoclate, tosylate, triethiodide, and valerate.

Other dyslipidemic agents which can be used in therapeutic combination with a compound described herein include: HMG-CoA synthase inhibitors such as L-659,699 ((E E)-I 1-[3′R-(hydroxy-methyl)-4′-oxo-2′R-oxetanyl]-3,5,7R-trimethyl-2,4-undecadienoic acid) and those disclosed in U.S. Pat. No. 5,120,729, U.S. Pat. No. 5,064,856, and U.S. Pat. No. 4,847,271; cholesterol absorption inhibitors such as plant sterols, plant stanols and/or fatty acid esters of plant stanols such as sitostanol ester used in BENECOL® margarine, stanol esters, beta-sitosterol, and sterol glycosides such as tiqueside. Other cholesterol absorption inhibitors include 1,4-Diphenylazetidin-2-ones; 4-biarylyl-1-phenylazetidin-2-ones; 4-(hydroxyphenyl)azetidin-2-ones; 1,4-diphenyl-3-hydroxyalkyl-2-azetidinones; 4-biphenyl-1-phenylazetidin-2-ones; 4-biarylyl-1-phenylazetidin-2-ones; and 4-biphenylylazetidinones.acyl coenzyme A-cholesterol acyl transferase (ACAT) inhibitors such as avasimibe (Current Opinion in Investigational Drugs. 3(9):291-297 (2003)), eflucimibe, HL-004, lecimibe, DuP-128, KY505, SMP 797, CL-277,082 (Clin Pharmacol Ther. 48(2): 189-94 (1990)) and the like; and those disclosed in US55 10379, WO96/26948 and WO96/10559; CETP inhibitors such as JTT 705 identified as in Nature 406, (6792):203-7 (2000), torcetrapib (CP-529,414 described in US20030186952 and WO00/017164), CP 532,632, BAY63-2149, SC 591, SC 795, and the like including those described in Current Opinion in Investigational Drugs. 4(3):291-297 (2003) and those disclosed in J. Antibiot, 49(8): 815-816 (1996), and Bioorg. Med. Chem. Lett, 6:1951-1954 (1996) and patent publications US55 12548, U.S. Pat. No. 6,147,090, WO99/20302, WO99/14204, WO99/41237, WO95/04755, WO96/15141, WO96/05227, WO038721, EP796846, EP818197, EP818448, DE19704244, DE19741051, DE19741399, DE197042437, DE19709125, DE19627430, DE19832159, DE19741400, JP 11049743, and JP 09059155; squalene synthetase inhibitors such as squalestatin-1, TAK-475, and those disclosed in U.S. Pat. No. 4,871,721, U.S. Pat. No. 4,924,024, US57 12396 (α-phosphono-sulfonates), Biller et al (1988) J. Med. Chem., 31:1869 (e.g. isoprenoid (phosphinyl-methyl)phosphonates), Biller et al (1996) Current Pharmaceutical Design, 2:1, P. Ortiz de Montellano et al (1977) J. Med. Chem. 20:243 (terpenoid pyrophosphates), Corey and Volante (1976) J. Am. Chem. Soc, 98:1291 (farnesyl diphosphate analog A and presqualene pyrophosphate (PSQ-PP) analogs), McClard et al (1987) J.A.C.S., 109:5544 (phosphinylphosphonates), Capson, T. L., PhD dissertation, June, 1987, Dept. Med. Chem. U of Utah, Abstract, Table of Contents, pp 16, 17, 40-43, 48-51, Summary, (cyclopropanes), Curr. Op. Ther. Patents (1993) 861, and patent publications EP0567026A1, EP0645378A1, EP0645377A1, EP0611749A1, EP0705607A2, EP0701725A1, and WO96/09827; antioxidants such as probucol (and related compounds disclosed in U.S. Pat. No. 3,674,836), probucol derivatives such as AGI-1067 (and other derivatives disclosed in U.S. Pat. No. 6,121,319 and U.S. Pat. No. 6,147,250), tocopherol, ascorbic acid, β-carotene, selenium and vitamins such as vitamin B6 or vitamin B12 and pharmaceutically acceptable salts and esters thereof; PPARα agonists such as those disclosed in U.S. Pat. No. 6,028,109 (fluorophenyl compounds), WO00/75103 (substituted phenylpropionic compounds), WO98/43081 and fibric acid derivatives (fibrates) such as beclofibrate, benzafibrate, bezafibrate (C.A.S. Registry No. 41859-67-0, see U.S. Pat. No. 3,781,328), binifibrate (C.A.S. Registry No. 69047-39-8, see BE884722), ciprofibrate (C.A.S. Registry No. 52214-84-3, see U.S. Pat. No. 3,948,973), clinofibrate (C.A.S. Registry No. 30299-08-2, see U.S. Pat. No. 3,716,583), clofibrate (such as ethyl 2-(p-chlorophenoxy)-2-methyl-propionate, e.g. Atromid-S® capsules (Wyeth-Ayerst), etofibrate, fenofibrate (such as Tricor® micronized fenofibrate ((2-[4-(4-chlorobenzoyl)phenoxy]-2-methyl-propanoic acid, 1-methylethyl ester; Abbott Laboratories) or Lipanthyl® micronized fenofibrate (Labortoire Founier, France)), gemcabene, gemfibrozil (such as 5-(2,5-dimethylphenoxy)-2,2-dimethylpentanoic acid, e.g. Lopid® tablets (Parke Davis)), lifibrol, GW 7647, BM 170744, LY5 18674 and those fibrate and fibrate acid derivatives disclosed in WO03/033456, WO03/033481, WO03/043997, WO03/048116, WO03/053974, WO03/059864, and WO03/05875; FXR receptor modulators such as GW 4064, SR 103912, and the like; LXR receptor modulators such as GW 3965, T9013137, and XTC0179628, and those disclosed in US20030125357, WO03/045382, WO03/053352, WO03/059874, and the like; HM74 and HM74A (human HM74A is Genbank Accession No. AY148884 and rat HM74A is EMM_patAR09 8624) receptor agonists such as nicotinic acid (niacin) and derivatives thereof (e.g. compounds comprising a pyridine-3-carboxylate structure or a pyrazine-2-carboxylate structure, including acid forms, salts, esters, zwitterions and tautomers, where available) including but not limited to those disclosed in Wise et al (2003) J. Biol. Chem. 278: 9869 (e.g. 5-methylpyrazole-3-carboxylic acid and acifran (4,5-dihydro-5-methyl-4-oxo-5-phenyl-2-furan carboxylic acid pyridine-3-acetic acid)), as well as 5-methyl nicotinic acid, nicotinic acid, niceritrol, nicofuranose, acipimox (5-methylpyrazine-2-carboxylic acid 4-oxide), Niaspan® (niacin extended-release tablets; Kos) and those which can be easily identified by one skilled in the art which bind to and agonize the HM74A or HM74 receptor (for example using the assays disclosed in Wise et al (2003) J. Biol. Chem 278:9869 (nicotine binding and [355]-GTPyS binding assays), Soga et al (2003) Biochem. Biophys. Res. Comm. 303:364 (radiolabel binding assay using the HM74 receptor which could be adapted to the HM74A receptor), Tunaru et al (2003) Nature Medicine 9:352 (calcium mobilization assay using the HM74 receptor which could be adapted to the HM74A receptor) and U.S. Pat. No. 6,420,183 (FLIPR assays are described generally in and may be adapted to the HM74A or HM74 receptor); renin angiotensin system inhibitors; bile acid reabsorption inhibitors (bile acid reuptake inhibitors), such as BARI 1453, SC435, PHA384640, 58921, AZD7706, and the like; PPAR agonists (including partial agonists) such as GW 501516, and GW 590735, and those disclosed in U.S. Pat. No. 5,859,051 (acetophenols), WO03/024395, WO97/28149, WO01/79197, WO02/14291, WO02/46154, WO02/46176, WO02/076957, WO03/0 16291, WO03/033493, WO99/20275 (quinoline phenyl compounds), WO99/38845 (aryl compounds), WO00/63161 (1,4-disubstituted phenyl compounds), WO01/00579 (aryl compounds), WO01/12612 & WO01/12187 (benzoic acid compounds), and WO97/31907 (substituted 4-hydroxy-phenylalconic acid compound); sterol biosynthesis inhibitors such as DMP-565; triglyceride synthesis inhibitors; microsomal triglyceride transport (MTTP) inhibitors, such as inplitapide, LAB687, and CP346086, AEGR 733, implitapide and the like; HMG-CoA reductase gene expression inhibitors (e.g. compounds that decrease HMG-CoA reductase expression by affecting (e.g. blocking) transcription or translation of HMG-CoA reductase into protein or compounds that maybe biotransformed into compounds that have the aforementioned attributes by one or more enzymes in the cholesterol biosynthetic cascade or may lead to the accumulation of an isoprene metabolite that has the aforementioned activities (such regulation is readily determined by those skilled in the art according to standard assays (Methods of Enzymology, 110:9-19 1985))) such as those disclosed in U.S. Pat. No. 5,041,432 (certain 15-substituted lanosterol derivatives) and E. I. Mercer (1993) Prog. Lip. Res. 32:357 (oxygenated sterols that suppress the biosynthesis of HMG-CoA reductase); squalene epoxidase inhibitors such as NB-598 ((E)-N-ethyl-N-(6,6-dimethyl-2-hepten-4-y-nyl)-3-[(3,3′-bithiophen-5-yl)methoxy]benzene-methanamine hydrochloride); low density lipoprotein (LDL) receptor inducers such as HOE-402 (an imidazolidinyl-pyrimidine derivative that directly stimulates LDL receptor activity, see Huettinger et al (1993) Arterioscler. Thromb. 13:1005); platelet aggregation inhibitors; 5-LO or FLAP inhibitors; PPAR modulators (including compounds that may have multiple functionality for activating various combinations of PPARα, PPARγ, and PPARδ) such as those disclosed in U.S. Pat. No. 6,008,237, U.S. Pat. No. 6,248,781, U.S. Pat. No. 6,166,049, WO00/12491, WO00/218355, WO00/23415, WO00/23416, WO00/23425, WO00/23442, WO00/23445, WO00/23451, WO00/236331, WO00/236332, WO00/238553, WO00/50392, WO00/53563, WO00/63153, WO00/63190, WO00/63196, WO00/63209, WO00/78312, WO00/78313, WOO 1/04351, WO01/14349, WO01/14350, WO01/16120, WO01/17994, WO01/21181, WO01/21578, WOO 1/25 181, WO01/25225, WO01/25226, WO01/40192, WO01/79150, WO02/081428, WO02/100403, WO02/102780, WO02/79162, WO03/016265, WO03/033453, WO03/042194, WO03/043997, WO03/066581, WO97/25042, WO99/07357, WO99/11255, WO99/12534, WO99/15520, WO99/46232, and WO98/05331 (including GW233 1 or (2-(4-[difluorophenyl]-1 heptylureido)ethyl]phenoxy)-2-methylbutyric)); niacin-bound chromium, as disclosed in WO03/039535; substituted acid derivatives disclosed in WO03/040114; apolipoprotein B inhibitors such as those disclosed in WO02/090347, WO02/28835, WO03/045921, WO03/047575; Factor Xa modulators such as those disclosed in WO03/047517, WO03/047520, WO03/048081; ileal bile acid transport (“IBAT”) inhibitors (or apical sodium co-dependent bile acid transport (“ASBT”) inhibitors) such as benzothiepines (including 1,2-benzothiazepines; 1,4-benzodiazepines; 1,5-benzothiazepines; 1,2, 5-benzothiadiazepines); PPARδ activators such as disclosed in WOO 1/00603 (thiazole and oxazole derivates (e.g. C.A.S. Registry No. 317318-32-4), WO97/28149 (fluoro, chloro and thio phenoxy phenylacetic), U.S. Pat. No. 5,093,365 (non-1-oxidizable fatty acid analogues), and WO99/04815. Tests showing the efficacy of the therapy and the rationale for the combination therapy with a dyslipidemic agent are presented in US2003 0069221 (where the dyslipidemic agents are called ‘cardiovascular agents’)

Dosage

Dosage levels of active ingredients in a pharmaceutical composition can also be varied so as to achieve a transient or sustained concentration of the compound in a subject, especially in and around the site of inflammation or disease area, and to result in the desired response. It is well within the skill of the art to start doses of the compound at levels lower than required to achieve the desired effect and to gradually increase the dosage until the desired effect is achieved. It will be understood that the specific dose level for any particular subject will depend on a variety of factors, including body weight, general health, diet, natural history of disease, route and scheduling of administration, combination with one or more other drugs, and severity of disease.

An effective dosage of the composition will typically be between about 1 μg and about 10 mg per kilogram body weight, preferably between about 10 μg to 5 mg of the compound per kilogram body weight. Adjustments in dosage will be made using methods that are routine in the art and will be based upon the particular composition being used and clinical considerations.

The guanylate cyclase receptor agonists used in the methods described above may be administered orally, systemically or locally. Dosage forms include preparations for inhalation or injection, solutions, suspensions, emulsions, tablets, capsules, topical salves and lotions, transdermal compositions, other known peptide formulations and pegylated peptide analogs. Agonists may be administered as either the sole active agent or in combination with other drugs, e.g., an inhibitor of cGMP-dependent phosphodiesterase and anti-inflammatory agent. In all cases, additional drugs should be administered at a dosage that is therapeutically effective using the existing art as a guide. Drugs may be administered in a single composition or sequentially.

Dosage levels of the GCR agonist for use in methods of this invention typically are from about 0.001 mg to about 10,000 mg daily, preferably from about 0.005 mg to about 1,000 mg daily. For example, an effective dosage of the GCR agonist for use in methods of this invention is 0.1, 0.2. 0.3, 0.4. 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 mg per day or optionally twice a day. Preferably, the dosage of the GCR agonist (SEQ ID NO: 1) is 0.3, 1.0 or 3.0 mg per day. Preferably the GCR agonist is given after a meal (i.e, 30 minutes). In some embodiments a second agent useful for treating a lipid metabolism disorder, a binary disorder, a cardiovascular disease, obesity or an endocrine disorder is administered. Suitable second agents are described herein. In some aspects the second agent is administered at less than the standard does for treating the particular disorder because the GCR agonist acts synergistically with the second agent. For example, 2.5, 5. 7.5 or 10 mg of Liptor is given twice a day after a meal (i.e, 30 minutes). On the basis of mg/kg daily dose, either given in single or divided doses, dosages typically range from about 0.001/75 mg/kg to about 10,000/75 mg/kg, preferably from about 0.005/75 mg/kg to about 1,000/75 mg/kg.

The total daily dose of each inhibitor can be administered to the patient in a single dose, or in multiple subdoses. Typically, subdoses can be administered two to six times per day, preferably two to four times per day, and even more preferably two to three times per day. Doses can be in immediate release form or sustained release form sufficiently effective to obtain the desired control over the medical condition.

The dosage regimen to prevent, treat, give relief from, or ameliorate a medical condition or disorder, or to otherwise protect against or treat a medical condition with the combinations and compositions of the present invention is selected in accordance with a variety of factors. These factors include, but are not limited to, the type, age, weight, sex, diet, and medical condition of the subject, the severity of the disease, the route of administration, pharmacological considerations such as the activity, efficacy, pharmacokinetics and toxicology profiles of the particular inhibitors employed, whether a drug delivery system is utilized, and whether the inhibitors are administered with other active ingredients. Thus, the dosage regimen actually employed may vary widely and therefore deviate from the preferred dosage regimen set forth above. An exemplary dosage regimen is 3 mg per day for a 12-week treatment period.

### EXAMPLES

### Example 1

### Positive Results from Plecanatide PhasIII Trial in Patients with Chronic Idiopathic Constipation

Topline Data Show that Plecanatide Met Primary Endpoints

Plecanatide, an investigational oral drug for the treatment of chronic idiopathic constipation (CIC), was well tolerated and met the primary endpoints of a Phase III clinical study. The results are shown in Tables 1-33.

### The Plecanatide Phase 3 CIC Program

### Design

The plecanatide phase 3 CIC program includes two randomized, 12-week, double-blind, placebo-controlled pivotal trials evaluating the efficacy and safety of two different plecanatide treatment doses (3.0 mg and 6.0 mg), taken as a tablet once-a-day, in patients with CIC. Both trials include a two-week pre-treatment baseline period, a 12-week treatment period, and a two-week post-treatment period. The phase 3 CIC program was designed to support regulatory submission in the U. S.

The first phase 3 CIC trial was conducted in North America and assessed 1346 adult patients (19.2% males and 80.8% females) that were randomly assigned to take 3.0 mg or 6.0 mg plecanatide or placebo once-a-day during the 12 week treatment period (453 patients in the 3 mg dose group, 441 patients in the 6.0 mg dose group and 452 patients in the placebo group).

The second phase 3 CIC trial was conducted in the United States and assessed 1337 adult patients (21.6% males and 78.4% females) that were randomly assigned to take 3.0 mg or 6.0 mg plecanatide or placebo once-a-day during the 12 week treatment period (443 patients in the 3 mg dose group, 449 patients in the 6.0 mg dose group and 445 patients in the placebo group).

The first phase 3 CIC trial was conducted in North America and assessed 1,346 adult patients (19.2% males and 80.8% females) that were randomly assigned to take 3.0 mg or 6.0 mg plecanatide or placebo once-a-day during the 12 week treatment period (453 patients in the 3 mg dose group, 441 patients in the 6.0 mg dose group and 452 patients in the placebo group).

### Primary Endpoint

The primary endpoint for both trials is the proportion of durable overall responders (%), which is the current regulatory endpoint required for U.S. approval in CIC. The FDA has defined a durable overall responder as a patient who fulfills both ≧3 complete spontaneous bowel movements (CSBMs) per week plus an increase of ≧1 CSBM from baseline in the same week, for 9 out of the 12 treatment weeks. In addition, the same patient must be an overall responder for at least 3 of the last 4 treatment weeks in order to be considered a durable overall responder.

### Patient Population

Patients were selected using Rome 3 criteria modified for CIC and had (1) fewer than 3 defecations per week, (2) loose stools occurring rarely without laxatives, (3) inadequate criteria for irritable bowel syndrome with constipation (IBS-C), and (4) at least two of the following applied to at least 25% of defecations: (a) straining during evacuation, (b) lumpy or hard stools, (c) sensation of anorectal obstruction or blockage. Rome 3 requires patients to fulfill the criteria for the last 3 months with symptom onset at least 6 months prior to diagnosis.

### Results

Preliminary analysis of the data indicates that both plecanatide 3.0 mg and 6.0 mg doses met the study's primary endpoint and demonstrated statistical significance in the proportion of patients in the intention-to-treat population who were durable overall responders compared to placebo during the 12-week treatment period (21.0% in 3.0 mg and 19.5% in 6.0 mg dose groups compared to 10.2% in placebo; p<0.001 for both doses). The durable overall responder endpoint is the current FDA endpoint required for US approval in CIC. Plecanatide would be the first drug approved for CIC using the more stringent regulatory requirement for durability in the response. Notably, plecanatide was safe and well tolerated at both doses; the most common adverse event was diarrhea, which occurred in 5.9% of patients in 3.0 mg and 5.5% of patients in 6.0 mg dose groups compared to 1.3% of placebo-treated patients.

“We are very pleased with these results and how well they confirm earlier plecanatide data observed in the phase 2b/3 trial,” said Gary S. Jacob, Ph.D., Chairman and CEO of Synergy. “These results strengthen our belief that plecanatide has the potential to not only effectively treat constipation but with a durability and tolerability profile that is ideal for chronic use. We look forward to the results of our second pivotal trial in the coming weeks.”

Stool consistency was the key secondary endpoint reported with top-line analyses; both 3.0 mg and 6.0 mg plecanatide doses showed statistically significant improvement from baseline in Bristol Stool Form Scale (BSFS) scores compared to placebo (mean increase of 1.53 in 3.0 mg and 1.52 in 6.0 mg dose groups compared to a mean increase of 0.77 in placebo; p<0.001 for both doses). The observed improvements began at Week 1, continued throughout the 12-week treatment period, and returned towards baseline with no indication of an exaggerated or rebound effect following discontinuation of treatment.

15 patients in the trial (1.1%) experienced serious adverse events but there was no imbalance across treatment groups in either incidences or individual serious adverse events. Overall, the rates of withdrawal from treatment because of an adverse event were low (5.1% in 3.0 mg and 5.0% in 6.0 mg dose groups compared to 1.3% in placebo) and discontinuations due to diarrhea were infrequent (2.7% in 3.0 mg and 2.4% in 6.0 mg dose groups compared to 0.4% in placebo). No clinically relevant abnormalities were observed in serum chemistries, hematology, urinalysis, ECG or vital signs measurements.

### About Chronic Constipation

Chronic constipation is the most common digestive complaint in the United States and the world. About 15%, or 45 million people, suffer from chronic constipation in the U.S., with a similar prevalence in other developed countries. Although chronic constipation affects both men and women of every age, it disproportionately impacts women as well as the elderly, a large and growing population.

Current treatments provide temporary relief, but because they fail to address the underlying causes of chronic constipation, they do not normalize patients' bowel function. Such treatments are also associated with unpleasant side effects, the most common of which is diarrhea, causing patients to see-saw between extremes. As a result, most doctors and their patients are dissatisfied with current treatments for chronic constipation.

Chronic constipation is also a significant driver of healthcare costs. Healthcare systems are spending millions of dollars annually to diagnose and treat this disorder, including $820 million annually on over-the-counter laxatives in the U.S. alone.

About Plecanatide

Plecanatide is a uroguanylin analogue in pivotal phase 3 clinical development to treat patients with CIC and 1113 S-C. Uroguanylin is a naturally occurring gastrointestinal (GI) peptide produced by humans in the small intestine and plays a key role in regulating normal GI activity. Orally administered plecanatide is designed to mimic uroguanylin's natural activity and regulate the movement of fluid required for normal digestion.

### REFERENCES

1. Currie, et al., Proc. Nat'l Acad. Sci. USA 89:947-951 (1992).
2. Hamra, et al., Proc. Nat'l Acad. Sci. USA 90:10464-10468 (1993).
3. Forte, L., Reg. Pept. 81:25-39 (1999).
4. Schulz, et al., Cell 63:941-948 (1990).
5. Guba, et al., Gastroenterology 111:1558-1568 (1996).
6. Joo, et al., Am. J. Physiol. 274:G633-G644 (1998).
7. Evan, et al., Nature (London) 411:342-348 (2001).
8. Eastwood, G., J. Clin. Gastroenterol. 14:S29-33 (1992).
9. Lipkin, M. Arch. Fr. Mal. Appl Dig. 61:691-693 (1972).
10. Wong, et al., Gut 50:212-217 (2002).
11. Potten, et al., Stem Cells 15:82-93.
12. Basoglu, et al., in: Proceedings of the Second FEPS Congress, Jun. 29-Jul. 4, 1999, Prague, Czech Republic., lf2.cuni.cz/physiolres/feps/basoglu
13. Sindic, et al., J. Biol. Chem. Mar. 11, 2002, manuscript M110627200 (in press).
14. Askling, J., Dickman, P. W., Karlen, P., Brostrom, O., Lapidus, A., Lofberg, R., and Ekbom, A. Colorectal cancer rates among first-degree relatives of patients with inflammatory bowel disease: a population-based cohort study. Lancet, 357: 262-266,
15. Provenzale, D. and Onken, J. Surveillance issues in inflammatory bowel disease: Ulcerative colitis. J Clin Gastroenterol, 32:99-105 (2001).
16. Ettorre, G. M, Pescatori, M., Panis, Y., Nemeth, J., Crescenzi, A., and Valleur, P. Mucosal changes in ileal pouches after restorative proctocolectomy for ulcerative and Crohn's colitis. Dis Colon Rectum, 43:1743-1748 (2000).
17. Shinozaki M, Watanabe T, Kubota Y, Sawada T, Nagawa H, Muto T. High proliferative activity is associated with dysplasia in ulcerative colitis. Dis Colon Rectum, 43:S34-S39 (2000).
18. Deschner, E. E., Winawer, S. J., Katz, S., Katzka, I., and Kahn, E. Proliferative defects in ulcerative colitis patients. Cancer Invest, 1:41-47 (1983).
19. Wong, W. M., and Wright, N. A. Cell proliferation and gastrointestinal mucosa. J Clin Pathol, 52:321-333.
20. Potten, C. S., Wilson, J. W., and Booth, C. Regulation and significance of apoptosis in the stem cells. Stem Cells, 15:82-93.
21. Bhakdi, et al., Infect. Immun. 57:3512-3519 (1989).
22. Hughes, et al., J. Biol. Chem. 272:30567-30576 (1997).
23. Cermak, et al., Pflugers Arch. 43:571-577 (1996).
24. Wu, et al., J. Biol. Chem. 272:14860-14866 (1997).
25. Shailubhai et al., Cancer Research 60, 5151-5157 (2000)
26. Shailubhai et al., Curr. Opin. Drug Disc. Dev. 5(2): 261-268 (2002).
27. Collins, S M. J Clin Gastroenterol. 41 Suppl 1:S30-32 (2007)
28. Ramamoorthy S et al., J Biol Chem. 282(16):11639-11647 (2007)
29. Delvaux M, et al., (1998) Aliment Pharmacol Ther. 12:849-55 (1998).
30. Muller-Lissner S, et al., Am J Gastroenterol. 101: 2558-69 (2006).
31. Barbara G, et al., Gut; 51(suppl 1):141-144 (2002).
32. Lam D D, et al., Expert Rev Mol Med; 9:1-24 (2007).
33. de Luca C, Olefsky J M. FEBS Lett. 582:97-105 (2008).
34. Tilg H, Moschen A R. Mol Med. 14:222-31 (2008).
35. Gulcan E et. al., Am J Med Sci. 338:116-9 (2009).
36. Spranger J, et al., Diabetes. 52:812-17 (2003).
37. GiAlcan E, Am J Med Sci. 334: 444-51 (2007).
38. Brown M S, Goldstein J L., Science. 232: 34-47 (1986).
39. Samuel, P., et al., J. Clin. Invest. 47: 2070-2078 (1968).
40. Turley S D and Dietschy J M., The Liver: Biology and Pathobiology, 617-641 (1988).
41. Gylling H, Miettinen T A, Atherosclerosis. 117: 305-308 (1995).
42. Meier P J, Stieger B., Annu Rev Physiol. 64: 635-61 (2002).
43. Clader J W., J Med Chem. 47: 1-9 (2004).
44. Shailubhai, K., et al., Cancer Research 60: 5151-5157 (2000).

TABLE 1
Summary of Treatment Emergent Adverse Events Leading to Discontinuation
of Study Drug by System Organ Class and Preferred Term
(Safety Population)

| | | | | Plecanatide | | | Plecanatide | | | Active | | | | | |
|---|---|---|---|---|---|---|---|---|---|---|---|---|---|---|---|
| | Placebo | | | 3 mg | | | 6 mg | | | Combined | | | Overall | | |
| System Organ Class | (N = 458) | | | (N = 474) | | | (N = 457) | | | (N = 931) | | | (N = 1389) | | |
| Preferred Term | N | (%) | E | N | (%) | E | N | (%) | E | N | (%) | E | N | (%) | E |
| Number of Treatment Emergent | 6 | (1.3) | 6 | 24 | (5.1) | 24 | 23 | (5.0) | 23 | 47 | (5.0) | 47 | 53 | (3.8) | 53 |
| Adverse Events Leading to | | | | | | | | | | | | | | | |
| Discontinuation of Study Drug | | | | | | | | | | | | | | | |
| Endocrine disorders | 0 | | 0 | 1 | (0.2) | 1 | 0 | | 0 | 1 | (0.1) | 1 | 1 | (0.1) | 1 |
| Hypothyroidism | 0 | | 0 | 1 | (0.2) | 1 | 0 | | 0 | 1 | (0.1) | 1 | 1 | (0.1) | 1 |
| Gastrointestinal disorders | 3 | (0.7) | 3 | 19 | (4.0) | 19 | 19 | (4.2) | 19 | 38 | (4.1) | 38 | 41 | (3.0) | 41 |
| Abdominal distension | 0 | | 0 | 1 | (0.2) | 1 | 1 | (0.2) | 1 | 2 | (0.2) | 2 | 2 | (0.1) | 2 |
| Abdominal pain | 0 | | 0 | 2 | (0.4) | 2 | 3 | (0.7) | 3 | 5 | (0.5) | 5 | 5 | (0.4) | 5 |
| Abdominal pain upper | 0 | | 0 | 1 | (0.2) | 1 | 0 | | 0 | 1 | (0.1) | 1 | 1 | (0.1) | 1 |
| Diarrhoea | 2 | (0.4) | 2 | 13 | (2.7) | 13 | 11 | (2.4) | 11 | 24 | (2.6) | 24 | 26 | (1.9) | 26 |
| Faecal incontinence | 0 | | 0 | 0 | | 0 | 1 | (0.2) | 1 | 1 | (0.1) | 1 | 1 | (0.1) | 1 |
| Gastrointestinal hypermotility | 0 | | 0 | 0 | | 0 | 1 | (0.2) | 1 | 1 | (0.1) | 1 | 1 | (0.1) | 1 |
| Intestinal obstruction | 0 | | 0 | 1 | (0.2) | 1 | 0 | | 0 | 1 | (0.1) | 1 | 1 | (0.1) | 1 |
| Mouth ulceration | 0 | | 0 | 0 | | 0 | 1 | (0.2) | 1 | 1 | (0.1) | 1 | 1 | (0.1) | 1 |
| Nausea | 1 | (0.2) | 1 | 0 | | 0 | 1 | (0.2) | 1 | 1 | (0.1) | 1 | 2 | (0.1) | 2 |
| Vomiting | 0 | | 0 | 1 | (0.2) | 1 | 0 | | 0 | 1 | (0.1) | 1 | 1 | (0.1) | 1 |
| Infections and infestations | 2 | (0.4) | 2 | 0 | | 0 | 1 | (0.2) | 1 | 1 | (0.1) | 1 | 3 | (0.2) | 3 |
| Diverticulitis | 1 | (0.2) | 1 | 0 | | 0 | 0 | | 0 | 0 | | 0 | 1 | (0.1) | 1 |
| Post procedural infection | 1 | (0.2) | 1 | 0 | | 0 | 0 | | 0 | 0 | | 0 | 1 | (0.1) | 1 |
| Staphylococcal infection | 0 | | 0 | 0 | | 0 | 1 | (0.2) | 1 | 1 | (0.1) | 1 | 1 | (0.1) | 1 |
| Investigations | 0 | | 0 | 1 | (0.2) | 1 | 0 | | 0 | 1 | (0.1) | 1 | 1 | (0.1) | 1 |
| Alanine aminotransferase increased | 0 | | 0 | 1 | (0.2) | 1 | 0 | | 0 | 1 | (0.1) | 1 | 1 | (0.1) | 1 |

Source: Listing 16.2.10
Note:
Patients are only summarized for the primary AE leading to discontinuation of study drug. Only primary adverse events are counted in the event (E) column. Adverse events are coded using MedDRA version 14.1.
Chiltern * JUN2015:8:32 AM * I:\Synergy\34196\21_Stat\Programs\Tables\t_ae.sas * ZScott

TABLE 2
Demographics and Baseline Characteristics (ITT Population)

| | | Plecanatide | Plecanatide | Active | |
|---|---|---|---|---|---|
| | Placebo | 3 mg | 6 mg | Combined | Overall |
| | (N = 452) | (N = 453) | (N = 441) | (N = 894) | (N = 1346) |
| Age | | | | | |
| (years) | | | | | |
| n | 452 | 453 | 441 | 894 | 1346 |
| Mean | 46.4 | 45.0 | 45.1 | 45.1 | 45.5 |
| SD | 13.92 | 14.62 | 13.77 | 14.20 | 14.12 |
| Median | 46.0 | 45.0 | 45.0 | 45.0 | 46.0 |
| Minimum | 18 | 18 | 18 | 18 | 18 |
| Maximum | 78 | 79 | 79 | 79 | 79 |
| Gender, | | | | | |
| n (%) | | | | | |
| Male | 95 (21.0) | 85 (18.8) | 79 (17.9) | 164 (18.3) | 259 (19.2) |
| Female | 357 (79.0) | 368 (81.2) | 362 (82.1) | 730 (81.7) | 1087 (80.8) |

Source: Listings 16.2.4.1, 16.2.12.1
Note:
Percentages are based on the numbers of patients in the ITT Populations in each treatment group.
Chiltern * 16JUN2015:8:35 AM * I:\Synergy\34196\21_Stat\Programs\Tables\t_demog.sas * ZScott

TABLE 3
Number and Percentage of Durable Overall CSBM Responders,
Mean Replacement Approach (MRA) (ITT Population)

| | | Plecanatide | Plecanatide | Active |
|---|---|---|---|---|
| | Placebo | 3 mg | 6 mg | Combined |
| | (N = 452) | (N = 453) | (N = 441) | (N = 894) |
| Durable Overall CSBM | 46 (10.2) | 95 (21.0) | 86 (19.5) | 181 (20.2) |
| Responders, n (%) [1] | | | | |
| 95% CI (%) [2] | (7.5, 13.3) | (17.3, 25.0) | (15.9, 23.5) | (17.7, 23.0) |
| Non-Responders, n (%) [3] | 406 (89.8) | 358 (79.0) | 355 (80.5) | 713 (79.8) |
| CMH p-value [4] | — | <0.001 | <0.001 | — |
| Odds Ratio (adjusted) [5] | — | 0.429 | 0.469 | — |
| 95% CI | — | (0.294, 0.627) | (0.319, 0.688) | — |
| Difference in Proportions | — | 0.107 | 0.094 | — |
| (adjusted) [6] | | | | |
| 95% CI | — | (0.061, 0.154) | (0.047, 0.140) | — |
| Breslow-Day p-value [7] | — | 0.609 | 0.096 | — |

Source: Listing 16.2.6.1
[1] A durable overall CSBM responder is a patient who is a weekly CSBM responder for at least 9 of the 12 treatment weeks, including at least 3 of the last 4 weeks. (A CSBM weekly responder is defined as patient who has >= 3 CSBMs per week and an increase from baseline of >= 1 CSBM for that week, determined using mean replacement approach (MRA) methodology as defined in Section 4.1.2 of the statistical analysis plan.)
[2] Clopper-Pearson method
[3] Patients missing with respect to the endpoint were scored as non-responders.
[4] CMH p-value for the comparison of treatment group to placebo, stratified by gender.
[5] Common odds ratio, treatment/placebo adjusted for stratification factor (gender) in the CMH analysis.
[6] Proportion of responders in treatment group minus proportion in Placebo group, adjusted for stratification factor (gender) in the CMH analysis.
[7] Breslow-Day p-value for the test of consistency of the treatment effect across the stratification.
Chiltern *16JUN2015:8:43 AM * I:\Synergy\34196\21_Stat\Programs\Tables\t_respond.sas * ZScott

TABLE 4
Change from Baseline in Stool Consistency by Time Point; Linear Mixed-effects
Model, Mean Replacement Approach (MRA) (ITT Population)

| | | Plecanatide | Plecanatide | Active |
|---|---|---|---|---|
| | Placebo | 3 mg | 6 mg | Combined |
| | (N = 452) | (N = 453) | (N = 441) | (N = 894) |
| Change from Baseline [1], | | | | |
| (overall average estimate across | | | | |
| the 12-week treatment period) | | | | |
| LS Mean (SE) | 0.77 (0.058) | 1.53 (0.058) | 1.52 (0.059) | — |
| Difference from Placebo | — | 0.76 | 0.75 | — |
| 95% CI [2] | — | 0.61, 0.90 | 0.60, 0.89 | — |
| p-value vs. Placebo [3] | — | <0.001 | <0.001 | — |
| Baseline [1] | | | | |
| n | 441 | 438 | 422 | 860 |
| Mean (SD) | 2.56 (1.114) | 2.52 (1.046) | 2.59 (1.171) | 2.55 (1.109) |
| Week 1 Change from Baseline [1] | | | | |
| n | 417 | 414 | 399 | 813 |
| Mean (SD) | 0.60 (1.275) | 1.57 (1.563) | 1.42 (1.640) | 1.49 (1.602) |
| LS Mean (SE) | 0.57 (0.070) | 1.50 (0.071) | 1.42 (0.072) | — |
| Difference from Placebo | — | 0.92 | 0.85 | — |
| 95% CI [2] | — | 0.74, 1.11 | 0.66, 1.03 | — |
| p-value vs. Placebo [3] | — | <0.001 | <0.001 | — |

Source: Listing 16.2.6.1
[1] Baseline is the mean BSFS score recorded during the 2-week baseline diary assessment period prior to the first dose of study drug. The weekly mean BSFS score per patient is derived from the BSFS entries reported during the Treatment Period in the Daily Symptom Diary. The time points reported are Treatment Period study weeks defined as follows: Week 1 = study days 1-7; Week 2 = study days 8-14; Week 3 = study days 15-21; Week 4 = study days 22-28; Week 5 = study days 29-35; Week 6 = study days 36-42; Week 7 = study days 43-49; Week 8 = study days 50-56; Week 9 = study days 57-63; Week 10 = study days 64-70; Week 11 = study days 71-77; Week 12 = study days 78-84. Changes from baseline are based on mean replacement approach (MRA) methodology as defined in Section 4.1.2 of the statistical analysis plan.
[2] The 95% confidence interval for the difference between the placebo group and the specified treatment group LS means.
[3] P-value vs. Placebo is from the pairwise comparison of LS means between the placebo group and the specified treatment group using a linear mixed-effects model with fixed effects for gender (stratification variable), treatment, week, the interaction of treatment and week, and the corresponding baseline value, and a random intercept for patient. The model takes into account the repeated measurements of stool consistency for each patient.
Chiltern · 16JUN2015:8:44 AM · I:\Synergy\34196\21_Stat\Programs\Tables\t_sbm_cfb.sas · ZScott

TABLE 5
Summary of Treatment Emergent Serious Adverse Events by System Organ Class,
Preferred Term, and Relationship to Study Drug
(Safety Population)

| | | | | Plecanatide | | | Plecanatide | | | Active | | | | | |
|---|---|---|---|---|---|---|---|---|---|---|---|---|---|---|---|
| System Organ Class | Placebo | | | 3 mg | | | 6 mg | | | Combined | | | Overall | | |
| Preferred Term | (N = 458) | | | (N = 474) | | | (N = 457) | | | (N = 931) | | | (N = 1389) | | |
| Relationship | N | (%) | E | N | (%) | E | N | (%) | E | N | (%) | E | N | (%) | E |
| Number of Treatment Emergent | 4 | (0.9) | 4 | 6 | (1.3) | 6 | 5 | (1.1) | 5 | 11 | (1.2) | 11 | 15 | (1.1) | 15 |
| Serious Adverse Events | | | | | | | | | | | | | | | |
| Reasonable Possibility [1] | 1 | (0.2) | 1 | 0 | | 0 | 0 | | 0 | 0 | | 0 | 1 | (0.1) | 1 |
| No Reasonable Possibility | 3 | (0.7) | 3 | 6 | (1.3) | 6 | 5 | (1.1) | 5 | 11 | (1.2) | 11 | 14 | (1.0) | 14 |
| Gastrointestinal disorders | 0 | | 0 | 1 | (0.2) | 1 | 1 | (0.2) | 1 | 2 | (0.2) | 2 | 2 | (0.1) | 2 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 1 | (0.2) | 1 | 2 | (0.2) | 2 | 2 | (0.1) | 2 |
| Intestinal obstruction | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Pancreatitis acute | 0 | | 0 | 0 | | 0 | 1 | (0.2) | 1 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 0 | | 0 | 1 | (0.2) | 1 | 1 | (0.1) | 1 | 1 | (0.1) | 1 |
| Hepatobiliary disorders | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Cholelithiasis | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Infections and infestations | 3 | (0.7) | 3 | 0 | | 0 | 2 | (0.4) | 2 | 2 | (0.2) | 2 | 5 | (0.4) | 5 |
| Reasonable Possibility [1] | 1 | (0.2) | 1 | 0 | | 0 | 0 | | 0 | 0 | | 0 | 1 | (0.1) | 1 |
| No Reasonable Possibility | 2 | (0.4) | 2 | 0 | | 0 | 2 | (0.4) | 2 | 2 | (0.2) | 2 | 4 | (0.3) | 4 |
| Diverticulitis | 1 | (0.2) | 1 | 0 | | 0 | 0 | | 0 | 0 | | 0 | 1 | (0.1) | 1 |
| Reasonable Possibility [1] | 1 | (0.2) | 1 | 0 | | 0 | 0 | | 0 | 0 | | 0 | 1 | (0.1) | 1 |
| No Reasonable Possibility | 0 | | 0 | 0 | | 0 | 0 | | 0 | 0 | | 0 | 0 | | 0 |
| Gastroenteritis | 0 | | 0 | 0 | | 0 | 1 | (0.2) | 1 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 0 | | 0 | 1 | (0.2) | 1 | 1 | (0.1) | 1 | 1 | (0.1) | 1 |
| Mastitis | 1 | (0.2) | 1 | 0 | | 0 | 0 | | 0 | 0 | | 0 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Pneumonia | 1 | (0.2) | 1 | 0 | | 0 | 0 | | 0 | 0 | | 0 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Staphylococcal infection | 0 | | 0 | 0 | | 0 | 1 | (0.2) | 1 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 0 | | 0 | 1 | (0.2) | 1 | 1 | (0.1) | 1 | 1 | (0.1) | 1 |
| Injury, poisoning and procedural | 0 | | 0 | 0 | | 0 | 1 | (0.2) | 1 | 1 | (0.1) | 1 | 1 | (0.1) | 1 |
| complications | | | | | | | | | | | | | | | |
| Reasonable Possibility [1] | 0 | | 0 | 0 | | 0 | 0 | | 0 | 0 | | 0 | 0 | | 0 |
| No Reasonable Possibility | 0 | | 0 | 0 | | 0 | 1 | (0.2) | 1 | 1 | (0.1) | 1 | 1 | (0.1) | 1 |
| Ankle fracture | 0 | | 0 | 0 | | 0 | 1 | (0.2) | 1 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 0 | | 0 | 1 | (0.2) | 1 | 1 | (0.1) | 1 | 1 | (0.1) | 1 |
| Investigations | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Aspartate aminotransferase increased | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Nervous system disorders | 0 | | 0 | 1 | (0.2) | 1 | 1 | (0.2) | 1 | 2 | (0.2) | 2 | 2 | (0.1) | 2 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 1 | (0.2) | 1 | 2 | (0.2) | 2 | 2 | (0.1) | 2 |
| Cerebral infarction | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Headache | 0 | | 0 | 0 | | 0 | 1 | (0.2) | 1 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 0 | | 0 | 1 | (0.2) | 1 | 1 | (0.1) | 1 | 1 | (0.1) | 1 |

Source: Listing 16.2.10
Note:
If a patient has multiple occurrences of an SAE, the patient is presented only once at the maximum relationship in the respective patient count. Events are counted each time in the event (E) column. Adverse events are coded using MedDRA version 14.1.
[1] There is evidence to suggest a causal relationship between the drug and the SAE.
Chiltern * 16JUN2015:8:32 AM * I:\Synergy\34196\21_stat\Programs\Tables\t_aeby.sas * ZScott

TABLE 6
Patient Disposition
(All Randomized (includes Duplicate Patients))

| | | | Plecanatide | | Plecanatide | | Active | | | |
|---|---|---|---|---|---|---|---|---|---|---|
| | Placebo | | 3 mg | | 6 mg | | Combined | | Overall | |
| | (N = 469) | | (N = 470) | | (N = 471) | | (N = 941) | | (N = 1410) | |
| Analysis Populations, n (%) | | | | | | | | | | |
| Intention-to-treat (ITT) Population [1] | 445 | (94.9) | 443 | (94.3) | 449 | (95.3) | 892 | (94.8) | 1337 | (94.8) |
| Randomized, not treated | 1 | (0.2) | 4 | (0.9) | 2 | (0.4) | 6 | (0.6) | 7 | (0.5) |
| Non-duplicate patients* | 437 | (93.2) | 434 | (92.3) | 443 | (94.1) | 877 | (93.2) | 1314 | (93.2) |
| Index case patients* | 8 | (1.7) | 9 | (1.9) | 6 | (1.3) | 15 | (1.6) | 23 | (1.6) |
| Safety Population [2] | 467 | (99.6) | 466 | (99.1) | 469 | (99.6) | 935 | (99.4) | 1402 | (99.4) |
| Non-duplicate patients* | 436 | (93.0) | 430 | (91.5) | 441 | (93.6) | 871 | (92.6) | 1307 | (92.7) |
| Duplicate patients* | 31 | (6.6) | 36 | (7.7) | 28 | (5.9) | 64 | (6.8) | 95 | (6.7) |
| Index case patients* | 8 | (1.7) | 9 | (1.9) | 6 | (1.3) | 15 | (1.6) | 23 | (1.6) |
| Per Protocol (PP) Population [3] | 353 | (75.3) | 340 | (72.3) | 355 | (75.4) | 695 | (73.9) | 1048 | (74.3) |
| PK Population [4] | 32 | (6.8) | 31 | (6.6) | 32 | (6.8) | 63 | (6.7) | 95 | (6.7) |
| Completion Status, n (%) | | | | | | | | | | |
| Discontinued from the Study during the Treatment | 59 | (12.6) | 76 | (16.2) | 63 | (13.4) | 139 | (14.8) | 198 | (14.0) |
| Phase | | | | | | | | | | |
| Completed Study Treatment Phase (Week 12 (EOT)) | 410 | (87.4) | 394 | (83.8) | 408 | (86.6) | 802 | (85.2) | 1212 | (86.0) |
| Discontinued from the Study after the Treatment Phase | 4 | (0.9) | 2 | (0.4) | 3 | (0.6) | 5 | (0.5) | 9 | (0.6) |
| Completed the Study (Week 14 (EOS)) | 406 | (86.6) | 392 | (83.4) | 405 | (86.0) | 797 | (84.7) | 1203 | (85.3) |
| Primary Reason for Discontinuation in Patients | | | | | | | | | | |
| who Completed Study Treatment Phase (Week 12 | | | | | | | | | | |
| (EOT)), n (%) | | | | | | | | | | |
| Adverse Event | 0 | | 0 | | 0 | | 0 | | 0 | |
| Death | 0 | | 0 | | 0 | | 0 | | 0 | |
| Insufficient Therapeutic Effect | 0 | | 0 | | 0 | | 0 | | 0 | |
| Lost to Follow-Up | 1 | (0.2) | 0 | | 1 | (0.2) | 1 | (0.1) | 2 | (0.1) |
| Non-compliance with Study Drug | 1 | (0.2) | 0 | | 0 | | 0 | | 1 | (0.1) |
| Physician Decision | 0 | | 0 | | 0 | | 0 | | 0 | |
| Protocol Violation | 1 | (0.2) | 0 | | 1 | (0.2) | 1 | (0.1) | 2 | (0.1) |
| Withdrawal of Consent by Patient | 1 | (0.2) | 1 | (0.2) | 1 | (0.2) | 2 | (0.2) | 3 | (0.2) |
| Other | 0 | | 1 | (0.2) | 0 | | 1 | (0.1) | 1 | (0.1) |
| Primary Reason for Discontinuation in Patients who | | | | | | | | | | |
| did not Complete Study Treatment Phase (Week 12 | | | | | | | | | | |
| (EOT)), n (%) | | | | | | | | | | |
| Adverse Event | 14 | (3.0) | 14 | (3.0) | 17 | (3.6) | 31 | (3.3) | 45 | (3.2) |
| Death | 0 | | 0 | | 0 | | 0 | | 0 | |
| Insufficient Therapeutic Effect | 8 | (1.7) | 3 | (0.6) | 1 | (0.2) | 4 | (0.4) | 12 | (0.9) |
| Lost to Follow-Up | 8 | (1.7) | 7 | (1.5) | 6 | (1.3) | 13 | (1.4) | 21 | (1.5) |
| Non-compliance with Study Drug | 1 | (0.2) | 0 | | 0 | | 0 | | 1 | (0.1) |
| Physician Decision | 0 | | 0 | | 0 | | 0 | | 0 | |
| Protocol Violation | 17 | (3.6) | 18 | (3.8) | 14 | (3.0) | 32 | (3.4) | 49 | (3.5) |
| Withdrawal of Consent by Patient | 8 | (1.7) | 31 | (6.6) | 23 | (4.9) | 54 | (5.7) | 62 | (4.4) |
| Other | 3 | (0.6) | 3 | (0.6) | 2 | (0.4) | 5 | (0.5) | 8 | (0.6) |

Source: Listings 16.2.1.3, 16.2.3
Note:
Percentages are based on the number of enrolled patients in each treatment group.
*Note: ‘Duplicate patients’ are patients who have more than one unique patient identifier in this study and/or any previous or concurrent plecanatide study. For duplicate patients, the earliest instance of screening in any study is designated as the ‘index case’ for purposes of proper assignment of duplicate patients to analysis populations.
[1] The ITT population consists of all randomized patients. Duplicate patients can appear at most once in the ITT Population as the index case.
[2] The Safety Population consists of all randomized patients who have received at least one dose of the study drug. Patients in the Safety Population will be analyzed according to the treatment received. Duplicate patients can appear more than once in the Safety Population.
[3] The PP Population consists of all patients in the ITT population who completed the 12-week Treatment Period or discontinued from study treatment due to adverse event(s) or lack of efficacy (insufficient therapeutic response), were diary and treatment compliant, and had no major protocol violations. Duplicate patients have a major protocol violation and are not included in the PP Population.
[4] The PK Population consists of all randomized patients who received study drug and have at least 1 post-dose PK collection completed. PK results were analyzed/retained by a separate PK vendor.
Chiltern · 29JUL2015:5:34 PM · I:\Synergy\33610\21 Stat\Programs\Tables\t disp.sas · ZScott

TABLE 7
Patient Disposition
(Safety Population)

| | | | Plecanatide | | Plecanatide | | Active | | | |
|---|---|---|---|---|---|---|---|---|---|---|
| | Placebo | | 3 mg | | 6 mg | | Combined | | Overall | |
| | (N = 466) | | (N = 467) | | (N = 469) | | (N = 936) | | (N = 1402) | |
| Analysis Populations, n (%) | | | | | | | | | | |
| Intention-to-treat (ITT) Population [1] | 444 | (95.3) | 439 | (94.0) | 447 | (95.3) | 886 | (94.7) | 1330 | (94.9) |
| Randomized, not treated | 0 | | 0 | | 0 | | 0 | | 0 | |
| Non-duplicate patients* | 436 | (93.6) | 430 | (92.1) | 441 | (94.0) | 871 | (93.1) | 1307 | (93.2) |
| Index case patients* | 8 | (1.7) | 9 | (1.9) | 6 | (1.3) | 15 | (1.6) | 23 | (1.6) |
| Safety Population [2] | 466 | (100.0) | 467 | (100.0) | 469 | (100.0) | 936 | (100.0) | 1402 | (100.0) |
| Non-duplicate patients* | 436 | (93.6) | 430 | (92.1) | 441 | (94.0) | 871 | (93.1) | 1307 | (93.2) |
| Duplicate patients* | 30 | (6.4) | 37 | (7.9) | 28 | (6.0) | 65 | (6.9) | 95 | (6.8) |
| Index case patients* | 8 | (1.7) | 9 | (1.9) | 6 | (1.3) | 15 | (1.6) | 23 | (1.6) |
| Per Protocol (PP) Population [3] | 353 | (75.8) | 340 | (72.8) | 355 | (75.7) | 695 | (74.3) | 1048 | (74.8) |
| PK Population [4] | 32 | (6.9) | 31 | (6.6) | 32 | (6.8) | 63 | (6.7) | 95 | (6.8) |
| Completion Status, n (%) | | | | | | | | | | |
| Discontinued from the Study during the Treatment | 57 | (12.2) | 72 | (15.4) | 61 | (13.0) | 133 | (14.2) | 190 | (13.6) |
| Phase | | | | | | | | | | |
| Completed Study Treatment Phase (Week 12 (EOT)) | 409 | (87.8) | 395 | (84.6) | 408 | (87.0) | 803 | (85.8) | 1212 | (86.4) |
| Discontinued from the Study after the Treatment Phase | 3 | (0.6) | 3 | (0.6) | 3 | (0.6) | 6 | (0.6) | 9 | (0.6) |
| Completed the Study (Week 14 (EOS)) | 406 | (87.1) | 392 | (83.9) | 405 | (86.4) | 797 | (85.1) | 1203 | (85.8) |
| Primary Reason for Discontinuation in Patients | | | | | | | | | | |
| who Completed Study Treatment Phase (Week 12 | | | | | | | | | | |
| (EOT)), n (%) | | | | | | | | | | |
| Adverse Event | 0 | | 0 | | 0 | | 0 | | 0 | |
| Death | 0 | | 0 | | 0 | | 0 | | 0 | |
| Insufficient Therapeutic Effect | 0 | | 0 | | 0 | | 0 | | 0 | |
| Lost to Follow-Up | 1 | (0.2) | 0 | | 1 | (0.2) | 1 | (0.1) | 2 | (0.1) |
| Non-compliance with Study Drug | 1 | (0.2) | 0 | | 0 | | 0 | | 1 | (0.1) |
| Physician Decision | 0 | | 0 | | 0 | | 0 | | 0 | |
| Protocol Violation | 0 | | 1 | (0.2) | 1 | (0.2) | 2 | (0.2) | 2 | (0.1) |
| Withdrawal of Consent by Patient | 1 | (0.2) | 1 | (0.2) | 1 | (0.2) | 2 | (0.2) | 3 | (0.2) |
| Other | 0 | | 1 | (0.2) | 0 | | 1 | (0.1) | 1 | (0.1) |
| Primary Reason for Discontinuation in Patients who | | | | | | | | | | |
| did not Complete Study Treatment Phase (Week 12 | | | | | | | | | | |
| (EOT)), n (%) | | | | | | | | | | |
| Adverse Event | 14 | (3.0) | 14 | (3.0) | 17 | (3.6) | 31 | (3.3) | 45 | (3.2) |
| Death | 0 | | 0 | | 0 | | 0 | | 0 | |
| Insufficient Therapeutic Effect | 8 | (1.7) | 3 | (0.6) | 1 | (0.2) | 4 | (0.4) | 12 | (0.9) |
| Lost to Follow-Up | 8 | (1.7) | 7 | (1.5) | 6 | (1.3) | 13 | (1.4) | 21 | (1.5) |
| Non-compliance with Study Drug | 1 | (0.2) | 0 | | 0 | | 0 | | 1 | (0.1) |
| Physician Decision | 0 | | 0 | | 0 | | 0 | | 0 | |
| Protocol Violation | 15 | (3.2) | 16 | (3.4) | 14 | (3.0) | 30 | (3.2) | 45 | (3.2) |
| Withdrawal of Consent by Patient | 8 | (1.7) | 29 | (6.2) | 22 | (4.7) | 51 | (5.4) | 59 | (4.2) |
| Other | 3 | (0.6) | 3 | (0.6) | 1 | (0.2) | 4 | (0.4) | 7 | (0.5) |

Source: Listings 16.2.1.3, 16.2.3
Note:
Percentages are based on the number of enrolled patients in each treatment group.
*Note: ‘Duplicate patients’ are patients who have more than one unique patient identifier in this study and/or any previous or concurrent plecanatide study. For duplicate patients, the earliest instance of screening in any study is designated as the ‘index case’ for purposes of proper assignment of duplicate patients to analysis populations.
[1] The ITT population consists of all randomized patients. Duplicate patients can appear at most once in the ITT Population as the index case.
[2] The Safety Population consists of all randomized patients who have received at least one dose of the study drug. Patients in the Safety Population will be analyzed according to the treatment received. Duplicate patients can appear more than once in the Safety Population.
[3] The PP Population consists of all patients in the ITT population who completed the 12-week Treatment Period or dicontinued from study treatment due to adverse event(s) or lack of efficacy (insufficient therapeutic response), were diary and treatment compliant, and had no major protocol violations. Duplicate patients have a major protocol violation and are not included in the PP Population.
[4] The PK Population consists of all randomized patients who received study drug and have at least 1 post-dose PK collection completed. PK results were analyzed/retained by a separate PK vendor.
Chiltern · 29JUL2015:5:34 PM · I:\Synergy\33610\21 Stat\Programs\Tables\t disp.sas · ZScott

TABLE 8
Demographics and Baseline Characteristics
(ITT Population)

| | | Plecanatide | Plecanatide | Active | |
|---|---|---|---|---|---|
| | Placebo | 3 mg | 6 mg | Combined | Overall |
| | (N = 445) | (N = 443) | (N = 449) | (N = 892) | (N = 1337) |
| Age (years) | | | | | |
| n | 445 | 443 | 449 | 892 | 1337 |
| Mean | 44.6 | 45.5 | 45.3 | 45.4 | 45.1 |
| SD | 14.59 | 14.38 | 14.47 | 14.42 | 14.47 |
| Median | 44.0 | 46.0 | 45.0 | 45.0 | 45.0 |
| Minimum | 18 | 18 | 18 | 18 | 18 |
| Maximum | 80 | 80 | 80 | 80 | 80 |
| Gender, n (%) | | | | | |
| Male | 95 (21.3) | 98 (22.1) | 96 (21.4) | 194 (21.7) | 289 (21.6) |
| Female | 350 (78.7) | 345 (77.9) | 353 (78.6) | 698 (78.3) | 1048 (78.4) |
| Race, n (%) | | | | | |
| American Indian or Alaskan Native | 0 | 0 | 2 (0.4) | 2 (0.2) | 2 (0.1) |
| Asian | 14 (3.1) | 7 (1.6) | 11 (2.4) | 18 (2.0) | 32 (2.4) |
| Black or African American | 91 (20.4) | 88 (19.9) | 102 (22.7) | 190 (21.3) | 281 (21.0) |
| Native Hawaiian or Other Pacific Islander | 3 (0.7) | 0 | 1 (0.2) | 1 (0.1) | 4 (0.3) |
| White/Caucasian | 331 (74.4) | 341 (77.0) | 324 (72.2) | 665 (74.6) | 996 (74.5) |
| Other | 6 (1.3) | 7 (1.6) | 9 (2.0) | 16 (1.8) | 22 (1.6) |
| African American and Caucasian | 0 | 0 | 1 (0.2) | 1 (0.1) | 1 (0.1) |
| African American/Asian | 0 | 1 (0.2) | 0 | 1 (0.1) | 1 (0.1) |
| American Indian/Alaskan Native | 1 (0.2) | 0 | 0 | 0 | 1 (0.1) |
| Asian & White | 1 (0.2) | 0 | 0 | 0 | 1 (0.1) |
| Asian and Native American | 0 | 1 (0.2) | 0 | 1 (0.1) | 1 (0.1) |
| Biracial (African American and White) | 0 | 0 | 1 (0.2) | 1 (0.1) | 1 (0.1) |
| Black and White | 0 | 0 | 1 (0.2) | 1 (0.1) | 1 (0.1) |
| Black or African American and American Indian | 1 (0.2) | 0 | 0 | 0 | 1 (0.1) |
| Black/Creole | 1 (0.2) | 0 | 0 | 0 | 1 (0.1) |
| Creole | 0 | 0 | 1 (0.2) | 1 (0.1) | 1 (0.1) |
| Indian Non-American | 0 | 0 | 1 (0.2) | 1 (0.1) | 1 (0.1) |
| Mexican | 1 (0.2) | 1 (0.2) | 0 | 1 (0.1) | 2 (0.1) |
| Mixed Black and White/Caucasian | 0 | 0 | 1 (0.2) | 1 (0.1) | 1 (0.1) |
| Mixed White and Black | 0 | 1 (0.2) | 0 | 1 (0.1) | 1 (0.1) |
| Moroccan | 0 | 0 | 1 (0.2) | 1 (0.1) | 1 (0.1) |
| Multiracial | 1 (0.2) | 1 (0.2) | 0 | 1 (0.1) | 2 (0.1) |
| Pakistan | 0 | 0 | 1 (0.2) | 1 (0.1) | 1 (0.1) |
| Pakistan Indian | 0 | 1 (0.2) | 0 | 1 (0.1) | 1 (0.1) |
| White & American Indian | 0 | 1 (0.2) | 0 | 1 (0.1) | 1 (0.1) |
| White, Black and American Indian | 0 | 0 | 1 (0.2) | 1 (0.1) | 1 (0.1) |
| Ethnicity, n (%) | | | | | |
| Hispanic or Latino | 254 (57.1) | 246 (55.5) | 234 (52.1) | 480 (53.8) | 734 (54.9) |
| Non Hispanic or Latino | 191 (42.9) | 197 (44.5) | 215 (47.9) | 412 (46.2) | 603 (45.1) |
| Height (cm) | | | | | |
| n | 445 | 443 | 449 | 892 | 1337 |
| Mean | 164.3 | 164.8 | 165.5 | 165.2 | 164.9 |
| SD | 8.25 | 9.36 | 9.29 | 9.32 | 8.99 |
| Median | 163.0 | 164.0 | 165.1 | 165.0 | 165.0 |
| Minimum | 142 | 137 | 143 | 137 | 137 |
| Maximum | 193 | 195 | 200 | 200 | 200 |
| Weight (kg) | | | | | |
| n | 445 | 443 | 449 | 892 | 1337 |
| Mean | 75.77 | 77.95 | 77.99 | 77.97 | 77.23 |
| SD | 16.310 | 15.889 | 16.072 | 15.972 | 16.113 |
| Median | 73.94 | 76.30 | 77.00 | 76.58 | 75.90 |
| Minimum | 40.9 | 44.0 | 45.0 | 44.0 | 40.9 |
| Maximum | 135.6 | 132.3 | 126.6 | 132.3 | 135.6 |
| Body Mass Index (BMI) (kg/m²) | | | | | |
| n | 445 | 443 | 449 | 892 | 1337 |
| Mean | 27.97 | 28.59 | 28.38 | 28.48 | 28.31 |
| SD | 5.081 | 4.718 | 4.864 | 4.791 | 4.893 |
| Median | 27.35 | 28.20 | 27.82 | 28.07 | 27.76 |
| Minimum | 18.3 | 18.3 | 18.5 | 18.3 | 18.3 |
| Maximun | 40.0 | 39.9 | 40.0 | 40.0 | 40.0 |

Source: Listing 16.2.4.1
Note:
Percentages are based on the number of patients in the ITT Population in each treatment group.
Chiltern · 29JUL2015:5:34 PM · I:\Synergy\33610\21_Stat\Programs\Tables\t_demog.sas · ZScott

TABLE 9
Demographics and Baseline Characteristics
(Safety Population)

| | | Plecanatide | Plecanatide | Active | |
|---|---|---|---|---|---|
| | Placebo | 3 mg | 6 mg | Combined | Overall |
| | (N = 466) | (N = 467) | (N = 469) | (N = 936) | (N = 1402) |
| Age (years) | | | | | |
| n | 466 | 467 | 469 | 936 | 1402 |
| Mean | 44.7 | 45.7 | 45.8 | 45.7 | 45.4 |
| SD | 14.43 | 14.16 | 14.52 | 14.34 | 14.37 |
| Median | 44.5 | 46.0 | 46.0 | 46.0 | 45.0 |
| Minimum | 18 | 18 | 18 | 18 | 18 |
| Maximum | 80 | 80 | 80 | 80 | 80 |
| Gender, n (%) | | | | | |
| Male | 101 (21.7 ) | 105 (22.5) | 103 (22.0) | 208 (22.2) | 309 (22.0) |
| Female | 365 (78.3) | 362 (77.5) | 366 (78.0) | 728 (77.8) | 1093 (78.0) |
| Race, n (%) | | | | | |
| American Indian or Alaskan Native | 0 | 0 | 2 (0.4) | 2 (0.2) | 2 (0.1) |
| Asian | 14 (3.0) | 7 (1.5) | 11 (2.3) | 18 (1.9) | 32 (2.3) |
| Black or African American | 96 (20.6) | 97 (20.8) | 104 (22.2) | 201 (21.5) | 297 (21.2) |
| Native Hawaiian or Other Pacific Islander | 3 (0.6) | 0 | 1 (0.2) | 1 (0.1) | 4 (0.3) |
| White/Caucasian | 347 (74.5) | 356 (76.2) | 341 (72.7) | 697 (74.5) | 1044 (74.5) |
| Other | 6 (1.3) | 7 (1.5) | 10 (2.1) | 17 (1.8) | 23 (1.6) |
| African American and Caucasian | 0 | 0 | 1 (0.2) | 1 (0.1) | 1 (0.1) |
| African American/Asian | 0 | 1 (0.2) | 0 | 1 (0.1) | 1 (0.1) |
| American Indian/Alaskan Native | 1 (0.2) | 0 | 0 | 0 | 1 (0.1) |
| Asian & White | 1 (0.2) | 0 | 0 | 0 | 1 (0.1) |
| Asian and Native American | 0 | 1 (0.2) | 0 | 1 (0.1) | 1 (0.1) |
| Biracial (African American and White) | 0 | 0 | 1 (0.2) | 1 (0.1) | 1 (0.1) |
| Black and White | 0 | 0 | 1 (0.2) | 1 (0.1) | 1 (0.1) |
| Black or African American and American Indian | 1 (0.2) | 0 | 0 | 0 | 1 (0.1) |
| Black/Creole | 1 (0.2) | 0 | 0 | 0 | 1 (0.1) |
| Creole | 0 | 0 | 1 (0.2) | 1 (0.1) | 1 (0.1) |
| Hispanic/White | 0 | 0 | 1 (0.2) | 1 (0.1) | 1 (0.1) |
| Indian Non-American | 0 | 0 | 1 (0.2) | 1 (0.1) | 1 (0.1) |
| Mexican | 1 (0.2) | 1 (0.2) | 0 | 1 (0.1) | 2 (0.1) |
| Mixed Black and White/Caucasian | 0 | 0 | 1 (0.2) | 1 (0.1) | 1 (0.1) |
| Mixed White and Black | 0 | 1 (0.2) | 0 | 1 (0.1) | 1 (0.1) |
| Moroccan | 0 | 0 | 1 (0.2) | 1 (0.1) | 1 (0.1) |
| Multiracial | 1 (0.2) | 1 (0.2) | 0 | 1 (0.1) | 2 (0.1) |
| Pakistan | 0 | 0 | 1 (0.2) | 1 (0.1) | 1 (0.2) |
| Pakistan Indian | 0 | 1 (0.2) | 0 | 1 (0.1) | 1 (0.1) |
| White & American Indian | 0 | 1 (0.2) | 0 | 1 (0.1) | 1 (0.1) |
| White, Black and American Indian | 0 | 0 | 1 (0.2) | 1 (0.1) | 1 (0.1) |
| Ethnicity, n (%) | | | | | |
| Hispanic or Latino | 269 (57.7) | 261 (55.9) | 253 (53.9) | 514 (54.9) | 783 (55.8) |
| Non Hispanic or Latino | 197 (42.3) | 206 (44.1) | 216 (46.1) | 422 (45.1) | 619 (44.2) |
| Height (cm) | | | | | |
| n | 466 | 467 | 469 | 936 | 1402 |
| Mean | 164.4 | 164.8 | 165.5 | 165.1 | 164.9 |
| SD | 8.22 | 9.37 | 9.25 | 9.31 | 8.97 |
| Median | 163.8 | 164.0 | 165.1 | 165.0 | 165.0 |
| Minimum | 142 | 137 | 143 | 137 | 137 |
| Maximum | 193 | 195 | 200 | 200 | 200 |
| Weight (kg) | | | | | |
| n | 466 | 467 | 469 | 936 | 1402 |
| Mean | 75.82 | 77.80 | 77.99 | 77.89 | 77.20 |
| SD | 16.080 | 15.698 | 16.000 | 15.842 | 15.946 |
| Median | 74.20 | 76.20 | 77.10 | 76.40 | 75.75 |
| Minimum | 40.9 | 44.0 | 45.0 | 44.0 | 40.9 |
| Maximum | 135.6 | 132.3 | 126.6 | 132.3 | 135.6 |
| Body Mass Index (BMI) (kg/m²) | | | | | |
| n | 466 | 467 | 469 | 936 | 1402 |
| Mean | 27.95 | 28.56 | 28.38 | 28.47 | 28.30 |
| SD | 5.020 | 4.625 | 4.861 | 4.744 | 4.842 |
| Median | 27.36 | 28.22 | 27.85 | 28.08 | 27.77 |
| Minimum | 18.3 | 18.3 | 18.5 | 18.3 | 18.3 |
| Maximum | 40.0 | 39.9 | 40.0 | 40.0 | 40.0 |

Source: Listing 16.2.4.1
Note:
Percentages are based on the number of patients in the Safety Population in each treatment group.
Chiltern · 29JUL2015:5:34 PM · I:\Synergy\33610\21_Stat\Programs\Tables\t_demog.sas · ZScott

TABLE 10
Patient Compliance with Study Medication (Treatment Compliance)
Missing Returned Tablet Count Imputed as Zero
(ITT Population)

| | | Plecanatide | Plecanatide | Active | |
|---|---|---|---|---|---|
| | Placebo | 3 mg | 6 mg | Combined | Overall |
| | (N = 445) | (N = 443) | (N = 449) | (N = 892) | (N = 1337) |
| Number (%) of patients | 441/445 | 432/443 | 434/449 | 866/892 | 1307/1337 |
| who were compliant | (99.1) | (97.5) | (96.7) | (97.1) | (97.8) |
| with study treatment [1] | | | | | |
| Chi-square p-value [2] | | | | | |
| Plecanatide 3 mg | 0.067 | | | | |
| Plecanatide 6 mg | 0.011 | 0.446 | | | |
| Active Combined | 0.019 | | | | |

Source: Listing 16.2.5
Note:
Percentages are based on the number of patients in the ITT Population in each treatment group.
[1] Treatment compliance is defined as the number of doses actually taken by the patient divided by duration of exposure (date of last dose of study drug − date of first dose of study drug + 1) multiplied by 100. A patient will be considered compliant with study treatment if the treatment compliance calculated is equal to or greater than 80%.
[2] Chi-square test for similar compliance between treatment groups.
Chiltern · 29JUL2015:5:34 PM · I:\Synergy\33610\21 Stat\Programs\Tables\t comp med.sas · ZScott

TABLE 11
Patient Compliance with Study Medication (Treatment Compliance)
in Patients who Completed Treatment Missing Returned Tablet
Count Imputed as Zero (ITT Population)

| | | Plecanatide | Plecanatide | Active | |
|---|---|---|---|---|---|
| | Placebo | 3 mg | 6 mg | Combined | Overall |
| | (N = 445) | (N = 443) | (N = 449) | (N = 892) | (N = 1337) |
| Number (%) of patients who | 394/396 | 375/380 | 389/395 | 764/775 | 1158/1171 |
| completed study treatment | (99.5) | (98.7) | (98.5) | (98.6) | (98.9) |
| who were compliant with | | | | | |
| study treatment [1] | | | | | |
| Chi-square p-value [2] | | | | | |
| Plecanatide 3 mg | 0.232 | | | | |
| Plecanatide 6 mg | 0.154 | 0.811 | | | |
| Active Combined | 0.158 | | | | |

Source: Listing 16.2.5
Note:
Percentages are based on the number of patients in the ITT Population in each treatment group.
[1] Treatment compliance is defined as the number of doses actually taken by the patient divided by duration of exposure (date of last dose of study drug − date of first dose of study drug + 1) multiplied by 100. A patient will be considered compliant with study treatment if the treatment compliance calculated is equal to or greater than 80%.
[2] Chi-square test for similar compliance between treatment groups.
Chiltern · 29JUL2015:5:34 PM · I:\Synergy\33610\21 Stat\Programs\Tables\t comp med.sas · ZScott

TABLE 12
Patient Compliance with Study Medication (Treatment Compliance)
in Patients who did not Complete Treatment Missing Returned Tablet
Count Imputed as Zero (ITT Population)

| | | Plecanatide | Plecanatide | Active | |
|---|---|---|---|---|---|
| | Placebo | 3 mg | 6 mg | Combined | Overall |
| | (N = 445) | (N = 443) | (N = 449) | (N = 392) | (N = 1337) |
| Number (%) of patients who | 47/49 | 57/63 | 45/54 | 102/117 | 149/166 |
| did not complete study | (95.9) | (90.5) | (83.3) | (87.2) | (89.8) |
| treatment who were compliant | | | | | |
| with study treatment [1] | | | | | |
| Chi-square p-value [2] | | | | | |
| Plecanatide 3 mg | 0.267 | | | | |
| Plecanatide 6 mg | 0.039 | 0.249 | | | |
| Active Combined | 0.090 | | | | |

Source: Listing 16.2.5
Note:
Percentages are based on the number of patients in the ITT Population in each treatment group.
[1] Treatment compliance is defined as the number of doses actually taken by the patient divided by duration of exposure (date of last dose of study drug − date of first dose of study drug + 1) multiplied by 100. A patient will be considered compliant with study treatment if the treatment compliance calculated is equal to or greater than 80%.
[2] Chi-square test for similar compliance between treatment groups.
Chiltern · 29JUL2015:5:34 PM · I:\Synergy\33610\21 Stat\Programs\Tales\t comp med.sas · ZScott

TABLE 13
Patient Compliance with Study Medication (Treatment Compliance)
Missing Returned Tablet Count Imputed as Number of Pills Dispensed
(ITT Population)

| | | Plecanatide | Plecanatide | Active | |
|---|---|---|---|---|---|
| | Placebo | 3 mg | 6 mg | Combined | Overall |
| | (N = 445) | (N = 443) | (N = 449) | (N = 892) | (N = 1337) |
| Number (%) of patients who were compliant | 420/445 | 411/443 | 412/449 | 823/892 | 1243/1337 |
| with study treatment [1] | (94.4) | (92.8) | (91.8) | (92.3) | (93.0) |
| Chi-square p-value [2] | | | | | |
| Plecanatide 3 mg | 0.329 | | | | |
| Plecanatide 6 mg | 0.123 | 0.570 | | | |
| Active Combined | 0.154 | | | | |

Source: Listing 16.2.5
Note:
Percentages are based on the number of patients in the ITT Population in each treatment group.
[1] Treatment compliance is defined as the number of doses actually taken by the patient divided by duration of exposure (date of last dose of study drug − date of first dose of study drug + 1) multiplied by 100. A patient will be considered compliant with study treatment if the treatment compliance calculated is equal to or greater than 80%.
[2] Chi-square test for similar compliance between treatment groups.
Chiltern · 29JUL2015:5:34 PM · I:\Synergy\33610\21 Stat\Programs\Tables\t comp med.sas · ZScott

TABLE 14
Patient Compliance with Study Medication (Treatment Compliance)
in Patients who Completed Treatment Missing Returned Tablet Count
Imputed as Number of Pills Dispensed (ITT Population)

| | | Plecanatide | Plecanatide | Active | |
|---|---|---|---|---|---|
| | Placebo | 3 mg | 6 mg | Combined | Overall |
| | (N = 445) | (N = 443) | (N = 449) | (N = 892) | (N = 1337) |
| Number (%) of patients who | 379/396 | 360/380 | 379/393 | 739/775 | 1118/1171 |
| completed study treatment | (95.7) | (94.7) | (95.9) | (95.4) | (95.5) |
| who were compliant with | | | | | |
| study treatment [1] | | | | | |
| Chi-square p-value [2] | | | | | |
| Plecanatide 3 mg | 0.526 | | | | |
| Plecanatide 6 mg | 0.865 | 0.423 | | | |
| Active Combined | 0.784 | | | | |

Source: Listing 16.2.5
Note:
Percentages are based on the number of patients in the ITT Population in each treatment group.
[1] Treatment compliance is defined as the number of doses actually taken by the patient divided by duration of exposure (date of last dose of study drug − date of first dose of study drug + 1) multiplied by 100. A patient will be considered compliant with study treatment if the treatment compliance calculated is equal to or greater than 80%.
[2] Chi-square test for similar compliance between treatment groups.
Chiltern · 29JUL2015:5:34 PM · I:\Synergy\33610\21 Stat\Programs\Talles\t comp med.sas · ZScott

TABLE 15
Patient Compliance with Study Medication (Treatment Compliance)
in Patients who did not Complete Treatment Missing Returned Tablet
Count Imputed as Number of Pills Dispensed (ITT Population)

| | | Plecanatide | Plecanatide | Active | |
|---|---|---|---|---|---|
| | Placebo | 3 mg | 6 mg | Combined | Overall |
| | (N = 445) | (N = 443) | (N = 449) | (N = 892) | (N = 1337) |
| Number (%) of patients who | 41/49 | 51/63 | 33/54 | 84/117 | 125/166 |
| did not complete study | (83.7) | (81.0) | (61.1) | (71.8) | (75.3) |
| treatment who were compliant | | | | | |
| with study treatment [1] | | | | | |
| Chi-square p-value [2] | | | | | |
| Plecanatide 3 mg | 0.709 | | | | |
| Plecanatide 6 mg | 0.011 | 0.017 | | | |
| Active Combined | 0.106 | | | | |

Source: Listing 16.2.5
Note:
Percentages are based on the number of patients in the ITT Population in each treatment group.
[1] Treatment compliance is defined as the number of doses actually taken by the patient divided by duration of exposure (date of last dose of study drug − date of first dose of study drug + 1) multiplied by 100. A patient will be considered compliant with study treatment if the treatment compliance calculated is equal to or greater than 80%.
[2] Chi-square test for similar compliance between treatment groups.
Chiltern · 29JUL2015:5:34 PM · I:\Synergy\33610\21 Stat\Programs\Tables\t comp med.sas · ZScott

TABLE 16
Number and Percentage of Durable Overall CSBM Responders,
Mean Replacement Approach (MRA) (ITT Population)

| | | Plecanatide | Plecanatide | Active |
|---|---|---|---|---|
| | Placebo | 3 mg | 6 mg | Combined |
| | (N = 445) | (N = 443) | (N = 449) | (N = 892) |
| Durable Overall CSBM Responders, n (%) [1] | 57 (12.8) | 89 (20.1) | 90 (20.0) | 179 (20.1) |
| 95% CI (%) [2] | (9.8, 16.3) | (16.5, 24.1) | (16.4, 24.1) | (17.5, 22.8) |
| Non-Responders, n (%) [3] | 388 (87.2) | 354 (79.9) | 359 (80.0) | 713 (79.9) |
| CMH p-value [4] | — | 0.004 | 0.004 | — |
| Odds Ratio (adjusted) [5] | — | 0.586 | 0.587 | — |
| 95% CI | — | (0.408, 0.841) | (0.409, 0.842) | — |
| Difference in Proportions (adjusted) [6] | — | 0.073 | 0.072 | — |
| 95% CI | — | (0.024, 0.121) | (0.024, 0.121) | — |
| Breslow-Day p-value [7] | — | 0.081 | 0.064 | — |

Source: Listings 16.2.6.1, 16.2.6.2
[1] A durable overall CSBM responder is a patient who is a weekly CSBM responder for at least 9 of the 12 treatment weeks, including at least 3 of the last 4 weeks. (A CSBM weekly responder is defined as a patient who has >= 3 CSBMs per week and an increase from baseline of >= 1 CSBM for that week, determined using mean replacement approach (MRA) methodology as defined in Section 4.1.2 of the statistical analysis plan.)
[2] Clopper-Pearson method
[3] Patients missing with respect to the endpoint were scored as non-responders.
[4] CMH p-value for the comparison of treatment group to placebo, stratified by gender.
[5] Common odds ratio, treatment/placebo adjusted for stratification factor (gender) in the CMH analysis.
[6] Proportion of responders in treatment group minus proportion in Placebo group, adjusted for stratification factor (gender) in the CMH analysis.
[7] Breslow-Day p-value for test of consistency of the treatment effect across the stratification.
Chiltern · 29JUL2015:5:34 PM · I:\Synergy\33610\21_Stat\Programs\Tables\t_respond.sas · ZScott

TABLE 17
Number and Percentage of Durable Overall CSBM Responders,
Mean Replacement Approach (MRA) (ITT Population-Excluding
Duplicate Patients)

| | | Plecanatide | Plecanatide | Active |
|---|---|---|---|---|
| | Placebo | 3 mg | 6 mg | Combined |
| | (N = 437) | (N = 434) | (N = 443) | (N = 877) |
| Durable Overall CSBM Responders, n (%) [1] | 57 (13.0) | 89 (20.5) | 90 (20.3) | 179 (20.4) |
| 95% CI (%) [2] | (10.0, 16.6) | (16.8, 24.6) | (16.7, 24.4) | (17.8, 23.2) |
| Non-Responders, n (%) [3] | 380 (87.0) | 345 (79.5) | 353 (79.7) | 698 (79.6) |
| CMH p-value [4] | — | 0.003 | 0.004 | — |
| Odds Ratio (adjusted) [5] | — | 0.583 | 0.589 | — |
| 95% CI | — | (0.406, 0.838) | (0.410, 0.846) | — |
| Difference in Proportions (adjusted) [6] | — | 0.075 | 0.073 | — |
| 95% CI | — | (0.025, 0.124) | (0.024, 0.122) | — |
| Breslow-Day p-value [7] | — | 0.086 | 0.072 | — |

Source: Listings 16.2.6.1, 16.2.6.2
[1] A durable overall CSBM responder is a patient who is a weekly CSBM responder for at least 9 of the 12 treatment weeks, including at least 3 of the last 4 weeks. (A CSBM weekly responder is defined as a patient who has >= 3 CSBMs per week and an increase from baseline of >= 1 CSBM for that week, determined using mean replacement approach (MRA) methodology as defined in Section 4.1.2 of the statistical analysis plan.)
[2] Clopper-Pearson method
[3] Patients missing with respect to the endpoint were scored as non-responders.
[4] CMH p-value for the comparison of treatment group to placebo, stratified by gender.
[5] Common odds ratio, treatment/placebo adjusted for stratification factor (gender) in the CMH analysis.
[6] Proportion of responders in treatment group minus proportion in Placebo group, adjusted for stratification factor (gender) in the CMH analysis.
[7] Breslow-Day p-value for the test of consistency of the treatment effect across the stratification.
Chiltern · 29JUL2015:5:34 PM · I:\Synergy\33610\21_Stat\Programs\Tables\t_respond.sas · ZScott

TABLE 18
Number and Percentage of Durable Overall CSBM Responders, Mean Replacement Approach (MRA)
(All Randomized (includes Duplicate Patients))

| | | Plecanatide | Plecanatide | Active |
|---|---|---|---|---|
| | Placebo | 3 mg | 6 mg | Combined |
| | (N = 469) | (N = 470) | (N = 471) | (N = 941) |
| Durable Overall CSBM Responders, n (%) [1] | 57 (12.2) | 93 (19.8) | 92 (19.5) | 185 (19.7) |
| 95% CI (%) [2] | (9.3, 15.5) | (16.3, 23.7) | (16.0, 23.4) | (17.2, 22.3) |
| Non-Responders, n (%) [3] | 412 (87.8) | 377 (80.2) | 379 (80.5) | 756 (80.3) |
| CMH p-value [4] | — | 0.001 | 0.002 | — |
| Odds Ratio (adjusted) [5] | — | 0.562 | 0.571 | — |
| 95% CI | — | (0.393, 0.804) | (0.399, 0.817) | — |
| Difference in Proportions (adjusted) [6] | — | 0.076 | 0.074 | — |
| 95% CI | — | (0.030, 0.123) | (0.027, 0.120) | — |
| Breslow-Day p-value [7] | — | 0.061 | 0.080 | — |

Source: Listings 16.2.6.1 16.2.6.2
[1] A durable overall CSBM responder is a patient who is a weekly CSBM responder for at least 9 of the 12 treatment weeks, including at least 3 of the last 4 weeks. (A CSBM weekly responder is defined as a patient who has >= 3 CSBMs per week and an increase from baseline of >= 1 CSBM for that week, determined using mean replacement approach (MRA) methodology as defined in Section 4.1.2 of the statistical analysis plan.)
[2] Clopper-Pearson method
[3] Patients missing with respect to the endpoint were scored as non-responders.
[4] CMH p-value for the comparison of treatment group to placebo, stratified by gender.
[5] Common odds ratio, treatment/placebo adjusted for stratification factor (gender) in the CMH analysis.
[6] Proportion of responders in treatment group minus proportion in Placebo group, adjusted for stratifiatiion factor (gender) in the CMH analysis.
[7] Breslow-Day p-value for the test of consistency of the treatment effect across the stratification. Chiltern · 29JUL2015:5:34 PM · I:\Synergy\33610\21_Stat\Programs\Tables\t_respond.sas · ZScott

TABLE 19
Number and Percentage of CSBM Weekly Responders by Week, Mean Replacement Approach (MRA)
(ITT Population)

| | | Plecanatide | Plecanatide | Active |
|---|---|---|---|---|
| | Placebo | 3 mg | 6 mg | Combined |
| | (N = 445) | (N = 443) | (N = 449) | (N = 892) |
| Week 1 | | | | |
| CSBM Weekly Responders, n (%) [1] | 69 (15.5) | 121 (27.3) | 114 (25.4) | 235 (26.3) |
| 95% CI (%) [2] | (12.3, 19.2) | (23.2, 31.7) | (21.4, 29.7) | (23.5, 29.4) |
| Non-Responders, n (%) [3] | 376 (84.5) | 322 (72.7) | 335 (74.6) | 657 (73.7) |
| CMH p-value [4] | — | <0.001 | <0.001 | — |
| Odds Ratio (adjusted) [5] | — | 0.488 | 0.539 | — |
| 95% CI | — | (0.351, 0.680) | (0.387, 0.752) | — |
| Difference in Proportions (adjusted) [6] | — | 0.118 | 0.099 | — |
| 95% CI | — | (0.065, 0.172) | (0.047, 0.151) | — |
| Breslow-Day p-value [7] | — | 0.723 | 0.077 | — |
| Week 2 | | | | |
| CSBM Weekly Responders, n (%) [1] | 74 (16.6) | 112 (25.3) | 123 (27.4) | 235 (26.3) |
| 95% CI (%) [2] | (13.3, 20.4) | (21.3, 29.6) | (23.3, 31.8) | (23.5, 29.4) |
| Non-Responders, n (%) [3] | 371 (83.4) | 331 (74.7) | 326 (72.6) | 657 (73.7) |
| CMH p-value [4] | — | 0.002 | <0.001 | — |
| Odds Ratio (adjusted) [5] | — | 0.59 | 0.53 | — |
| 95% CI | — | (0.425, 0.820) | (0.383, 0.733) | — |
| Difference in Proportions (adjusted) [6] | — | 0.087 | 0.108 | — |
| 95% CI | — | (0.033, 0.140) | (0.054, 0.161) | — |
| Breslow-Day p-value [7] | — | 0.159 | 0.066 | — |
| Week 3 | | | | |
| CSBM Weekly Responders, n (%) [1] | 100 (22.5) | 125 (28.2) | 120 (26.7) | 245 (27.5) |
| 95% CI (%) [2] | (18.7, 26.6) | (24.1, 32.7) | (22.7, 31.1) | (24.6, 30.5) |
| Non-Responders, n (%) [3] | 345 (77.5) | 318 (71.8) | 329 (73.3) | 647 (72.5) |
| CMH p-value [4] | — | 0.05 | 0.14 | — |
| Odds Ratio (adjusted) [5] | — | 0.739 | 0.796 | — |
| 95% CI | — | (0.545, 1.001) | (0.587, 1.079) | — |
| Difference in Proportions (adjusted) [6] | — | 0.057 | 0.043 | — |
| 95% CI | — | (0.000, 0.114) | (−0.014, 0.099) | — |
| Breslow-Day p-value [7] | — | 0.158 | 0.032 | — |
| Week 4 | | | | |
| CSBM Weekly Responders, n (%) [1] | 96 (21.6) | 132 (29.8) | 127 (28.3) | 259 (29.0) |
| 95% CI (%) [2] | (17.8, 25.7) | (25.6, 34.3) | (24.2, 32.7) | (26.1, 32.1) |
| Non-Responders, n (%) [3] | 349 (78.4) | 311 (70.2) | 322 (71.7) | 633 (71.0) |
| CMH p-value [4] | — | 0.005 | 0.021 | — |
| Odds Ratio (adjusted) [5] | — | 0.648 | 0.697 | — |
| 95% CI | — | (0.479, 0.879) | (0.514, 0.946) | — |
| Difference in Proportions (adjusted) [6] | — | 0.082 | 0.067 | — |
| 95% CI | — | (0.025, 0.139) | (0.011, 0.124) | — |
| Breslow-Day p-value [7] | — | 0.373 | 0.843 | — |
| Week 5 | | | | |
| CSBM Weekly Responders, n (%) [1] | 108 (24.3) | 154 (34.8) | 133 (29.6) | 287 (32.2) |
| 95% CI (%) [2] | (20.4, 28.5) | (30.3, 39.4) | (25.4, 34.1) | (29.1, 35.4) |
| Non-Responders, n (%) [3] | 337 (75.7) | 289 (65.2) | 316 (70.4) | 605 (67.8) |
| CMH p-value [4] | — | <0.001 | 0.072 | — |
| Odds Ratio (adjusted) [5] | — | 0.602 | 0.762 | — |
| 95% CI | — | (0.450, 0.806) | (0.566, 1.025) | — |
| Difference in Proportions (adjusted) [6] | — | 0.105 | 0.054 | — |
| 95% CI | — | (0.045, 0.164) | (−0.004, 0.112) | — |
| Breslow-Day p-value [7] | — | 0.528 | 0.234 | — |
| Week 6 | | | | |
| CSBM Weekly Responders, n (%) [1] | 96 (21.6) | 137 (30.9) | 146 (32.5) | 283 (31.7) |
| 95% CI (%) [2] | (17.8, 25.7) | (26.6, 35.5) | (28.2, 37.1) | (28.7, 34.9) |
| Non-Responders, n (%) [3] | 349 (78.4) | 306 (69.1) | 303 (67.5) | 609 (68.3) |
| CMH p-value [4] | — | 0.002 | <0.001 | — |
| Odds Ratio (adjusted) [5] | — | 0.615 | 0.571 | — |
| 95% CI | — | (0.455, 0.833) | (0.423, 0.771) | — |
| Difference in Proportions (adjusted) [6] | — | 0.093 | 0.109 | — |
| 95% CI | — | (0.036, 0.151) | (0.052, 0.167) | — |
| Breslow-Day p-value [7] | — | 0.189 | 0.194 | — |
| Week 7 | | | | |
| CSBM Weekly Responders, n (%) [1] | 108 (24.3) | 145 (32.7) | 140 (31.2) | 285 (32.0) |
| 95% CI (%) [2] | (20.4, 28.5) | (28.4, 37.3) | (26.9, 35.7) | (28.9, 35.1) |
| Non-Responders, n (%) [3] | 337 (75.7) | 298 (67.3) | 309 (68.8) | 607 (68.0) |
| CMH p-value [4] | — | 0.005 | 0.021 | — |
| Odds Ratio (adjusted) [5] | — | 0.659 | 0.707 | — |
| 95% CI | — | (0.491, 0.884) | (0.527, 0.950) | — |
| Difference in Proportions (adjusted) [6] | — | 0.085 | 0.069 | — |
| 95% CI | — | (0.025, 0.144) | (0.011, 0.128) | — |
| Breslow-Day p-value [7] | — | 0.97 | 0.873 | — |
| Week 8 | | | | |
| CSBM Weekly Responders, n (%) [1] | 115 (25.8) | 145 (32.7) | 144 (32.1) | 289 (32.4) |
| 95% CI (%) [2] | (21.8, 30.2) | (28.4, 37.3) | (27.8, 36.6) | (29.3, 35.6) |
| Non-Responders, n (%) [3] | 330 (74.2) | 298 (67.3) | 305 (67.9) | 603 (67.6) |
| CMH p-value [4] | — | 0.024 | 0.04 | — |
| Odds Ratio (adjusted) [5] | — | 0.717 | 0.738 | — |
| 95% CI | — | (0.536, 0.958) | (0.552, 0.987) | — |
| Difference in Proportions (adjusted) [6] | — | 0.069 | 0.062 | — |
| 95% CI | — | (0.009, 0.128) | (0.003, 0.122) | — |
| Breslow-Day p-value [7] | — | 0.313 | 0.775 | — |
| Week 9 | | | | |
| CSBM Weekly Responders, n (%) [1] | 111 (24.9) | 147 (33.2) | 141 (31.4) | 288 (32.3) |
| 95% CI (%) [2] | (21.0, 29.2) | (28.8, 37.8) | (27.1, 35.9) | (29.2, 35.5) |
| Non-Responders, n (%) [3] | 334 (75.1) | 296 (66.8) | 308 (68.6) | 604 (67.7) |
| CMH p-value [4] | — | 0.007 | 0.032 | — |
| Odds Ratio (adjusted) [5] | — | 0.67 | 0.727 | — |
| 95% CI | — | (0.501, 0.898) | (0.543, 0.974) | — |
| Difference in Proportions (adjusted) [6] | — | 0.082 | 0.065 | — |
| 95% CI | — | (0.023, 0.142) | (0.006, 0.123) | — |
| Breslow-Day p-value [7] | — | 0.142 | 0.036 | — |
| Week 10 | | | | |
| CSBM Weekly Responders, n (%) [1] | 110 (24.7) | 137 (30.9) | 154 (34.3) | 291 (32.6) |
| 95% CI (%) [2] | (20.8, 29.0) | (26.6, 35.5) | (29.9, 38.9) | (29.6, 35.8) |
| Non-Responders, n (%) [3] | 335 (75.3) | 306 (69.1) | 295 (65.7) | 601 (67.4) |
| CMH p-value [4] | — | 0.04 | 0.002 | — |
| Odds Ratio (adjusted) [5] | — | 0.734 | 0.629 | — |
| 95% CI | — | (0.547, 0.986) | (0.471, 0.841) | — |
| Difference in Proportions (adjusted) [6] | — | 0.062 | 0.096 | — |
| 95% CI | — | (0.003, 0.121) | (0.036, 0.155) | — |
| Breslow-Day p-value [7] | — | 0.691 | 0.314 | — |
| Week 11 | | | | |
| CSBM Weekly Responders, n (%) [1] | 109 (24.5) | 147 (33.2) | 145 (32.3) | 292 (32.7) |
| 95% CI (%) [2] | (20.6, 28.8) | (28.8, 37.8) | (28.0, 36.8) | (29.7, 35.9) |
| Non-Responders, n (%) [3] | 336 (75.5) | 296 (66.8) | 304 (67.7) | 600 (67.3) |
| CMH p-value [4] | — | 0.004 | 0.01 | — |
| Odds Ratio (adjusted) [5] | — | 0.654 | 0.68 | — |
| 95% CI | — | (0.488, 0.877) | (0.508, 0.912) | — |
| Difference in Proportions (adjusted) [6] | — | 0.086 | 0.078 | — |
| 95% CI | — | (0.027, 0.146) | (0.019, 0.137) | — |
| Breslow-Day p-value [7] | — | 0.997 | 0.37 | — |
| Week 12 | | | | |
| CSBM Weekly Responders, n (%) [1] | 113 (25.4) | 140 (31.6) | 151 (33.6) | 291 (32.6) |
| 95% CI (%) [2] | (21.4, 29.7) | (27.3, 36.2) | (29.3, 38.2) | (29.6, 35.8) |
| Non-Responders, n (%) [3] | 332 (74.6) | 303 (68.4) | 298 (66.4) | 601 (67.4) |
| CMH p-value [4] | — | 0.041 | 0.007 | — |
| Odds Ratio (adjusted) [5] | — | 0.737 | 0.672 | — |
| 95% CI | — | (0.550, 0.988) | (0.503, 0.897) | — |
| Difference in Proportions (adjusted) [6] | — | 0.062 | 0.082 | — |
| 95% CI | — | (0.003, 0.121) | (0.023, 0.142) | — |
| Breslow-Day p-value [7] | — | 0.999 | 0.827 | — |
| Post-Treatment Week 13 | | | | |
| CSBM Weekly Responders, n (%) [1] | 102 (22.9) | 121 (27.3) | 106 (23.6) | 227 (25.4) |
| 95% CI (%) [2] | (19.1, 27.1) | (23.2, 31.7) | (19.8, 27.8) | (22.6, 28.4) |
| Non-Responders, n (%) [3] | 343 (77.1) | 322 (72.7) | 343 (76.4) | 665 (74.6) |
| CMH p-value [4] | — | 0.133 | 0.808 | — |
| Odds Ratio (adjusted) [5] | — | 0.792 | 0.962 | — |
| 95% CI | — | (0.584, 1.074) | (0.705, 1.313) | — |
| Difference in Proportions (adjusted) [6] | — | 0.044 | 0.007 | — |
| 95% CI | — | (−0.013, 0.101) | (−0.048, 0.062) | — |
| Breslow-Day p-value [7] | — | 0.821 | 0.847 | — |
| Post-Treatment Week 14 | | | | |
| CSBM Weekly Responders, n (%) [1] | 83 (18.7) | 94 (21.2) | 83 (18.5) | 177 (19.8) |
| 95% CI (%) [2] | (15.1, 22.6) | (17.5, 25.3) | (15.0, 22.4) | (17.3, 22.6) |
| Non-Responders, n (%) [3] | 362 (81.3) | 349 (78.8) | 366 (81.5) | 715 (80.2) |
| CMH p-value [4] | — | 0.339 | 0.949 | — |
| Odds Ratio (adjusted) [5] | — | 0.852 | 1.011 | — |
| 95% CI | — | (0.612, 1.184) | (0.722, 1.416) | — |
| Difference in Proportions (adjusted) [6] | — | 0.026 | −0.002 | — |
| 95% CI | — | (−0.027, 0.078) | (−0.053, 0.049) | — |
| Breslow-Day p-value [7] | — | 0.23 | 0.399 | — |

Source: Listings 16.2.6.1, 16.2.6.2
[1] A CSBM weekly responder is defined as a patient who has >=3 CSBMs per week and an increase from baseline of >=1 CSBM for that week, determined using mean replacement approach (MRA) methodology as defined in Section 4.1.2 of the statistical analysis plan.
[2] Clopper-Pearson method
[3] Patients missing with respect to the endpoint were scored as non-responders.
[4] CMH p-value for the comparison of treatment group to placebo, stratified by gender.
[5] Common odds ratio, treatment/placebo adjusted for stratification factor (gender) in the CMH analysis.
[6] Proportion of responders in treatment group minus proportion in Placebo group, adjusted for stratification factor (gender) in the CMH analysis.
[7] Breslow-Day p-value for the test of consistency of the treatment effect across the stratification.
Chiltern • 29JUL2015:5:34 PM • I:\Synergy\33610\21_Stat\Programs\Tables\t_respond.sas • ZScott

TABLE 20
Number and Percentage of CSBM Weekly Responders by Week, Mean Replacement Approach (MRA)
(ITT Population-Excluding Duplicate Patients)

| | | Plecanatide | Plecanatide | Active |
|---|---|---|---|---|
| | Placebo | 3 mg | 6 mg | Combined |
| | (N = 437) | (N = 434) | (N = 443) | (N = 877) |
| Week 1 | | | | |
| CSBM Weekly Responders, n (%) [1] | 68 (15.6) | 121 (27.9) | 114 (25.7) | 235 (26.8) |
| 95% CI (%) [2] | (12.3, 19.3) | (23.7, 32.4) | (21.7, 30.1) | (23.9, 29.9) |
| Non-Responders, n (%) [3] | 369 (84.4) | 313 (72.1) | 329 (74.3) | 642 (73.2) |
| CMH p-value [4] | — | <0.001 | <0.001 | — |
| Odds Ratio (adjusted) [5] | — | 0.477 | 0.533 | — |
| 95% CI | — | (0.342, 0.665) | (0.381, 0.744) | — |
| Difference in Proportions (adjusted) [6] | — | 0.123 | 0.102 | — |
| 95% CI | — | (0.069, 0.177) | (0.049, 0.154) | — |
| Breslow-Day p-value [7] | — | 0.707 | 0.078 | — |
| Week 2 | | | | |
| CSBM Weekly Responders, n (%) [1] | 73 (16.7) | 110 (25.3) | 123 (27.8) | 233 (26.6) |
| 95% CI (%) [2] | (13.3, 20.5) | (21.3, 29.7) | (23.6, 32.2) | (23.7, 29.6) |
| Non-Responders, n (%) [3] | 364 (83.3) | 324 (74.7) | 320 (72.2) | 644 (73.4) |
| CMH p-value [4] | — | 0.002 | <0.001 | — |
| Odds Ratio (adjusted) [5] | — | 0.592 | 0.523 | — |
| 95% CI | — | (0.425, 0.824) | (0.378, 0.724) | — |
| Difference in Proportions (adjusted) [6] | — | 0.086 | 0.111 | — |
| 95% CI | — | (0.033, 0.140) | (0.056, 0.165) | — |
| Breslow-Day p-value [7] | — | 0.175 | 0.067 | — |
| Week 3 | | | | |
| CSBM Weekly Responders, n (%) [1] | 99 (22.7) | 122 (28.1) | 119 (26.9) | 241 (27.5) |
| 95% CI (%) [2] | (18.8, 26.9) | (23.9, 32.6) | (22.8, 31.2) | (24.5, 30.6) |
| Non-Responders, n (%) [3] | 338 (77.3) | 312 (71.9) | 324 (73.1) | 636 (72.5) |
| CMH p-value [4] | — | 0.066 | 0.149 | — |
| Odds Ratio (adjusted) [5] | — | 0.750 | 0.798 | — |
| 95% CI | — | (0.552, 1.019) | (0.588, 1.084) | — |
| Difference in Proportions (adjusted) [6] | — | 0.054 | 0.042 | — |
| 95% CI | — | (−0.003, 0.112) | (−0.015, 0.099) | — |
| Breslow-Day p-value [7] | — | 0.192 | 0.036 | — |
| Week 4 | | | | |
| CSBM Weekly Responders, n (%) [1] | 95 (21.7) | 130 (30.0) | 126 (28.4) | 256 (29.2) |
| 95% CI (%) [2] | (18.0, 25.9) | (25.7, 34.5) | (24.3, 32.9) | (26.2, 32.3) |
| Non-Responders, n (%) [3] | 342 (78.3) | 304 (70.0) | 317 (71.6) | 621 (70.8) |
| CMH p-value [4] | — | 0.006 | 0.022 | — |
| Odds Ratio (adjusted) [5] | — | 0.650 | 0.698 | — |
| 95% CI | — | (0.479, 0.883) | (0.513, 0.949) | — |
| Difference in Proportions (adjusted) [6] | — | 0.082 | 0.067 | — |
| 95% CI | — | (0.024, 0.140) | (0.010, 0.124) | — |
| Breslow-Day p-value [7] | — | 0.240 | 0.896 | — |
| Week 5 | | | | |
| CSBM Weekly Responders, n (%) [1] | 107 (24.5) | 153 (35.3) | 132 (29.8) | 285 (32.5) |
| 95% CI (%) [2] | (20.5, 28.8) | (30.8, 40.0) | (25.6, 34.3) | (29.4, 35.7) |
| Non-Responders, n (%) [3] | 330 (75.5) | 281 (64.7) | 311 (70.2) | 592 (67.5) |
| CMH p-value [4] | — | <0.001 | 0.077 | — |
| Odds Ratio (adjusted) [5] | — | 0.596 | 0.764 | — |
| 95% CI | — | (0.444, 0.800) | (0.567, 1.029) | — |
| Difference in Proportions (adjusted) [6] | — | 0.107 | 0.053 | — |
| 95% CI | — | (0.047, 0.168) | (−0.005, 0.112) | — |
| Breslow-Day p-value [7] | — | 0.552 | 0.261 | — |
| Week 6 | | | | |
| CSBM Weekly Responders, n (%) [1] | 95 (21.7) | 136 (31.3) | 146 (33.0) | 282 (32.2) |
| 95% CI (%) [2] | (18.0, 25.9) | (27.0, 35.9) | (28.6, 37.6) | (29.1, 35.4) |
| Non-Responders, n (%) [3] | 342 (78.3) | 298 (68.7) | 297 (67.0) | 595 (67.8) |
| CMH p-value [4] | — | 0.001 | <0.001 | — |
| Odds Ratio (adjusted) [5] | — | 0.610 | 0.565 | — |
| 95% CI | — | (0.450, 0.826) | (0.418, 0.764) | — |
| Difference in Proportions (adjusted) [6] | — | 0.096 | 0.112 | — |
| 95% CI | — | (0.038, 0.154) | (0.054, 0.171) | — |
| Breslow-Day p-value [7] | — | 0.197 | 0.204 | — |
| Week 7 | | | | |
| CSBM Weekly Responders, n (%) [1] | 106 (24.3) | 144 (33.2) | 140 (31.6) | 284 (32.4) |
| 95% CI (%) [2] | (20.3, 28.6) | (28.8, 37.8) | (27.3, 36.2) | (29.3, 35.6) |
| Non-Responders, n (%) [3] | 331 (75.7) | 290 (66.8) | 303 (68.4) | 593 (67.6) |
| CMH p-value [4] | — | 0.004 | 0.015 | — |
| Odds Ratio (adjusted) [5] | — | 0.645 | 0.693 | — |
| 95% CI | — | (0.480, 0.868) | (0.515, 0.932) | — |
| Difference in Proportions (adjusted) [6] | — | 0.089 | 0.074 | — |
| 95% CI | — | (0.029, 0.149) | (0.014, 0.133) | — |
| Breslow-Day p-value [7] | — | 0.964 | 0.878 | — |
| Week 8 | | | | |
| CSBM Weekly Responders, n (%) [1] | 113 (25.9) | 144 (33.2) | 143 (32.3) | 287 (32.7) |
| 95% CI (%) [2] | (21.8, 30.2) | (28.8, 37.8) | (27.9, 36.9) | (29.6, 35.9) |
| Non-Responders, n (%) [3] | 324 (74.1) | 290 (66.8) | 300 (67.7) | 590 (67.3) |
| CMH p-value [4] | — | 0.018 | 0.036 | — |
| Odds Ratio (adjusted) [5] | — | 0.703 | 0.731 | — |
| 95% CI | — | (0.524, 0.942) | (0.545, 0.979) | — |
| Difference in Proportions (adjusted) [6] | — | 0.073 | 0.064 | — |
| 95% CI | — | (0.013, 0.134) | (0.005, 0.124) | — |
| Breslow-Dry p-value [7] | — | 0.407 | 0.950 | — |
| Week 9 | | | | |
| CSBM Weekly Responders, n (%) [1] | 111 (25.4) | 146 (33.6) | 140 (31.6) | 286 (32.6) |
| 95% CI (%) [2] | (21.4, 29.8) | (29.2, 38.3) | (27.3, 36.2) | (29.5, 35.8) |
| Non-Responders, n (%) [3] | 326 (74.6) | 288 (66.4) | 303 (68.4) | 591 (67.4) |
| CMH p-value [4] | — | 0.008 | 0.042 | — |
| Odds Ratio (adjusted) [5] | — | 0.673 | 0.738 | — |
| 95% CI | — | (0.502, 0.902) | (0.550, 0.989) | — |
| Difference in Proportions (adjusted) [6] | — | 0.082 | 0.062 | — |
| 95% CI | — | (0.022, 0.142) | (0.003, 0.121) | — |
| Breslow-Day p-value [7] | — | 0.164 | 0.047 | — |
| Week 10 | | | | |
| CSBM Weekly Responders, n (%) [1] | 109 (24.9) | 136 (31.3) | 153 (34.5) | 289 (33.0) |
| 95% CI (%) [2] | (21.0, 29.3) | (27.0, 35.9) | (30.1, 39.2) | (29.8, 36.2) |
| Non-Responders, n (%) [3] | 328 (75.1) | 298 (68.7) | 290 (65.5) | 588 (67.0) |
| CMH p-value [4] | — | 0.037 | 0.002 | — |
| Odds Ratio (adjusted) [5] | — | 0.729 | 0.630 | — |
| 95% CI | — | (0.542, 0.981) | (0.470, 0.843) | — |
| Difference in Proportions (adjusted) [6] | — | 0.064 | 0.096 | — |
| 95% CI | — | (0.004, 0.123) | (0.036, 0.156) | — |
| Breslow-Day p-value [7] | — | 0.718 | 0.350 | — |
| Week 11 | | | | |
| CSBM Weekly Responders, n (%) [1] | 109 (24.9) | 147 (33.9) | 144 (32.5) | 291 (33.2) |
| 95% CI (%) [2] | (21.0, 29.3) | (29.4, 38.5) | (28.2, 37.1) | (30.1, 36.4) |
| Non-Responders, n (%) [3] | 328 (75.1) | 287 (66.1) | 299 (67.5) | 586 (66.8) |
| CMH p-value [4] | — | 0.004 | 0.013 | — |
| Odds Ratio (adjusted) [5] | — | 0.649 | 0.690 | — |
| 95% CI | — | (0.484, 0.871) | (0.514, 0.926) | — |
| Difference in Proportions (adjusted) [6] | — | 0.089 | 0.076 | — |
| 95% CI | — | (0.029, 0.149) | (0.016, 0.135) | — |
| Breslow-Day p-value [7] | — | 0.964 | 0.434 | — |
| Week 12 | | | | |
| CSBM Weekly Responders, n (%) [1] | 112 (25.6) | 140 (32.3) | 150 (33.9) | 290 (33.1) |
| 95% CI (%) [2] | (21.6, 30.0) | (27.9, 36.9) | (29.5, 38.5) | (30.0, 36.3) |
| Non-Responders, n (%) [3] | 325 (74.4) | 294 (67.7) | 293 (66.1) | 587 (66.9) |
| CMH p-value [4] | — | 0.031 | 0.008 | — |
| Odds Ratio (adjusted) [5] | — | 0.724 | 0.673 | — |
| 95% CI | — | (0.539, 0.972) | (0.503, 0.901) | — |
| Difference in Proportions (adjusted) [6] | — | 0.066 | 0.082 | — |
| 95% CI | — | (0.006, 0.126) | (0.022, 0.142) | — |
| Breslow-Day p-value [7] | — | >0.999 | 0.886 | — |
| Post-Treatment Week 13 | | | | |
| CSBM Weekly Responders, n (%) [1] | 100 (22.9) | 119 (27.4) | 105 (23.7) | 224 (25.5) |
| 95% CI (%) [2] | (19.0, 27.1) | (23.3, 31.9) | (19.8, 27.9) | (22.7, 28.6) |
| Non-Responders, n (%) [3] | 337 (77.1) | 315 (72.6) | 338 (76.3) | 653 (74.5) |
| CMH p-value [4] | — | 0.124 | 0.770 | — |
| Odds Ratio (adjusted) [5] | — | 0.786 | 0.954 | — |
| 95% CI | — | (0.578, 1.069) | (0.698, 1.305) | — |
| Difference in Proportions (adjusted) [6] | — | 0.045 | 0.008 | — |
| 95% CI | — | (−0.012, 0.103) | (−0.047, 0.064) | — |
| Breslow-Day p-value [7] | — | 0.707 | 0.828 | — |
| Post-Treatment Week 14 | | | | |
| CSBM Weekly Responders, n (%) [1] | 83 (19.0) | 93 (21.4) | 82 (18.5) | 175 (20.0) |
| 95% CI (%) [2] | (15.4, 23.0) | (17.7, 25.6) | (15.0, 22.4) | (17.4, 22.8) |
| Non-Responders, n (%) [3] | 354 (81.0) | 341 (78.6) | 361 (81.5) | 702 (80.0) |
| CMH p-value [4] | — | 0.372 | 0.854 | — |
| Odds Ratio (adjusted) [5] | — | 0.860 | 1.032 | — |
| 95% CI | — | (0.618, 1.198) | (0.736, 1.448) | — |
| Difference in Proportions (adjusted) [6] | — | 0.024 | −0.005 | — |
| 95% CI | — | (−0.029, 0.078) | (−0.056, 0.047) | — |
| Breslow-Day p-value [7] | — | 0.202 | 0.344 | — |

Source: Listings 16.2.6.1, 16.2.6.2
[1] A CSBM weekly responder is defined as a patient who has >=3 CSBMs per week and an increase from baseline of >=1 CSBM for that week, determined using mean replacement approach (MRA) methodology as defined in Section 4.1.2 of the statistical analysis plan.
[2] Clopper-Pearson method
[3] Patients missing with respect to the endpoint were scored as non responders
[4] CMH p-value for the comparison of treatment group to placebo, stratified by gender.
[5] Common odds ratio, treatment/placebo adjusted for stratification factor (gender) in the CMH analysis.
[6] Proportion of responders in treatment group minus proportion in Placebo group, adjusted for stratification factor (gender) in the CMH analysis.
[7] Breslow-Day p-value for the test of consistency of the treatment effect across the stratification.
Chiltern • 29JUL2015:5:34 PM • I:\Synergy\33610\21_Stat\Programs\Tables\t_respond.sas • ZScott

TABLE 21
Number and Percentage of CSBM Weekly Responders by Week, Mean Replacement Approach (MRA)
(All Randomized (includes Duplicate Patients))

| | | Plecanatide | Plecanatide | Active |
|---|---|---|---|---|
| | Placebo | 3 mg | 6 mg | Combined |
| | (N = 469) | (N = 470) | (N = 471) | (N = 941) |
| Week 1 | | | | |
| CSBM Weekly Responders, n (%) [1] | 70 (14.9) | 128 (27.2) | 120 (25.5) | 248 (26.4) |
| 95% CI (%) [2] | (11.8, 18.5) | (23.3, 31.5) | (21.6, 29.7) | (23.6, 29.3) |
| Non-Responders, n (%) [3] | 399 (85.1) | 342 (72.8) | 351 (74.5) | 693 (73.6) |
| CMH p-value [4] | — | <0.001 | <0.001 | — |
| Odds Ratio (adjusted) [5] | — | 0.468 | 0.513 | — |
| 95% CI | — | (0.338, 0.648) | (0.370, 0.712) | — |
| Difference in Proportions (adjusted) [6] | — | 0.123 | 0.106 | — |
| 95% CI | — | (0.072, 0.175) | (0.055, 0.156) | — |
| Breslow-Day p-value [7] | — | 0.610 | 0.125 | — |
| Week 2 | | | | |
| CSBM Weekly Responders, n (%) [1] | 78 (16.6) | 120 (25.5) | 132 (28.0) | 252 (26.8) |
| 95% CI (%) [2] | (13.4, 20.3) | (21.6, 29.7) | (24.0, 32.3) | (24.0, 29.7) |
| Non-Responders, n (%) [3] | 391 (83.4) | 350 (74.5) | 339 (72.0) | 689 (73.2) |
| CMH p-value [4] | — | <0.001 | <0.001 | — |
| Odds Ratio (adjusted) [5] | — | 0.582 | 0.513 | — |
| 95% CI | — | (0.423, 0.801) | (0.374, 0.703) | — |
| Difference in Proportions (adjusted) [6] | — | 0.089 | 0.114 | — |
| 95% CI | — | (0.037, 0.141) | (0.061, 0.167) | — |
| Breslow-Day p-value [7] | — | 0.201 | 0.207 | — |
| Week 3 | | | | |
| CSBM Weekly Responders, n (%) [1] | 102 (21.7) | 135 (28.7) | 129 (27.4) | 264 (28.1) |
| 95% CI (%) [2] | (18.1, 25.8) | (24.7, 33.0) | (23.4, 31.7) | (25.2, 31.0) |
| Non-Responders, n (%) [3] | 367 (78.3) | 335 (71.3) | 342 (72.6) | 677 (71.9) |
| CMH p-value [4] | — | 0.014 | 0.045 | — |
| Odds Ratio (adjusted) [5] | — | 0.691 | 0.738 | — |
| 95% CI | — | (0.514, 0.929) | (0.548, 0.994) | — |
| Difference in Proportions (adjusted) [6] | — | 0.070 | 0.056 | — |
| 95% CI | — | (0.014, 0.125) | (0.002, 0.111) | — |
| Breslow-Day p-value [7] | — | 0.134 | 0.063 | — |
| Week 4 | | | | |
| CSBM Weekly Responders, n (%) [1] | 98 (20.9) | 142 (30.2) | 137 (29.1) | 279 (29.6) |
| 95% CI (%) [2] | (17.3, 24.9) | (26.1, 34.6) | (25.0, 33.4) | (26.7, 32.7) |
| Non-Responders, n (%) [3] | 371 (79.1) | 328 (69.8) | 334 (70.9) | 662 (70.4) |
| CMH p-value [4] | — | 0.001 | 0.004 | — |
| Odds Ratio (adjusted) [5] | — | 0.611 | 0.644 | — |
| 95% CI | — | (0.454, 0.822) | (0.478, 0.868) | — |
| Difference in Proportions (adjusted) [6] | — | 0.093 | 0.082 | — |
| 95% CI | — | (0.038, 0.149) | (0.027, 0.137) | — |
| Breslow-Day p-value [7] | — | 0.249 | 0.960 | — |
| Week 5 | | | | |
| CSBM Weekly Responders, n (%) [1] | 109 (23.2) | 165 (35.1) | 138 (29.3) | 303 (32.2) |
| 95% CI (%) [2] | (19.5, 27.3) | (30.8, 39.6) | (25.2, 33.6) | (29.2, 35.3) |
| Non-Responders, n (%) [3] | 360 (76.8) | 305 (64.9) | 333 (70.7) | 638 (67.8) |
| CMH p-value [4] | — | <0.001 | 0.035 | — |
| Odds Ratio (adjusted) [5] | — | 0.560 | 0.731 | — |
| 95% CI | — | (0.421, 0.746) | (0.546, 0.979) | — |
| Difference in Proportions (adjusted) [6] | — | 0.119 | 0.061 | — |
| 95% CI | — | (0.061, 0.176) | (0.004, 0.117) | — |
| Breslow-Day p-value [7] | — | 0.330 | 0.235 | — |
| Week 6 | | | | |
| CSBM Weekly Responders, n (%) [1] | 98 (20.9) | 145 (30.9) | 150 (31.8) | 295 (31.3) |
| 95% CI (%) [2] | (17.3, 24.9) | (26.7, 33.2) | (27.7, 36.3) | (28.4, 34.4) |
| Non-Responders, n (%) [3] | 371 (79.1) | 325 (69.1) | 321 (68.2) | 646 (68.7) |
| CMH p-value [4] | — | <0.001 | <0.001 | — |
| Odds Ratio (adjusted) [5] | — | 0.593 | 0.566 | — |
| 95% CI | — | (0.441, 0.797) | (0.421, 0.760) | — |
| Difference in Proportions (adjusted) [6] | — | 0.100 | 0.110 | — |
| 95% CI | — | (0.044, 0.155) | (0.054, 0.165) | — |
| Breslow-Day p-value [7] | — | 0.096 | 0.161 | — |
| Week 7 | | | | |
| CSBM Weekly Responders, n (%) [1] | 109 (23.2) | 152 (32.3) | 145 (30.8) | 297 (31.6) |
| 95% CI (%) [2] | (19.5, 27.3) | (28.1, 36.8) | (26.6, 35.2) | (28.6, 34.6) |
| Non-Responders, n (%) [3] | 360 (76.8) | 318 (67.7) | 326 (69.2) | 644 (68.4) |
| CMH p-value [4] | — | 0.002 | 0.009 | — |
| Odds Ratio (adjusted) [5] | — | 0.634 | 0.681 | — |
| 95% CI | — | (0.475, 0.845) | (0.509, 0.910) | — |
| Difference in Proportions (adjusted) [6] | — | 0.091 | 0.075 | — |
| 95% CI | — | (0.034, 0.148) | (0.019, 0.132) | — |
| Breslow-Day p-value [7] | — | 0.685 | 0.780 | — |
| Week 8 | | | | |
| CSBM Weekly Responders, n (%) [1] | 115 (24.5) | 151 (32.1) | 147 (31.2) | 298 (31.7) |
| 95% CI (%) [2] | (20.7, 28.7) | (27.9, 36.6) | (27.0, 35.6) | (28.7, 34.7) |
| Non-Responders, n (%) [3] | 354 (75.5) | 319 (67.9) | 324 (68.8) | 643 (68.3) |
| CMH p-value [4] | — | 0.010 | 0.022 | — |
| Odds Ratio (adjusted) [5] | — | 0.687 | 0.716 | — |
| 95% CI | — | (0.516, 0.914) | (0.537, 0.954) | — |
| Difference in Proportions (adjusted) [6] | — | 0.076 | 0.067 | — |
| 95% CI | — | (0.019, 0.134) | (0.010, 0.124) | — |
| Breslow-Day p-value [7] | — | 0.240 | 0.921 | — |
| Week 9 | | | | |
| CSBM Weekly Responders, n (%) [1] | 111 (23.7) | 151 (32.1) | 146 (31.0) | 297 (31.6) |
| 95% CI (%) [2] | (19.9, 27.8) | (27.9, 36.6) | (26.8, 35.4) | (28.6, 34.6) |
| Non-Responders, n (%) [3] | 358 (76.3) | 319 (67.9) | 325 (69.0) | 644 (68.4) |
| CMH p-value [4] | — | 0.004 | 0.012 | — |
| Odds Ratio (adjusted) [5] | — | 0.656 | 0.691 | — |
| 95% CI | — | (0.492, 0.875) | (0.518, 0.922) | — |
| Difference in Proportions (adjusted) [6] | — | 0.084 | 0.073 | — |
| 95% CI | — | (0.027, 0.142) | (0.017, 0.130) | — |
| Breslow-Day p-value [7] | — | 0.119 | 0.078 | — |
| Week 10 | | | | |
| CSBM Weekly Responders, n (%) [1] | 110 (23.5) | 142 (30.2) | 156 (33.1) | 298 (31.7) |
| 95% CI (%) [2] | (19.7, 27.6) | (26.1, 34.6) | (28.9, 37.6) | (28.7, 34.7) |
| Non-Responders, n (%) [3] | 359 (76.5) | 328 (69.8) | 315 (66.9) | 643 (68.3) |
| CMH p-value [4] | — | 0.020 | 0.001 | — |
| Odds Ratio (adjusted) [5] | — | 0.708 | 0.619 | — |
| 95% CI | — | (0.530, 0.947) | (0.465, 0.825) | — |
| Difference in Proportions (adjusted) [6] | — | 0.067 | 0.097 | — |
| 95% CI | — | (0.011, 0.124) | (0.039, 0.154) | — |
| Breslow-Day p-value [7] | — | 0.585 | 0.349 | — |
| Week 11 | | | | |
| CSBM Weekly Responders, n (%) [1] | 111 (23.7) | 153 (32.6) | 150 (31.8) | 303 (32.2) |
| 95% CI (%) [2] | (19.9, 27.8) | (28.3, 37.0) | (27.7, 36.3) | (29.2, 35.3) |
| Non-Responders, n (%) [3] | 358 (76.3) | 317 (67.4) | 321 (68.2) | 638 (67.8) |
| CMH p-value [4] | — | 0.003 | 0.005 | — |
| Odds Ratio (adjusted) [5] | — | 0.643 | 0.664 | — |
| 95% CI | — | (0.482, 0.857) | (0.498, 0.885) | — |
| Difference in Proportions (adjusted) [6] | — | 0.089 | 0.082 | — |
| 95% CI | — | (0.031, 0.146) | (0.025, 0.139) | — |
| Breslow-Day p-value [7] | — | 0.887 | 0.384 | — |
| Week 12 | | | | |
| CSBM Weekly Responders, n (%) [1] | 113 (24.1) | 145 (30.9) | 155 (32.9) | 300 (31.9) |
| 95% CI (%) [2] | (20.3, 28.2) | (26.7, 35.2) | (28.7, 37.4) | (28.9, 35.0) |
| Non-Responders, n (%) [3] | 356 (75.9) | 325 (69.1) | 316 (67.1) | 641 (68.1) |
| CMH p-value [4] | — | 0.021 | 0.003 | — |
| Odds Ratio (adjusted) [5] | — | 0.711 | 0.647 | — |
| 95% CI | — | (0.533, 0.949) | (0.486, 0.861) | — |
| Difference in Proportions (adjusted) [6] | — | 0.068 | 0.088 | — |
| 95% CI | — | (0.011, 0.125) | (0.031, 0.146) | — |
| Breslow-Day p-value [7] | — | 0.884 | 0.948 | — |
| Post-Treatment Week 13 | | | | |
| CSBM Weekly Responders, n (%) [1] | 105 (22.4) | 125 (26.6) | 107 (22.7) | 232 (24.7) |
| 95% CI (%) [2] | (18.7, 26.4) | (22.7, 30.8) | (19.0, 26.8) | (21.9, 27.5) |
| Non-Responders, n (%) [3] | 364 (77.6) | 345 (73.4) | 364 (77.3) | 709 (75.3) |
| CMH p-value [4] | — | 0.135 | 0.905 | — |
| Odds Ratio (adjusted) [5] | — | 0.796 | 0.981 | — |
| 95% CI | — | (0.591, 1.073) | (0.723, 1.333) | — |
| Difference in Proportions (adjusted) [6] | — | 0.042 | 0.003 | — |
| 95% CI | — | (−0.013, 0.097) | (−0.050, 0.057) | — |
| Breslow-Day p-value [7] | — | 0.830 | 0.659 | — |
| Post-Treatment Week 14 | | | | |
| CSBM Weekly Responders, n (%) [1] | 86 (18.3) | 95 (20.2) | 85 (18.0) | 180 (19.1) |
| 95% CI (%) [2] | (14.9, 22.1) | (16.7, 24.1) | (14.7, 21.8) | (16.7, 21.8) |
| Non-Responders, n (%) [3] | 383 (81.7) | 375 (79.8) | 386 (82.0) | 761 (80.9) |
| CMH p-value [4] | — | 0.466 | 0.909 | — |
| Odds Ratio (adjusted) [5] | — | 0.886 | 1.020 | — |
| 95% CI | — | (0.641, 1.226) | (0.732, 1.420) | — |
| Difference in Proportions (adjusted) [6] | — | 0.019 | −0.003 | — |
| 95% CI | — | (−0.032, 0.069) | (−0.052, 0.046) | — |
| Breslow-Day p-value [7] | — | 0.189 | 0.287 | — |

Source: Listings 16.2.6.1, 16.2.6.2
[1] A CSBM weekly responder is defined as a patient who has >=3 CSBMs per week and an increase from baseline of >=1 CSBM for that week, determined using mean replacement approach (MRA) methodology as defined in Section 4.1.2 of the statistical analysis plan.
[2] Clopper-Pearson method
[3] Patients missing with respect to the endpoint were scored as non-responders.
[4] CMH p-value for the comparison of treatment group to placebo, stratified by gender.
[5] Common odds ratio, treatment/placebo adjusted for stratification factor (gender) in the CMH analysis.
[6] Proportion of responders in treatment group minus proportion in Placebo group, adjustead for stratification facor (gender) in the CMH analysis.
[7] Breslow-Day p-value for the test of consistency of the treatment effect across the stratification.
Chiltern • 29JUL2015:5:34 PM • I:\Synergy\33610\21_Stat\Programs\Tables\t_respond.sas • ZScott

TABLE 22
Change from Baseline in CSBMs (CSBMs/week), by Time Point;
Linear Mixed-effects Model, Mean Replacement Approach (MRA) (ITT Population)

| | | Plecanatide | Plecanatide | Active |
|---|---|---|---|---|
| | Placebo | 3 mg | 6 mg | Combined |
| | (N = 445) | (N = 443) | (N = 449) | (N = 892) |
| Change from Baseline [1], (overall average estimate | | | | |
| across the 12-week treatment period) | | | | |
| LS Mean (SE) | 1.37 (0.134) | 2.27 (0.135) | 2.16 (0.133) | — |
| Difference from Placebo | — | 0.90 | 0.79 | — |
| 95% CI [2] | — | 0.55, 1.24 | 0.45, 1.13 | — |
| p-value vs. Placebo [3] | — | <0.001 | <0.001 | — |
| Baseline [1] | | | | |
| n | 444 | 439 | 447 | 886 |
| Mean (SD) | 0.31 (0.496) | 0.28 (0.550) | 0.25 (0.442) | 0.27 (0.498) |
| Week 1 Change from Baseline [1] | | | | |
| n | 433 | 422 | 431 | 853 |
| Mean (SD) | 0.84 (1.793) | 1.75 (2.732) | 1.72 (2.897) | 1.74 (2.815) |
| LS Mean (SE) | 0.84 (0.152) | 1.72 (0.153) | 1.71 (0.151) | — |
| Difference from Placebo | — | 0.88 | 0.88 | — |
| 95% CI [2] | — | 0.48, 1.28 | 0.48, 1.27 | — |
| p-value vs. Placebo [3] | — | <0.001 | <0.001 | — |
| Week 2 Change from Baseline [1] | | | | |
| n | 432 | 420 | 427 | 847 |
| Mean (SD) | 0.97 (1.876) | 1.81 (2.963) | 1.84 (3.158) | 1.82 (3.061) |
| LS Mean (SE) | 0.96 (0.152) | 1.82 (0.153) | 1.81 (0.152) | — |
| Difference from Placebo | — | 0.86 | 0.85 | — |
| 95% CI [2] | — | 0.46, 1.25 | 0.46, 1.25 | — |
| p-value vs. Placebo [3] | — | <0.001 | <0.001 | — |
| Week 3 Change from Baseline [1] | | | | |
| n | 428 | 414 | 422 | 836 |
| Mean (SD) | 1.21 (2.141) | 1.94 (3.117) | 1.82 (2.949) | 1.88 (3.032) |
| LS Mean (SE) | 1.20 (0.152) | 1.94 (0.153) | 1.81 (0.152) | — |
| Difference from Placebo | — | 0.74 | 0.61 | — |
| 95% CI [2] | — | 0.34, 1.14 | 0.22, 1.01 | — |
| p-value vs. Placebo [3] | — | <0.001 | 0.002 | — |
| Week 4 Change from Baseline [1] | | | | |
| n | 418 | 411 | 416 | 827 |
| Mean (SD) | 1.28 (2.133) | 2.19 (3.395) | 2.03 (3.243) | 2.11 (3.318) |
| LS Mean (SE) | 1.25 (0.153) | 2.16 (0.153) | 2.00 (0.152) | — |
| Difference from Placebo | — | 0.91 | 0.75 | — |
| 95% CI [2] | — | 0.51, 1.31 | 0.35, 1.15 | — |
| p-value vs. Placebo [3] | — | <0.001 | <0.001 | — |
| Week 5 Change from Baseline [1] | | | | |
| n | 409 | 395 | 412 | 807 |
| Mean (SD) | 1.51 (2.495) | 2.39 (3.375) | 1.99 (3.019) | 2.18 (3.202) |
| LS Mean (SE) | 1.47 (0.153) | 2.31 (0.154) | 1.95 (0.153) | — |
| Difference from Placebo | — | 0.84 | 0.48 | — |
| 95% CI [2] | — | 0.44, 1.24 | 0.08, 0.88 | — |
| p-value vs. Placebo [3] | — | <0.001 | 0.019 | — |
| Week 6 Change from Baseline [1] | | | | |
| n | 403 | 384 | 407 | 791 |
| Mean (SD) | 1.36 (2.312) | 2.43 (3.548) | 2.29 (3.384) | 2.36 (3.464) |
| LS Mean (SE) | 1.31 (0.154) | 2.32 (0.155) | 2.24 (0.153) | — |
| Difference from Placebo | — | 1.01 | 0.92 | — |
| 95% CI [2] | — | 0.60, 1.41 | 0.52, 1.33 | — |
| p-value vs. Placebo [3] | — | <0.001 | <0.001 | — |
| Week 7 Change from Baseline [1] | | | | |
| n | 400 | 378 | 396 | 774 |
| Mean (SD) | 1.55 (2.422) | 2.40 (3.413) | 2.38 (3.361) | 2.39 (3.384) |
| LS Mean (SE) | 1.49 (0.154) | 2.29 (0.155) | 2.31 (0.154) | — |
| Difference from Placebo | — | 0.81 | 0.82 | — |
| 95% CI [2] | — | 0.40, 1.21 | 0.42, 1.23 | — |
| p-value vs. Placebo [3] | — | <0.001 | <0.001 | — |
| Week 8 Change from Baseline [1] | | | | |
| n | 395 | 373 | 395 | 768 |
| Mean (SD) | 1.57 (2.439) | 2.67 (3.747) | 2.36 (3.243) | 2.51 (3.498) |
| LS Mean (SE) | 1.51 (0.154) | 2.52 (0.156) | 2.33 (0.154) | — |
| Difference from Placebo | — | 1.01 | 0.82 | — |
| 95% CI [2] | — | 0.60, 1.42 | 0.42, 1.22 | — |
| p-value vs. Placebo [3] | — | <0.001 | <0.001 | — |
| Week 9 Change from Baseline [1] | | | | |
| n | 390 | 367 | 391 | 758 |
| Mean (SD) | 1.61 (2.467) | 2.59 (3.459) | 2.38 (3.176) | 2.48 (3.316) |
| LS Mean (SE) | 1.55 (0.154) | 2.49 (0.156) | 2.37 (0.154) | — |
| Difference from Placebo | — | 0.94 | 0.81 | — |
| 95% CI [2] | — | 0.53, 1.35 | 0.41, 1.22 | — |
| p-value vs. Placebo [3] | — | <0.001 | <0.001 | — |
| Week 10 Change from Baseline [1] | | | | |
| n | 387 | 366 | 393 | 759 |
| Mean (SD) | 1.69 (2.529) | 2.63 (3.617) | 2.51 (3.288) | 2.57 (3.449) |
| LS Mean (SE) | 1.64 (0.154) | 2.49 (0.156) | 2.50 (0.154) | — |
| Difference from Placebo | — | 0.85 | 0.86 | — |
| 95% CI [2] | — | 0.44, 1.26 | 0.46, 1.27 | — |
| p-value vs. Placebo [3] | — | <0.001 | <0.001 | — |
| Week 11 Change from Baseline [1] | | | | |
| n | 384 | 358 | 380 | 738 |
| Mean (SD) | 1.65 (2.516) | 2.71 (3.710) | 2.44 (3.276) | 2.57 (3.493) |
| LS Mean (SE) | 1.60 (0.155) | 2.56 (0.157) | 2.40 (0.155) | — |
| Difference from Placebo | — | 0.96 | 0.80 | — |
| 95% CI [2] | — | 0.55, 1.37 | 0.40, 1.20 | — |
| p-value vs. Placebo [3] | — | <0.001 | <0.001 | — |
| Week 12 Change from Baseline [1] | | | | |
| n | 372 | 356 | 372 | 728 |
| Mean (SD) | 1.71 (2.535) | 2.76 (3.785) | 2.57 (3.409) | 2.66 (3.596) |
| LS Mean (SE) | 1.66 (0.155) | 2.60 (0.157) | 2.51 (0.155) | — |
| Difference from Placebo | — | 0.94 | 0.86 | — |
| 95% CI [2] | — | 0.53, 1.35 | 0.45, 1.26 | — |
| p-value vs. Placebo [3] | — | <0.001 | <0.001 | — |
| Post-Treatment Week 13 Change from Baseline [1] | | | | |
| n | 386 | 369 | 380 | 749 |
| Mean (SD) | 1.43 (2.208) | 1.88 (2.733) | 1.64 (2.393) | 1.76 (2.567) |
| LS Mean (SE) | 1.50 (0.133) | 1.94 (0.134) | 1.70 (0.134) | — |
| Difference from Placebo | — | 0.43 | 0.20 | — |
| 95% CI [2] | — | 0.08, 0.78 | −0.14, 0.55 | — |
| p-value vs. Placebo [3] | — | 0.015 | 0.254 | — |
| Post-Treatment Week 14 Change from Baseline [1] | | | | |
| n | 358 | 339 | 337 | 676 |
| Mean (SD) | 1.33 (2.134) | 1.78 (2.620) | 1.67 (2.616) | 1.72 (2.616) |
| LS Mean (SE) | 1.39 (0.135) | 1.82 (0.136) | 1.73 (0.137) | — |
| Difference from Placebo | — | 0.43 | 0.34 | — |
| 95% CI [2] | — | 0.07, 0.78 | −0.02, 0.69 | — |
| p-value vs. Placebo [3] | — | 0.019 | 0.061 | — |

Source: Listings 16.2.6.1, 16.2.6.2
Note:
Statistics are calculated with 12 week on-treatment and 2 week post-treatment results modeled separately.
[1] Baseline is the mean number of CSBMs recorded during the 2-week baseline diary assessment period prior to the first dose of study drug. The weekly CSBM totals are derived from the daily diary entries reported during the Treatment Period in the BM and Symptom Diary. The time points reported are Treatment Period study weeks defined as follows: Week 1 = study days 1-7; Week 2 = study days 8-14; Week 3 = study days 15-21; Week 4 = study days 22-28; Week 5 = study days 29-35; Week 6 = study days 36-42; Week 7 = study days 43-49; Week 8 = study days 50-56; Week 9 = study days 57-63; Week 10 = study days 64-70; Week 11 = study days 71-77; Week 12 = study days 78-84; Post-Treatment Week 13 = days 1-7 post treatment; Post-Treatment Week 14 = days 8-14 post treatment. Changes from baseline are based on mean replacement approach (MRA) methodology as defined in Section 4.1.2 of the statistical analysis plan.
[2] The 95% confidence interval for the difference between the placebo group and the specified treatment group LS means.
[3] P-value vs. Placebo is from the pairwise comparison of LS means between the placebo group and the specified treatment group using a linear mixed-effects model with fixed effects for gender (stratification variable), treatment, week, the interaction of treatment and week, and the corresponding baseline value, and a random intercept for patient. The model takes into account the repeated measurements of CSBMs for each patient.
Chiltern • 29JUL2015:5:35 PM • I:\Synergy\33610\21_Stat\Programs\Tables\t_sbm_cfb.sas • ZScott

TABLE 23
Change from Baseline in CSBMs (CSBMs/week), by Time Point; Linear Mixed-effects
Model, Mean Replacement Approach (MRA) (ITT Population-Excluding
Duplicate Patients)

| | | Plecanatide | Plecanatide | Active |
|---|---|---|---|---|
| | Placebo | 3 mg | 6 mg | Combined |
| | (N = 437) | (N = 434) | (N = 443) | (N = 877) |
| Change from Baseline [1], (overall average estimate | | | | |
| across the 12-week treatment period) | | | | |
| LS Mean (SE) | 1.39 (0.136) | 2.30 (0.137) | 2.18 (0.135) | — |
| Difference from Placebo | — | 0.91 | 0.79 | — |
| 95% CI [2] | — | 0.56, 1.26 | 0.44, 1.13 | — |
| p-value vs. Placebo [3] | — | <0.001 | <0.001 | — |
| Baseline [1] | | | | |
| n | 436 | 430 | 441 | 871 |
| Mean (SD) | 0.31 (0.489) | 0.29 (0.552) | 0.25 (0.444) | 0.27 (0.500) |
| Week 1 Change from Baseline [1] | | | | |
| n | 425 | 414 | 425 | 839 |
| Mean (SD) | 0.86 (1.799) | 1.78 (2.752) | 1.74 (2.911) | 1.76 (2.832) |
| LS Mean (SE) | 0.87 (0.154) | 1.75 (0.155) | 1.74 (0.153) | — |
| Difference from Placebo | — | 0.88 | 0.87 | — |
| 95% CI [2] | — | 0.48, 1.29 | 0.47, 1.28 | — |
| p-value vs. Placebo [3] | — | <0.001 | <0.001 | — |
| Week 2 Change from Baseline [1] | | | | |
| n | 424 | 412 | 422 | 834 |
| Mean (SD) | 0.98 (1.883) | 1.82 (2.986) | 1.85 (3.171) | 1.84 (3.079) |
| LS Mean (SE) | 0.99 (0.154) | 1.84 (0.155) | 1.84 (0.154) | — |
| Difference from Placebo | — | 0.85 | 0.85 | — |
| 95% CI [2] | — | 0.45, 1.26 | 0.45, 1.26 | — |
| p-value vs. Placebo [3] | — | <0.001 | <0.001 | — |
| Week 3 Change from Baseline [1] | | | | |
| n | 420 | 405 | 418 | 823 |
| Mean (SD) | 1.23 (2.142) | 1.95 (3.143) | 1.83 (2.958) | 1.89 (3.049) |
| LS Mean (SE) | 1.22 (0.154) | 1.96 (0.156) | 1.83 (0.154) | — |
| Difference from Placebo | — | 0.74 | 0.61 | — |
| 95% CI [2] | — | 0.34, 1.15 | 0.21, 1.01 | — |
| p value vs. Placebo [3] | — | <0.001 | 0.003 | — |
| Week 4 Change from Baseline [1] | | | | |
| n | 411 | 403 | 413 | 816 |
| Mean (SD) | 1.29 (2.133) | 2.22 (3.418) | 2.03 (3.254) | 2.13 (3.335) |
| LS Mean (SE) | 1.27 (0.155) | 2.20 (0.156) | 2.02 (0.154) | — |
| Difference from Placebo | — | 0.92 | 0.75 | — |
| 95% CI [2] | — | 0.52, 1.33 | 0.35, 1.15 | — |
| p-value vs. Placebo [3] | — | <0.001 | <0.001 | — |
| Week 5 Change from Baseline [1] | | | | |
| n | 402 | 387 | 409 | 796 |
| Mean (SD) | 1.53 (2.502) | 2.43 (3.395) | 1.99 (3.025) | 2.20 (3.216) |
| LS Mean (SE) | 1.50 (0.155) | 2.35 (0.157) | 1.97 (0.154) | — |
| Difference from Placebo | — | 0.85 | 0.46 | — |
| 95% CI [2] | — | 0.44, 1.25 | 0.06, 0.86 | — |
| p-value vs. Placebo [3] | — | <0.001 | 0.025 | — |
| Week 6 Change from Baseline [1] | | | | |
| n | 396 | 377 | 404 | 781 |
| Mean (SD) | 1.38 (2.318) | 2.45 (3.571) | 2.31 (3.391) | 2.38 (3.478) |
| LS Mean (SE) | 1.34 (0.156) | 2.35 (0.158) | 2.27 (0.155) | — |
| Difference from Placebo | — | 1.01 | 0.93 | — |
| 95% CI [2] | — | 0.60, 1.42 | 0.52, 1.33 | — |
| p-value vs. Placebo [3] | — | <0.001 | <0.001 | — |
| Week 7 Change from Baseline [1] | | | | |
| n | 393 | 371 | 393 | 764 |
| Mean (SD) | 1.56 (2.428) | 2.44 (3.435) | 2.40 (3.369) | 2.42 (3.399) |
| LS Mean (SE) | 1.51 (0.156) | 2.33 (0.158) | 2.34 (0.155) | — |
| Difference from Placebo | — | 0.83 | 0.83 | — |
| 95% CI [2] | — | 0.42, 1.24 | 0.42, 1.24 | — |
| p-value vs. Placebo [3] | — | <0.001 | <0.001 | — |
| Week 8 Change from Baseline [1] | | | | |
| n | 388 | 368 | 392 | 760 |
| Mean (SD) | 1.54 (2.418) | 2.68 (3.765) | 2.37 (3.250) | 2.52 (3.510) |
| LS Mean (SE) | 1.50 (0.156) | 2.56 (0.158) | 2.35 (0.155) | — |
| Difference from Placebo | — | 1.06 | 0.85 | — |
| 95% CI [2] | — | 0.65, 1.47 | 0.44, 1.26 | — |
| p-value vs. Placebo [3] | — | <0.001 | <0.001 | |
| Week 9 Change from Baseline [1] | | | | |
| n | 385 | 360 | 388 | 748 |
| Mean (SD) | 1.63 (2.476) | 2.62 (3.479) | 2.39 (3.184) | 2.50 (3.329) |
| LS Mean (SE) | 1.58 (0.156) | 2.53 (0.159) | 2.39 (0.156) | — |
| Difference from Placebo | — | 0.95 | 0.81 | — |
| 95% CI [2] | — | 0.54, 1.37 | 0.40, 1.22 | — |
| p value vs. Placebo [3] | — | <0.001 | <0.001 | — |
| Week 10 Change from Baseline [1] | | | | |
| n | 382 | 360 | 390 | 750 |
| Mean (SD) | 1.70 (2.539) | 2.65 (3.637) | 2.51 (3.288) | 2.58 (3.458) |
| LS Mean (SE) | 1.65 (0.156) | 2.52 (0.159) | 2.52 (0.156) | — |
| Difference from Placebo | — | 0.87 | 0.87 | — |
| 95% CI [2] | — | 0.46, 1.28 | 0.46, 1.27 | — |
| p-value vs. Placebo [3] | — | <0.001 | <0.001 | — |
| Week 11 Change from Baseline [1] | | | | |
| n | 379 | 352 | 377 | 729 |
| Mean (SD) | 1.66 (2.527) | 2.75 (3.728) | 2.43 (3.266) | 2.58 (3.498) |
| LS Mean (SE) | 1.62 (0.157) | 2.61 (0.159) | 2.41 (0.156) | — |
| Difference from Placebo | — | 0.99 | 0.79 | — |
| 95% CI [2] | — | 0.58, 1.40 | 0.38, 1.19 | — |
| p-value vs. Placebo [3] | — | <0.001 | <0.001 | — |
| Week 12 Change from Baseline [1] | | | | |
| n | 367 | 351 | 369 | 720 |
| Mean (SD) | 1.72 (2.548) | 2.78 (3.803) | 2.56 (3.366) | 2.67 (3.585) |
| LS Mean (SE) | 1.67 (0.157) | 2.64 (0.159) | 2.51 (0.157) | — |
| Difference from Placebo | — | 0.97 | 0.84 | — |
| 95% CI [2] | — | 0.56, 1.38 | 0.42, 1.25 | — |
| p-value vs. Placebo [3] | — | <0.001 | <0.001 | — |
| Post-Treatment Week 13 Change from Baseline [1] | | | | |
| n | 381 | 362 | 377 | 739 |
| Mean (SD) | 1.43 (2.217) | 1.90 (2.743) | 1.62 (2.339) | 1.76 (2.547) |
| LS Mean (SE) | 1.50 (0.132) | 1.96 (0.135) | 1.69 (0.133) | — |
| Difference from Placebo | — | 0.46 | 0.18 | — |
| 95% CI [2] | — | 0.11, 0.81 | −0.16, 0.53 | — |
| p-value vs. Placebo [3] | — | 0.01 | 0.293 | — |
| Post-Treatment Week 14 Change from Baseline [1] | | | | |
| n | 354 | 335 | 334 | 669 |
| Mean (SD) | 1.34 (2.143) | 1.78 (2.628) | 1.64 (2.536) | 1.71 (2.582) |
| LS Mean (SE) | 1.40 (0.135) | 1.83 (0.137) | 1.71 (0.136) | — |
| Difference from Placebo | — | 0.43 | 0.31 | — |
| 95% CI [2] | — | 0.07, 0.78 | −0.04, 0.66 | — |
| p-value vs. Placebo [3] | — | 0.019 | 0.087 | — |

Source: Listings 16.2.6.1, 16.2.6.2
Note:
Statistics are calculated with 12 week on-treatment and 2 week post-treatment results modeled separately.
[1] Baseline is the mean number of CSBMs recorded during the 2-week baseline diary assessment period prior to the first dose of study drug. The weekly CSBM totals are derived from the daily diary entries reported during the Treatment Period in the BM and Symptom Diary. The time points reported are Treatment Period study weeks defined as follows: Week 1 = study days 1-7; Week 2 = study days 8-14; Week 3 = study days 15-21; Week 4 = study days 22-28; Week 5 = study days 29-35; Week 6 = study days 36-42; Week 7 = study days 43-49; Week 8 = study days 50-56; Week 9 = study days 57-63; Week 10 = study days 64-70; Week 11 = study days 71-77; Week 12 = study days 78-84; Post-Treatment Week 13 = days 1-7 post treatment; Post-Treatment Week 14 = days 8-14 post treatment. Changes from baseline are based on mean replacement approach (MRA) methodology as defined in Section 4.1.2 of the statistical analysis plan.
[2] The 95% confidence interval for the difference between the placebo group and the specified treatment group LS means.
[3] P-value vs. Placebo is from the pairwise comparison of LS means between the placebo group and the specified treatment group using a linear mixed-effects model with fixed effects for gender (stratification variable), treatment, week, the interaction of treatment and week, and the corresponding baseline value, and a random intercept for patient. The model takes into account the repeated measurements of CSBMs for each patient.
Chiltern • 29JUL2015:5:35 PM • I:\Synergy\33610\21_Stat\Programs\Tables\t_sbm_cfb.sas • ZScott

TABLE 24
Change from Baseline in CSBMs (CSBMs/week), by Time Point; Linear Mixed-effects
Model, Mean Replacement Approach (MRA) (All Randomized (includes Duplicate Patients))

| | | Plecanatide | Plecanatide | Active |
|---|---|---|---|---|
| | Placebo | 3 mg | 6 mg | Combined |
| | (N = 469) | (N = 470) | (N = 471) | (N = 941) |
| Change from Baseline [1], (overall average estimate | | | | |
| across the 12-week treatment period) | | | | |
| LS Mean (SE) | 1.35 (0.129) | 2.25 (0.129) | 2.14 (0.129) | — |
| Difference from Placebo | — | 0.91 | 0.80 | — |
| 95% CI [2] | — | 0.57, 1.24 | 0.46, 1.13 | — |
| p-value vs. Placebo [3] | — | <0.001 | <0.001 | — |
| Baseline [1] | | | | |
| n | 467 | 466 | 469 | 935 |
| Mean (SD) | 0.30 (0.489) | 0.27 (0.542) | 0.25 (0.455) | 0.26 (0.500) |
| Week 1 Change from Baseline [1] | | | | |
| n | 455 | 448 | 452 | 900 |
| Mean (SD) | 0.83 (1.769) | 1.75 (2.737) | 1.71 (2.870) | 1.73 (2.803) |
| LS Mean (SE) | 0.82 (0.146) | 1.72 (0.147) | 1.70 (0.146) | — |
| Difference from Placebo | — | 0.90 | 0.88 | — |
| 95% CI [2] | — | 0.51, 1.28 | 0.49, 1.26 | — |
| p-value vs. Placebo [3] | — | <0.001 | <0.001 | — |
| Week 2 Change from Baseline [1] | | | | |
| n | 453 | 446 | 448 | 894 |
| Mean (SD) | 0.96 (1.865) | 1.82 (2.956) | 1.83 (3.118) | 1.83 (3.037) |
| LS Mean (SE) | 0.96 (0.146) | 1.83 (0.147) | 1.81 (0.146) | — |
| Difference from Placebo | — | 0.87 | 0.85 | — |
| 95% CI [2] | — | 0.49, 1.25 | 0.47, 1.23 | — |
| p-value vs. Placebo [3] | — | <0.001 | <0.001 | — |
| Week 3 Change from Baseline [1] | | | | |
| n | 448 | 440 | 442 | 882 |
| Mean (SD) | 1.18 (2.113) | 1.94 (3.084) | 1.83 (2.927) | 1.89 (3.005) |
| LS Mean (SE) | 1.17 (0.147) | 1.94 (0.147) | 1.81 (0.147) | — |
| Difference from Placebo | — | 0.77 | 0.65 | — |
| 95% CI [2] | — | 0.39, 1.16 | 0.26, 1.03 | — |
| p-value vs. Placebo [3] | — | <0.001 | <0.001 | — |
| Week 4 Change from Baseline [1] | | | | |
| n | 437 | 436 | 436 | 872 |
| Mean (SD) | 1.25 (2.118) | 2.20 (3.352) | 2.05 (3.223) | 2.13 (3.287) |
| LS Mean (SE) | 1.22 (0.147) | 2.17 (0.148) | 2.02 (0.147) | — |
| Difference from Placebo | — | 0.95 | 0.80 | — |
| 95% CI [2] | — | 0.56, 1.33 | 0.41, 1.18 | — |
| p-value vs. Placebo [3] | — | <0.001 | <0.001 | — |
| Week 5 Change from Baseline [1] | | | | |
| n | 427 | 417 | 430 | 847 |
| Mean (SD) | 1.47 (2.473) | 2.40 (3.334) | 1.97 (3.003) | 2.18 (3.176) |
| LS Mean (SE) | 1.43 (0.148) | 2.30 (0.149) | 1.93 (0.147) | — |
| Difference from Placebo | — | 0.87 | 0.50 | — |
| 95% CI [2] | — | 0.48, 1.26 | 0.11, 0.88 | — |
| p-value vs. Placebo [3] | — | <0.001 | 0.012 | — |
| Week 6 Change from Baseline [1] | | | | |
| n | 421 | 406 | 423 | 829 |
| Mean (SD) | 1.35 (2.291) | 2.42 (3.524) | 2.26 (3.359) | 2.34 (3.440) |
| LS Mean (SE) | 1.30 (0.148) | 2.30 (0.149) | 2.21 (0.148) | — |
| Difference from Placebo | — | 1.00 | 0.91 | — |
| 95% CI [2] | — | 0.61, 1.39 | 0.52, 1.30 | — |
| p-value vs. Placebo [3] | — | <0.001 | <0.001 | — |
| Week 7 Change from Baseline [1] | | | | |
| n | 418 | 398 | 413 | 811 |
| Mean (SD) | 1.51 (2.400) | 2.41 (3.388) | 2.33 (3.330) | 2.37 (3.357) |
| LS Mean (SE) | 1.46 (0.148) | 2.28 (0.150) | 2.27 (0.148) | — |
| Difference from Placebo | — | 0.82 | 0.81 | — |
| 95% CI [2] | — | 0.43, 1.22 | 0.42, 1.20 | — |
| p-value vs. Placebo [3] | — | <0.001 | <0.001 | — |
| Week 8 Change from Baseline [1] | | | | |
| n | 411 | 392 | 411 | 803 |
| Mean (SD) | 1.52 (2.404) | 2.64 (3.709) | 2.32 (3.221) | 2.48 (3.469) |
| LS Mean (SE) | 1.48 (0.149) | 2.49 (0.150) | 2.29 (0.148) | — |
| Difference from Placebo | — | 1.01 | 0.81 | — |
| 95% CI [2] | — | 0.62, 1.41 | 0.42, 1.20 | — |
| p-value vs. Placebo [3] | — | <0.001 | <0.001 | — |
| Week 9 Change from Baseline [1] | | | | |
| n | 405 | 385 | 407 | 792 |
| Mean (SD) | 1.58 (2.435) | 2.55 (3.428) | 2.35 (3.152) | 2.45 (3.288) |
| LS Mean (SE) | 1.53 (0.149) | 2.46 (0.150) | 2.34 (0.149) | — |
| Difference from Placebo | — | 0.93 | 0.81 | — |
| 95% CI [2] | — | 0.54, 1.32 | 0.42, 1.20 | — |
| p-value vs. Placebo [3] | — | <0.001 | <0.001 | — |
| Week 10 Change from Baseline [1] | | | | |
| n | 402 | 384 | 408 | 792 |
| Mean (SD) | 1.65 (2.496) | 2.59 (3.582) | 2.47 (3.274) | 2.52 (3.425) |
| LS Mean (SE) | 1.61 (0.149) | 2.46 (0.150) | 2.47 (0.149) | — |
| Difference from Placebo | — | 0.84 | 0.85 | — |
| 95% CI [2] | — | 0.45, 1.24 | 0.46, 1.24 | — |
| p-value vs. Placebo [3] | — | <0.001 | <0.001 | — |
| Week 11 Change from Baseline [1] | | | | |
| n | 399 | 374 | 394 | 768 |
| Mean (SD) | 1.61 (2.483) | 2.67 (3.667) | 2.43 (3.259) | 2.55 (3.463) |
| LS Mean (SE) | 1.58 (0.149) | 2.53 (0.151) | 2.40 (0.149) | — |
| Difference from Placebo | — | 0.96 | 0.83 | — |
| 95% CI [2] | — | 0.56, 1.35 | 0.43, 1.22 | — |
| p-value vs. Placebo [3] | — | <0.001 | <0.001 | — |
| Week 12 Change from Baseline [1] | | | | |
| n | 384 | 370 | 386 | 756 |
| Mean (SD) | 1.68 (2.505) | 2.74 (3.758) | 2.54 (3.382) | 2.64 (3.570) |
| LS Mean (SE) | 1.63 (0.150) | 2.58 (0.151) | 2.49 (0.150) | — |
| Difference from Placebo | — | 0.95 | 0.86 | — |
| 95% CI [2] | — | 0.55, 1.34 | 0.46, 1.25 | — |
| p-value vs. Placebo [3] | — | <0.001 | <0.001 | — |
| Post-Treatment Week 13 Change from Baseline [1] | | | | |
| n | 399 | 382 | 392 | 774 |
| Mean (SD) | 1.43 (2.197) | 1.87 (2.717) | 1.59 (2.376) | 1.73 (2.552) |
| LS Mean (SE) | 1.48 (0.129) | 1.91 (0.131) | 1.64 (0.130) | — |
| Difference from Placebo | — | 0.43 | 0.16 | — |
| 95% CI [2] | — | 0.09, 0.77 | −0.17, 0.50 | — |
| p-value vs. Placebo [3] | — | 0.013 | 0.346 | — |
| Post-Treatment Week 14 Change from Baseline [1] | | | | |
| n | 369 | 348 | 349 | 697 |
| Mean (SD) | 1.33 (2.125) | 1.75 (2.596) | 1.62 (2.588) | 1.69 (2.591) |
| LS Mean (SE) | 1.38 (0.131) | 1.78 (0.133) | 1.67 (0.133) | — |
| Difference from Placebo | — | 0.40 | 0.30 | — |
| 95% CI [2] | — | 0.05, 0.75 | −0.05, 0.64 | — |
| p-value vs. Placebo [3] | — | 0.024 | 0.091 | — |

Source: Listings 16.2.6.1, 16.2.6.2
Note:
Statistics are calculated with 12 week on-treatment and 2 week post-treatment results modeled separately.
[1] Baseline is the mean number of CSBMs recorded during the 2-week baseline diary assessment period prior to the first dose of study drug. The weekly CSBM totals are derived from the daily diary entries reported during the Treatment Period in the BM and Symptom Diary. The time points reported are Treatment Period study weeks defined as follows: Week 1 = study days 1-7; Week 2 = study days 8-14; Week 3 = study days 15-21; Week 4 = study days 22-28; Week 5 = study days 29-35; Week 6 = study days 36-42; Week 7 = study days 43-49; Week 8 = study days 50-56; Week 9 = study days 57-63; Week 10 = study days 64-70; Week 11 = study days 71-77; Week 12 =study days 78-84; Post-Treatment Week 13 = days 1-7 post treatment; Post-Treatment Week 14 = days 8-14 post treatment. Changes from baseline are based on mean replacement approach (MRA) methodology as defined in Section 4.1.2 of the statistical analysis plan.
[2] The 95% confidence interval for the difference between the placebo group and the specified treatment group LS means.
[3] P-value vs. Placebo is from the pairwise comparison of LS means between the placebo group and the specified treatment group using a linear mixed-effects model with fixed effects for gender (stratification variable), treatment, week, the interaction of treatment and week, and the corresponding baseline value, and a random intercept for patient. The model takes into account the repeated measurements of CSBMs for each patient.
Chiltern • 29JUL2015:5:35 PM • I:\Synergy\33610\21_Stat\Programs\Tables\t_sbm_cfb.sas • ZScott

TABLE 25
Change from Baseline in Stool Consistency by Time Point;
Linear Mixed-effects Model, Mean Replacement Approach (MRA)
(ITT Population)

| | | Plecanatide | Plecanatide | Active |
|---|---|---|---|---|
| | Placebo | 3 mg | 6 mg | Combined |
| | (N = 445) | (N = 443) | (N = 449) | (N = 892) |
| Change from Baseline [1], | | | | |
| (overall average estimate | | | | |
| across the 12-week treatment period) | | | | |
| LS Mean (SE) | 0.87 (0.061) | 1.49 (0.060) | 1.50 (0.061) | — |
| Difference from Placebo | — | 0.62 | 0.63 | — |
| 95% CI [2] | — | 0.47, 0.78 | 0.47, 0.79 | — |
| p-value vs. Placebo [3] | — | <0.001 | <0.001 | — |
| Baseline [1] | | | | |
| n | 407 | 424 | 419 | 843 |
| Mean (SD) | 2.35 (1.090) | 2.16 (1.025) | 2.28 (1.112) | 2.22 (1.070) |
| Week 1 Change from Baseline [1] | | | | |
| n | 377 | 392 | 390 | 782 |
| Mean (SD) | 0.55 (1.347) | 1.39 (1.479) | 1.45 (1.501) | 1.42 (1.490) |
| LS Mean (SE) | 0.60 (0.074) | 1.28 (0.072) | 1.44 (0.073) | — |
| Difference from Placebo | — | 0.68 | 0.84 | — |
| 95% CI [2] | — | 0.49, 0.87 | 0.65, 1.03 | — |
| p-value vs. Placebo [3] | — | <0.001 | <0.001 | — |
| Week 2 Change from Baseline [1] | | | | |
| n | 374 | 390 | 378 | 768 |
| Mean (SD) | 0.67 (1.343) | 1.46 (1.583) | 1.38 (1.560) | 1.42 (1.571) |
| LS Mean (SE) | 0.71 (0.074) | 1.38 (0.073) | 1.39 (0.073) | — |
| Difference from Placebo | — | 0.66 | 0.68 | — |
| 95% CI [2] | — | 0.47, 0.86 | 0.48, 0.87 | — |
| p-value vs. Placebo [3] | — | <0.001 | <0.001 | — |
| Week 3 Change from Baseline [1] | | | | |
| n | 364 | 383 | 373 | 756 |
| Mean (SD) | 0.88 (1.463) | 1.51 (1.573) | 1.46 (1.480) | 1.49 (1.527) |
| LS Mean (SE) | 0.90 (0.074) | 1.41 (0.073) | 1.44 (0.074) | — |
| Difference from Placebo | — | 0.51 | 0.54 | — |
| 95% CI [2] | — | 0.32, 0.71 | 0.35, 0.74 | — |
| p-value vs. Placebo [3] | — | <0.001 | <0.001 | — |
| Week 4 Change from Baseline [1] | | | | |
| n | 359 | 381 | 375 | 756 |
| Mean (SD) | 0.87 (1.393) | 1.55 (1.494) | 1.42 (1.603) | 1.48 (1.550) |
| LS Mean (SE) | 0.90 (0.074) | 1.45 (0.073) | 1.42 (0.074) | — |
| Difference from Placebo | — | 0.55 | 0.52 | — |
| 95% CI [2] | — | 0.36, 0.75 | 0.33, 0.72 | — |
| p-value vs. Placebo [3] | — | <0.001 | <0.001 | — |
| Week 5 Change from Baseline [1] | | | | |
| n | 350 | 363 | 362 | 725 |
| Mean (SD) | 0.86 (1.392) | 1.69 (1.462) | 1.57 (1.594) | 1.63 (1.530) |
| LS Mean (SE) | 0.87 (0.075) | 1.55 (0.074) | 1.55 (0.074) | — |
| Difference from Placebo | — | 0.68 | 0.68 | — |
| 95% CI [2] | — | 0.48, 0.87 | 0.48, 0.87 | — |
| p-value vs. Placebo [3] | — | <0.001 | <0.001 | — |
| Week 6 Change from Baseline [1] | | | | |
| n | 344 | 355 | 361 | 716 |
| Mean (SD) | 0.82 (1.290) | 1.67 (1.533) | 1.57 (1.517) | 1.62 (1.525) |
| LS Mean (SE) | 0.82 (0.075) | 1.56 (0.074) | 1.55 (0.074) | — |
| Difference from Placebo | — | 0.74 | 0.72 | — |
| 95% CI [2] | — | 0.54, 0.94 | 0.53, 0.92 | — |
| p-value vs. Placebo [3] | — | <0.001 | <0.001 | — |
| Week 7 Change from Baseline [1] | | | | |
| n | 347 | 349 | 359 | 708 |
| Mean (SD) | 0.89 (1.342) | 1.58 (1.447) | 1.56 (1.540) | 1.57 (1.494) |
| LS Mean (SE) | 0.89 (0.075) | 1.49 (0.074) | 1.54 (0.074) | — |
| Difference from Placebo | — | 0.60 | 0.64 | — |
| 95% CI [2] | — | 0.40, 0.79 | 0.45, 0.84 | — |
| p-value vs. Placebo [3] | — | <0.001 | <0.001 | — |
| Week 8 Change from Baseline [1] | | | | |
| n | 337 | 343 | 352 | 695 |
| Mean (SD) | 0.93 (1.313) | 1.71 (1.496) | 1.53 (1.539) | 1.62 (1.520) |
| LS Mean (SE) | 0.93 (0.075) | 1.60 (0.075) | 1.51 (0.074) | — |
| Difference from Placebo | — | 0.66 | 0.58 | — |
| 95% CI [2] | — | 0.46, 0.86 | 0.38, 0.77 | — |
| p-value vs. Placebo [3] | — | <0.001 | <0.001 | — |
| Week 9 Change from Baseline [1] | | | | |
| n | 333 | 332 | 350 | 682 |
| Mean (SD) | 0.91 (1.377) | 1.68 (1.468) | 1.62 (1.507) | 1.65 (1.487) |
| LS Mean (SE) | 0.89 (0.076) | 1.57 (0.075) | 1.57 (0.075) | — |
| Difference from Placebo | — | 0.68 | 0.68 | — |
| 95% CI [2] | — | 0.48, 0.88 | 0.48, 0.88 | — |
| p-value vs. Placebo [3] | — | <0.001 | <0.001 | — |
| Week 10 Change from Baseline [1] | | | | |
| n | 336 | 336 | 351 | 687 |
| Mean (SD) | 0.93 (1.375) | 1.65 (1.539) | 1.59 (1.432) | 1.62 (1.485) |
| LS Mean (SE) | 0.93 (0.075) | 1.54 (0.075) | 1.55 (0.075) | — |
| Difference from Placebo | — | 0.61 | 0.62 | — |
| 95% CI [2] | — | 0.41, 0.81 | 0.42, 0.81 | — |
| p-value vs. Placebo [3] | — | <0.001 | <0.001 | — |
| Week 11 Change from Baseline [1] | | | | |
| n | 328 | 329 | 339 | 668 |
| Mean (SD) | 0.97 (1.347) | 1.58 (1.598) | 1.58 (1.499) | 1.58 (1.547) |
| LS Mean (SE) | 0.96 (0.076) | 1.50 (0.075) | 1.53 (0.075) | — |
| Difference from Placebo | — | 0.54 | 0.57 | — |
| 95% CI [2] | — | 0.34, 0.74 | 0.37, 0.77 | — |
| p-value vs. Placebo [3] | — | <0.001 | <0.001 | — |
| Week 12 Change from Baseline [1] | | | | |
| n | 320 | 328 | 327 | 655 |
| Mean (SD) | 1.02 (1.344) | 1.69 (1.480) | 1.58 (1.487) | 1.64 (1.483) |
| LS Mean (SE) | 1.02 (0.076) | 1.60 (0.075) | 1.51 (0.076) | — |
| Difference from Placebo | — | 0.58 | 0.49 | — |
| 95% CI [2] | — | 0.38, 0.78 | 0.29, 0.69 | — |
| p-value vs. Placebo [3] | — | <0.001 | <0.001 | — |
| Post-Treatment Week 13 | | | | |
| Change from Baseline [1] | | | | |
| n | 323 | 333 | 339 | 672 |
| Mean (SD) | 0.94 (1.373) | 1.25 (1.368) | 1.04 (1.410) | 1.14 (1.392) |
| LS Mean (SE) | 1.00 (0.072) | 1.22 (0.071) | 1.08 (0.071) | — |
| Difference from Placebo | — | 0.23 | 0.09 | — |
| 95% CI [2] | — | 0.04, 0.42 | −0.10, 0.27 | — |
| p-value vs. Placebo [3] | — | 0.017 | 0.355 | — |
| Post-Treatment Week 14 | | | | |
| Change from Baseline [1] | | | | |
| n | 302 | 304 | 299 | 603 |
| Mean (SD) | 0.92 (1.376) | 1.19 (1.402) | 1.06 (1.354) | 1.12 (1.379) |
| LS Mean (SE) | 1.01 (0.073) | 1.15 (0.073) | 1.07 (0.074) | — |
| Difference from Placebo | — | 0.14 | 0.06 | — |
| 95% CI [2] | — | −0.05, 0.33 | −0.13, 0.25 | — |
| p-value vs. Placebo [3] | — | 0.149 | 0.546 | — |

Source: Listing 16.2.6.1
Note:
Statistics are calculated with 12 week on-treatment and 2 week post-treatment results modeled separately.
[1] Baseline is the mean BSFS score recorded during the 2-week baseline diary assessment period prior to the first dose of study drug. The weekly mean BSFS score per patient is derived from the BSFS entries reported during the Treatment Period in the Daily Symptom Diary. The time points reported are Treatment Period study weeks defined as follows: Week 1 = study days 1-7; Week 2 = study days 8-14; Week 3 = study days 15-21; Week 4 = study days 22-28; Week 5 = study days 29-35; Week 6 = study days 36-42; Week 7 = study days 43-49; Week 8 = study days 50-56; Week 9 = study days 57-63; Week 10 = study days 64-70; Week 11 = study days 71-77; Week 12 = study days 78-84; Post-Treatment Week 13 = days 1-7 post treatment; Post-Treatment Week 14 = days 8-14 post treatment. Changes from baseline are based on mean replacement approach (MRA) methodology as defined in Section 4.1.2 of the statistical analysis plan.
[2] The 95% confidence interval for the difference between the placebo group and the specified treatment group LS means.
[3] P-value vs. Placebo is from the pairwise comparison of LS means between the placebo group and the specified treatment group using a linear mixed-effects model with fixed effects for gender (stratification variable), treatment, week, the interaction of treatment and week, and the corresponding baseline value, and a random intercept for patient. The model takes into account the repeated measurements of stool consistency for each patient.
Chiltern • 29JUL2015:5:35 PM • I:\Synergy\33610\21_Stat\Programs\Tables\t_sbm_cfb.sas • ZScott

TABLE 26
Change from Baseline in Stool Consistency by Time Point;
Linear Mixed-effects Model, Mean Replacement Approach (MRA)
(ITT Population-Excluding Duplicate Patients)

| | | Plecanatide | Plecanatide | Active |
|---|---|---|---|---|
| | Placebo | 3 mg | 6 mg | Combined |
| | (N = 437) | (N = 434) | (N = 443) | (N = 877) |
| Change from Baseline [1], | | | | |
| (overall average estimate | | | | |
| across the 12-week treatment period) | | | | |
| LS Mean (SE) | 0.88 (0.062) | 1.50 (0.061) | 1.52 (0.061) | — |
| Difference from Placebo | — | 0.62 | 0.64 | — |
| 95% CI [2] | — | 0.46, 0.78 | 0.49, 0.80 | — |
| p-value vs. Placebo [3] | — | <0.001 | <0.001 | — |
| Baseline [1] | | | | |
| n | 399 | 415 | 413 | 828 |
| Mean (SD) | 2.35 (1.093) | 2.17 (1.029) | 2.29 (1.116) | 2.23 (1.074) |
| Week 1 Change from Baseline [1] | | | | |
| n | 370 | 384 | 385 | 769 |
| Mean (SD) | 0.56 (1.353) | 1.39 (1.469) | 1.47 (1.495) | 1.43 (1.482) |
| LS Mean (SE) | 0.61 (0.074) | 1.30 (0.073) | 1.47 (0.073) | — |
| Difference from Placebo | — | 0.69 | 0.86 | — |
| 95% CI [2] | — | 0.49, 0.88 | 0.66, 1.05 | — |
| p-value vs. Placebo [3] | — | <0.001 | <0.001 | — |
| Week 2 Change from Baseline [1] | | | | |
| n | 368 | 382 | 374 | 756 |
| Mean (SD) | 0.68 (1.338) | 1.46 (1.578) | 1.39 (1.562) | 1.43 (1.570) |
| LS Mean (SE) | 0.73 (0.074) | 1.39 (0.073) | 1.41 (0.074) | — |
| Difference from Placebo | — | 0.65 | 0.68 | — |
| 95% CI [2] | — | 0.46, 0.85 | 0.49, 0.88 | — |
| p-value vs. Placebo [3] | — | <0.001 | <0.001 | — |
| Week 3 Change from Baseline [1] | | | | |
| n | 358 | 375 | 370 | 745 |
| Mean (SD) | 0.89 (1.470) | 1.52 (1.578) | 1.47 (1.485) | 1.49 (1.532) |
| LS Mean (SE) | 0.91 (0.075) | 1.42 (0.074) | 1.46 (0.074) | — |
| Difference from Placebo | — | 0.51 | 0.55 | — |
| 95% CI [2] | — | 0.32, 0.71 | 0.35, 0.75 | — |
| p-value vs. Placebo [3] | — | <0.001 | <0.001 | — |
| Week 4 Change from Baseline [1] | | | | |
| n | 352 | 374 | 372 | 746 |
| Mean (SD) | 0.87 (1.394) | 1.56 (1.504) | 1.42 (1.608) | 1.49 (1.557) |
| LS Mean (SE) | 0.91 (0.075) | 1.45 (0.074) | 1.44 (0.074) | — |
| Difference from Placebo | — | 0.54 | 0.53 | — |
| 95% CI [2] | — | 0.35, 0.74 | 0.34, 0.73 | — |
| p-value vs. Placebo [3] | — | <0.001 | <0.001 | — |
| Week 5 Change from Baseline [1] | | | | |
| n | 343 | 357 | 359 | 716 |
| Mean (SD) | 0.86 (1.394) | 1.70 (1.469) | 1.58 (1.598) | 1.64 (1.535) |
| LS Mean (SE) | 0.89 (0.075) | 1.56 (0.075) | 1.58 (0.075) | — |
| Difference from Placebo | — | 0.67 | 0.69 | — |
| 95% CI [2] | — | 0.47, 0.87 | 0.49, 0.89 | — |
| p-value vs. Placebo [3] | — | <0.001 | <0.001 | — |
| Week 6 Change from Baseline [1] | | | | |
| n | 338 | 349 | 358 | 707 |
| Mean (SD) | 0.82 (1.298) | 1.67 (1.529) | 1.58 (1.520) | 1.62 (1.524) |
| LS Mean (SE) | 0.83 (0.076) | 1.56 (0.075) | 1.57 (0.075) | — |
| Difference from Placebo | — | 0.73 | 0.74 | — |
| 95% CI [2] | — | 0.53, 0.93 | 0.55, 0.94 | — |
| p-value vs. Placebo [3] | — | <0.001 | <0.001 | — |
| Week 7 Change from Baseline [1] | | | | |
| n | 341 | 343 | 356 | 699 |
| Mean (SD) | 0.88 (1.349) | 1.60 (1.452) | 1.57 (1.544) | 1.58 (1.498) |
| LS Mean (SE) | 0.89 (0.076) | 1.50 (0.075) | 1.56 (0.075) | — |
| Difference from Placebo | — | 0.61 | 0.67 | — |
| 95% CI [2] | — | 0.41, 0.81 | 0.47, 0.86 | — |
| p-value vs. Placebo [3] | — | <0.001 | <0.001 | — |
| Week 8 Change from Baseline [1] | | | | |
| n | 330 | 338 | 349 | 687 |
| Mean (SD) | 0.91 (1.315) | 1.71 (1.500) | 1.54 (1.543) | 1.62 (1.523) |
| LS Mean (SE) | 0.93 (0.076) | 1.60 (0.075) | 1.53 (0.075) | — |
| Difference from Placebo | — | 0.67 | 0.60 | — |
| 95% CI [2] | — | 0.47, 0.87 | 0.40, 0.80 | — |
| p-value vs. Placebo [3] | — | <0.001 | <0.001 | — |
| Week 9 Change from Baseline [1] | | | | |
| n | 329 | 327 | 347 | 674 |
| Mean (SD) | 0.92 (1.368) | 1.68 (1.468) | 1.63 (1.510) | 1.65 (1.489) |
| LS Mean (SE) | 0.92 (0.076) | 1.57 (0.076) | 1.59 (0.075) | — |
| Difference from Placebo | — | 0.66 | 0.68 | — |
| 95% CI [2] | — | 0.45, 0.86 | 0.48, 0.88 | — |
| p-value vs. Placebo [3] | — | <0.001 | <0.001 | — |
| Week 10 Change from Baseline [1] | | | | |
| n | 331 | 330 | 348 | 678 |
| Mean (SD) | 0.93 (1.380) | 1.65 (1.548) | 1.60 (1.437) | 1.62 (1.491) |
| LS Mean (SE) | 0.93 (0.076) | 1.54 (0.076) | 1.57 (0.075) | — |
| Difference from Placebo | — | 0.61 | 0.63 | — |
| 95% CI [2] | — | 0.41, 0.81 | 0.43, 0.83 | — |
| p-value vs. Placebo [3] | — | <0.001 | <0.001 | — |
| Week 11 Change from Baseline [1] | | | | |
| n | 323 | 324 | 336 | 660 |
| Mean (SD) | 0.97 (1.355) | 1.59 (1.595) | 1.59 (1.504) | 1.59 (1.548) |
| LS Mean (SE) | 0.97 (0.076) | 1.51 (0.076) | 1.55 (0.076) | — |
| Difference from Placebo | — | 0.54 | 0.59 | — |
| 95% CI [2] | — | 0.34, 0.75 | 0.38, 0.79 | — |
| p-value vs. Placebo [3] | — | <0.001 | <0.001 | — |
| Week 12 Change from Baseline [1] | | | | |
| n | 315 | 323 | 324 | 647 |
| Mean (SD) | 1.01 (1.348) | 1.70 (1.476) | 1.59 (1.492) | 1.64 (1.484) |
| LS Mean (SE) | 1.02 (0.077) | 1.61 (0.076) | 1.53 (0.076) | — |
| Difference from Placebo | — | 0.58 | 0.51 | — |
| 95% CI [2] | — | 0.38, 0.79 | 0.31, 0.71 | — |
| p-value vs. Placebo [3] | — | <0.001 | <0.001 | — |
| Post-Treatment Week 13 | | | | |
| Change from Baseline [1] | | | | |
| n | 318 | 329 | 336 | 665 |
| Mean (SD) | 0.94 (1.363) | 1.25 (1.374) | 1.04 (1.414) | 1.14 (1.397) |
| LS Mean (SE) | 0.99 (0.072) | 1.22 (0.072) | 1.08 (0.072) | — |
| Difference from Placebo | — | 0.23 | 0.09 | — |
| 95% CI [2] | — | 0.04, 0.41 | −0.10, 0.28 | — |
| p-value vs. Placebo [3] | — | 0.020 | 0.361 | — |
| Post-Treatment Week 14 | | | | |
| Change from Baseline [1] | | | | |
| n | 298 | 300 | 296 | 596 |
| Mean (SD) | 0.93 (1.380) | 1.18 (1.408) | 1.05 (1.358) | 1.12 (1.384) |
| LS Mean (SE) | 1.01 (0.074) | 1.15 (0.074) | 1.07 (0.074) | — |
| Difference from Placebo | — | 0.14 | 0.06 | — |
| 95% CI [2] | — | −0.06, 0.33 | −0.14, 0.25 | — |
| p-value vs. Placebo [3] | — | 0.167 | 0.564 | — |

Source: Listing 16.2.6.1
Note:
Statistics are calculated with 12 week on-treatment and 2 week post-treatment results modeled separately.
[1] Baseline is the mean BSFS score recorded during the 2-week baseline diary assessment period prior to the first dose of study drug. The weekly mean BSFS score per patient is derived from the BSFS entries reported during the Treatment Period in the Daily Symptom Diary. The time points reported are Treatment Period study weeks defined as follows: Week 1 = study days 1-7; Week 2 = study days 8-14; Week 3 = study days 15-21; Week 4 = study days 22-28; Week 5 = study days 29-35; Week 6 = study days 36-42; Week 7 = study days 43-49; Week 8 = study days 50-56; Week 9 = study days 57-63; Week 10 = study days 64-70; Week 11 = study days 71-77; Week 12 = study days 78-84; Post-Treatment Week 13 = days 1-7 post treatment; Post-Treatment Week 14 = days 8-14 post treatment. Changes from baseline are based on mean replacement approach (MRA) methodology as defined in Section 4.1.2 of the statistical analysis plan.
[2] The 95% confidence interval for the difference between the placebo group and the specified treatment group LS means.
[3] P-value vs. Placebo is from the pairwise comparison of LS means between the placebo group and the specified treatment group using a linear mixed-effects model with fixed effects for gender (stratification variable), treatment, week, the interaction of treatment and week, and the corresponding baseline value, and a random intercept for patient. The model takes into account the repeated measurements of stool consistency for each patient.
Chiltern • 29JUL2015:5:35 PM • I:\Synergy\33610\21_Stat\Programs\Tables\t_sbm_cfb.sas • ZScott

TABLE 27
Change from Baseline in Stool Consistency by Time Point;
Linear Mixed-effects Model, Mean Replacement Approach (MRA)
(All Randomized (includes Duplicate Patients))

| | | Plecanatide | Plecanatide | Active |
|---|---|---|---|---|
| | Placebo | 3 mg | 6 mg | Combined |
| | (N = 469) | (N = 470) | (N = 471) | (N = 941) |
| Change from Baseline [1], | | | | |
| (overall average estimate | | | | |
| across the 12-week treatment period) | | | | |
| LS Mean (SE) | 0.85 (0.059) | 1.48 (0.058) | 1.47 (0.059) | — |
| Difference from Placebo | — | 0.63 | 0.63 | — |
| 95% CI [2] | — | 0.48, 0.78 | 0.47, 0.78 | — |
| p-value vs. Placebo [3] | — | <0.001 | <0.001 | — |
| Baseline [1] | | | | |
| n | 430 | 451 | 439 | 890 |
| Mean (SD) | 2.33 (1.081) | 2.14 (1.024) | 2.28 (1.123) | 2.21 (1.076) |
| Week 1 Change from Baseline [1] | | | | |
| n | 399 | 416 | 409 | 825 |
| Mean (SD) | 0.54 (1.325) | 1.38 (1.464) | 1.43 (1.501) | 1.41 (1.482) |
| LS Mean (SE) | 0.58 (0.071) | 1.26 (0.070) | 1.41 (0.071) | — |
| Difference from Placebo | — | 0.68 | 0.84 | — |
| 95% CI [2] | — | 0.49, 0.87 | 0.65, 1.02 | — |
| p-value vs. Placebo [3] | — | <0.001 | <0.001 | — |
| Week 2 Change from Baseline [1] | | | | |
| n | 395 | 412 | 394 | 806 |
| Mean (SD) | 0.66 (1.333) | 1.46 (1.566) | 1.36 (1.560) | 1.41 (1.563) |
| LS Mean (SE) | 0.69 (0.071) | 1.35 (0.070) | 1.36 (0.071) | — |
| Difference from Placebo | — | 0.66 | 0.67 | — |
| 95% CI [2] | — | 0.47, 0.85 | 0.48, 0.86 | — |
| p-value vs. Placebo [3] | — | <0.001 | <0.001 | — |
| Week 3 Change from Baseline [1] | | | | |
| n | 382 | 408 | 389 | 797 |
| Mean (SD) | 0.89 (1.462) | 1.52 (1.570) | 1.44 (1.479) | 1.48 (1.526) |
| LS Mean (SE) | 0.89 (0.072) | 1.41 (0.070) | 1.41 (0.072) | — |
| Difference from Placebo | — | 0.52 | 0.53 | — |
| 95% CI [2] | — | 0.33, 0.71 | 0.34, 0.71 | — |
| p-value vs. Placebo [3] | — | <0.001 | <0.001 | — |
| Week 4 Change from Baseline [1] | | | | |
| n | 377 | 406 | 391 | 797 |
| Mean (SD) | 0.85 (1.376) | 1.56 (1.509) | 1.42 (1.596) | 1.49 (1.553) |
| LS Mean (SE) | 0.87 (0.072) | 1.45 (0.071) | 1.41 (0.071) | — |
| Difference from Placebo | — | 0.58 | 0.54 | — |
| 95% CI [2] | — | 0.39, 0.77 | 0.35, 0.73 | — |
| p-value vs. Placebo [3] | — | <0.001 | <0.001 | — |
| Week 5 Change from Baseline [1] | | | | |
| n | 367 | 384 | 375 | 759 |
| Mean (SD) | 0.85 (1.373) | 1.69 (1.456) | 1.56 (1.598) | 1.63 (1.528) |
| LS Mean (SE) | 0.85 (0.073) | 1.53 (0.071) | 1.52 (0.072) | — |
| Difference from Placebo | — | 0.68 | 0.67 | — |
| 95% CI [2] | — | 0.49, 0.87 | 0.48, 0.86 | — |
| p-value vs. Placebo [3] | — | <0.001 | <0.001 | — |
| Week 6 Change from Baseline [1] | | | | |
| n | 361 | 374 | 375 | 749 |
| Mean (SD) | 0.82 (1.275) | 1.68 (1.530) | 1.54 (1.525) | 1.61 (1.528) |
| LS Mean (SE) | 0.81 (0.073) | 1.55 (0.072) | 1.51 (0.072) | — |
| Difference from Placebo | — | 0.74 | 0.70 | — |
| 95% CI [2] | — | 0.55, 0.93 | 0.51, 0.90 | — |
| p-value vs. Placebo [3] | — | <0.001 | <0.001 | — |
| Week 7 Change from Baseline [1] | | | | |
| n | 364 | 369 | 371 | 740 |
| Mean (SD) | 0.87 (1.327) | 1.59 (1.435) | 1.54 (1.543) | 1.56 (1.489) |
| LS Mean (SE) | 0.86 (0.073) | 1.46 (0.072) | 1.50 (0.072) | — |
| Difference from Placebo | — | 0.60 | 0.64 | — |
| 95% CI [2] | — | 0.41, 0.79 | 0.45, 0.83 | — |
| p-value vs. Placebo [3] | — | <0.001 | <0.001 | — |
| Week 8 Change from Baseline [1] | | | | |
| n | 352 | 362 | 365 | 727 |
| Mean (SD) | 0.91 (1.297) | 1.71 (1.489) | 1.51 (1.538) | 1.61 (1.516) |
| LS Mean (SE) | 0.91 (0.073) | 1.58 (0.072) | 1.48 (0.072) | — |
| Difference from Placebo | — | 0.67 | 0.57 | — |
| 95% CI (2) | — | 0.48, 0.86 | 0.38, 0.76 | — |
| p-value vs. Placebo [3] | — | <0.001 | <0.001 | — |
| Week 9 Change from Baseline [1] | | | | |
| n | 348 | 348 | 365 | 713 |
| Mean (SD) | 0.89 (1.360) | 1.67 (1.455) | 1.60 (1.511) | 1.63 (1.483) |
| LS Mean (SE) | 0.87 (0.073) | 1.55 (0.073) | 1.54 (0.073) | — |
| Difference from Placebo | — | 0.68 | 0.67 | — |
| 95% CI [2] | — | 0.49, 0.88 | 0.48, 0.86 | — |
| p-value vs. Placebo [3] | — | <0.001 | <0.001 | — |
| Week 10 Change from Baseline [1] | | | | |
| n | 350 | 353 | 362 | 715 |
| Mean (SD) | 0.92 (1.357) | 1.64 (1.524) | 1.58 (1.439) | 1.61 (1.481) |
| LS Mean (SE) | 0.90 (0.073) | 1.52 (0.073) | 1.52 (0.073) | — |
| Difference from Placebo | — | 0.62 | 0.62 | — |
| 95% CI [2] | — | 0.42, 0.81 | 0.43, 0.81 | — |
| p-value vs. Placebo [3] | — | <0.001 | <0.001 | — |
| Week 11 Change from Baseline [1] | | | | |
| n | 343 | 345 | 350 | 695 |
| Mean (SD) | 0.94 (1.331) | 1.57 (1.579) | 1.57 (1.499) | 1.57 (1.538) |
| LS Mean (SE) | 0.93 (0.074) | 1.48 (0.073) | 1.50 (0.073) | — |
| Difference from Placebo | — | 0.55 | 0.58 | — |
| 95% CI [2] | — | 0.36, 0.75 | 0.38, 0.77 | — |
| p-value vs. Placebo [3] | — | <0.001 | <0.001 | — |
| Week 12 Change from Baseline [1] | | | | |
| n | 331 | 342 | 339 | 681 |
| Mean (SD) | 1.01 (1.328) | 1.69 (1.468) | 1.55 (1.488) | 1.62 (1.478) |
| LS Mean (SE) | 1.00 (0.074) | 1.57 (0.073) | 1.48 (0.074) | — |
| Difference from Placebo | — | 0.57 | 0.48 | — |
| 95% CI [2] | — | 0.38, 0.77 | 0.28, 0.68 | — |
| p-value vs. Placebo [3] | — | <0.001 | <0.001 | — |
| Post-Treatment Week 13 | | | | |
| Change from Baseline [1] | | | | |
| n | 334 | 345 | 350 | 695 |
| Mean (SD) | 0.95 (1.362) | 1.27 (1.358) | 1.03 (1.408) | 1.15 (1.387) |
| LS Mean (SE) | 0.99 (0.070) | 1.23 (0.069) | 1.08 (0.070) | — |
| Difference from Placebo | — | 0.24 | 0.09 | — |
| 95% CI [2] | — | 0.06, 0.42 | −0.09, 0.27 | — |
| p-value vs. Placebo [3] | — | 0.010 | 0.331 | — |
| Post-Treatment Week 14 | | | | |
| Change from Baseline [1] | | | | |
| n | 313 | 313 | 308 | 621 |
| Mean (SD) | 0.92 (1.361) | 1.19 (1.393) | 1.04 (1.393) | 1.12 (1.394) |
| LS Mean (SE) | 0.99 (0.072) | 1.14 (0.071) | 1.06 (0.072) | — |
| Difference from Placebo | — | 0.15 | 0.07 | — |
| 95% CI [2] | — | −0.04, 0.34 | −0.12, 0.25 | — |
| p-value vs. Placebo [3] | — | 0.111 | 0.496 | — |

Source: Listing 16.2.6.1
Note:
Statistics are calculated with 12 week on-treatment and 2 week post-treatment results modeled separately.
[1] Baseline is the mean BSFS score recorded during the 2-week baseline diary assessment period prior to the first dose of study drug. The weekly mean BSFS score per patient is derived from the BSFS entries reported during the Treatment Period in the Daily Symptom Diary. The time points reported are Treatment Period study weeks defined as follows: Week 1 = study days 1-7; Week 2 = study days 8-14; Week 3 = study days 15-21; Week 4 = study days 22-28; Week 5 = study days 29-35; Week 6 = study days 36-42; Week 7 = study days 43-49; Week 8 = study days 50-56; Week 9 = study days 57-63; Week 10 = study days 64-70; Week 11 = study days 71-77; Week 12 = study days 78-84; Post-Treatment Week 13 = days 1-7 post treatment; Post-Treatment Week 14 = days 8-14 post treatment. Changes from baseline are based on mean replacement approach (MRA) methodology as defined in Section 4.1.2 of the statistical analysis plan.
[2] The 95% confidence interval for the difference between the placebo group and the specified treatment group LS means.
[3] P-value vs. Placebo is from the pairwise comparison of LS means between the placebo group and the specified treatment group using a linear mixed-effects model with fixed effects for gender (stratification variable), treatment, week, the interaction of treatment and week, and the corresponding baseline value, and a random intercept for patient. The model takes into account the repeated measurements of stool consistency for each patient.
Chiltern • 29JUL2015:5:35 PM • I:\Synergy\33610\21_Stat\Programs\Tables\t_sbm_cfb.sas • ZScott

TABLE 28
Overall Summary of Treatment Emergent Adverse Events
(Safety Population)

| | | | Plecanatide | | Plecanatide | | Active | | | |
|---|---|---|---|---|---|---|---|---|---|---|
| | Placebo | | 3 mg | | 6 mg | | Combined | | Overall | |
| | (N = 466) | | (N = 467) | | (N = 469) | | (N = 936) | | (N = 1402) | |
| Nuraber (%) of patients with at least one TEAE | 115 | (24.7) | 120 | (25.7) | 137 | (29.2) | 257 | (27.5) | 372 | (26.5) |
| Number (%) of patients with at least one TEAE by | | | | | | | | | | |
| maximum severity | | | | | | | | | | |
| Mild | 65 | (13.9) | 67 | (14.3) | 81 | (17.3) | 148 | (15.8) | 213 | (15.2) |
| Moderate | 43 | (9.2) | 45 | (9.6) | 49 | (10.4) | 94 | (10.0) | 137 | (9.8) |
| Severe | 6 | (1.3) | 8 | (1.7) | 6 | (1.3) | 14 | (1.5) | 20 | (1.4) |
| Grade is missing | 1 | (0.2) | 0 | | 1 | (0.2) | 1 | (0.1) | 2 | (0.1) |
| Number (%) of patients with at least one TEAE by | | | | | | | | | | |
| most recent attribution to study drug | | | | | | | | | | |
| Reasonable Possibility [1] | 17 | (3.6) | 27 | (5.8) | 31 | (6.6) | 58 | (6.2) | 75 | (5.3) |
| No Reasonable Possibility | 98 | (21.0) | 93 | (19.9) | 106 | (22.6) | 199 | (21.3) | 297 | (21.2) |
| Number (%) of patients with at least one TEAE | 14 | (3.0) | 15 | (3.2) | 18 | (3.8) | 33 | (3.5) | 47 | (3.4) |
| leading to discontinuation of Plecanatide | | | | | | | | | | |
| Number (%) of patients with at least one TE SAE | 8 | (1.7) | 8 | (1.7) | 4 | (0.9) | 12 | (1.3) | 20 | (1.4) |

Source: Listing 16.2.10.1
[1] There is evidence to suggest a causal relationship between the drug and the AE.
Chiltern • 29JUL2015:5:34 PM • I:\Synergy\33610\21_Stat\Programs\Tables\t_aeovr.sas • ZScott

TABLE 29
Summary of Treatment Emergent Adverse Events by System Organ Class and Preferred Term
(Safety Population)

| | | | | Plecanatide | | | Plecanatide | | | Active | | | | | |
|---|---|---|---|---|---|---|---|---|---|---|---|---|---|---|---|
| | Placebo | | | 3 mg | | | 6 mg | | | Combined | | | Overall | | |
| System Organ Class | (N = 466) | | | (N = 467) | | | (N = 469) | | | (N = 936) | | | (N = 1402) | | |
| Preferred Term | N | (%) | E | N | (%) | E | N | (%) | E | N | (%) | E | N | (%) | E |
| Number of Treatment | 115 | (24.7) | 196 | 120 | (25.7) | 196 | 137 | (29.2) | 226 | 257 | (27.5) | 422 | 372 | (26.5) | 618 |
| Emergent Adverse Events | | | | | | | | | | | | | | | |
| Blood and lymphatic | 7 | (1.5) | 8 | 5 | (1.1) | 5 | 5 | (1.1) | 5 | 10 | (1.1) | 10 | 17 | (1.2) | 18 |
| system disorders | | | | | | | | | | | | | | | |
| Anaemia | 1 | (0.2) | 1 | 3 | (0.6) | 3 | 4 | (0.9) | 4 | 7 | (0.7) | 7 | 8 | (0.6) | 8 |
| Hypochromic anaemia | 0 | | 0 | 1 | (0.2) | 1 | 0 | | 0 | 1 | (0.1) | 1 | 1 | (0.1) | 1 |
| Iron deficiency anaemia | 2 | (0.4) | 2 | 0 | | 0 | 0 | | 0 | 0 | | 0 | 2 | (0.1) | 2 |
| Leukocytosis | 1 | (0.2) | 2 | 0 | | 0 | 0 | | 0 | 0 | | 0 | 1 | (0.1) | 2 |
| Lymphadenopathy | 2 | (0.4) | 2 | 1 | (0.2) | 1 | 1 | (0.2) | 1 | 2 | (0.2) | 2 | 4 | (0.3) | 4 |
| Thrombocytopenia | 1 | (0.2) | 1 | 0 | | 0 | 0 | | 0 | 0 | | 0 | 1 | (0.1) | 1 |
| Cardiac disorders | 3 | (0.6) | 6 | 1 | (0.2) | 1 | 0 | | 0 | 1 | (0.1) | 1 | 4 | (0.3) | 7 |
| Atrioventricular block first | 1 | (0.2) | 1 | 0 | | 0 | 0 | | 0 | 0 | | 0 | 1 | (0.1) | 1 |
| degree | | | | | | | | | | | | | | | |
| Atrioventricular dissociation | 1 | (0.2) | 1 | 0 | | 0 | 0 | | 0 | 0 | | 0 | 1 | (0.1) | 1 |
| Cardiac failure congestive | 0 | | 0 | 1 | (0.2) | 1 | 0 | | 0 | 1 | (0.1) | 1 | 1 | (0.1) | 1 |
| Nodal rhythm | 1 | (0.2) | 1 | 0 | | 0 | 0 | | 0 | 0 | | 0 | 1 | (0.1) | 1 |
| Palpitations | 1 | (0.2) | 1 | 0 | | 0 | 0 | | 0 | 0 | | 0 | 1 | (0.1) | 1 |
| Sinus bradycardia | 1 | (0.2) | 1 | 0 | | 0 | 0 | | 0 | 0 | | 0 | 1 | (0.1) | 1 |
| Tachycardia | 1 | (0.2) | 1 | 0 | | 0 | 0 | | 0 | 0 | | 0 | 1 | (0.1) | 1 |
| Ear and labyrinth disorders | 0 | | 0 | 3 | (0.6) | 3 | 1 | (0.2) | 1 | 4 | (0.4) | 4 | 4 | (0.3) | 4 |
| Deafness | 0 | | 0 | 0 | | 0 | 1 | (0.2) | 1 | 1 | (0.1) | 1 | 1 | (0.1) | 1 |
| Ear pain | 0 | | 0 | 1 | (0.2) | 1 | 0 | | 0 | 1 | (0.1) | 1 | 1 | (0.1) | 1 |
| External ear pain | 0 | | 0 | 1 | (0.2) | 1 | 0 | | 0 | 1 | (0.1) | 1 | 1 | (0.1) | 1 |
| Vertigo positional | 0 | | 0 | 1 | (0.2) | 1 | 0 | | 0 | 1 | (0.1) | 1 | 1 | (0.1) | 1 |
| Endocrine disorders | 1 | (0.2) | 1 | 0 | | 0 | 0 | | 0 | 0 | | 0 | 1 | (0.1) | 1 |
| Goitre | 1 | (0.2) | 1 | 0 | | 0 | 0 | | 0 | 0 | | 0 | 1 | (0.1) | 1 |
| Eye disorders | 1 | (0.2) | 2 | 1 | (0.2) | 1 | 0 | | 0 | 1 | (0.1) | 1 | 2 | (0.1) | 3 |
| Dry eye | 0 | | 0 | 1 | (0.2) | 1 | 0 | | 0 | 1 | (0.1) | 1 | 1 | (0.1) | 1 |
| Ocular discomfort | 1 | (0.2) | 1 | 0 | | 0 | 0 | | 0 | 0 | | 0 | 1 | (0.1) | 1 |
| Ocular hyperaemia | 1 | (0.2) | 1 | 0 | | 0 | 0 | | 0 | 0 | | 0 | 1 | (0.1) | 1 |
| Gastrointestinal disorders | 21 | (4.5) | 33 | 34 | (7.3) | 42 | 43 | (9.2) | 61 | 77 | (8.2) | 103 | 98 | (7.0) | 136 |
| Abdominal discomfort | 1 | (0.2) | 1 | 0 | | 0 | 2 | (0.4) | 2 | 2 | (0.2) | 2 | 3 | (0.2) | 3 |
| Abdominal distension | 1 | (0.2) | 2 | 3 | (0.6) | 3 | 3 | (0.6) | 3 | 6 | (0.6) | 6 | 7 | (0.5) | 8 |
| Abdominal pain | 4 | (0.9) | 5 | 2 | (0.4) | 2 | 5 | (1.1) | 5 | 7 | (0.7) | 7 | 11 | (0.8) | 12 |
| Abdominal pain lower | 0 | | 0 | 0 | | 0 | 1 | (0.2) | 1 | 1 | (0.1) | 1 | 1 | (0.1) | 1 |
| Abdominal pain upper | 1 | (0.2) | 1 | 0 | | 0 | 0 | | 0 | 0 | | 0 | 1 | (0.1) | 1 |
| Abdominal tenderness | 0 | | 0 | 1 | (0.2) | 1 | 0 | | 0 | 1 | (0.1) | 1 | 1 | (0.1) | 1 |
| Anorectal discomfort | 0 | | 0 | 0 | | 0 | 3 | (0.6) | 3 | 3 | (0.3) | 3 | 3 | (0.2) | 3 |
| Defaecation urgency | 0 | | 0 | 1 | (0.2) | 1 | 2 | (0.4) | 2 | 3 | (0.3) | 3 | 3 | (0.2) | 3 |
| Diarrhoea | 6 | (1.3) | 7 | 15 | (3.2) | 15 | 21 | (4.5) | 25 | 36 | (3.8) | 40 | 42 | (3.0) | 47 |
| Dry mouth | 1 | (0.2) | 1 | 1 | (0.2) | 1 | 1 | (0.2) | 1 | 2 | (0.2) | 2 | 3 | (0.2) | 3 |
| Dyspepsia | 1 | (0.2) | 2 | 4 | (0.9) | 4 | 0 | | 0 | 4 | (0.4) | 4 | 5 | (0.4) | 6 |
| Epigastric discomfort | 0 | | 0 | 0 | | 0 | 1 | (0.2) | 1 | 1 | (0.1) | 1 | 1 | (0.1) | 1 |
| Faecal incontinence | 0 | | 0 | 0 | | 0 | 1 | (0.2) | 2 | 1 | (0.1) | 2 | 1 | (0.1) | 2 |
| Flatulence | 4 | (0.9) | 4 | 1 | (0.2) | 1 | 4 | (0.9) | 4 | 5 | (0.5) | 5 | 9 | (0.6) | 9 |
| Gastrointestinal motility | 0 | | 0 | 1 | (0.2) | 1 | 0 | | 0 | 1 | (0.1) | 1 | 1 | (0.1) | 1 |
| disorder | | | | | | | | | | | | | | | |
| Gastrooesophageal reflux | 0 | | 0 | 2 | (0.4) | 2 | 0 | | 0 | 2 | (0.2) | 2 | 2 | (0.1) | 2 |
| disease | | | | | | | | | | | | | | | |
| Gingival pain | 0 | | 0 | 0 | | 0 | 1 | (0.2) | 1 | 1 | (0.1) | 1 | 1 | (0.1) | 1 |
| Haematochezia | 0 | | 0 | 1 | (0.2) | 1 | 2 | (0.4) | 2 | 3 | (0.3) | 3 | 3 | (0.2) | 3 |
| Haemorrhoidal haemorrhage | 0 | | 0 | 0 | | 0 | 1 | (0.2) | 1 | 1 | (0.1) | 1 | 1 | (0.1) | 1 |
| Haemorrhoids | 1 | (0.2) | 1 | 1 | (0.2) | 1 | 1 | (0.2) | 1 | 2 | (0.2) | 2 | 3 | (0.2) | 3 |
| Ileus | 1 | (0.2) | 1 | 0 | | 0 | 0 | | 0 | 0 | | 0 | 1 | (0.1) | 1 |
| Nausea | 5 | (1.1) | 5 | 4 | (0.9) | 4 | 5 | (1.1) | 5 | 9 | (1.0) | 9 | 14 | (1.0) | 14 |
| Painful defaecation | 0 | | 0 | 1 | (0.2) | 1 | 0 | | 0 | 1 | (0.1) | 1 | 1 | (0.1) | 1 |
| Toothache | 0 | | 0 | 1 | (0.2) | 1 | 0 | | 0 | 1 | (0.1) | 1 | 1 | (0.1) | 1 |
| Vomiting | 3 | (0.6) | 3 | 3 | (0.6) | 3 | 2 | (0.4) | 2 | 5 | (0.5) | 5 | 8 | (0.6) | 8 |
| General disorders and | 11 | (2.4) | 11 | 9 | (1.9) | 10 | 5 | (1.1) | 7 | 14 | (1.5) | 17 | 25 | (1.8) | 28 |
| administration site conditions | | | | | | | | | | | | | | | |
| Chest discomfort | 0 | | 0 | 0 | | 0 | 1 | (0.2) | 1 | 1 | (0.1) | 1 | 1 | (0.1) | 1 |
| Chest pain | 0 | | 0 | 1 | (0.2) | 1 | 1 | (0.2) | 1 | 2 | (0.2) | 2 | 2 | (0.1) | 2 |
| Chills | 0 | | 0 | 1 | (0.2) | 1 | 0 | | 0 | 1 | (0.1) | 1 | 1 | (0.1) | 1 |
| Fatigue | 2 | (0.4) | 2 | 1 | (0.2) | 1 | 1 | (0.2) | 1 | 2 | (0.2) | 2 | 4 | (0.3) | 4 |
| Influenza like illness | 1 | (0.2) | 1 | 0 | | 0 | 1 | (0.2) | 1 | 1 | (0.1) | 1 | 2 | (0.1) | 2 |
| Non-cardiac chest pain | 1 | (0.2) | 1 | 1 | (0.2) | 1 | 1 | (0.2) | 1 | 2 | (0.2) | 2 | 3 | (0.2) | 3 |
| Oedema | 0 | | 0 | 1 | (0.2) | 1 | 0 | | 0 | 1 | (0.1) | 1 | 1 | (0.1) | 1 |
| Oedema peripheral | 3 | (0.6) | 3 | 1 | (0.2) | 1 | 0 | | 0 | 1 | (0.1) | 1 | 4 | (0.3) | 4 |
| Pain | 1 | (0.2) | 1 | 3 | (0.6) | 3 | 1 | (0.2) | 1 | 4 | (0.4) | 4 | 5 | (0.4) | 5 |
| Pyrexia | 3 | (0.6) | 3 | 1 | (0.2) | 1 | 1 | (0.2) | 1 | 2 | (0.2) | 2 | 5 | (0.4) | 5 |
| Hepatobiliary disorders | 0 | | 0 | 2 | (0.4) | 3 | 0 | | 0 | 2 | (0.2) | 3 | 2 | (0.1) | 3 |
| Cholecystitis | 0 | | 0 | 1 | (0.2) | 1 | 0 | | 0 | 1 | (0.1) | 1 | 1 | (0.1) | 1 |
| Cholelithiasis | 0 | | 0 | 1 | (0.2) | 1 | 0 | | 0 | 1 | (0.1) | 1 | 1 | (0.1) | 1 |
| Hepatic steatosis | 0 | | 0 | 1 | (0.2) | 1 | 0 | | 0 | 1 | (0.1) | 1 | 1 | (0.1) | 1 |
| Infections and infestations | 36 | (7.7) | 42 | 34 | (7.3) | 38 | 43 | (9.2) | 51 | 77 | (8.2) | 89 | 113 | (8.1) | 131 |
| Acute sinusitis | 1 | (0.2) | 1 | 0 | | 0 | 0 | | 0 | 0 | | 0 | 1 | (0.1) | 1 |
| Asymptomatic bacteriuria | 0 | | 0 | 0 | | 0 | 1 | (0.2) | 1 | 1 | (0.1) | 1 | 1 | (0.1) | 1 |
| Bronchitis | 3 | (0.6) | 3 | 2 | (0.4) | 2 | 3 | (0.6) | 3 | 5 | (0.5) | 5 | 8 | (0.6) | 8 |
| Cellulitis | 0 | | 0 | 0 | | 0 | 2 | (0.4) | 2 | 2 | (0.2) | 2 | 2 | (0.1) | 2 |
| Cystitis | 1 | (0.2) | 1 | 1 | (0.2) | 1 | 0 | | 0 | 1 | (0.1) | 1 | 2 | (0.1) | 2 |
| Ear infection | 2 | (0.4) | 2 | 1 | (0.2) | 1 | 1 | (0.2) | 1 | 2 | (0.2) | 2 | 4 | (0.3) | 4 |
| Folliculitis | 1 | (0.2) | 1 | 0 | | 0 | 0 | | 0 | 0 | | 0 | 1 | (0.1) | 1 |
| Gastroenteritis | 1 | (0.2) | 1 | 2 | (0.4) | 2 | 0 | | 0 | 2 | (0.2) | 2 | 3 | (0.2) | 3 |
| Gastroenteritis viral | 1 | (0.2) | 1 | 0 | | 0 | 0 | | 0 | 0 | | 0 | 1 | (0.1) | 1 |
| Gastrointestinal viral infection | 0 | | 0 | 0 | | 0 | 1 | (0.2) | 1 | 1 | (0.1) | 1 | 1 | (0.1) | 1 |
| Herpes zoster | 0 | | 0 | 1 | (0.2) | 1 | 1 | (0.2) | 1 | 2 | (0.2) | 2 | 2 | (0.1) | 2 |
| Influenza | 2 | (0.4) | 2 | 2 | (0.4) | 2 | 7 | (1.5) | 7 | 9 | (1.0) | 9 | 11 | (0.8) | 11 |
| Localised infection | 0 | | 0 | 0 | | 0 | 1 | (0.2) | 1 | 1 | (0.1) | 1 | 1 | (0.1) | 1 |
| Nasopharyngitis | 6 | (1.3) | 6 | 7 | (1.5) | 7 | 9 | (1.9) | 10 | 16 | (1.7) | 17 | 22 | (1.6) | 23 |
| Otitis media | 1 | (0.2) | 1 | 0 | | 0 | 1 | (0.2) | 1 | 1 | (0.1) | 1 | 2 | (0.1) | 2 |
| Pharyngitis | 1 | (0.2) | 1 | 0 | | 0 | 0 | | 0 | 0 | | 0 | 1 | (0.1) | 1 |
| Pharyngitis streptococcal | 2 | (0.4) | 2 | 0 | | 0 | 1 | (0.2) | 1 | 1 | (0.1) | 1 | 3 | (0.2) | 3 |
| Pneumonia | 0 | | 0 | 0 | | 0 | 1 | (0.2) | 1 | 1 | (0.1) | 1 | 1 | (0.1) | 1 |
| Pyelonephritis | 1 | (0.2) | 1 | 0 | | 0 | 0 | | 0 | 0 | | 0 | 1 | (0.1) | 1 |
| Rhinitis | 2 | (0.4) | 2 | 0 | | 0 | 1 | (0.2) | 1 | 1 | (0.1) | 1 | 3 | (0.2) | 3 |
| Sinusitis | 1 | (0.2) | 1 | 2 | (0.4) | 2 | 3 | (0.6) | 3 | 5 | (0.5) | 5 | 6 | (0.4) | 6 |
| Subcutaneous abscess | 0 | | 0 | 0 | | 0 | 1 | (0.2) | 2 | 1 | (0.1) | 2 | 1 | (0.1) | 2 |
| Tinea versicolour | 1 | (0.2) | 1 | 0 | | 0 | 0 | | 0 | 0 | | 0 | 1 | (0.1) | 1 |
| Tooth abscess | 1 | (0.2) | 1 | 0 | | 0 | 0 | | 0 | 0 | | 0 | 1 | (0.1) | 1 |
| Tooth infection | 2 | (0.4) | 2 | 0 | | 0 | 0 | | 0 | 0 | | 0 | 2 | (0.1) | 2 |
| Upper respiratory tract | 3 | (0.6) | 3 | 8 | (1.7) | 8 | 4 | (0.9) | 5 | 12 | (1.3) | 13 | 15 | (1.1) | 16 |
| infection | | | | | | | | | | | | | | | |
| Urinary tract infection | 6 | (1.3) | 6 | 8 | (1.7) | 8 | 8 | (1.7) | 8 | 16 | (1.7) | 16 | 22 | (1.6) | 22 |
| Vaginal infection | 2 | (0.4) | 2 | 0 | | 0 | 0 | | 0 | 0 | | 0 | 2 | (0.1) | 2 |
| Vaginitis bacterial | 0 | | 0 | 1 | (0.2) | 1 | 0 | | 0 | 1 | (0.1) | 1 | 1 | (0.1) | 1 |
| Viral infection | 1 | (0.2) | 1 | 1 | (0.2) | 1 | 1 | (0.2) | 1 | 2 | (0.2) | 2 | 3 | (0.2) | 3 |
| Viral pharyngitis | 0 | | 0 | 1 | (0.2) | 1 | 0 | | 0 | 1 | (0.1) | 1 | 1 | (0.1) | 1 |
| Viral upper respiratory tract | 0 | | 0 | 1 | (0.2) | 1 | 1 | (0.2) | 1 | 2 | (0.2) | 2 | 2 | (0.1) | 2 |
| infection | | | | | | | | | | | | | | | |
| Injury, poisoning and | 12 | (2.6) | 14 | 7 | (1.5) | 10 | 9 | (1.9) | 9 | 16 | (1.7) | 19 | 28 | (2.0) | 33 |
| procedural complications | | | | | | | | | | | | | | | |
| Back injury | 0 | | 0 | 1 | (0.2) | 1 | 0 | | 0 | 1 | (0.1) | 1 | 1 | (0.1) | 1 |
| Burns second degree | 1 | (0.2) | 1 | 0 | | 0 | 0 | | 0 | 0 | | 0 | 1 | (0.1) | 1 |
| Contusion | 1 | (0.2) | 1 | 0 | | 0 | 2 | (0.4) | 2 | 2 | (0.2) | 2 | 3 | (0.2) | 3 |
| Corneal abrasion | 0 | | 0 | 1 | (0.2) | 1 | 0 | | 0 | 1 | (0.1) | 1 | 1 | (0.1) | 1 |
| Ear abrasion | 0 | | 0 | 1 | (0.2) | 1 | 0 | | 0 | 1 | (0.1) | 1 | 1 | (0.1) | 1 |
| Excoriation | 1 | (0.2) | 1 | 1 | (0.2) | 2 | 0 | | 0 | 1 | (0.1) | 2 | 2 | (0.1) | 3 |
| Fall | 2 | (0.4) | 2 | 0 | | 0 | 0 | | 0 | 0 | | 0 | 2 | (0.1) | 2 |
| Femoral neck fracture | 1 | (0.2) | 1 | 0 | | 0 | 0 | | 0 | 0 | | 0 | 1 | (0.1) | 1 |
| Foot fracture | 0 | | 0 | 0 | | 0 | 1 | (0.2) | 1 | 1 | (0.1) | 1 | 1 | (0.1) | 1 |
| Hand fracture | 0 | | 0 | 0 | | 0 | 2 | (0.4) | 2 | 2 | (0.2) | 2 | 2 | (0.1) | 2 |
| Joint dislocation | 0 | | 0 | 1 | (0.2) | 1 | 0 | | 0 | 1 | (0.1) | 1 | 1 | (0.1) | 1 |
| Joint injury | 0 | | 0 | 2 | (0.4) | 2 | 0 | | 0 | 2 | (0.2) | 2 | 2 | (0.1) | 2 |
| Laceration | 0 | | 0 | 0 | | 0 | 1 | (0.2) | 1 | 1 | (0.1) | 1 | 1 | (0.1) | 1 |
| Ligament rupture | 1 | (0.2) | 1 | 0 | | 0 | 0 | | 0 | 0 | | 0 | 1 | (0.1) | 1 |
| Ligament sprain | 2 | (0.4) | 2 | 2 | (0.4) | 2 | 0 | | 0 | 2 | (0.2) | 2 | 4 | (0.3) | 4 |
| Lower limb fracture | 1 | (0.2) | 1 | 0 | | 0 | 0 | | 0 | 0 | | 0 | 1 | (0.1) | 1 |
| Muscle strain | 1 | (0.2) | 1 | 0 | | 0 | 1 | (0.2) | 1 | 1 | (0.1) | 1 | 2 | (0.1) | 2 |
| Procedural pain | 1 | (0.2) | 1 | 0 | | 0 | 1 | (0.2) | 1 | 1 | (0.1) | 1 | 2 | (0.1) | 2 |
| Skeletal injury | 0 | | 0 | 0 | | 0 | 1 | (0.2) | 1 | 1 | (0.1) | 1 | 1 | (0.1) | 1 |
| Spinal column injury | 1 | (0.2) | 1 | 0 | | 0 | 0 | | 0 | 0 | | 0 | 1 | (0.1) | 1 |
| Wound | 1 | (0.2) | 1 | 0 | | 0 | 0 | | 0 | 0 | | 0 | 1 | (0.1) | 1 |
| Investigations | 6 | (1.3) | 7 | 11 | (2.4) | 12 | 12 | (2.6) | 17 | 23 | (2.3) | 29 | 29 | (2.1) | 36 |
| Alanine aminotransferase | 1 | (0.2) | 1 | 2 | (0.4) | 2 | 0 | | 0 | 2 | (0.2) | 2 | 3 | (0.2) | 3 |
| increased | | | | | | | | | | | | | | | |
| Aspartate aminotransferase | 0 | | 0 | 2 | (0.4) | 2 | 0 | | 0 | 2 | (0.2) | 2 | 2 | (0.1) | 2 |
| increased | | | | | | | | | | | | | | | |
| Blood amylase increased | 1 | (0.2) | 1 | 1 | (0.2) | 1 | 3 | (0.6) | 3 | 4 | (0.4) | 4 | 5 | (0.4) | 5 |
| Blood creatinine increased | 0 | | 0 | 0 | | 0 | 1 | (0.2) | 1 | 1 | (0.1) | 1 | 1 | (0.1) | 1 |
| Blood glucose increased | 1 | (0.2) | 1 | 0 | | 0 | 0 | | 0 | 0 | | 0 | 1 | (0.1) | 1 |
| Blood sodium increased | 1 | (0.2) | 1 | 0 | | 0 | 0 | | 0 | 0 | | 0 | 1 | (0.1) | 1 |
| Blood triglycerides increased | 0 | | 0 | 1 | (0.2) | 1 | 2 | (0.4) | 2 | 3 | (0.3) | 3 | 3 | (0.2) | 3 |
| Blood uric acid increased | 0 | | 0 | 0 | | 0 | 1 | (0.2) | 1 | 1 | (0.1) | 1 | 1 | (0.1) | 1 |
| Blood urine present | 0 | | 0 | 0 | | 0 | 1 | (0.2) | 1 | 1 | (0.1) | 1 | 1 | (0.1) | 1 |
| Electrocardiogram QT | 0 | | 0 | 1 | (0.2) | 1 | 0 | | 0 | 1 | (0.1) | 1 | 1 | (0.1) | 1 |
| prolonged | | | | | | | | | | | | | | | |
| Electrocardiogram T wave | 0 | | 0 | 0 | | 0 | 1 | (0.2) | 1 | 1 | (0.1) | 1 | 1 | (0.1) | 1 |
| abnormal | | | | | | | | | | | | | | | |
| Eosinophil count increased | 0 | | 0 | 1 | (0.2) | 1 | 0 | | 0 | 1 | (0.1) | 1 | 1 | (0.1) | 1 |
| Hepatic enzyme increased | 0 | | 0 | 0 | | 0 | 1 | (0.2) | 1 | 1 | (0.1) | 1 | 1 | (0.1) | 1 |
| Hormone level abnormal | 0 | | 0 | 0 | | 0 | 1 | (0.2) | 1 | 1 | (0.1) | 1 | 1 | (0.1) | 1 |
| Lipase increased | 1 | (0.2) | 1 | 1 | (0.2) | 1 | 1 | (0.2) | 1 | 2 | (0.2) | 2 | 3 | (0.2) | 3 |
| Liver function test abnormal | 1 | (0.2) | 1 | 1 | (0.2) | 1 | 1 | (0.2) | 2 | 2 | (0.2) | 3 | 3 | (0.2) | 4 |
| Pancreatic enzymes increased | 0 | | 0 | 1 | (0.2) | 1 | 0 | | 0 | 1 | (0.1) | 1 | 1 | (0.1) | 1 |
| Transaminases increased | 0 | | 0 | 1 | (0.2) | 1 | 0 | | 0 | 1 | (0.1) | 1 | 1 | (0.1) | 1 |
| Weight decreased | 0 | | 0 | 0 | | 0 | 1 | (0.2) | 2 | 1 | (0.1) | 2 | 1 | (0.1) | 2 |
| Weight increased | 1 | (0.2) | 1 | 0 | | 0 | 1 | (0.2) | 1 | 1 | (0.1) | 1 | 2 | (0.1) | 2 |
| Metabolism and nutrition | 9 | (1.9) | 11 | 5 | (1.1) | 7 | 12 | (2.6) | 16 | 17 | (1.8) | 23 | 26 | (1.9) | 34 |
| disorders | | | | | | | | | | | | | | | |
| Decreased appetite | 2 | (0.4) | 2 | 0 | | 0 | 0 | | 0 | 0 | | 0 | 2 | (0.1) | 2 |
| Dehydration | 1 | (0.2) | 1 | 0 | | 0 | 0 | | 0 | 0 | | 0 | 1 | (0.1) | 1 |
| Hypercholesterolaemia | 0 | | 0 | 0 | | 0 | 1 | (0.2) | 1 | 1 | (0.1) | 1 | 1 | (0.1) | 1 |
| Hyperglycaemia | 3 | (0.6) | 3 | 0 | | 0 | 2 | (0.4) | 2 | 2 | (0.2) | 2 | 5 | (0.4) | 5 |
| Hyperkalaemia | 0 | | 0 | 0 | | 0 | 1 | (0.2) | 1 | 1 | (0.1) | 1 | 1 | (0.1) | 1 |
| Hyperlipidaemia | 1 | (0.2) | 1 | 1 | (0.2) | 1 | 0 | | 0 | 1 | (0.1) | 1 | 2 | (0.1) | 2 |
| Hyperphosphataemia | 0 | | 0 | 0 | | 0 | 1 | (0.2) | 1 | 1 | (0.1) | 1 | 1 | (0.1) | 1 |
| Hypertriglyceridaemia | 0 | | 0 | 1 | (0.2) | 1 | 4 | (0.9) | 4 | 5 | (0.5) | 5 | 5 | (0.4) | 5 |
| Hyperuricaemia | 0 | | 0 | 1 | (0.2) | 1 | 0 | | 0 | 1 | (0.1) | 1 | 1 | (0.1) | 1 |
| Hypocalcaemia | 1 | (0.2) | 1 | 1 | (0.2) | 1 | 0 | | 0 | 1 | (0.1) | 1 | 2 | (0.1) | 2 |
| Hypokalaemia | 2 | (0.4) | 2 | 1 | (0.2) | 1 | 2 | (0.4) | 2 | 3 | (0.3) | 3 | 5 | (0.4) | 5 |
| Hypomagnesaemia | 0 | | 0 | 2 | (0.4) | 2 | 0 | | 0 | 2 | (0.2) | 2 | 2 | (0.1) | 2 |
| Hyponatraemia | 0 | | 0 | 0 | | 0 | 2 | (0.4) | 2 | 2 | (0.2) | 2 | 2 | (0.1) | 2 |
| Hypophosphataemia | 0 | | 0 | 0 | | 0 | 2 | (0.4) | 2 | 2 | (0.2) | 2 | 2 | (0.1) | 2 |
| Increased appetite | 1 | (0.2) | 1 | 0 | | 0 | 1 | (0.2) | 1 | 1 | (0.1) | 1 | 2 | (0.1) | 2 |
| Musculoskeletal and | 7 | (1.5) | 8 | 8 | (1.7) | 8 | 11 | (2.3) | 11 | 19 | (2.0) | 19 | 26 | (1.9) | 27 |
| connective tissue disorders | | | | | | | | | | | | | | | |
| Arthralgia | 1 | (0.2) | 1 | 2 | (0.4) | 2 | 2 | (0.4) | 2 | 4 | (0.4) | 4 | 5 | (0.4) | 5 |
| Arthritis | 0 | | 0 | 0 | | 0 | 1 | (0.2) | 1 | 1 | (0.1) | 1 | 1 | (0.1) | 1 |
| Back pain | 3 | (0.6) | 3 | 2 | (0.4) | 2 | 4 | (0.9) | 4 | 6 | (0.6) | 6 | 9 | (0.6) | 9 |
| Bursitis | 0 | | 0 | 0 | | 0 | 1 | (0.2) | 1 | 1 | (0.1) | 1 | 1 | (0.1) | 1 |
| Intervertebral disc disorder | 1 | (0.2) | 1 | 0 | | 0 | 0 | | 0 | 0 | | 0 | 1 | (0.1) | 1 |
| Intervertebral disc protrusion | 1 | (0.2) | 1 | 0 | | 0 | 0 | | 0 | 0 | | 0 | 1 | (0.1) | 1 |
| Joint swelling | 0 | | 0 | 0 | | 0 | 1 | (0.2) | 1 | 1 | (0.1) | 1 | 1 | (0.1) | 1 |
| Muscle spasms | 0 | | 0 | 1 | (0.2) | 1 | 0 | | 0 | 1 | (0.1) | 1 | 1 | (0.1) | 1 |
| Musculoskeletal chest pain | 0 | | 0 | 1 | (0.2) | 1 | 0 | | 0 | 1 | (0.1) | 1 | 1 | (0.1) | 1 |
| Musculoskeletal pain | 0 | | 0 | 1 | (0.2) | 1 | 1 | (0.2) | 1 | 2 | (0.2) | 2 | 2 | (0.1) | 2 |
| Osteoarthritis | 1 | (0.2) | 1 | 0 | | 0 | 0 | | 0 | 0 | | 0 | 1 | (0.1) | 1 |
| Pain in extremity | 1 | (0.2) | 1 | 1 | (0.2) | 1 | 0 | | 0 | 1 | (0.1) | 1 | 2 | (0.1) | 2 |
| Plantar fasciitis | 0 | | 0 | 0 | | 0 | 1 | (0.2) | 1 | 1 | (0.1) | 1 | 1 | (0.1) | 1 |
| Neoplasms benign, malignant | 0 | | 0 | 1 | (0.2) | 1 | 2 | (0.4) | 3 | 3 | (0.3) | 4 | 3 | (0.2) | 4 |
| and unspecified (incl | | | | | | | | | | | | | | | |
| cysts and polyps) | | | | | | | | | | | | | | | |
| Fibroadenoma of breast | 0 | | 0 | 0 | | 0 | 1 | (0.2) | 2 | 1 | (0.1) | 2 | 1 | (0.1) | 2 |
| Meningioma | 0 | | 0 | 0 | | 0 | 1 | (0.2) | 1 | 1 | (0.1) | 1 | 1 | (0.1) | 1 |
| Renal cancer | 0 | | 0 | 1 | (0.2) | 1 | 0 | | 0 | 1 | (0.1) | 1 | 1 | (0.1) | 1 |
| Nervous system disorders | 15 | (3.2) | 16 | 18 | (3.9) | 20 | 15 | (3.2) | 17 | 33 | (3.5) | 37 | 48 | (3.4) | 53 |
| Burning sensation | 0 | | 0 | 1 | (0.2) | 1 | 0 | | 0 | 1 | (0.1) | 1 | 1 | (0.1) | 1 |
| Convulsion | 0 | | 0 | 0 | | 0 | 1 | (0.2) | 1 | 1 | (0.1) | 1 | 1 | (0.1) | 1 |
| Dizziness | 4 | (0.9) | 4 | 4 | (0.9) | 4 | 2 | (0.4) | 2 | 6 | (0.6) | 6 | 10 | (0.7) | 10 |
| Headache | 9 | (1.9) | 10 | 10 | (2.1) | 10 | 10 | (2.1) | 10 | 20 | (2.1) | 20 | 29 | (2.1) | 30 |
| Hypoaesthesia | 0 | | 0 | 2 | (0.4) | 2 | 0 | | 0 | 2 | (0.2) | 2 | 2 | (0.1) | 2 |
| Loss of consciousness | 0 | | 0 | 1 | (0.2) | 1 | 0 | | 0 | 1 | (0.1) | 1 | 1 | (0.1) | 1 |
| Memory impairment | 0 | | 0 | 0 | | 0 | 1 | (0.2) | 1 | 1 | (0.1) | 1 | 1 | (0.1) | 1 |
| Migraine | 0 | | 0 | 1 | (0.2) | 1 | 1 | (0.2) | 1 | 2 | (0.2) | 2 | 2 | (0.1) | 2 |
| Nystagmus | 1 | (0.2) | 1 | 0 | | 0 | 0 | | 0 | 0 | | 0 | 1 | (0.1) | 1 |
| Restless legs syndrome | 1 | (0.2) | 1 | 0 | | 0 | 0 | | 0 | 0 | | 0 | 1 | (0.1) | 1 |
| Sciatica | 0 | | 0 | 1 | (0.2) | 1 | 0 | | 0 | 1 | (0.1) | 1 | 1 | (0.1) | 1 |
| Somnolence | 0 | | 0 | 0 | | 0 | 1 | (0.2) | 1 | 1 | (0.1) | 1 | 1 | (0.1) | 1 |
| Syncope | 0 | | 0 | 0 | | 0 | 1 | (0.2) | 1 | 1 | (0.1) | 1 | 1 | (0.1) | 1 |
| Pregnancy, puerperium and | 2 | (0.4) | 3 | 1 | (0.2) | 1 | 1 | (0.2) | 1 | 2 | (0.2) | 2 | 4 | (0.3) | 5 |
| perinatal conditions | | | | | | | | | | | | | | | |
| Abortion spontaneous | 1 | (0.2) | 1 | 0 | | 0 | 0 | | 0 | 0 | | 0 | 1 | (0.1) | 1 |
| Pregnancy | 2 | (0.4) | 2 | 1 | (0.2) | 1 | 1 | (0.2) | 1 | 2 | (0.2) | 2 | 4 | (0.3) | 4 |
| Psychiatric disorders | 3 | (0.6) | 4 | 4 | (0.9) | 5 | 3 | (0.6) | 3 | 7 | (0.7) | 8 | 10 | (0.7) | 12 |
| Attention deficit/hyperactivity | 0 | | 0 | 0 | | 0 | 1 | (0.2) | 1 | 1 | (0.1) | 1 | 1 | (0.1) | 1 |
| disorder | | | | | | | | | | | | | | | |
| Bradyphrenia | 0 | | 0 | 1 | (0.2) | 1 | 0 | | 0 | 1 | (0.1) | 1 | 1 | (0.1) | 1 |
| Depression | 2 | (0.4) | 2 | 0 | | 0 | 0 | | 0 | 0 | | 0 | 2 | (0.1) | 2 |
| Insomnia | 2 | (0.4) | 2 | 1 | (0.2) | 1 | 2 | (0.4) | 2 | 3 | (0.3) | 3 | 5 | (0.4) | 5 |
| Panic attack | 0 | | 0 | 1 | (0.2) | 1 | 0 | | 0 | 1 | (0.1) | 1 | 1 | (0.1) | 1 |
| Stress | 0 | | 0 | 2 | (0.4) | 2 | 0 | | 0 | 2 | (0.2) | 2 | 2 | (0.1) | 2 |
| Renal and urinary disorders | 1 | (0.2) | 1 | 1 | (0.2) | 1 | 3 | (0.6) | 3 | 4 | (0.4) | 4 | 5 | (0.4) | 5 |
| Haematuria | 0 | | 0 | 0 | | 0 | 2 | (0.4) | 2 | 2 | (0.2) | 2 | 2 | (0.1) | 2 |
| Nephrolithiasis | 1 | (0.2) | 1 | 0 | | 0 | 1 | (0.2) | 1 | 1 | (0.1) | 1 | 2 | (0.1) | 2 |
| Stress urinary incontinence | 0 | | 0 | 1 | (0.2) | 1 | 0 | | 0 | 1 | (0.1) | 1 | 1 | (0.1) | 1 |
| Reproductive system and | 0 | | 0 | 1 | (0.2) | 1 | 5 | (1.1) | 6 | 6 | (0.6) | 7 | 6 | (0.4) | 7 |
| breast disorders | | | | | | | | | | | | | | | |
| Balanitis | 0 | | 0 | 1 | (0.2) | 1 | 0 | | 0 | 1 | (0.1) | 1 | 1 | (0.1) | 1 |
| Dysmenorrhoea | 0 | | 0 | 0 | | 0 | 2 | (0.4) | 2 | 2 | (0.2) | 2 | 2 | (0.1) | 2 |
| Menorrhagia | 0 | | 0 | 0 | | 0 | 2 | (0.4) | 3 | 2 | (0.2) | 3 | 2 | (0.1) | 3 |
| Vaginal discharge | 0 | | 0 | 0 | | 0 | 1 | (0.2) | 1 | 1 | (0.1) | 1 | 1 | (0.1) | 1 |
| Respiratory, thoracic and | 9 | (1.9) | 10 | 10 | (2.1) | 11 | 9 | (1.9) | 9 | 19 | (2.0) | 20 | 28 | (2.0) | 30 |
| mediastinal disorders | | | | | | | | | | | | | | | |
| Asthma | 2 | (0.4) | 2 | 1 | (0.2) | 1 | 1 | (0.2) | 1 | 2 | (0.2) | 2 | 4 | (0.3) | 4 |
| Chronic obstructive | 0 | | 0 | 1 | (0.2) | 1 | 1 | (0.2) | 1 | 2 | (0.2) | 2 | 2 | (0.1) | 2 |
| pulmonary disease | | | | | | | | | | | | | | | |
| Cough | 2 | (0.4) | 2 | 5 | (1.1) | 5 | 2 | (0.4) | 2 | 7 | (0.7) | 7 | 9 | (0.6) | 9 |
| Dyspnoea | 1 | (0.2) | 1 | 0 | | 0 | 0 | | 0 | 0 | | 0 | 1 | (0.1) | 1 |
| Epistaxis | 1 | (0.2) | 1 | 0 | | 0 | 0 | | 0 | 0 | | 0 | 1 | (0.1) | 1 |
| Nasal congestion | 1 | (0.2) | 1 | 1 | (0.2) | 1 | 1 | (0.2) | 1 | 2 | (0.2) | 2 | 3 | (0.2) | 3 |
| Oropharyngeal pain | 3 | (0.6) | 3 | 0 | | 0 | 1 | (0.2) | 1 | 1 | (0.1) | 1 | 4 | (0.3) | 4 |
| Pharyngeal oedema | 0 | | 0 | 0 | | 0 | 1 | (0.2) | 1 | 1 | (0.1) | 1 | 1 | (0.1) | 1 |
| Productive cough | 0 | | 0 | 0 | | 0 | 1 | (0.2) | 1 | 1 | (0.1) | 1 | 1 | (0.1) | 1 |
| Pulmonary congestion | 0 | | 0 | 1 | (0.2) | 1 | 0 | | 0 | 1 | (0.1) | 1 | 1 | (0.1) | 1 |
| Rhinitis allergic | 0 | | 0 | 0 | | 0 | 1 | (0.2) | 1 | 1 | (0.1) | 1 | 1 | (0.1) | 1 |
| Sinus congestion | 0 | | 0 | 1 | (0.2) | 1 | 0 | | 0 | 1 | (0.1) | 1 | 1 | (0.1) | 1 |
| Throat irritation | 0 | | 0 | 1 | (0.2) | 1 | 0 | | 0 | 1 | (0.1) | 1 | 1 | (0.1) | 1 |
| Skin and subcutaneous | 11 | (2.4) | 12 | 8 | (1.7) | 9 | 3 | (0.6) | 3 | 11 | (1.2) | 12 | 22 | (1.6) | 24 |
| tissue disorders | | | | | | | | | | | | | | | |
| Acne | 0 | | 0 | 1 | (0.2) | 1 | 0 | | 0 | 1 | (0.1) | 1 | 1 | (0.1) | 1 |
| Acne cystic | 1 | (0.2) | 1 | 0 | | 0 | 0 | | 0 | 0 | | 0 | 1 | (0.1) | 1 |
| Dermatitis | 0 | | 0 | 0 | | 0 | 1 | (0.2) | 1 | 1 | (0.1) | 1 | 1 | (0.1) | 1 |
| Dermatitis contact | 1 | (0.2) | 1 | 1 | (0.2) | 2 | 0 | | 0 | 1 | (0.1) | 2 | 2 | (0.1) | 3 |
| Eczema | 1 | (0.2) | 1 | 1 | (0.2) | 1 | 0 | | 0 | 1 | (0.1) | 1 | 2 | (0.1) | 2 |
| Erythema | 0 | | 0 | 0 | | 0 | 1 | (0.2) | 1 | 1 | (0.1) | 1 | 1 | (0.1) | 1 |
| Pruritus | 1 | (0.2) | 1 | 0 | | 0 | 0 | | 0 | 0 | | 0 | 1 | (0.1) | 1 |
| Rash | 6 | (1.3) | 7 | 3 | (0.6) | 3 | 0 | | 0 | 3 | (0.3) | 3 | 9 | (0.6) | 10 |
| Rash erythematous | 1 | (0.2) | 1 | 1 | (0.2) | 1 | 0 | | 0 | 1 | (0.1) | 1 | 2 | (0.1) | 2 |
| Skin ulcer | 0 | | 0 | 0 | | 0 | 1 | (0.2) | 1 | 1 | (0.1) | 1 | 1 | (0.1) | 1 |
| Urticaria | 0 | | 0 | 1 | (0.2) | 1 | 0 | | 0 | 1 | (0.1) | 1 | 1 | (0.1) | 1 |
| Surgical and medical | 2 | (0.4) | 3 | 4 | (0.9) | 4 | 2 | (0.4) | 2 | 6 | (0.6) | 6 | 8 | (0.6) | 9 |
| procedures | | | | | | | | | | | | | | | |
| Intervertebral disc operation | 1 | (0.2) | 1 | 0 | | 0 | 0 | | 0 | 0 | | 0 | 1 | (0.1) | 1 |
| Skin cosmetic procedure | 0 | | 0 | 1 | (0.2) | 1 | 0 | | 0 | 1 | (0.1) | 1 | 1 | (0.1) | 1 |
| Skin neoplasm excision | 0 | | 0 | 1 | (0.2) | 1 | 0 | | 0 | 1 | (0.1) | 1 | 1 | (0.1) | 1 |
| Spinal fusion surgery | 1 | (0.2) | 1 | 0 | | 0 | 0 | | 0 | 0 | | 0 | 1 | (0.1) | 1 |
| Tooth extraction | 1 | (0.2) | 1 | 2 | (0.4) | 2 | 2 | (0.4) | 2 | 4 | (0.4) | 4 | 5 | (0.4) | 5 |
| Vascular disorders | 3 | (0.6) | 4 | 3 | (0.6) | 3 | 1 | (0.2) | 1 | 4 | (0.4) | 4 | 7 | (0.5) | 8 |
| Arterial occlusive disease | 1 | (0.2) | 1 | 0 | | 0 | 0 | | 0 | 0 | | 0 | 1 | (0.1) | 1 |
| Haematoma | 0 | | 0 | 1 | (0.2) | 1 | 0 | | 0 | 1 | (0.1) | 1 | 1 | (0.1) | 1 |
| Hypertension | 2 | (0.4) | 2 | 2 | (0.4) | 2 | 1 | (0.2) | 1 | 3 | (0.3) | 3 | 5 | (0.4) | 5 |
| Peripheral vascular disorder | 1 | (0.2) | 1 | 0 | | 0 | 0 | | 0 | 0 | | 0 | 1 | (0.1) | 1 |

Source: Listing 16.2.10.1
Note:
If a patient has multiple occurrences of an AE, the patient is presented only once in the patient count for a given SOC and PT. Events are counted each time in the event (E) column. Adverse events are coded using MedDRA version 14.1.
Chiltern · 29JUL2015:5:34 PM · I:\Synergy\33610\21_Stat\Programs\Tables\t_ae.sas · ZScott

TABLE 30
Sunmary of Treatment Emergent Serious Adverse Events by System Organ Class and Preferred Term
(Safety Population)

| | | | | Plecanatide | | | Plecanatide | | | Active | | | | | |
|---|---|---|---|---|---|---|---|---|---|---|---|---|---|---|---|
| | Placebo | | | 3 mg | | | 6 mg | | | Combined | | | Overall | | |
| System Organ Class | (N = 466) | | | (N = 467) | | | (N = 469) | | | (N = 936) | | | (N = 1402) | | |
| Preferred Term | N | (%) | E | N | (%) | E | N | (%) | E | N | (%) | E | N | (%) | E |
| Number of Treatment Emergent | 8 | (1.7) | 9 | 8 | (1.7) | 9 | 4 | (0.9) | 4 | 12 | (1.3) | 13 | 20 | (1.4) | 22 |
| Serious Adverse Events | | | | | | | | | | | | | | | |
| Cardiac disorders | 0 | | 0 | 1 | (0.2) | 1 | 0 | | 0 | 1 | (0.1) | 1 | 1 | (0.1) | 1 |
| Cardiac failure congestive | 0 | | 0 | 1 | (0.2) | 1 | 0 | | 0 | 1 | (0.1) | 1 | 1 | (0.1) | 1 |
| Ear and labyrinth disorders | 0 | | 0 | 1 | (0.2) | 1 | 0 | | 0 | 1 | (0.1) | 1 | 1 | (0.1) | 1 |
| Vertigo positional | 0 | | 0 | 1 | (0.2) | 1 | 0 | | 0 | 1 | (0.1) | 1 | 1 | (0.1) | 1 |
| General disorders and administration site | 1 | (0.2) | 1 | 0 | | 0 | 0 | | 0 | 0 | | 0 | 1 | (0.1) | 1 |
| conditions | | | | | | | | | | | | | | | |
| Non-cardiac chest pain | 1 | (0.2) | 1 | 0 | | 0 | 0 | | 0 | 0 | | 0 | 1 | (0.1) | 1 |
| Hepatobiliary disorders | 0 | | 0 | 1 | (0.2) | 1 | 0 | | 0 | 1 | (0.1) | 1 | 1 | (0.1) | 1 |
| Cholecystitis | 0 | | 0 | 1 | (0.2) | 1 | 0 | | 0 | 1 | (0.1) | 1 | 1 | (0.1) | 1 |
| Injury, poisoning and procedural | 1 | (0.2) | 1 | 0 | | 0 | 0 | | 0 | 0 | | 0 | 1 | (0.1) | 1 |
| complications | | | | | | | | | | | | | | | |
| Femoral neck fracture | 1 | (0.2) | 1 | 0 | | 0 | 0 | | 0 | 0 | | 0 | 1 | (0.1) | 1 |
| Investigations | 1 | (0.2) | 1 | 3 | (0.6) | 3 | 1 | (0.2) | 1 | 4 | (0.4) | 4 | 5 | (0.4) | 5 |
| Alanine aminotransferase increased | 1 | (0.2) | 1 | 1 | (0.2) | 1 | 0 | | 0 | 1 | (0.1) | 1 | 2 | (0.1) | 2 |
| Aspartate aminotransferase increased | 0 | | 0 | 1 | (0.2) | 1 | 0 | | 0 | 1 | (0.1) | 1 | 1 | (0.1) | 1 |
| Liver function test abnormal | 0 | | 0 | 1 | (0.2) | 1 | 1 | (0.2) | 1 | 2 | (0.2) | 2 | 2 | (0.1) | 2 |
| Musculoskeletal and connective tissue | 1 | (0.2) | 1 | 0 | | 0 | 0 | | 0 | 0 | | 0 | 1 | (0.1) | 1 |
| disorders | | | | | | | | | | | | | | | |
| Intervertebral disc protrusion | 1 | (0.2) | 1 | 0 | | 0 | 0 | | 0 | 0 | | 0 | 1 | (0.1) | 1 |
| Neoplasms benign, malignant and | 0 | | 0 | 1 | (0.2) | 1 | 0 | | 0 | 1 | (0.1) | 1 | 1 | (0.1) | 1 |
| unspecified (incl cysts and polyps) | | | | | | | | | | | | | | | |
| Renal cancer | 0 | | 0 | 1 | (0.2) | 1 | 0 | | 0 | 1 | (0.1) | 1 | 1 | (0.1) | 1 |
| Nervous system disorders | 0 | | 0 | 0 | | 0 | 1 | (0.2) | 1 | 1 | (0.1) | 1 | 1 | (0.1) | 1 |
| Convulsion | 0 | | 0 | 0 | | 0 | 1 | (0.2) | 1 | 1 | (0.1) | 1 | 1 | (0.1) | 1 |
| Pregnancy, puerperium and perinatal | 2 | (0.4) | 3 | 1 | (0.2) | 1 | 1 | (0.2) | 1 | 2 | (0.2) | 2 | 4 | (0.3) | 5 |
| conditions | | | | | | | | | | | | | | | |
| Abortion spontaneous | 1 | (0.2) | 1 | 0 | | 0 | 0 | | 0 | 0 | | 0 | 1 | (0.1) | 1 |
| Pregnancy | 2 | (0.4) | 2 | 1 | (0.2) | 1 | 1 | (0.2) | 1 | 2 | (0.2) | 2 | 4 | (0.3) | 4 |
| Renal and urinary disorders | 1 | (0.2) | 1 | 0 | | 0 | 0 | | 0 | 0 | | 0 | 1 | (0.1) | 1 |
| Nephrolithiasis | 1 | (0.2) | 1 | 0 | | 0 | 0 | | 0 | 0 | | 0 | 1 | (0.1) | 1 |
| Respiratory, thoracic and mediastinal | 0 | | 0 | 1 | (0.2) | 1 | 1 | (0.2) | 1 | 2 | (0.2) | 2 | 2 | (0.1) | 2 |
| disorders | | | | | | | | | | | | | | | |
| Chronic obstructive pulmonary disease | 0 | | 0 | 1 | (0.2) | 1 | 1 | (0.2) | 1 | 2 | (0.2) | 2 | 2 | (0.1) | 2 |
| Vascular disorders | 1 | (0.2) | 1 | 0 | | 0 | 0 | | 0 | 0 | | 0 | 1 | (0.1) | 1 |
| Arterial occlusive disease | 1 | (0.2) | 1 | 0 | | 0 | 0 | | 0 | 0 | | 0 | 1 | (0.1) | 1 |

Source: Listing 16.2.10.3
Note:
If a patient has multiple occurrences of an SAE, the patient is presented only once in the patient count for a given SOC and PT. Events are counted each time in the event (E) column. Adverse events are coded using MedDRA version 14.1.
Chiltern · 29JUL2015:5:34 PM · I:\Synergy\33610\21_Stat\Programs\Tables\t_ae.sas · ZScott

TABLE 31
Sunmary of Treatment Emergent Adverse Events Leading to Discontinuation
of Study Drug by System Organ Class and Preferred Term
(Safety Population)

| | | | | Plecanatide | | | Plecanatide | | | Active | | | | | |
|---|---|---|---|---|---|---|---|---|---|---|---|---|---|---|---|
| | Placebo | | | 3 mg | | | 6 mg | | | Combined | | | Overall | | |
| System Organ Class | (N = 466) | | | (N = 467) | | | (N = 469) | | | (N = 936) | | | (N = 1402) | | |
| Preferred Term | N | (%) | E | N | (%) | E | N | (%) | E | N | (%) | E | N | (%) | E |
| Number of Treatment Emergent | 14 | (3.0) | 14 | 15 | (3.2) | 15 | 18 | (3.8) | 18 | 33 | (3.5) | 33 | 47 | (3.4) | 47 |
| Adverse Events Leading to | | | | | | | | | | | | | | | |
| Discontinuation of Study Drug | | | | | | | | | | | | | | | |
| Ear and labyrinth disorders | 0 | | 0 | 1 | (0.2) | 1 | 0 | | 0 | 1 | (0.1) | 1 | 1 | (0.1) | 1 |
| Vertigo positional | 0 | | 0 | 1 | (0.2) | 1 | 0 | | 0 | 1 | (0.1) | 1 | 1 | (0.1) | 1 |
| Gastrointestinal disorders | 3 | (0.6) | 3 | 8 | (1.7) | 8 | 10 | (2.1) | 10 | 18 | (1.9) | 18 | 21 | (1.5) | 21 |
| Abdominal discomfort | 0 | | 0 | 0 | | 0 | 1 | (0.2) | 1 | 1 | (0.1) | 1 | 1 | (0.1) | 1 |
| Abdominal distension | 0 | | 0 | 1 | (0.2) | 1 | 0 | | 0 | 1 | (0.1) | 1 | 1 | (0.1) | 1 |
| Abdominal pain | 1 | (0.2) | 1 | 1 | (0.2) | 1 | 1 | (0.2) | 1 | 2 | (0.2) | 2 | 3 | (0.2) | 3 |
| Defaecation urgency | 0 | | 0 | 0 | | 0 | 1 | (0.2) | 1 | 1 | (0.1) | 1 | 1 | (0.1) | 1 |
| Diarrhoea | 2 | (0.4) | 2 | 5 | (1.1) | 5 | 5 | (1.1) | 5 | 10 | (1.1) | 10 | 12 | (0.9) | 12 |
| Epigastric discomfort | 0 | | 0 | 0 | | 0 | 1 | (0.2) | 1 | 1 | (0.1) | 1 | 1 | (0.1) | 1 |
| Haemorrhoidal haemorrhage | 0 | | 0 | 0 | | 0 | 1 | (0.2) | 1 | 1 | (0.1) | 1 | 1 | (0.1) | 1 |
| Vomiting | 0 | | 0 | 1 | (0.2) | 1 | 0 | | 0 | 1 | (0.1) | 1 | 1 | (0.1) | 1 |
| General disorders and administration site | 1 | (0.2) | 1 | 0 | | 0 | 1 | (0.2) | 1 | 1 | (0.1) | 1 | 2 | (0.1) | 2 |
| conditions | | | | | | | | | | | | | | | |
| Fatigue | 1 | (0.2) | 1 | 0 | | 0 | 0 | | 0 | 0 | | 0 | 1 | (0.1) | 1 |
| Non-cardiac chest pain | 0 | | 0 | 0 | | 0 | 1 | (0.2) | 1 | 1 | (0.1) | 1 | 1 | (0.1) | 1 |
| Infections and infestations | 0 | | 0 | 0 | | 0 | 1 | (0.2) | 1 | 1 | (0.1) | 1 | 1 | (0.1) | 1 |
| Viral infection | 0 | | 0 | 0 | | 0 | 1 | (0.2) | 1 | 1 | (0.1) | 1 | 1 | (0.1) | 1 |
| Injury, poisoning and procedural | 1 | (0.2) | 1 | 2 | (0.4) | 2 | 0 | | 0 | 2 | (0.2) | 2 | 3 | (0.2) | 3 |
| complications | | | | | | | | | | | | | | | |
| Femoral neck fracture | 1 | (0.2) | 1 | 0 | | 0 | 0 | | 0 | 0 | | 0 | 1 | (0.1) | 1 |
| Joint dislocation | 0 | | 0 | 1 | (0.2) | 1 | 0 | | 0 | 1 | (0.1) | 1 | 1 | (0.1) | 1 |
| Joint injury | 0 | | 0 | 1 | (0.2) | 1 | 0 | | 0 | 1 | (0.1) | 1 | 1 | (0.1) | 1 |
| Investigations | 0 | | 0 | 0 | | 0 | 1 | (0.2) | 1 | 1 | (0.1) | 1 | 1 | (0.1) | 1 |
| Liver function test abnormal | 0 | | 0 | 0 | | 0 | 1 | (0.2) | 1 | 1 | (0.1) | 1 | 1 | (0.1) | 1 |
| Metabolism and nutrition disorders | 1 | (0.2) | 1 | 0 | | 0 | 0 | | 0 | 0 | | 0 | 1 | (0.1) | 1 |
| Hyperglycaemia | 1 | (0.2) | 1 | 0 | | 0 | 0 | | 0 | 0 | | 0 | 1 | (0.1) | 1 |
| Musculoskeletal and connective tissue | 1 | (0.2) | 1 | 0 | | 0 | 0 | | 0 | 0 | | 0 | 1 | (0.1) | 1 |
| disorders | | | | | | | | | | | | | | | |
| Intervertebral disc protrusion | 1 | (0.2) | 1 | 0 | | 0 | 0 | | 0 | 0 | | 0 | 1 | (0.1) | 1 |
| Neoplasms benign, malignant and | 0 | | 0 | 1 | (0.2) | 1 | 0 | | 0 | 1 | (0.1) | 1 | 1 | (0.1) | 1 |
| unspecified (incl cysts and polyps) | | | | | | | | | | | | | | | |
| Renal cancer | 0 | | 0 | 1 | (0.2) | 1 | 0 | | 0 | 1 | (0.1) | 1 | 1 | (0.1) | 1 |
| Nervous system disorders | 1 | (0.2) | 1 | 0 | | 0 | 2 | (0.4) | 2 | 2 | (0.2) | 2 | 3 | (0.2) | 3 |
| Headache | 1 | (0.2) | 1 | 0 | | 0 | 1 | (0.2) | 1 | 1 | (0.1) | 1 | 2 | (0.1) | 2 |
| Migraine | 0 | | 0 | 0 | | 0 | 1 | (0.2) | 1 | 1 | (0.1) | 1 | 1 | (0.1) | 1 |
| Pregnancy, puerperium and perinatal | 1 | (0.2) | 1 | 1 | (0.2) | 1 | 1 | (0.2) | 1 | 2 | (0.2) | 2 | 3 | (0.2) | 3 |
| conditions | | | | | | | | | | | | | | | |
| Pregnancy | 1 | (0.2) | 1 | 1 | (0.2) | 1 | 1 | (0.2) | 1 | 2 | (0.2) | 2 | 3 | (0.2) | 3 |
| Renal and urinary disorders | 1 | (0.2) | 1 | 0 | | 0 | 0 | | 0 | 0 | | 0 | 1 | (0.1) | 1 |
| Nephrolithiasis | 1 | (0.2) | 1 | 0 | | 0 | 0 | | 0 | 0 | | 0 | 1 | (0.1) | 1 |
| Pespiratory, thoracic and mediastinal | 1 | (0.2) | 1 | 0 | | 0 | 2 | (0.4) | 2 | 2 | (0.2) | 2 | 3 | (0.2) | 3 |
| disorders | | | | | | | | | | | | | | | |
| Chronic obstructive pulmonary disease | 0 | | 0 | 0 | | 0 | 1 | (0.2) | 1 | 1 | (0.1) | 1 | 1 | (0.1) | 1 |
| Nasal congestion | 1 | (0.2) | 1 | 0 | | 0 | 0 | | 0 | 0 | | 0 | 1 | (0.1) | 1 |
| Pharyngeal oedema | 0 | | 0 | 0 | | 0 | 1 | (0.2) | 1 | 1 | (0.1) | 1 | 1 | (0.1) | 1 |
| Skin and subcutaneous tissue disorders | 2 | (0.4) | 2 | 2 | (0.4) | 2 | 0 | | 0 | 2 | (0.2) | 2 | 4 | (0.3) | 4 |
| Rash | 2 | (0.4) | 2 | 1 | (0.2) | 1 | 0 | | 0 | 1 | (0.1) | 1 | 3 | (0.2) | 3 |
| Urticaria | 0 | | 0 | 1 | (0.2) | 1 | 0 | | 0 | 1 | (0.1) | 1 | 1 | (0.1) | 1 |
| Vascular disorders | 1 | (0.2) | 1 | 0 | | 0 | 0 | | 0 | 0 | | 0 | 1 | (0.1) | 1 |
| Arterial occlusive disease | 1 | (0.2) | 1 | 0 | | 0 | 0 | | 0 | 0 | | 0 | 1 | (0.1) | 1 |

Source: Listing 16.2.10.1
Note:
Patients are only summarized for the primary AE leading to discontinuation of study drug. Only primary adverse events are counted in the event (E) column. Adverse events are coded using MedDRA version 14.1.
Chiltern · 29JUL2015:5:34 PM · I:\Synergy\33610\21_Stat\Programs\Tables\t_ae.sas · ZScott

TABLE 32
Summary of Treatment Emergent Adverse Events by System Organ Class,
Preferred Term, and Maximum Severity
(Safety Population)

| | | | | Plecanatide | | | Plecanatide | | | Active | | | | | |
|---|---|---|---|---|---|---|---|---|---|---|---|---|---|---|---|
| System Organ Class | Placebo | | | 3 mg | | | 6 mg | | | Combined | | | Overall | | |
| Preferred Term | (N = 466) | | | (N = 467) | | | (N = 469) | | | (N = 936) | | | (N = 1402) | | |
| Severity | N | (%) | E | N | (%) | E | N | (%) | E | N | (%) | E | N | (%) | E |

| | | | | | | | | | | | | | | | |
|---|---|---|---|---|---|---|---|---|---|---|---|---|---|---|---|
| Number of Treatment | 115 | (24.7) | 196 | 120 | (23.7) | 196 | 137 | (29.2) | 226 | 257 | (27.5) | 422 | 372 | (26.5) | 618 |
| Emergent Adverse Events | | | | | | | | | | | | | | | |
| Mild | 65 | (13.9) | 85 | 67 | (14.3) | 95 | 81 | (17.3) | 112 | 148 | (15.8) | 207 | 213 | (15.2) | 292 |
| Moderate | 43 | (9.2) | 62 | 45 | (9.6) | 63 | 49 | (10.4) | 74 | 94 | (10.0) | 137 | 137 | (9.8) | 199 |
| Severe | 6 | (1.3) | 14 | 8 | (1.7) | 8 | 6 | (1.3) | 6 | 14 | (1.5) | 14 | 20 | (1.4) | 28 |
| Grade is missing | 1 | (0.2) | 1 | 0 | | 0 | 1 | (0.2) | 1 | 1 | (0.1) | 1 | 2 | (0.1) | 2 |
| Blood and lymphatic | 7 | (1.5) | 8 | 5 | (1.1) | 5 | 5 | (1.1) | 5 | 10 | (1.1) | 10 | 17 | (1.2) | 18 |
| system disorders | | | | | | | | | | | | | | | |
| Mild | 4 | (0.9) | 4 | 4 | (0.9) | 4 | 3 | (0.6) | 3 | 7 | (0.7) | 7 | 11 | (0.8) | 11 |
| Moderate | 3 | (0.6) | 3 | 1 | (0.2) | 1 | 2 | (0.4) | 2 | 3 | (0.3) | 3 | 6 | (0.4) | 6 |
| Severe | 0 | | 0 | 0 | | 0 | 0 | | 0 | 0 | | 0 | 0 | | 0 |
| Anaemia | 1 | (0.2) | 1 | 3 | (0.6) | 3 | 4 | (0.9) | 4 | 7 | (0.7) | 7 | 8 | (0.6) | 8 |
| Mild | 1 | (0.2) | 1 | 2 | (0.4) | 2 | 2 | (0.4) | 2 | 4 | (0.4) | 4 | 5 | (0.4) | 5 |
| Moderate | 0 | | 0 | 1 | (0.2) | 1 | 2 | (0.4) | 2 | 3 | (0.3) | 3 | 3 | (0.2) | 3 |
| Severe | 0 | | 0 | 0 | | 0 | 0 | | 0 | 0 | | 0 | 0 | | 0 |
| Hypochromic anaemia | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Iron deficiency anaemia | 2 | (0.4) | 2 | 0 | | 0 | 0 | | 0 | 0 | | 0 | 2 | (0.1) | 2 |
| Mild | 1 | (0.2) | 1 | 0 | | 0 | 0 | | 0 | 0 | | 0 | 1 | (0.1) | 1 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Leukocytosis | 1 | (0.2) | 2 | 0 | | 0 | 0 | | 0 | 0 | | 0 | 1 | (0.1) | 2 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Lymphadenopathy | 2 | (0.4) | 2 | 1 | (0.2) | 1 | 1 | (0.2) | 1 | 2 | (0.2) | 2 | 4 | (0.3) | 4 |
| Mild | 1 | (0.2) | 1 | 1 | (0.2) | 1 | 1 | (0.2) | 1 | 2 | (0.2) | 2 | 3 | (0.2) | 3 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Thrombocytopenia | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Cardiac disorders | 3 | (0.6) | 6 | 1 | (0.2) | 1 | 0 | | 0 | 1 | (0.1) | 1 | 4 | (0.3) | 7 |
| Mild | 3 | (0.6) | 6 | 0 | | 0 | 0 | | 0 | 0 | | 0 | 3 | (0.2) | 6 |
| Moderate | 0 | | 0 | 0 | | 0 | 0 | | 0 | 0 | | 0 | 0 | | 0 |
| Severe | 0 | | 0 | 1 | (0.2) | 1 | 0 | | 0 | 1 | (0.1) | 1 | 1 | (0.1) | 1 |
| Atrioventricular block first | 1 | (0.2) | 1 | 0 | | 0 | 0 | | 0 | 0 | | 0 | 1 | (0.1) | 1 |
| degree | | | | | | | | | | | | | | | |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Atrioventricular dissociation | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Cardiac failure congestive | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 0 | | 0 | 1 | (0.2) | 1 | 0 | | 0 | 1 | (0.1) | 1 | 1 | (0.1) | 1 |
| Nodal rhythm | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Palpitations | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Sinus bradycardia | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Tachycardia | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Ear and labyrinth disorders | 0 | | 0 | 3 | (0.6) | 3 | 1 | (0.2) | 1 | 4 | (0.4) | 4 | 4 | (0.3) | 4 |
| Mild | 0 | | 0 | 0 | | 0 | 0 | | 0 | 0 | | 0 | 0 | | 0 |
| Moderate | 0 | | 0 | 2 | (0.4) | 2 | 1 | (0.2) | 1 | 3 | (0.3) | 3 | 3 | (0.2) | 3 |
| Severe | 0 | | 0 | 1 | (0.2) | 1 | 0 | | 0 | 1 | (0.1) | 1 | 1 | (0.1) | 1 |
| Deafness | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Ear pain | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| External ear pain | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Vertigo positional | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 0 | | 0 | 1 | (0.2) | 1 | 0 | | 0 | 1 | (0.1) | 1 | 1 | (0.1) | 1 |
| Endocrine disorders | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Goitre | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Eye disorders | 1 | (0.2) | 2 | 1 | (0.2) | 1 | 0 | | 0 | 1 | (0.1) | 1 | 2 | (0.1) | 3 |
| Mild | 1 | (0.2) | 2 | 0 | | 0 | 0 | | 0 | 0 | | 0 | 1 | (0.1) | 2 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Dry eye | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Ocular discomfort | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Ocular hyperaemia | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Gastrointestinal disorders | 21 | (4.5) | 33 | 34 | (7.3) | 42 | 43 | (9.2) | 61 | 77 | (8.2) | 103 | 98 | (7.0) | 136 |
| Mild | 14 | (3.0) | 16 | 19 | (4.1) | 23 | 21 | (4.5) | 27 | 40 | (4.3) | 50 | 54 | (3.9) | 66 |
| Moderate | 5 | (1.1) | 10 | 14 | (3.0) | 17 | 20 | (4.3) | 28 | 34 | (3.6) | 45 | 39 | (2.8) | 55 |
| Severe | 2 | (0.4) | 7 | 1 | (0.2) | 1 | 2 | (0.4) | 2 | 3 | (0.3) | 3 | 5 | (0.4) | 10 |
| Abdominal discomfort | 1 | (0.2) | 1 | 0 | | 0 | 2 | (0.4) | 2 | 2 | (0.2) | 2 | 3 | (0.2) | 3 |
| Mild | 1 | (0.2) | 1 | 0 | | 0 | 1 | (0.2) | 1 | 1 | (0.1) | 1 | 2 | (0.1) | 2 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Abdominal distension | 1 | (0.2) | 2 | 3 | (0.6) | 3 | 3 | (0.6) | 3 | 6 | (0.6) | 6 | 7 | (0.5) | 8 |
| Mild | 0 | | 0 | 0 | | 0 | 1 | (0.2) | 1 | 1 | (0.1) | 1 | 1 | (0.1) | 1 |
| Moderate | 1 | (0.2) | 2 | 3 | (0.6) | 3 | 2 | (0.4) | 2 | 5 | (0.5) | 5 | 6 | (0.4) | 7 |
| Severe | 0 | | 0 | 0 | | 0 | 0 | | 0 | 0 | | 0 | 0 | | 0 |
| Abdominal pain | 4 | (0.9) | 5 | 2 | (0.4) | 2 | 5 | (1.1) | 5 | 7 | (0.7) | 7 | 11 | (0.8) | 12 |
| Mild | 2 | (0.4) | 2 | 0 | | 0 | 3 | (0.6) | 3 | 3 | (0.3) | 3 | 5 | (0.4) | 5 |
| Moderate | 1 | (0.2) | 1 | 1 | (0.2) | 1 | 2 | (0.4) | 2 | 3 | (0.3) | 3 | 4 | (0.3) | 4 |
| Severe | 1 | (0.2) | 2 | 1 | (0.2) | 1 | 0 | | 0 | 1 | (0.1) | 1 | 2 | (0.1) | 3 |
| Abdominal pain lower | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Abdominal pain upper | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 1 | (0.2) | 1 | 0 | | 0 | 0 | | 0 | 0 | | 0 | 1 | (0.1) | 1 |
| Abdominal tenderness | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Anorectal discomfort | 0 | | 0 | 0 | | 0 | 3 | (0.6) | 3 | 3 | (0.3) | 3 | 3 | (0.2) | 3 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 2 | (0.4) | 2 | 2 | (0.2) | 2 | 2 | (0.1) | 2 |
| Severe | 0 | | 0 | 0 | | 0 | 0 | | 0 | 0 | | 0 | 0 | | 0 |
| Defaecation urgency | 0 | | 0 | 1 | (0.2) | 1 | 2 | (0.4) | 2 | 3 | (0.3) | 3 | 3 | (0.2) | 3 |
| Mild | 0 | | 0 | 0 | | 0 | 0 | | 0 | 0 | | 0 | 0 | | 0 |
| Moderate | 0 | | 0 | 1 | (0.2) | 1 | 2 | (0.4) | 2 | 3 | (0.3) | 3 | 3 | (0.2) | 3 |
| Severe | 0 | | 0 | 0 | | 0 | 0 | | 0 | 0 | | 0 | 0 | | 0 |
| Diarrhoea | 6 | (1.3) | 7 | 15 | (3.2) | 15 | 21 | (4.5) | 25 | 36 | (3.8) | 40 | 42 | (3.0) | 47 |
| Mild | 3 | (0.6) | 3 | 5 | (1.1) | 5 | 9 | (1.9) | 10 | 14 | (1.5) | 15 | 17 | (1.2) | 18 |
| Moderate | 2 | (0.4) | 2 | 10 | (2.1) | 10 | 10 | (2.1) | 13 | 20 | (2.1) | 23 | 22 | (1.6) | 25 |
| Severe | 1 | (0.2) | 2 | 0 | | 0 | 2 | (0.4) | 2 | 2 | (0.2) | 2 | 3 | (0.2) | 4 |
| Dry mouth | 1 | (0.2) | 1 | 1 | (0.2) | 1 | 1 | (0.2) | 1 | 2 | (0.2) | 2 | 3 | (0.2) | 3 |
| Mild | 1 | (0.2) | 1 | 1 | (0.2) | 1 | 1 | (0.2) | 1 | 2 | (0.2) | 2 | 3 | (0.2) | 3 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Dyspepsia | 1 | (0.2) | 2 | 4 | (0.9) | 4 | 0 | | 0 | 4 | (0.4) | 4 | 5 | (0.4) | 6 |
| Mild | 1 | (0.2) | 2 | 3 | (0.6) | 3 | 0 | | 0 | 3 | (0.3) | 3 | 4 | (0.3) | 5 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Epigastric discomfort | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Faecal incontinence | 0 | | 0 | 0 | | 0 | 1 | (0.2) | 2 | 1 | (0.1) | 2 | 1 | (0.1) | 2 |
| Mild | 0 | | 0 | 0 | | 0 | 1 | (0.2) | 2 | 1 | (0.1) | 2 | 1 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Flatulence | 4 | (0.9) | 4 | 1 | (0.2) | 1 | 4 | (0.9) | 4 | 5 | (0.5) | 5 | 9 | (0.6) | 9 |
| Mild | 4 | (0.9) | 4 | 1 | (0.2) | 1 | 3 | (0.6) | 3 | 4 | (0.4) | 4 | 8 | (0.6) | 8 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Gastrointestinal motility | 0 | | 0 | 1 | (0.2) | 1 | 0 | | 0 | 1 | (0.1) | 1 | 1 | (0.1) | 1 |
| disorder | | | | | | | | | | | | | | | |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Gastrooesophageal | 0 | | 0 | 2 | (0.4) | 2 | 0 | | 0 | 2 | (0.2) | 2 | 2 | (0.1) | 2 |
| reflux disease | | | | | | | | | | | | | | | |
| Mild | 0 | | 0 | 2 | (0.4) | 2 | 0 | | 0 | 2 | (0.2) | 2 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Gingival pain | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Haematochezia | 0 | | 0 | 1 | (0.2) | 1 | 2 | (0.4) | 2 | 3 | (0.3) | 3 | 3 | (0.2) | 3 |
| Mild | 0 | | 0 | 1 | (0.2) | 1 | 2 | (0.4) | 2 | 3 | (0.3) | 3 | 3 | (0.2) | 3 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Haemorrhoidal haemorrhage | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Haemorrhoids | 1 | (0.2) | 1 | 1 | (0.2) | 1 | 1 | (0.2) | 1 | 2 | (0.2) | 2 | 3 | (0.2) | 3 |
| Mild | 1 | (0.2) | 1 | 0 | | 0 | 0 | | 0 | 0 | | 0 | 1 | (0.1) | 1 |
| Moderate | 0 | | 0 | 1 | (0.2) | 1 | 1 | (0.2) | 1 | 2 | (0.2) | 2 | 2 | (0.1) | 2 |
| Severe | 0 | | 0 | 0 | | 0 | 0 | | 0 | 0 | | 0 | 0 | | 0 |
| Ileus | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 1 | (0.2) | 1 | 0 | | 0 | 0 | | 0 | 0 | | 0 | 1 | (0.1) | 1 |
| Nausea | 5 | (1.1) | 5 | 4 | (0.9) | 4 | 5 | (1.1) | 5 | 9 | (1.0) | 9 | 14 | (1.0) | 14 |
| Mild | 2 | (0.4) | 2 | 4 | (0.9) | 4 | 3 | (0.6) | 3 | 7 | (0.7) | 7 | 9 | (0.6) | 9 |
| Moderate | 3 | (0.6) | 3 | 0 | | 0 | 2 | (0.4) | 2 | 2 | (0.2) | 2 | 5 | (0.4) | 5 |
| Severe | 0 | | 0 | 0 | | 0 | 0 | | 0 | 0 | | 0 | 0 | | 0 |
| Painful defaecation | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Toothache | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Vomiting | 3 | (0.6) | 3 | 3 | (0.6) | 3 | 2 | (0.4) | 2 | 5 | (0.5) | 5 | 8 | (0.6) | 8 |
| Mild | 0 | | 0 | 3 | (0.6) | 3 | 1 | (0.2) | 1 | 4 | (0.4) | 4 | 4 | (0.3) | 4 |
| Moderate | 2 | (0.4) | 2 | 0 | | 0 | 1 | (0.2) | 1 | 1 | (0.1) | 1 | 3 | (0.2) | 3 |
| Severe | 1 | (0.2) | 1 | 0 | | 0 | 0 | | 0 | 0 | | 0 | 1 | (0.1) | 1 |
| General disorders and | 11 | (2.4) | 11 | 9 | (1.9) | 10 | 5 | (1.1) | 7 | 14 | (1.5) | 17 | 25 | (1.8) | 28 |
| administration site conditions | | | | | | | | | | | | | | | |
| Mild | 4 | (0.9) | 4 | 8 | (1.7) | 9 | 3 | (0.6) | 3 | 11 | (1.2) | 12 | 15 | (1.1) | 16 |
| Moderate | 6 | (1.3) | 6 | 1 | (0.2) | 1 | 2 | (0.4) | 3 | 3 | (0.3) | 4 | 9 | (0.6) | 10 |
| Severe | 1 | (0.2) | 1 | 0 | | 0 | 0 | | 0 | 0 | | 0 | 1 | (0.1) | 1 |
| Chest discomfort | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Chest pain | 0 | | 0 | 1 | (0.2) | 1 | 1 | (0.2) | 1 | 2 | (0.2) | 2 | 2 | (0.1) | 2 |
| Mild | 0 | | 0 | 1 | (0.2) | 1 | 1 | (0.2) | 1 | 2 | (0.2) | 2 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Chills | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Fatigue | 2 | (0.4) | 2 | 1 | (0.2) | 1 | 1 | (0.2) | 1 | 2 | (0.2) | 2 | 4 | (0.3) | 4 |
| Mild | 1 | (0.2) | 1 | 1 | (0.2) | 1 | 1 | (0.2) | 1 | 2 | (0.2) | 2 | 3 | (0.2) | 3 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Influenza like illness | 1 | (0.2) | 1 | 0 | | 0 | 1 | (0.2) | 1 | 1 | (0.1) | 1 | 2 | (0.1) | 2 |
| Mild | 0 | | 0 | 0 | | 0 | 1 | (0.2) | 1 | 1 | (0.1) | 1 | 1 | (0.1) | 1 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Non-cardiac chest pain | 1 | (0.2) | 1 | 1 | (0.2) | 1 | 1 | (0.2) | 1 | 2 | (0.2) | 2 | 3 | (0.2) | 3 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 1 | (0.2) | 1 | 1 | (0.1) | 1 | 1 | (0.1) | 1 |
| Severe | 1 | (0.2) | 1 | 0 | | 0 | 0 | | 0 | 0 | | 0 | 1 | (0.1) | 1 |
| Oedema | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Oedema peripheral | 3 | (0.6) | 3 | 1 | (0.2) | 1 | 0 | | 0 | 1 | (0.1) | 1 | 4 | (0.3) | 4 |
| Mild | 2 | (0.4) | 2 | 1 | (0.2) | 1 | 0 | | 0 | 1 | (0.1) | 1 | 3 | (0.2) | 3 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Pain | 1 | (0.2) | 1 | 3 | (0.6) | 3 | 1 | (0.2) | 1 | 4 | (0.4) | 4 | 5 | (0.4) | 5 |
| Mild | 1 | (0.2) | 1 | 3 | (0.6) | 3 | 0 | | 0 | 3 | (0.3) | 3 | 4 | (0.3) | 4 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Pyrexia | 3 | (0.6) | 3 | 1 | (0.2) | 1 | 1 | (0.2) | 1 | 2 | (0.2) | 2 | 5 | (0.4) | 5 |
| Mild | 0 | | 0 | 1 | (0.2) | 1 | 0 | | 0 | 1 | (0.1) | 1 | 1 | (0.1) | 1 |
| Moderate | 3 | (0.6) | 3 | 0 | | 0 | 1 | (0.2) | 1 | 1 | (0.1) | 1 | 4 | (0.3) | 4 |
| Severe | 0 | | 0 | 0 | | 0 | 0 | | 0 | 0 | | 0 | 0 | | 0 |
| Hepatobiliary disorders | 0 | | 0 | 2 | (0.4) | 3 | 0 | | 0 | 2 | (0.2) | 3 | 2 | (0.1) | 3 |
| Mild | 0 | | 0 | 0 | | 0 | 0 | | 0 | 0 | | 0 | 0 | | 0 |
| Moderate | 0 | | 0 | 2 | (0.4) | 2 | 0 | | 0 | 2 | (0.2) | 2 | 2 | (0.1) | 2 |
| Severe | 0 | | 0 | 0 | | 0 | 0 | | 0 | 0 | | 0 | 0 | | 0 |
| Cholecystitis | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Cholelithiasis | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hepatic steatosis | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Infections and infestations | 36 | (7.7) | 42 | 34 | (7.3) | 38 | 43 | (9.2) | 51 | 77 | (8.2) | 89 | 113 | (8.1) | 131 |
| Mild | 21 | (4.5) | 23 | 22 | (4.7) | 22 | 24 | (5.1) | 28 | 46 | (4.9) | 50 | 67 | (4.8) | 73 |
| Moderate | 14 | (3.0) | 14 | 12 | (2.6) | 14 | 18 | (3.8) | 21 | 30 | (3.2) | 35 | 44 | (3.1) | 49 |
| Severe | 1 | (0.2) | 1 | 0 | | 0 | 1 | (0.2) | 1 | 1 | (0.1) | 1 | 2 | (0.1) | 2 |
| Acute sinusitis | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Asymptomatic bacteriuria | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Bronchitis | 3 | (0.6) | 3 | 2 | (0.4) | 2 | 3 | (0.6) | 3 | 5 | (0.5) | 5 | 8 | (0.6) | 8 |
| Mild | 1 | (0.2) | 1 | 1 | (0.2) | 1 | 1 | (0.2) | 1 | 2 | (0.2) | 2 | 3 | (0.2) | 3 |
| Moderate | 2 | (0.4) | 2 | 1 | (0.2) | 1 | 2 | (0.4) | 2 | 3 | (0.3) | 3 | 5 | (0.4) | 5 |
| Severe | 0 | | 0 | 0 | | 0 | 0 | | 0 | 0 | | 0 | 0 | | 0 |
| Cellulitis | 0 | | 0 | 0 | | 0 | 2 | (0.4) | 2 | 2 | (0.2) | 2 | 2 | (0.1) | 2 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Cystitis | 1 | (0.2) | 1 | 1 | (0.2) | 1 | 0 | | 0 | 1 | (0.1) | 1 | 2 | (0.1) | 2 |
| Mild | 0 | | 0 | 1 | (0.2) | 1 | 0 | | 0 | 1 | (0.1) | 1 | 1 | (0.1) | 1 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Ear infection | 2 | (0.4) | 2 | 1 | (0.2) | 1 | 1 | (0.2) | 1 | 2 | (0.2) | 2 | 4 | (0.3) | 4 |
| Mild | 1 | (0.2) | 1 | 0 | | 0 | 0 | | 0 | 0 | | 0 | 1 | (0.1) | 1 |
| Moderate | 1 | (0.2) | 1 | 1 | (0.2) | 1 | 1 | (0.2) | 1 | 2 | (0.2) | 2 | 3 | (0.2) | 3 |
| Severe | 0 | | 0 | 0 | | 0 | 0 | | 0 | 0 | | 0 | 0 | | 0 |
| Folliculitis | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Gastroenteritis | 1 | (0.2) | 1 | 2 | (0.4) | 2 | 0 | | 0 | 2 | (0.2) | 2 | 3 | (0.2) | 3 |
| Mild | 0 | | 0 | 1 | (0.2) | 1 | 0 | | 0 | 1 | (0.1) | 1 | 1 | (0.1) | 1 |
| Moderate | 1 | (0.2) | 1 | 1 | (0.2) | 1 | 0 | | 0 | 1 | (0.1) | 1 | 2 | (0.1) | 2 |
| Severe | 0 | | 0 | 0 | | 0 | 0 | | 0 | 0 | | 0 | 0 | | 0 |
| Gastroenteritis viral | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Gastrointestinal viral infection | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Herpes zoster | 0 | | 0 | 1 | (0.2) | 1 | 1 | (0.2) | 1 | 2 | (0.2) | 2 | 2 | (0.1) | 2 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Influenza | 2 | (0.4) | 2 | 2 | (0.4) | 2 | 7 | (1.5) | 7 | 9 | (1.0) | 9 | 11 | (0.8) | 11 |
| Mild | 1 | (0.2) | 1 | 2 | (0.4) | 2 | 4 | (0.9) | 4 | 6 | (0.6) | 6 | 7 | (0.5) | 7 |
| Moderate | 1 | (0.2) | 1 | 0 | | 0 | 3 | (0.6) | 3 | 3 | (0.3) | 3 | 4 | (0.3) | 4 |
| Severe | 0 | | 0 | 0 | | 0 | 0 | | 0 | 0 | | 0 | 0 | | 0 |
| Localised infection | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Nasopharyngitis | 6 | (1.3) | 6 | 7 | (1.5) | 7 | 9 | (1.9) | 10 | 16 | (1.7) | 17 | 22 | (1.6) | 23 |
| Mild | 4 | (0.9) | 4 | 5 | (1.1) | 5 | 9 | (1.9) | 10 | 14 | (1.5) | 15 | 18 | (1.3) | 19 |
| Moderate | 2 | (0.4) | 2 | 2 | (0.4) | 2 | 0 | | 0 | 2 | (0.2) | 2 | 4 | (0.3) | 4 |
| Severe | 0 | | 0 | 0 | | 0 | 0 | | 0 | 0 | | 0 | 0 | | 0 |
| Otitis media | 1 | (0.2) | 1 | 0 | | 0 | 1 | (0.2) | 1 | 1 | (0.1) | 1 | 2 | (0.1) | 2 |
| Mild | 0 | | 0 | 0 | | 0 | 1 | (0.2) | 1 | 1 | (0.1) | 1 | 1 | (0.1) | 1 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Pharyngitis | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Pharyngitis streptococcal | 2 | (0.4) | 2 | 0 | | 0 | 1 | (0.2) | 1 | 1 | (0.1) | 1 | 3 | (0.2) | 3 |
| Mild | 2 | (0.4) | 2 | 0 | | 0 | 0 | | 0 | 0 | | 0 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 1 | (0.2) | 1 | 1 | (0.1) | 1 | 1 | (0.1) | 1 |
| Pneumonia | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Pyelonephritis | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 1 | (0.2) | 1 | 0 | | 0 | 0 | | 0 | 0 | | 0 | 1 | (0.1) | 1 |
| Rhinitis | 2 | (0.4) | 2 | 0 | | 0 | 1 | (0.2) | 1 | 1 | (0.1) | 1 | 3 | (0.2) | 3 |
| Mild | 2 | (0.4) | 2 | 0 | | 0 | 1 | (0.2) | 1 | 1 | (0.1) | 1 | 3 | (0.2) | 3 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Sinusitis | 1 | (0.2) | 1 | 2 | (0.4) | 2 | 3 | (0.6) | 3 | 5 | (0.5) | 5 | 6 | (0.4) | 6 |
| Mild | 1 | (0.2) | 1 | 1 | (0.2) | 1 | 0 | | 0 | 1 | (0.1) | 1 | 2 | (0.1) | 2 |
| Moderate | 0 | | 0 | 1 | (0.2) | 1 | 3 | (0.6) | 3 | 4 | (0.4) | 4 | 4 | (0.3) | 4 |
| Severe | 0 | | 0 | 0 | | 0 | 0 | | 0 | 0 | | 0 | 0 | | 0 |
| Subcutaneous abscess | 0 | | 0 | 0 | | 0 | 1 | (0.2) | 2 | 1 | (0.1) | 2 | 1 | (0.1) | 2 |
| Mild | 0 | | 0 | 0 | | 0 | 0 | | 0 | 0 | | 0 | 0 | | 0 |
| Moderate | 0 | | 0 | 0 | | 0 | 1 | (0.2) | 2 | 1 | (0.1) | 2 | 1 | (0.1) | 2 |
| Severe | 0 | | 0 | 0 | | 0 | 0 | | 0 | 0 | | 0 | 0 | | 0 |
| Tinea versicolour | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Tooth abscess | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Tooth infection | 2 | (0.4) | 2 | 0 | | 0 | 0 | | 0 | 0 | | 0 | 2 | (0.1) | 2 |
| Mild | 1 | (0.2) | 1 | 0 | | 0 | 0 | | 0 | 0 | | 0 | 1 | (0.1) | 1 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Upper respiratory tract | 3 | (0.6) | 3 | 8 | (1.7) | 8 | 4 | (0.9) | 5 | 12 | (1.3) | 13 | 15 | (1.1) | 16 |
| infection | | | | | | | | | | | | | | | |
| Mild | 2 | (0.4) | 2 | 5 | (1.1) | 5 | 4 | (0.9) | 5 | 9 | (1.0) | 10 | 11 | (0.8) | 12 |
| Moderate | 1 | (0.2) | 1 | 3 | (0.6) | 3 | 0 | | 0 | 3 | (0.3) | 3 | 4 | (0.3) | 4 |
| Severe | 0 | | 0 | 0 | | 0 | 0 | | 0 | 0 | | 0 | 0 | | 0 |
| Urinary tract infection | 6 | (1.3) | 6 | 8 | (1.7) | 8 | 8 | (1.7) | 8 | 16 | (1.7) | 16 | 22 | (1.6) | 22 |
| Mild | 6 | (1.3) | 6 | 6 | (1.3) | 6 | 5 | (1.1) | 5 | 11 | (1.2) | 11 | 17 | (1.2) | 17 |
| Moderate | 0 | | 0 | 2 | (0.4) | 2 | 3 | (0.6) | 3 | 5 | (0.5) | 5 | 5 | (0.4) | 5 |
| Severe | 0 | | 0 | 0 | | 0 | 0 | | 0 | 0 | | 0 | 0 | | 0 |
| Vaginal infection | 2 | (0.4) | 2 | 0 | | 0 | 0 | | 0 | 0 | | 0 | 2 | (0.1) | 2 |
| Mild | 2 | (0.4) | 2 | 0 | | 0 | 0 | | 0 | 0 | | 0 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Vaginitis bacterial | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Viral infection | 1 | (0.2) | 1 | 1 | (0.2) | 1 | 1 | (0.2) | 1 | 2 | (0.2) | 2 | 3 | (0.2) | 3 |
| Mild | 0 | | 0 | 0 | | 0 | 0 | | 0 | 0 | | 0 | 0 | | 0 |
| Moderate | 1 | (0.2) | 1 | 1 | (0.2) | 1 | 1 | (0.2) | 1 | 2 | (0.2) | 2 | 3 | (0.2) | 3 |
| Severe | 0 | | 0 | 0 | | 0 | 0 | | 0 | 0 | | 0 | 0 | | 0 |
| Viral pharyngitis | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Viral upper respiratory | 0 | | 0 | 1 | (0.2) | 1 | 1 | (0.2) | 1 | 2 | (0.2) | 2 | 2 | (0.1) | 2 |
| tract infection | | | | | | | | | | | | | | | |
| Mild | 0 | | 0 | 0 | | 0 | 0 | | 0 | 0 | | 0 | 0 | | 0 |
| Moderate | 0 | | 0 | 1 | (0.2) | 1 | 1 | (0.2) | 1 | 2 | (0.2) | 2 | 2 | (0.1) | 2 |
| Severe | 0 | | 0 | 0 | | 0 | 0 | | 0 | 0 | | 0 | 0 | | 0 |
| Injury, poisoning and | 12 | (2.6) | 14 | 7 | (1.5) | 10 | 9 | (1.9) | 9 | 16 | (1.7) | 19 | 28 | (2.0) | 33 |
| procedural complications | | | | | | | | | | | | | | | |
| Mild | 6 | (1.3) | 7 | 4 | (0.9) | 6 | 4 | (0.9) | 4 | 8 | (0.9) | 10 | 14 | (1.0) | 17 |
| Moderate | 5 | (1.1) | 5 | 1 | (0.2) | 1 | 4 | (0.9) | 4 | 5 | (0.5) | 5 | 10 | (0.7) | 10 |
| Severe | 1 | (0.2) | 1 | 2 | (0.4) | 2 | 1 | (0.2) | 1 | 3 | (0.3) | 3 | 4 | (0.3) | 4 |
| Back injury | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 0 | | 0 | 1 | (0.2) | 1 | 0 | | 0 | 1 | (0.1) | 1 | 1 | (0.1) | 1 |
| Burns second degree | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Contusion | 1 | (0.2) | 1 | 0 | | 0 | 2 | (0.4) | 2 | 2 | (0.2) | 2 | 3 | (0.2) | 3 |
| Mild | 1 | (0.2) | 1 | 0 | | 0 | 2 | (0.4) | 2 | 2 | (0.2) | 2 | 3 | (0.2) | 3 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Corneal abrasion | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Ear abrasion | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Excoriation | 1 | (0.2) | 1 | 1 | (0.2) | 2 | 0 | | 0 | 1 | (0.1) | 2 | 2 | (0.1) | 3 |
| Mild | 1 | (0.2) | 1 | 1 | (0.2) | 2 | 0 | | 0 | 1 | (0.1) | 2 | 2 | (0.1) | 3 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Fall | 2 | (0.4) | 2 | 0 | | 0 | 0 | | 0 | 0 | | 0 | 2 | (0.1) | 2 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 1 | (0.2) | 1 | 0 | | 0 | 0 | | 0 | 0 | | 0 | 1 | (0.1) | 1 |
| Femoral neck fracture | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Foot fracture | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hand fracture | 0 | | 0 | 0 | | 0 | 2 | (0.4) | 2 | 2 | (0.2) | 2 | 2 | (0.1) | 2 |
| Mild | 0 | | 0 | 0 | | 0 | 0 | | 0 | 0 | | 0 | 0 | | 0 |
| Moderate | 0 | | 0 | 0 | | 0 | 2 | (0.4) | 2 | 2 | (0.2) | 2 | 2 | (0.1) | 2 |
| Severe | 0 | | 0 | 0 | | 0 | 0 | | 0 | 0 | | 0 | 0 | | 0 |
| Joint dislocation | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Joint injury | 0 | | 0 | 2 | (0.4) | 2 | 0 | | 0 | 2 | (0.2) | 2 | 2 | (0.1) | 2 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 1 | (0.2) | 1 | 0 | | 0 | 1 | (0.1) | 1 | 1 | (0.1) | 1 |
| Laceration | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Ligament rupture | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Ligament sprain | 2 | (0.4) | 2 | 2 | (0.4) | 2 | 0 | | 0 | 2 | (0.2) | 2 | 4 | (0.3) | 4 |
| Mild | 2 | (0.4) | 2 | 1 | (0.2) | 1 | 0 | | 0 | 1 | (0.1) | 1 | 3 | (0.2) | 3 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Lower limb fracture | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Muscle strain | 1 | (0.2) | 1 | 0 | | 0 | 1 | (0.2) | 1 | 1 | (0.1) | 1 | 2 | (0.1) | 2 |
| Mild | 1 | (0.2) | 1 | 0 | | 0 | 0 | | 0 | 0 | | 0 | 1 | (0.1) | 1 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Procedural pain | 1 | (0.2) | 1 | 0 | | 0 | 1 | (0.2) | 1 | 1 | (0.1) | 1 | 2 | (0.1) | 2 |
| Mild | 0 | | 0 | 0 | | 0 | 0 | | 0 | 0 | | 0 | 0 | | 0 |
| Moderate | 1 | (0.2) | 1 | 0 | | 0 | 1 | (0.2) | 1 | 1 | (0.1) | 1 | 2 | (0.1) | 2 |
| Severe | 0 | | 0 | 0 | | 0 | 0 | | 0 | 0 | | 0 | 0 | | 0 |
| Skeletal injury | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 0 | | 0 | 0 | | 0 | 1 | (0.2) | 1 | 1 | (0.1) | 1 | 1 | (0.1) | 1 |
| Spinal column injury | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Wound | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Investigations | 6 | (1.3) | 7 | 11 | (2.4) | 12 | 12 | (2.6) | 17 | 23 | (2.5) | 29 | 29 | (2.1) | 36 |
| Mild | 5 | (1.1) | 6 | 7 | (1.5) | 8 | 10 | (2.1) | 13 | 17 | (1.8) | 21 | 22 | (1.6) | 27 |
| Moderate | 1 | (0.2) | 1 | 4 | (0.9) | 4 | 1 | (0.2) | 1 | 5 | (0.5) | 5 | 6 | (0.4) | 6 |
| Severe | 0 | | 0 | 0 | | 0 | 1 | (0.2) | 1 | 1 | (0.1) | 1 | 1 | (0.1) | 1 |
| Alanine aminotransferase | 1 | (0.2) | 1 | 2 | (0.4) | 2 | 0 | | 0 | 2 | (0.2) | 2 | 3 | (0.2) | 3 |
| increased | | | | | | | | | | | | | | | |
| Mild | 1 | (0.2) | 1 | 1 | (0.2) | 1 | 0 | | 0 | 1 | (0.1) | 1 | 2 | (0.1) | 2 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Aspartate aminotransferase | 0 | | 0 | 2 | (0.4) | 2 | 0 | | 0 | 2 | (0.2) | 2 | 2 | (0.1) | 2 |
| increased | | | | | | | | | | | | | | | |
| Mild | 0 | | 0 | 1 | (0.2) | 1 | 0 | | 0 | 1 | (0.1) | 1 | 1 | (0.1) | 1 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Blood amylase increased | 1 | (0.2) | 1 | 1 | (0.2) | 1 | 3 | (0.6) | 3 | 4 | (0.4) | 4 | 5 | (0.4) | 5 |
| Mild | 1 | (0.2) | 1 | 0 | | 0 | 3 | (0.6) | 3 | 3 | (0.3) | 3 | 4 | (0.3) | 4 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Blood creatinine increased | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Blood glucose increased | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Blood sodium increased | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Blood triglycerides increased | 0 | | 0 | 1 | (0.2) | 1 | 2 | (0.4) | 2 | 3 | (0.3) | 3 | 3 | (0.2) | 3 |
| Mild | 0 | | 0 | 0 | | 0 | 1 | (0.2) | 1 | 1 | (0.1) | 1 | 1 | (0.1) | 1 |
| Moderate | 0 | | 0 | 1 | (0.2) | 1 | 1 | (0.2) | 1 | 2 | (0.2) | 2 | 2 | (0.1) | 2 |
| Severe | 0 | | 0 | 0 | | 0 | 0 | | 0 | 0 | | 0 | 0 | | 0 |
| Blood uric acid increased | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Blood urine present | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Electrocardiogram QT | 0 | | 0 | 1 | (0.2) | 1 | 0 | | 0 | 1 | (0.1) | 1 | 1 | (0.1) | 1 |
| prolonged | | | | | | | | | | | | | | | |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Electrocardiogram T | 0 | | 0 | 0 | | 0 | 1 | (0.2) | 1 | 1 | (0.1) | 1 | 1 | (0.1) | 1 |
| wave abnormal | | | | | | | | | | | | | | | |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Eosinophil count increased | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hepatic enzyme increased | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hormone level abnormal | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Lipase increased | 1 | (0.2) | 1 | 1 | (0.2) | 1 | 1 | (0.2) | 1 | 2 | (0.2) | 2 | 3 | (0.2) | 3 |
| Mild | 1 | (0.2) | 1 | 1 | (0.2) | 1 | 1 | (0.2) | 1 | 2 | (0.2) | 2 | 3 | (0.2) | 3 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Liver function test abnormal | 1 | (0.2) | 1 | 1 | (0.2) | 1 | 1 | (0.2) | 2 | 2 | (0.2) | 3 | 3 | (0.2) | 4 |
| Mild | 1 | (0.2) | 1 | 1 | (0.2) | 1 | 0 | | 0 | 1 | (0.1) | 1 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 1 | (0.2) | 1 | 1 | (0.1) | 1 | 1 | (0.1) | 1 |
| Pancreatic enzymes increased | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Transaminases increased | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Weight decreased | 0 | | 0 | 0 | | 0 | 1 | (0.2) | 2 | 1 | (0.1) | 2 | 1 | (0.1) | 2 |
| Mild | 0 | | 0 | 0 | | 0 | 1 | (0.2) | 2 | 1 | (0.1) | 2 | 1 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Weight increased | 1 | (0.2) | 1 | 0 | | 0 | 1 | (0.2) | 1 | 1 | (0.1) | 1 | 2 | (0.1) | 2 |
| Mild | 0 | | 0 | 0 | | 0 | 1 | (0.2) | 1 | 1 | (0.1) | 1 | 1 | (0.1) | 1 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Metabolism and nutrition | 9 | (1.9) | 11 | 5 | (1.1) | 7 | 12 | (2.6) | 16 | 17 | (1.8) | 23 | 26 | (1.9) | 34 |
| disorders | | | | | | | | | | | | | | | |
| Mild | 7 | (1.5) | 8 | 4 | (0.9) | 6 | 11 | (2.3) | 13 | 15 | (1.6) | 19 | 22 | (1.6) | 27 |
| Moderate | 2 | (0.4) | 3 | 1 | (0.2) | 1 | 1 | (0.2) | 2 | 2 | (0.2) | 3 | 4 | (0.3) | 6 |
| Severe | 0 | | 0 | 0 | | 0 | 0 | | 0 | 0 | | 0 | 0 | | 0 |
| Decreased appetite | 2 | (0.4) | 2 | 0 | | 0 | 0 | | 0 | 0 | | 0 | 2 | (0.1) | 2 |
| Mild | 2 | (0.4) | 2 | 0 | | 0 | 0 | | 0 | 0 | | 0 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Dehydration | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Hypercholesterolaemia | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hyperglycaemia | 3 | (0.6) | 3 | 0 | | 0 | 2 | (0.4) | 2 | 2 | (0.2) | 2 | 5 | (0.4) | 5 |
| Mild | 2 | (0.4) | 2 | 0 | | 0 | 1 | (0.2) | 1 | 1 | (0.1) | 1 | 3 | (0.2) | 3 |
| Moderate | 1 | (0.2) | 1 | 0 | | 0 | 1 | (0.2) | 1 | 1 | (0.1) | 1 | 2 | (0.1) | 2 |
| Severe | 0 | | 0 | 0 | | 0 | 0 | | 0 | 0 | | 0 | 0 | | 0 |
| Hyperkalaemia | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hyperlipidaemia | 1 | (0.2) | 1 | 1 | (0.2) | 1 | 0 | | 0 | 1 | (0.1) | 1 | 2 | (0.1) | 2 |
| Mild | 1 | (0.2) | 1 | 1 | (0.2) | 1 | 0 | | 0 | 1 | (0.1) | 1 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hyperphosphataemia | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hypertriglyceridaemia | 0 | | 0 | 1 | (0.2) | 1 | 4 | (0.9) | 4 | 5 | (0.5) | 5 | 5 | (0.4) | 5 |
| Mild | 0 | | 0 | 0 | | 0 | 3 | (0.6) | 3 | 3 | (0.3) | 3 | 3 | (0.2) | 3 |
| Moderate | 0 | | 0 | 1 | (0.2) | 1 | 1 | (0.2) | 1 | 2 | (0.2) | 2 | 2 | (0.1) | 2 |
| Severe | 0 | | 0 | 0 | | 0 | 0 | | 0 | 0 | | 0 | 0 | | 0 |
| Hyperuricaemia | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hypocalcaemia | 1 | (0.2) | 1 | 1 | (0.2) | 1 | 0 | | 0 | 1 | (0.1) | 1 | 2 | (0.1) | 2 |
| Mild | 1 | (0.2) | 1 | 1 | (0.2) | 1 | 0 | | 0 | 1 | (0.1) | 1 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hypokalaemia | 2 | (0.4) | 2 | 1 | (0.2) | 1 | 2 | (0.4) | 2 | 3 | (0.3) | 3 | 5 | (0.4) | 5 |
| Mild | 1 | (0.2) | 1 | 1 | (0.2) | 1 | 2 | (0.4) | 2 | 3 | (0.3) | 3 | 4 | (0.3) | 4 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Hypomagnesaemia | 0 | | 0 | 2 | (0.4) | 2 | 0 | | 0 | 2 | (0.2) | 2 | 2 | (0.1) | 2 |
| Mild | 0 | | 0 | 2 | (0.4) | 2 | 0 | | 0 | 2 | (0.2) | 2 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hyponatraemia | 0 | | 0 | 0 | | 0 | 2 | (0.4) | 2 | 2 | (0.2) | 2 | 2 | (0.1) | 2 |
| Mild | 0 | | 0 | 0 | | 0 | 2 | (0.4) | 2 | 2 | (0.2) | 2 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hypophosphataemia | 0 | | 0 | 0 | | 0 | 2 | (0.4) | 2 | 2 | (0.2) | 2 | 2 | (0.1) | 2 |
| Mild | 0 | | 0 | 0 | | 0 | 2 | (0.4) | 2 | 2 | (0.2) | 2 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Increased appetite | 1 | (0.2) | 1 | 0 | | 0 | 1 | (0.2) | 1 | 1 | (0.1) | 1 | 2 | (0.1) | 2 |
| Mild | 1 | (0.2) | 1 | 0 | | 0 | 1 | (0.2) | 1 | 1 | (0.1) | 1 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Musculoskeletal and | 7 | (1.5) | 8 | 8 | (1.7) | 8 | 11 | (2.3) | 11 | 19 | (2.0) | 19 | 26 | (1.9) | 27 |
| connective tissue disorders | | | | | | | | | | | | | | | |
| Mild | 2 | (0.4) | 3 | 4 | (0.9) | 4 | 6 | (1.3) | 6 | 10 | (1.1) | 10 | 12 | (0.9) | 13 |
| Moderate | 4 | (0.9) | 4 | 4 | (0.9) | 4 | 5 | (1.1) | 5 | 9 | (1.0) | 9 | 13 | (0.9) | 13 |
| Severe | 1 | (0.2) | 1 | 0 | | 0 | 0 | | 0 | 0 | | 0 | 1 | (0.1) | 1 |
| Arthralgia | 1 | (0.2) | 1 | 2 | (0.4) | 2 | 2 | (0.4) | 2 | 4 | (0.4) | 4 | 5 | (0.4) | 5 |
| Mild | 1 | (0.2) | 1 | 0 | | 0 | 2 | (0.4) | 2 | 2 | (0.2) | 2 | 3 | (0.2) | 3 |
| Moderate | 0 | | 0 | 2 | (0.4) | 2 | 0 | | 0 | 2 | (0.2) | 2 | 2 | (0.1) | 2 |
| Severe | 0 | | 0 | 0 | | 0 | 0 | | 0 | 0 | | 0 | 0 | | 0 |
| Arthritis | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Back pain | 3 | (0.6) | 3 | 2 | (0.4) | 2 | 4 | (0.9) | 4 | 6 | (0.6) | 6 | 9 | (0.6) | 9 |
| Mild | 1 | (0.2) | 1 | 2 | (0.4) | 2 | 2 | (0.4) | 2 | 4 | (0.4) | 4 | 5 | (0.4) | 5 |
| Moderate | 2 | (0.4) | 2 | 0 | | 0 | 2 | (0.4) | 2 | 2 | (0.2) | 2 | 4 | (0.3) | 4 |
| Severe | 0 | | 0 | 0 | | 0 | 0 | | 0 | 0 | | 0 | 0 | | 0 |
| Bursitis | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Intervertebral disc disorder | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Intervertebral disc protrusion | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 1 | (0.2) | 1 | 0 | | 0 | 0 | | 0 | 0 | | 0 | 1 | (0.1) | 1 |
| Joint swelling | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Muscle spasms | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Musculoskeletal chest pain | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Musculoskeletal pain | 0 | | 0 | 1 | (0.2) | 1 | 1 | (0.2) | 1 | 2 | (0.2) | 2 | 2 | (0.1) | 2 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Osteoarthritis | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Pain in extremity | 1 | (0.2) | 1 | 1 | (0.2) | 1 | 0 | | 0 | 1 | (0.1) | 1 | 2 | (0.1) | 2 |
| Mild | 0 | | 0 | 0 | | 0 | 0 | | 0 | 0 | | 0 | 0 | | 0 |
| Moderate | 1 | (0.2) | 1 | 1 | (0.2) | 1 | 0 | | 0 | 1 | (0.1) | 1 | 2 | (0.1) | 2 |
| Severe | 0 | | 0 | 0 | | 0 | 0 | | 0 | 0 | | 0 | 0 | | 0 |
| Plantar fasciitis | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Neoplasms benign, | 0 | | 0 | 1 | (0.2) | 1 | 2 | (0.4) | 3 | 3 | (0.3) | 4 | 3 | (0.2) | 4 |
| malignant and unspecified | | | | | | | | | | | | | | | |
| (incl cysts and polyps) | | | | | | | | | | | | | | | |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 1 | (0.2) | 2 | 1 | (0.1) | 2 | 1 | (0.1) | 2 |
| Severe | 0 | | 0 | 1 | (0.2) | 1 | 0 | | 0 | 1 | (0.1) | 1 | 1 | (0.1) | 1 |
| Fibroadenoma of breast | 0 | | 0 | 0 | | 0 | 1 | (0.2) | 2 | 1 | (0.1) | 2 | 1 | (0.1) | 2 |
| Mild | 0 | | 0 | 0 | | 0 | 0 | | 0 | 0 | | 0 | 0 | | 0 |
| Moderate | 0 | | 0 | 0 | | 0 | 1 | (0.2) | 2 | 1 | (0.1) | 2 | 1 | (0.1) | 2 |
| Severe | 0 | | 0 | 0 | | 0 | 0 | | 0 | 0 | | 0 | 0 | | 0 |
| Meningioma | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Renal cancer | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 0 | | 0 | 1 | (0.2) | 1 | 0 | | 0 | 1 | (0.1) | 1 | 1 | (0.1) | 1 |
| Nervous system disorders | 15 | (3.2) | 16 | 18 | (3.9) | 20 | 15 | (3.2) | 17 | 33 | (3.5) | 37 | 48 | (3.4) | 53 |
| Mild | 9 | (1.9) | 9 | 14 | (3.0) | 16 | 13 | (2.8) | 15 | 27 | (2.9) | 31 | 36 | (2.6) | 40 |
| Moderate | 6 | (1.3) | 6 | 3 | (0.6) | 3 | 1 | (0.2) | 1 | 4 | (0.4) | 4 | 10 | (0.7) | 10 |
| Severe | 0 | | 0 | 1 | (0.2) | 1 | 1 | (0.2) | 1 | 2 | (0.2) | 2 | 2 | (0.1) | 2 |
| Burning sensation | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Convulsion | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 0 | | 0 | 0 | | 0 | 1 | (0.2) | 1 | 1 | (0.1) | 1 | 1 | (0.1) | 1 |
| Dizziness | 4 | (0.9) | 4 | 4 | (0.9) | 4 | 2 | (0.4) | 2 | 6 | (0.6) | 6 | 10 | (0.7) | 10 |
| Mild | 2 | (0.4) | 2 | 4 | (0.9) | 4 | 2 | (0.4) | 2 | 6 | (0.6) | 6 | 8 | (0.6) | 8 |
| Moderate | 2 | (0.4) | 2 | 0 | | 0 | 0 | | 0 | 0 | | 0 | 2 | (0.1) | 2 |
| Severe | 0 | | 0 | 0 | | 0 | 0 | | 0 | 0 | | 0 | 0 | | 0 |
| Headache | 9 | (1.9) | 10 | 10 | (2.1) | 10 | 10 | (2.1) | 10 | 20 | (2.1) | 20 | 29 | (2.1) | 30 |
| Mild | 6 | (1.3) | 6 | 10 | (2.1) | 10 | 10 | (2.1) | 10 | 20 | (2.1) | 20 | 26 | (1.9) | 26 |
| Moderate | 3 | (0.6) | 3 | 0 | | 0 | 0 | | 0 | 0 | | 0 | 3 | (0.2) | 3 |
| Severe | 0 | | 0 | 0 | | 0 | 0 | | 0 | 0 | | 0 | 0 | | 0 |
| Hypoaesthesia | 0 | | 0 | 2 | (0.4) | 2 | 0 | | 0 | 2 | (0.2) | 2 | 2 | (0.1) | 2 |
| Mild | 0 | | 0 | 0 | | 0 | 0 | | 0 | 0 | | 0 | 0 | | 0 |
| Moderate | 0 | | 0 | 2 | (0.4) | 2 | 0 | | 0 | 2 | (0.2) | 2 | 2 | (0.1) | 2 |
| Severe | 0 | | 0 | 0 | | 0 | 0 | | 0 | 0 | | 0 | 0 | | 0 |
| Loss of consciousness | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 0 | | 0 | 1 | (0.2) | 1 | 0 | | 0 | 1 | (0.1) | 1 | 1 | (0.1) | 1 |
| Memory impairment | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Migraine | 0 | | 0 | 1 | (0.2) | 1 | 1 | (0.2) | 1 | 2 | (0.2) | 2 | 2 | (0.1) | 2 |
| Mild | 0 | | 0 | 0 | | 0 | 0 | | 0 | 0 | | 0 | 0 | | 0 |
| Moderate | 0 | | 0 | 1 | (0.2) | 1 | 1 | (0.2) | 1 | 2 | (0.2) | 2 | 2 | (0.1) | 2 |
| Severe | 0 | | 0 | 0 | | 0 | 0 | | 0 | 0 | | 0 | 0 | | 0 |
| Nystagmus | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Restless legs syndrome | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Sciatica | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Somnolence | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Syncope | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Pregnancy, puerperium | 2 | (0.4) | 3 | 1 | (0.2) | 1 | 1 | (0.2) | 1 | 2 | (0.2) | 2 | 4 | (0.3) | 5 |
| and perinatal conditions | | | | | | | | | | | | | | | |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Grade is missing | 1 | (0.2) | 1 | 1 | (0.2) | 1 | 1 | (0.2) | 1 | 2 | (0.2) | 2 | 3 | (0.2) | 3 |
| Abortion spontaneous | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Pregnancy | 2 | (0.4) | 2 | 1 | (0.2) | 1 | 1 | (0.2) | 1 | 2 | (0.2) | 2 | 4 | (0.3) | 4 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Grade is missing | 2 | (0.4) | 2 | 1 | (0.2) | 1 | 1 | (0.2) | 1 | 2 | (0.2) | 2 | 4 | (0.3) | 4 |
| Psychiatric disorders | 3 | (0.6) | 4 | 4 | (0.9) | 5 | 3 | (0.6) | 3 | 7 | (0.7) | 8 | 10 | (0.7) | 12 |
| Mild | 0 | | 0 | 3 | (0.6) | 4 | 3 | (0.6) | 3 | 6 | (0.6) | 7 | 6 | (0.4) | 7 |
| Moderate | 3 | (0.6) | 3 | 1 | (0.2) | 1 | 0 | | 0 | 1 | (0.1) | 1 | 4 | (0.3) | 4 |
| Severe | 0 | | 0 | 0 | | 0 | 0 | | 0 | 0 | | 0 | 0 | | 0 |
| Attention deficit/ | 0 | | 0 | 0 | | 0 | 1 | (0.2) | 1 | 1 | (0.1) | 1 | 1 | (0.1) | 1 |
| hyperactivity disorder | | | | | | | | | | | | | | | |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Bradyphrenia | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Depression | 2 | (0.4) | 2 | 0 | | 0 | 0 | | 0 | 0 | | 0 | 2 | (0.1) | 2 |
| Mild | 1 | (0.2) | 1 | 0 | | 0 | 0 | | 0 | 0 | | 0 | 1 | (0.1) | 1 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Insomnia | 2 | (0.4) | 2 | 1 | (0.2) | 1 | 2 | (0.4) | 2 | 3 | (0.3) | 3 | 5 | (0.4) | 5 |
| Mild | 0 | | 0 | 1 | (0.2) | 1 | 2 | (0.4) | 2 | 3 | (0.3) | 3 | 3 | (0.2) | 3 |
| Moderate | 2 | (0.4) | 2 | 0 | | 0 | 0 | | 0 | 0 | | 0 | 2 | (0.1) | 2 |
| Severe | 0 | | 0 | 0 | | 0 | 0 | | 0 | 0 | | 0 | 0 | | 0 |
| Panic attack | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Stress | 0 | | 0 | 2 | (0.4) | 2 | 0 | | 0 | 2 | (0.2) | 2 | 2 | (0.1) | 2 |
| Mild | 0 | | 0 | 2 | (0.4) | 2 | 0 | | 0 | 2 | (0.2) | 2 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Renal and urinary disorders | 1 | (0.2) | 1 | 1 | (0.2) | 1 | 3 | (0.6) | 3 | 4 | (0.4) | 4 | 5 | (0.4) | 5 |
| Mild | 0 | | 0 | 1 | (0.2) | 1 | 2 | (0.4) | 2 | 3 | (0.3) | 3 | 3 | (0.2) | 3 |
| Moderate | 0 | | 0 | 0 | | 0 | 1 | (0.2) | 1 | 1 | (0.1) | 1 | 1 | (0.1) | 1 |
| Severe | 1 | (0.2) | 1 | 0 | | 0 | 0 | | 0 | 0 | | 0 | 1 | (0.1) | 1 |
| Haematuria | 0 | | 0 | 0 | | 0 | 2 | (0.4) | 2 | 2 | (0.2) | 2 | 2 | (0.1) | 2 |
| Mild | 0 | | 0 | 0 | | 0 | 2 | (0.4) | 2 | 2 | (0.2) | 2 | 2 | (0.1) | 2 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Nephrolithiasis | 1 | (0.2) | 1 | 0 | | 0 | 1 | (0.2) | 1 | 1 | (0.1) | 1 | 2 | (0.1) | 2 |
| Mild | 0 | | 0 | 0 | | 0 | 0 | | 0 | 0 | | 0 | 0 | | 0 |
| Moderate | 0 | | 0 | 0 | | 0 | 1 | (0.2) | 1 | 1 | (0.1) | 1 | 1 | (0.1) | 1 |
| Severe | 1 | (0.2) | 1 | 0 | | 0 | 0 | | 0 | 0 | | 0 | 1 | (0.1) | 1 |
| Stress urinary incontinence | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Reproductive system and | 0 | | 0 | 1 | (0.2) | 1 | 5 | (1.1) | 6 | 6 | (0.6) | 7 | 6 | (0.4) | 7 |
| breast disorders | | | | | | | | | | | | | | | |
| Mild | 0 | | 0 | 1 | (0.2) | 1 | 3 | (0.6) | 3 | 4 | (0.4) | 4 | 4 | (0.3) | 4 |
| Moderate | 0 | | 0 | 0 | | 0 | 2 | (0.4) | 3 | 2 | (0.2) | 3 | 2 | (0.1) | 3 |
| Severe | 0 | | 0 | 0 | | 0 | 0 | | 0 | 0 | | 0 | 0 | | 0 |
| Balanitis | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Dysmenorrhoea | 0 | | 0 | 0 | | 0 | 2 | (0.4) | 2 | 2 | (0.2) | 2 | 2 | (0.1) | 2 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Menorrhagia | 0 | | 0 | 0 | | 0 | 2 | (0.4) | 3 | 2 | (0.2) | 3 | 2 | (0.1) | 3 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 1 | (0.2) | 2 | 1 | (0.1) | 2 | 1 | (0.1) | 2 |
| Severe | 0 | | 0 | 0 | | 0 | 0 | | 0 | 0 | | 0 | 0 | | 0 |
| Vaginal discharge | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Respiratory, thoracic and | 9 | (1.9) | 10 | 10 | (2.1) | 11 | 9 | (1.9) | 9 | 19 | (2.0) | 20 | 28 | (2.0) | 30 |
| mediastinal disorders | | | | | | | | | | | | | | | |
| Mild | 4 | (0.9) | 4 | 5 | (1.1) | 5 | 7 | (1.5) | 7 | 12 | (1.3) | 12 | 16 | (1.1) | 16 |
| Moderate | 5 | (1.1) | 6 | 5 | (1.1) | 6 | 2 | (0.4) | 2 | 7 | (0.7) | 8 | 12 | (0.9) | 14 |
| Severe | 0 | | 0 | 0 | | 0 | 0 | | 0 | 0 | | 0 | 0 | | 0 |
| Asthma | 2 | (0.4) | 2 | 1 | (0.2) | 1 | 1 | (0.2) | 1 | 2 | (0.2) | 2 | 4 | (0.3) | 4 |
| Mild | 0 | | 0 | 1 | (0.2) | 1 | 1 | (0.2) | 1 | 2 | (0.2) | 2 | 2 | (0.1) | 2 |
| Moderate | 2 | (0.4) | 2 | 0 | | 0 | 0 | | 0 | 0 | | 0 | 2 | (0.1) | 2 |
| Severe | 0 | | 0 | 0 | | 0 | 0 | | 0 | 0 | | 0 | 0 | | 0 |
| Chronic obstructive | 0 | | 0 | 1 | (0.2) | 1 | 1 | (0.2) | 1 | 2 | (0.2) | 2 | 2 | (0.1) | 2 |
| pulmonary disease | | | | | | | | | | | | | | | |
| Mild | 0 | | 0 | 0 | | 0 | 0 | | 0 | 0 | | 0 | 0 | | 0 |
| Moderate | 0 | | 0 | 1 | (0.2) | 1 | 1 | (0.2) | 1 | 1 | (0.2) | 2 | 2 | (0.1) | 2 |
| Severe | 0 | | 0 | 0 | | 0 | 0 | | 0 | 0 | | 0 | 0 | | 0 |
| Cough | 2 | (0.4) | 2 | 5 | (1.1) | 5 | 2 | (0.4) | 2 | 7 | (0.7) | 7 | 9 | (0.6) | 9 |
| Mild | 0 | | 0 | 3 | (0.6) | 3 | 2 | (0.4) | 2 | 5 | (0.5) | 5 | 5 | (0.4) | 5 |
| Moderate | 2 | (0.4) | 2 | 2 | (0.4) | 2 | 0 | | 0 | 2 | (0.2) | 2 | 4 | (0.3) | 4 |
| Severe | 0 | | 0 | 0 | | 0 | 0 | | 0 | 0 | | 0 | 0 | | 0 |
| Dyspnoea | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Epistaxis | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Nasal congestion | 1 | (0.2) | 1 | 1 | (0.2) | 1 | 1 | (0.2) | 1 | 2 | (0.2) | 2 | 3 | (0.2) | 3 |
| Mild | 0 | | 0 | 0 | | 0 | 1 | (0.2) | 1 | 1 | (0.1) | 1 | 1 | (0.1) | 1 |
| Moderate | 1 | (0.2) | 1 | 1 | (0.2) | 1 | 0 | | 0 | 1 | (0.1) | 1 | 2 | (0.1) | 2 |
| Severe | 0 | | 0 | 0 | | 0 | 0 | | 0 | 0 | | 0 | 0 | | 0 |
| Oropharyngeal pain | 3 | (0.6) | 3 | 0 | | 0 | 1 | (0.2) | 1 | 1 | (0.1) | 1 | 4 | (0.3) | 4 |
| Mild | 2 | (0.4) | 2 | 0 | | 0 | 1 | (0.2) | 1 | 1 | (0.1) | 1 | 3 | (0.2) | 3 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Pharyngeal oedema | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Productive cough | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Pulmonary congestion | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Rhinitis allergic | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Sinus congestion | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Throat irritation | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Skin and subcutaneous | 11 | (2.4) | 12 | 8 | (1.7) | 9 | 3 | (0.6) | 3 | 11 | (1.2) | 12 | 22 | (1.6) | 24 |
| tissue disorders | | | | | | | | | | | | | | | |
| Mild | 7 | (1.5) | 8 | 4 | (0.9) | 4 | 2 | (0.4) | 2 | 6 | (0.6) | 6 | 13 | (0.9) | 14 |
| Moderate | 4 | (0.9) | 4 | 4 | (0.9) | 5 | 1 | (0.2) | 1 | 5 | (0.5) | 6 | 9 | (0.6) | 10 |
| Severe | 0 | | 0 | 0 | | 0 | 0 | | 0 | 0 | | 0 | 0 | | 0 |
| Acne | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Acne cystic | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Dermatitis | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Dermatitis contact | 1 | (0.2) | 1 | 1 | (0.2) | 2 | 0 | | 0 | 1 | (0.1) | 2 | 2 | (0.1) | 3 |
| Mild | 1 | (0.2) | 1 | 0 | | 0 | 0 | | 0 | 0 | | 0 | 1 | (0.1) | 1 |
| Moderate | 0 | | 0 | 1 | (0.2) | 2 | 0 | | 0 | 1 | (0.1) | 2 | 1 | (0.1) | 2 |
| Severe | 0 | | 0 | 0 | | 0 | 0 | | 0 | 0 | | 0 | 0 | | 0 |
| Eczema | 1 | (0.2) | 1 | 1 | (0.2) | 1 | 0 | | 0 | 1 | (0.1) | 1 | 2 | (0.1) | 2 |
| Mild | 0 | | 0 | 0 | | 0 | 0 | | 0 | 0 | | 0 | 0 | | 0 |
| Moderate | 1 | (0.2) | 1 | 1 | (0.2) | 1 | 0 | | 0 | 1 | (0.1) | 1 | 2 | (0.1) | 2 |
| Severe | 0 | | 0 | 0 | | 0 | 0 | | 0 | 0 | | 0 | 0 | | 0 |
| Erythema | 0 | | 0 | 0 | | 0 | 1 | (0.2) | 1 | 1 | (0.1) | 1 | 1 | (0.1) | 1 |
| Mild | 0 | | 0 | 0 | | 0 | 1 | (0.2) | 1 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Pruritus | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Rash | 6 | (1.3) | 7 | 3 | (0.6) | 3 | 0 | | 0 | 3 | (0.3) | 3 | 9 | (0.6) | 10 |
| Mild | 3 | (0.6) | 4 | 2 | (0.4) | 2 | 0 | | 0 | 2 | (0.2) | 2 | 5 | (0.4) | 6 |
| Moderate | 3 | (0.6) | 3 | 1 | (0.2) | 1 | 0 | | 0 | 1 | (0.1) | 1 | 4 | (0.3) | 4 |
| Severe | 0 | | 0 | 0 | | 0 | 0 | | 0 | 0 | | 0 | 0 | | 0 |
| Rash erythematous | 1 | (0.2) | 1 | 1 | (0.2) | 1 | 0 | | 0 | 1 | (0.1) | 1 | 2 | (0.1) | 2 |
| Mild | 1 | (0.2) | 1 | 0 | | 0 | 0 | | 0 | 0 | | 0 | 1 | (0.1) | 1 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Skin ulcer | 0 | | 0 | 0 | | 0 | 1 | (0.2) | 1 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 1 | (0.2) | 1 | 1 | (0.1) | 1 | 1 | (0.1) | 1 |
| Severe | 0 | | 0 | 0 | | 0 | 0 | | 0 | 0 | | 0 | 0 | | 0 |
| Urticaria | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Surgical and medical | 2 | (0.4) | 3 | 4 | (0.9) | 4 | 2 | (0.4) | 2 | 6 | (0.6) | 6 | 8 | (0.6) | 9 |
| procedures | | | | | | | | | | | | | | | |
| Mild | 0 | | 0 | 1 | (0.2) | 1 | 1 | (0.2) | 1 | 2 | (0.2) | 2 | 2 | (0.1) | 2 |
| Moderate | 2 | (0.4) | 2 | 3 | (0.6) | 3 | 1 | (0.2) | 1 | 4 | (0.4) | 4 | 6 | (0.4) | 6 |
| Severe | 0 | | 0 | 0 | | 0 | 0 | | 0 | 0 | | 0 | 0 | | 0 |
| Intervertebral disc operation | 1 | (0.2) | 1 | 0 | | 0 | 0 | | 0 | 0 | | 0 | 1 | (0.1) | 1 |
| Mild | 1 | (0.2) | 1 | 0 | | 0 | 0 | | 0 | 0 | | 0 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Skin cosmetic procedure | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Skin neoplasm excision | 0 | | 0 | 1 | (0.2) | 1 | 0 | | 0 | 1 | (0.1) | 1 | 1 | (0.1) | 1 |
| Mild | 0 | | 0 | 0 | | 0 | 0 | | 0 | 0 | | 0 | 0 | | 0 |
| Moderate | 0 | | 0 | 1 | (0.2) | 1 | 0 | | 0 | 1 | (0.1) | 1 | 1 | (0.1) | 1 |
| Severe | 0 | | 0 | 0 | | 0 | 0 | | 0 | 0 | | 0 | 0 | | 0 |
| Spinal fusion surgery | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 1 | (0.2) | 1 | 0 | | 0 | 0 | | 0 | 0 | | 0 | 1 | (0.1) | 1 |
| Severe | 0 | | 0 | 0 | | 0 | 0 | | 0 | 0 | | 0 | 0 | | 0 |
| Tooth extraction | 1 | (0.2) | 1 | 2 | (0.4) | 2 | 2 | (0.4) | 2 | 4 | (0.4) | 4 | 5 | (0.4) | 5 |
| Mild | 0 | | 0 | 0 | | 0 | 1 | (0.2) | 1 | 1 | (0.1) | 1 | 1 | (0.1) | 1 |
| Moderate | 1 | (0.2) | 1 | 2 | (0.4) | 2 | 1 | (0.2) | 1 | 3 | (0.3) | 3 | 4 | (0.3) | 4 |
| Severe | 0 | | 0 | 0 | | 0 | 0 | | 0 | 0 | | 0 | 0 | | 0 |
| Vascular disorders | 3 | (0.6) | 4 | 3 | (0.6) | 3 | 1 | (0.2) | 1 | 4 | (0.4) | 4 | 7 | (0.5) | 8 |
| Mild | 1 | (0.2) | 1 | 1 | (0.2) | 1 | 0 | | 0 | 1 | (0.1) | 1 | 2 | (0.1) | 2 |
| Moderate | 1 | (0.2) | 1 | 1 | (0.2) | 1 | 1 | (0.2) | 1 | 2 | (0.2) | 2 | 3 | (0.2) | 3 |
| Severe | 1 | (0.2) | 2 | 1 | (0.2) | 1 | 0 | | 0 | 1 | (0.1) | 1 | 2 | (0.1) | 3 |
| Arterial occlusive disease | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 1 | (0.2) | 1 | 0 | | 0 | 0 | | 0 | 0 | | 0 | 1 | (0.1) | 1 |
| Haematoma | 0 | | 0 | 1 | (0.2) | 1 | 0 | | 0 | 1 | (0.1) | 1 | 1 | (0.1) | 1 |
| Mild | 0 | | 0 | 1 | (0.2) | 1 | 0 | | 0 | 1 | (0.1) | 1 | 1 | (0.1) | 1 |
| Moderate | 0 | | 0 | 0 | | 0 | 0 | | 0 | 0 | | 0 | 0 | | 0 |
| Severe | 0 | | 0 | 0 | | 0 | 0 | | 0 | 0 | | 0 | 0 | | 0 |
| Hypertension | 2 | (0.4) | 2 | 2 | (0.4) | 2 | 1 | (0.2) | 1 | 3 | (0.3) | 3 | 5 | (0.4) | 5 |
| Mild | 1 | (0.2) | 1 | 0 | | 0 | 0 | | 0 | 0 | | 0 | 1 | (0.1) | 1 |
| Moderate | 1 | (0.2) | 1 | 1 | (0.2) | 1 | 1 | (0.2) | 1 | 2 | (0.2) | 2 | 3 | (0.2) | 3 |
| Severe | 0 | | 0 | 1 | (0.2) | 1 | 0 | | 0 | 1 | (0.1) | 1 | 1 | (0.1) | 1 |
| Peripheral vascular disorder | 1 | (0.2) | 1 | 0 | | 0 | 0 | | 0 | 0 | | 0 | 1 | (0.1) | 1 |
| Mild | 0 | | 0 | 0 | | 0 | 0 | | 0 | 0 | | 0 | 0 | | 0 |
| Moderate | 0 | | 0 | 0 | | 0 | 0 | | 0 | 0 | | 0 | 0 | | 0 |
| Severe | 1 | (0.2) | 1 | 0 | | 0 | 0 | | 0 | 0 | | 0 | 1 | (0.1) | 1 |

Source: Listing 16.2.10.1
Note:
If a patient has multiple occurrences of an AE, the patient is only counted at the greatest severity for a given SOC and PT. Events are counted each time in the event (E) column. Adverse events are coded using MedDRA version 14.1.
Chiltern · 29JUL2015:5:34 PM · I:\Synergy\33610\21_Stat\Programs\Tables\t_aeby.sas · ZScott

TABLE 33
Summary of Treatment Emergent Serious Adverse Events by System Organ Class, Preferred Term, and Relationship to Study Drug
(Safety Population)

| | | | | Plecanatide | | | Plecanatide | | | Active | | | | | |
|---|---|---|---|---|---|---|---|---|---|---|---|---|---|---|---|
| System Organ Class | Placebo | | | 3 mg | | | 6 mg | | | Combined | | | Overall | | |
| Preferred Term | (N = 466) | | | (N = 467) | | | (N = 469) | | | (N = 936) | | | (N = 1402) | | |
| Relationship | N | (%) | E | N | (%) | E | N | (%) | E | N | (%) | E | N | (%) | E |
| Number of Treatment Emergent | 8 | (1.7) | 9 | 8 | (1.7) | 9 | 4 | (0.9) | 4 | 12 | (1.3) | 13 | 20 | (1.4) | 22 |
| Serious Adverse Events | | | | | | | | | | | | | | | |
| Reasonable Possibility [1] | 0 | | 0 | 0 | | 0 | 1 | (0.2) | 1 | 1 | (0.1) | 1 | 1 | (0.1) | 1 |
| No Reasonable Possibility | 8 | (1.7) | 9 | 8 | (1.7) | 9 | 3 | (0.6) | 3 | 11 | (1.2) | 12 | 19 | (1.4) | 21 |
| Cardiac disorders | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Cardiac failure congestive | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Ear and labyrinth disorders | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Vertigo positional | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| General disorders and | 1 | (0.2) | 0 | 0 | | 0 | 0 | | 0 | 0 | | 0 | 1 | (0.1) | 1 |
| administration site conditions | | | | | | | | | | | | | | | |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Non-cardiac chest pain | 1 | (0.2) | 1 | 0 | | 0 | 0 | | 0 | 0 | | 0 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Hepatobiliary disorders | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Cholecystitis | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Injury, poisoning and procedural | 1 | (0.2) | 1 | 0 | | 0 | 0 | | 0 | 0 | | 0 | 1 | (0.1) | 1 |
| complications | | | | | | | | | | | | | | | |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Femoral neck fracture | 1 | (0.2) | 1 | 0 | | 0 | 0 | | 0 | 0 | | 0 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Investigations | 1 | (0.2) | 1 | 3 | (0.6) | 3 | 1 | (0.2) | 1 | 4 | (0.4) | 4 | 5 | (0.4) | 5 |
| Reasonable Possibility [1] | 0 | | 0 | 0 | | 0 | 1 | (0.2) | 1 | 1 | (0.1) | 1 | 1 | (0.1) | 1 |
| No Reasonable Possibility | 1 | (0.2) | 1 | 3 | (0.6) | 3 | 0 | | 0 | 3 | (0.3) | 3 | 4 | (0.3) | 4 |
| Alanine aminotransferase increased | 1 | (0.2) | 1 | 1 | (0.2) | 1 | 0 | | 0 | 1 | (0.1) | 1 | 2 | (0.1) | 2 |
| Reasonable Possibility [1] | 0 | | 0 | 0 | | 0 | 0 | | 0 | 0 | | 0 | 0 | | 0 |
| No Reasonable Possibility | 1 | | 1 | 1 | (0.2) | 1 | 0 | | 0 | 1 | (0.1) | 1 | 2 | (0.1) | 2 |
| Aspartate aminotransferase increased | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Liver function test abnormal | 0 | | 0 | 1 | (0.2) | 1 | 1 | (0.2) | 1 | 2 | (0.2) | 2 | 2 | (0.1) | 2 |
| Reasonable Possibility [1] | 0 | | 0 | 0 | | 0 | 1 | (0.2) | 1 | 1 | (0.1) | 1 | 1 | (0.1) | 1 |
| No Reasonable Possibility | 0 | | 0 | 1 | (0.2) | 1 | 0 | | 0 | 1 | (0.1) | 1 | 1 | (0.1) | 1 |
| Musculoskeletal and connective | 1 | (0.2) | 1 | 0 | | 0 | 0 | | 0 | 0 | | 0 | 1 | (0.1) | 1 |
| tissue disorders | | | | | | | | | | | | | | | |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Intervertebral disc protrusion | 1 | (0.2) | 1 | 0 | | 0 | 0 | | 0 | 0 | | 0 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Neoplasms benign, malignant and | 0 | | 0 | 1 | (0.2) | 1 | 0 | | 0 | 1 | (0.1) | 1 | 1 | (0.1) | 1 |
| unspecified (incl cysts and polyps) | | | | | | | | | | | | | | | |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Renal cancer | 0 | | 0 | 1 | (0.2) | 1 | 0 | | 0 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 0 | | 0 | 1 | (0.1) | 1 | 1 | (0.1) | 1 |
| Nervous system disorders | 0 | | 0 | 0 | | 0 | 1 | (0.2) | 1 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 0 | | 0 | 1 | (0.2) | 1 | 1 | (0.1) | 1 | 1 | (0.1) | 1 |
| Convulsion | 0 | | 0 | 0 | | 0 | 1 | (0.2) | 1 | 1 | (0.1) | 1 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 0 | | 0 | 0 | | 0 | 1 | (0.2) | 1 | 1 | (0.1) | 1 | 1 | (0.1) | 1 |
| Pregnancy, puerperium and | 2 | (0.4) | 3 | 1 | (0.2) | 1 | 1 | (0.2) | 1 | 2 | (0.2) | 2 | 4 | (0.3) | 5 |
| perinatal conditions | | | | | | | | | | | | | | | |
| Reasonable Possibility [1] | 0 | | 0 | 0 | | 0 | 0 | | 0 | 0 | | 0 | 0 | | 0 |
| No Reasonable Possibility | 2 | (0.4) | 3 | 1 | (0.2) | 1 | 1 | (0.2) | 1 | 2 | (0.2) | 2 | 4 | (0.3) | 5 |
| Abortion spontaneous | 1 | (0.2) | 1 | 0 | | 0 | 0 | | 0 | 0 | | 0 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Pregnancy | 2 | (0.4) | 2 | 1 | (0.2) | 1 | 1 | (0.2) | 1 | 2 | (0.2) | 2 | 4 | (0.3) | 4 |
| Reasonable Possibility [1] | 0 | | 0 | 0 | | 0 | 0 | | 0 | 0 | | 0 | 0 | | 0 |
| No Reasonable Possibility | 2 | (0.4) | 2 | 1 | (0.2) | 1 | 1 | (0.2) | 1 | 2 | (0.2) | 2 | 4 | (0.3) | |
| Renal and urinary disorders | 1 | (0.2) | 1 | 0 | | 0 | 0 | | 0 | 0 | | 0 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Nephrolithiasis | 1 | (0.2) | 1 | 0 | | 0 | 0 | | 0 | 0 | | 0 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Respiratory, thoracic and | 0 | | 0 | 1 | (0.2) | 1 | 1 | (0.2) | 1 | 2 | (0.2) | 2 | 2 | (0.1) | 2 |
| mediastinal disorders | | | | | | | | | | | | | | | |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 1 | (0.2) | 1 | 2 | (0.2) | 2 | 2 | (0.1) | 2 |
| Chronic obstructive pulmonary | 0 | | 0 | 1 | (0.2) | 1 | 1 | (0.2) | 1 | 2 | (0.2) | 2 | 2 | (0.1) | 2 |
| disease | | | | | | | | | | | | | | | |
| Reasonable Possibility [1] | 0 | | 0 | 0 | | 0 | 0 | | 0 | 0 | | 0 | 0 | | 0 |
| No Reasonable Possibility | 0 | | 0 | 1 | (0.2) | 1 | 1 | (0.2) | 1 | 2 | (0.2) | 2 | 2 | (0.1) | 2 |
| Vascular disorders | 1 | (0.2) | 1 | 0 | | 0 | 0 | | 0 | 0 | | 0 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |
| Arterial occlusive disease | 1 | (0.2) | 1 | 0 | | 0 | 0 | | 0 | 0 | | 0 | 1 | (0.1) | 1 |
| Reasonable Possibility [1] | 0 | | 0 | 0 | | 0 | 0 | | 0 | 0 | | 0 | 0 | | 0 |
| No Reasonable Possibility | 1 | (0.2) | 1 | 0 | | 0 | 0 | | 0 | 0 | | 0 | 1 | (0.1) | 1 |

Source: Listing 16.2.10.3
Note:
If a patient has multiple occurrences of an SAE, the patient is presented only once at the maximum relationship in the respective patient count. Events are counted each time in the event (E) column. Adverse events are coded using MedDRA version 14.1.
[1] There is eveidence to suggest a causal relationship between the drug and the SAE.
Chiltern • 29JUL2015:5:34 PM • I:\Synergy\33610\21_Stat\Programs\Tables\t_aeby.sas • ZScott

We claim:

1. A method for preventing or treating chronic idiopathic constipation comprising administering to a patient in need thereof, an effective dosage of a guanylate cyclase receptor agonist having the sequence of SEQ ID NO:1, wherein said agonist is a [4:12, 7:15] bicycle peptide.

2. The method of claim 1, wherein said dosage is 3.0 mg or 6.0 mg per day.


SEQUENCE LISTING (25 sequences)

SEQ ID NO: 1 - protein, 16 aa, Artificial Sequence
  Synthesized peptide
NDECELCVNVACTGCL

SEQ ID NO: 2 - protein, 5 aa, Artificial Sequence
  Synthesized peptide
QHNPR

SEQ ID NO: 3 - protein, 5 aa, Artificial Sequence
  Synthesized peptide
QHNPR

SEQ ID NO: 4 - protein, 7 aa, Artificial Sequence
  Synthesized peptide
VRQHNPR

SEQ ID NO: 5 - protein, 8 aa, Artificial Sequence
  Synthesized peptide
VRGQHNPR

SEQ ID NO: 6 - protein, 9 aa, Artificial Sequence
  Synthesized peptide
VRGPQHNPR

SEQ ID NO: 7 - protein, 10 aa, Artificial Sequence
  Synthesized peptide
VRGPRQHNPR

SEQ ID NO: 8 - protein, 11 aa, Artificial Sequence
  Synthesized peptide
VRGPRRQHNPR

SEQ ID NO: 9 - protein, 6 aa, Artificial Sequence
  Snthesized peptide
RQHNPR

SEQ ID NO: 10 - protein, 5 aa, Artificial Sequence
  Synthesized peptide
YXGFX

SEQ ID NO: 11 - protein, 4 aa, Artificial Sequence
  Frakefamide
YXFF

SEQ ID NO: 12 - protein, 34 aa, Artificial Sequence
  Synthesized peptide
IKPEAPGEDASPEELNRYYASLRHYLNLVTRQRY

SEQ ID NO: 13 - protein, 27 aa, Artificial Sequence
  Synthesized peptide
HAEGTFTSDVSYLEGQAAKEFIAWLVK

SEQ ID NO: 14 - protein, 6 aa, Artificial Sequence
  Synthesized peptide
KYYRWR

SEQ ID NO: 15 - protein, 6 aa, Artificial Sequence
  Synthesized peptide
RYYRWR

SEQ ID NO: 16 - protein, 6 aa, Artificial Sequence
  Synthesized peptide
KWRYYR

SEQ ID NO: 17 - protein, 6 aa, Artificial Sequence
  Synthesized peptide
RYYRWK

SEQ ID NO: 18 - protein, 6 aa, Artificial Sequence
  Synthesized peptide
XXXXXX

SEQ ID NO: 19 - protein, 6 aa, Artificial Sequence
  Synthesized peptide
RYYRIK

SEQ ID NO: 20 - protein, 6 aa, Artificial Sequence
  Synthesized peptide
RYYRIR

SEQ ID NO: 21 - protein, 6 aa, Artificial Sequence
  Synthesized peptide
RYYKIK

SEQ ID NO: 22 - protein, 6 aa, Artificial Sequence
  Synthesized peptide
RYYKIR

SEQ ID NO: 23 - protein, 6 aa, Artificial Sequence
  Synthesized peptide
RYYKWR

SEQ ID NO: 24 - protein, 6 aa, Artificial Sequence
  Synthesized peptide
RYYKWK

SEQ ID NO: 25 - protein, 6 aa, Artificial Sequence
  Synthesized peptide
KYYRWK